SEQUENCE LISTING (304 sequences)

SEQ ID NO: 1 - protein, 114 aa, synthetic construct
  REGION 1..114: note = CD8alpha Ig-like domain-1
SQFRVSPLDRTWNLGETVELKCQVLLSNPTSGCSWLFQPRGAAASPTFLLYLSQNKPKAAEGLDTQRFSGKRLGDTFVLTLSDFRRENEGYYFCSALSNSIMYFSHFVPVFLPA

SEQ ID NO: 2 - protein, 38 aa, synthetic construct
  REGION 1..38: note = CD8beta stalk region
SVVDFLPTTAQPTKKSTLKKRVCRLPRPETQKGPLCSP

SEQ ID NO: 3 - protein, 21 aa, synthetic construct
  REGION 1..21: note = CD8alpha transmembrane domain
IYIWAPLAGTCGVLLLSLVIT

SEQ ID NO: 4 - protein, 32 aa, synthetic construct
  REGION 1..32: note = CD8alpha cytoplasmic tail
LYCNHRNRRRVCKCPRPVVKSGDKPSLSARYV

SEQ ID NO: 5 - protein, 205 aa, synthetic construct
  REGION 1..205: note = m1CD8alpha (signal-less)
SQFRVSPLDRTWNLGETVELKCQVLLSNPTSGCSWLFQPRGAAASPTFLLYLSQNKPKAAEGLDTQRFSGKRLGDTFVLTLSDFRRENEGYYFCSALSNSIMYFSHFVPVFLPASVVDFLPTTAQPTKKSTLKKRVCRLPRPETQKGPLCSPIYIWAPLAGTCGVLLLSLVITLYCNHRNRRRVCKCPRPVVKSGDKPSLSARYV

SEQ ID NO: 6 - protein, 21 aa, synthetic construct
  REGION 1..21: note = Signal peptide
MALPVTALLLPLALLLHAARP

SEQ ID NO: 7 - protein, 226 aa, synthetic construct
  REGION 1..226: note = m1CD8alpha
MALPVTALLLPLALLLHAARPSQFRVSPLDRTWNLGETVELKCQVLLSNPTSGCSWLFQPRGAAASPTFLLYLSQNKPKAAEGLDTQRFSGKRLGDTFVLTLSDFRRENEGYYFCSALSNSIMYFSHFVPVFLPASVVDFLPTTAQPTKKSTLKKRVCRLPRPETQKGPLCSPIYIWAPLAGTCGVLLLSLVITLYCNHRNRRRVCKCPRPVVKSGDKPSLSARYV

SEQ ID NO: 8 - protein, 243 aa, Homo sapiens
MRPRLWLLLAAQLTVLHGNSVLQQTPAYIKVQTNKMVMLSCEAKISLSNMRIYWLRQRQAPSSDSHHEFLALWDSAKGTIHGEEVEQEKIAVFRDASRFILNLTSVKPEDSGIYFCMIVGSPELTFGKGTQLSVVDFLPTTAQPTKKSTLKKRVCRLPRPETQKGPLCSPITLGLLVAGVLVLLVSLGVAIHLCCRRRRARLRFMKQPQGEGISGTFVPQCLHGYYSNTTTSQKLLNPWILKT

SEQ ID NO: 9 - protein, 198 aa, Homo sapiens
MRPRLWLLLAAQLTVLHGNSVLQQTPAYIKVQTNKMVMLSCEAKISLSNMRIYWLRQRQAPSSDSHHEFLALWDSAKGTIHGEEVEQEKIAVFRDASRFILNLTSVKPEDSGIYFCMIVGSPELTFGKGTQLSVVDFLPTTAQPTKKSTLKKRVCRLPRPETQKGLKGKVYQEPLSPNACMDTTAILQPHRSCLTHGS

SEQ ID NO: 10 - protein, 189 aa, Homo sapiens
LQQTPAYIKVQTNKMVMLSCEAKISLSNMRIYWLRQRQAPSSDSHHEFLALWDSAKGTIHGEEVEQEKIAVFRDASRFILNLTSVKPEDSGIYFCMIVGSPELTFGKGTQLSVVDFLPTTAQPTKKSTLKKRVCRLPRPETQKGPLCSPITLGLLVAGVLVLLVSLGVAIHLCCRRRRARLRFMKQFYK

SEQ ID NO: 11 - protein, 200 aa, Homo sapiens
LQQTPAYIKVQTNKMVMLSCEAKISLSNMRIYWLRQRQAPSSDSHHEFLALWDSAKGTIHGEEVEQEKIAVFRDASRFILNLTSVKPEDSGIYFCMIVGSPELTFGKGTQLSVVDFLPTTAQPTKKSTLKKRVCRLPRPETQKGPLCSPITLGLLVAGVLVLLVSLGVAIHLCCRRRRARLRFMKQLRLHPLEKCSRMDY

SEQ ID NO: 12 - protein, 225 aa, Homo sapiens
LQQTPAYIKVQTNKMVMLSCEAKISLSNMRIYWLRQRQAPSSDSHHEFLALWDSAKGTIHGEEVEQEKIAVFRDASRFILNLTSVKPEDSGIYFCMIVGSPELTFGKGTQLSVVDFLPTTAQPTKKSTLKKRVCRLPRPETQKGPLCSPITLGLLVAGVLVLLVSLGVAIHLCCRRRRARLRFMKQKFNIVCLKISGFTTCCCFQILQISREYGFGVLLQKDIGQ

SEQ ID NO: 13 - protein, 225 aa, Homo sapiens; identical to SEQ ID NO: 12

SEQ ID NO: 14 - protein, 222 aa, Homo sapiens
LQQTPAYIKVQTNKMVMLSCEAKISLSNMRIYWLRQRQAPSSDSHHEFLALWDSAKGTIHGEEVEQEKIAVFRDASRFILNLTSVKPEDSGIYFCMIVGSPELTFGKGTQLSVVDFLPTTAQPTKKSTLKKRVCRLPRPETQKGPLCSPITLGLLVAGVLVLLVSLGVAIHLCCRRRRARLRFMKQPQGEGISGTFVPQCLHGYYSNTTTSQKLLNPWILKT

SEQ ID NO: 15 - protein, 273 aa, synthetic construct
  REGION 1..273: note = R11KEA alpha chain
MEKNPLAAPLLILWFHLDCVSSILNVEQSPQSLHVQEGDSTNFTCSFPSSNFYALHWYRKETAKSPEALFVMTLNGDEKKKGRISATLNTKEGYSYLYIKGSQPEDSATYLCALYNNNDMRFGAGTRLTVKPNIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 16 - protein, 311 aa, synthetic construct
  REGION 1..311: note = R11KE beta chain
MDSWTFCCVSLCILVAKHTDAGVIQSPRHEVTEMGQEVTLRCKPISGHNSLFWYRETMMRGLELLIYFNNNVPIDDSGMPEDRFSAKMPNASFSTLKIQPSEPRDSAVYFCASSPGSTDTQYFGPGTRLTVLEDLKNVFPPEVAVFEPSEAEISHTQKATLVCLATGFYPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSESYQQGVLSATILYEILLGKATLYAVLVSALVLMAMVKRKDSRG

SEQ ID NO: 17 - protein, 274 aa, synthetic construct
  REGION 1..274: note = R20P1H7 alpha chain
MEKMLECAFIVLWLQLGWLSGEDQVTQSPEALRLQEGESSSLNCSYTVSGLRGLFWYRQDPGKGPEFLFTLYSAGEEKEKERLKATLTKKESFLHITAPKPEDSATYLCAVQGENSGYSTLTFGKGTMLLVSPDIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 18 - protein, 314 aa, synthetic construct
  REGION 1..314: note = R20P1H7 beta chain
MGPQLLGYVVLCLLGAGPLEAQVTQNPRYLITVTGKKLTVTCSQNMNHEYMSWYRQDPGLGLRQIYYSMNVEVTDKGDVPEGYKVSRKEKRNFPLILESPSPNQTSLYFCASSLGPGLAAYNEQFFGPGTRLTVLEDLKNVFPPEVAVFEPSEAEISHTQKATLVCLATGFYPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSESYQQGVLSATILYEILLGKATLYAVLVSALVLMAMVKRKDSRG

SEQ ID NO: 19 - protein, 272 aa, synthetic construct
  REGION 1..272: note = R7P1D5 alpha chain
MKTFAGFSFLFLWLQLDCMSRGEDVEQSLFLSVREGDSSVINCTYTDSSSTYLYWYKQEPGAGLQLLTYIFSNMDMKQDQRLTVLLNKKDKHLSLRIADTQTGDSAIYFCAEYSSASKIIFGSGTRLSIRPNIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 20 - protein, 310 aa, synthetic construct
  REGION 1..310: note = R7P1D5 beta chain
MGSWTLCCVSLCILVAKHTDAGVIQSPRHEVTEMGQEVTLRCKPISGHDYLFWYRQTMMRGLELLIYFNNNVPIDDSGMPEDRFSAKMPNASFSTLKIQPSEPRDSAVYFCASRANTGELFFGEGSRLTVLEDLKNVFPPEVAVFEPSEAEISHTQKATLVCLATGFYPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSESYQQGVLSATILYEILLGKATLYAVLVSALVLMAMVKRKDSRG

SEQ ID NO: 21 - protein, 277 aa, synthetic construct
  REGION 1..277: note = R10P2G12 alpha chain
MLTASLLRAVIASICVVSSMAQKVTQAQTEISVVEKEDVTLDCVYETRDTTYYLFWYKQPPSGELVFLIRRNSFDEQNEISGRYSWNFQKSTSSFNFTITASQVVDSAVYFCALSEGNSGNTPLVFGKGTRLSVIANIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 22 - protein, 313 aa, synthetic construct
  REGION 1..313: note = R10P2G12 beta chain
MGIRLLCRVAFCFLAVGLVDVKVTQSSRYLVKRTGEKVFLECVQDMDHENMFWYRQDPGLGLRLIYFSYDVKMKEKGDIPEGYSVSREKKERFSLILESASTNQTSMYLCASSLSSGSHQETQYFGPGTRLLVLEDLKNVFPPEVAVFEPSEAEISHTQKATLVCLATGFYPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSESYQQGVLSATILYEILLGKATLYAVLVSALVLMAMVKRKDSRG

SEQ ID NO: 23 - protein, 271 aa, synthetic construct
  REGION 1..271: note = R10P1A7 alpha chain
MKTFAGFSFLFLWLQLDCMSRGEDVEQSLFLSVREGDSSVINCTYTDSSSTYLYWYKQEPGAGLQLLTYIFSNMDMKQDQRLTVLLNKKDKHLSLRIADTQTGDSAIYFCAESKETRLMFGDGTQLVVKPNIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 24 - protein, 317 aa, synthetic construct
  REGION 1..317: note = R10P1A7 beta chain
MLLLLLLLGPGISLLLPGSLAGSGLGAWSQHPSVWICKSGTSVKIECRSLDFQATTMFWYRQFPKQSLMLMATSNEGSKATYEQGVEKDKFLINHASLTLSTLTVTSAHPEDSSFYICSARAGGHEQFFGPGTRLTVLEDLKNVFPPEVAVFEPSEAEISHTQKATLVCLATGFYPDHVELSWVWNGKEVHSGVSTDPQPLKEQPALNDSRYCLSSRLRVSATFWQNPRNHFRCQVQFYGLSENDEWTQDRAKPVTQIVSAEAWGRADCGFTSESYQQGVLSATILYEILLGKATLYAVLVSALVLMAMVKRKDSRG

SEQ ID NO: 25 - protein, 271 aa, synthetic construct
  REGION 1..271: note = R4P1D10 alpha chain
MKSLRVLLVILWLQLSWVWSQQKEVEQNSGPLSVPEGAIASLNCTYSDRGSQSFFWYRQYSGKSPELIMFIYSNGDKEDGRFTAQLNKASQYVSLLIRDSQPSDSATYLCAVNFHDKIIFGKGTRLHILPNIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 26 - protein, 308 aa, synthetic construct
  REGION 1..308: note = R4P1D10 beta chain
MGFRLLCCVAFCLLGAGPVDSGVTQTPKHLITATGQRVTLRCSPRSGDLSVYWYQQSLDQGLQFLIHYYNGEERAKGNILERFSAQQFPDLHSELNLSSLELGDSALYFCASSVASAYGYTFGSGTRLTVVEDLNKVFPPEVAVFEPSEAEISHTQKATLVCLATGFFPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSVSYQQGVLSATILYEILLGKATLYAVLVSALVLMAMVKRKDF

SEQ ID NO: 27 - protein, 274 aa, synthetic construct
  REGION 1..274: note = R4P3F9 alpha chain
MKSLRVLLVILWLQLSWVWSQQKEVEQNSGPLSVPEGAIASLNCTYSDRGSQSFFWYRQYSGKSPELIMFIYSNGDKEDGRFTAQLNKASQYVSLLIRDSQPSDSATYLCAAYSGAGSYQLTFGKGTKLSVIPNIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 28 - protein, 308 aa, synthetic construct
  REGION 1..308: note = R4P3F9 beta chain
MGFRLLCCVAFCLLGAGPVDSGVTQTPKHLITATGQRVTLRCSPRSGDLSVYWYQQSLDQGLQFLIQYYNGEERAKGNILERFSAQQFPDLHSELNLSSLELGDSALYFCASSVESSYGYTFGSGTRLTVVEDLNKVFPPEVAVFEPSEAEISHTQKATLVCLATGFFPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSVSYQQGVLSATILYEILLGKATLYAVLVSALVLMAMVKRKDF

SEQ ID NO: 29 - protein, 271 aa, synthetic construct
  REGION 1..271: note = R4P3H3 alpha chain
MKSLRVLLVILWLQLSWVWSQQKEVEQNSGPLSVPEGAIASLNCTYSDRGSQSFFWYRQYSGKSPELIMFIYSNGDKEDGRFTAQLNKASQYVSLLIRDSQPSDSATYLCAVKAGNQFYFGTGTSLTVIPNIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 30 - protein, 314 aa, synthetic construct
  REGION 1..314: note = R4P3H3 beta chain
MGTRLLCWVVLGFLGTDHTGAGVSQSPRYKVAKRGQDVALRCDPISGHVSLFWYQQALGQGPEFLTYFQNEAQLDKSGLPSDRFFAERPEGSVSTLKIQRTQQEDSAVYLCASSLLTSGGDNEQFFGPGTRLTVLEDLKNVFPPEVAVFEPSEAEISHTQKATLVCLATGFYPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSESYQQGVLSATILYEILLGKATLYAVLVSALVLMAMVKRKDSRG

SEQ ID NO: 31 - protein, 270 aa, synthetic construct
  REGION 1..270: note = R36P3F9 alpha chain
METLLGVSLVILWLQLARVNSQQGEEDPQALSIQEGENATMNCSYKTSINNLQWYRQNSGRGLVHLILIRSNEREKHSGRLRVTLDTSKKSSSLLITASRAADTASYFCATVSNYQLIWGAGTKLIIKPDIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 32 - protein, 314 aa, synthetic construct
  REGION 1..314: note = R36P3F9 beta chain
MGPQLLGYVVLCLLGAGPLEAQVTQNPRYLITVTGKKLTVTCSQNMNHEYMSWYRQDPGLGLRQIYYSMNVEVTDKGDVPEGYKVSRKEKRNFPLILESPSPNQTSLYFCASSSTSGGLSGETQYFGPGTRLLVLEDLKNVFPPEVAVFEPSEAEISHTQKATLVCLATGFYPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSESYQQGVLSATILYEILLGKATLYAVLVSALVLMAMVKRKDSRG

SEQ ID NO: 33 - protein, 272 aa, synthetic construct
  REGION 1..272: note = R52P2G11 alpha chain
MKKHLTTFLVILWLYFYRGNGKNQVEQSPQSLIILEGKNCTLQCNYTVSPFSNLRWYKQDTGRGPVSLTIMTFSENTKSNGRYTATLDADTKQSSLHITASQLSDSASYICVVSAYGKLQFGAGTQVVVTPDIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 34 - protein, 312 aa, synthetic construct
  REGION 1..312: note = R52P2G11 beta chain
MDSWTFCCVSLCILVAKHTDAGVIQSPRHEVTEMGQEVTLRCKPISGHNSLFWYRQTMMRGLELLIYFNNNVPIDDSGMPEDRFSAKMPNASFSTLKIQPSEPRDSAVYFCASSLGSPDGNQPQHFGDGTRLSILEDLNKVFPPEVAVFEPSEAEISHTQKATLVCLATGFFPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSVSYQQGVLSATILYEILLGKATLYAVLVSALVLMAMVKRKDF

SEQ ID NO: 35 - protein, 279 aa, synthetic construct
  REGION 1..279: note = R53P2A9 alpha chain
MACPGFLWALVISTCLEFSMAQTVTQSQPEMSVQEAETVTLSCTYDTSESDYYLFWYKQPPSRQMILVIRQEAYKQQNATENRFSVNFQKAAKSFSLKISDSQLGDAAMYFCAYNSYAGGTSYGKLTFGQGTILTVHPNIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 36 - protein, 310 aa, synthetic construct
  REGION 1..310: note = R53P2A9 beta chain
MGPGLLCWVLLCLLGAGPVDAGVTQSPTHLIKTRGQQVTLRCSPISGHKSVSWYQQVLGQGPQFIFQYYEKEERGRGNFPDRFSARQFPNYSSELNVNALLLGDSALYLCASSLDGTSEQYFGPGTRLTVTEDLKNVFPPEVAVFEPSEAEISHTQKATLVCLATGFYPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSESYQQGVLSATILYEILLGKATLYAVLVSALVLMAMVKRKDSRG

SEQ ID NO: 37 - protein, 271 aa, synthetic construct
  REGION 1..271: note = R26P1A9 alpha chain
METLLGVSLVILWLQLARVNSQQGEEDPQALSIQEGENATMNCSYKTSINNLQWYRQNSGRGLVHLILIRSNEREKHSGRLRVTLDTSKKSSSLLITASRAADTASYFCLIGASGSRLTFGEGTQLTVNPDIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 38 - protein, 310 aa, synthetic construct
  REGION 1..310: note = R26P1A9 beta chain
MGSWTLCCVSLCILVAKHTDAGVIQSPRHEVTEMGQEVTLRCKPISGHDYLFWYRQTMMRGLELLIYFNNNVPIDDSGMPEDRFSAKMPNASFSTLKIQPSEPRDSAVYFCASSYFGWNEKLFFGSGTQLSVLEDLNKVFPPEVAVFEPSEAEISHTQKATLVCLATGFFPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSVSYQQGVLSATILYEILLGKATLYAVLVSALVLMAMVKRKDF

SEQ ID NO: 39 - protein, 276 aa, synthetic construct
  REGION 1..276: note = R26P2A6 alpha chain
MMKSLRVLLVILWLQLSWVWSQQKEVEQDPGPLSVPEGAIVSLNCTYSNSAFQYFMWYRQYSRKGPELLMYTYSSGNKEDGRFTAQVDKSSKYISLFIRDSQPSDSATYLCAMSDVSGGYNKLIFGAGTRLAVHPYIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 40 - protein, 311 aa, synthetic construct
  REGION 1..311: note = R26P2A6 beta chain
MGPQLLGYVVLCLLGAGPLEAQVTQNPRYLITVTGKKLTVTCSQNMNHEYMSWYRQDPGLGLRQIYYSMNVEVTDKGDVPEGYKVSRKEKRNFPLILESPSPNQTSLYFCASTTPDGTDEQFFGPGTRLTVLEDLKNVFPPEVAVFEPSEAEISHTQKATLVCLATGFYPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSESYQQGVLSATILYEILLGKATLYAVLVSALVLMAMVKRKDSRG

SEQ ID NO: 41 - protein, 274 aa, synthetic construct
  REGION 1..274: note = R26P3H1 alpha chain
MASAPISMLAMLFTLSGLRAQSVAQPEDQVNVAEGNPLTVKCTYSVSGNPYLFWYVQYPNRGLQFLLKYITGDNLVKGSYGFEAEFNKSQTSFHLKKPSALVSDSALYFCAVRDMNRDDKIIFGKGTRLHILPNIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 42 - protein, 310 aa, synthetic construct
  REGION 1..310: note = R26P3H1 beta chain
MSNQVLCCVVLCFLGANTVDGGITQSPKYLFRKEGQNVTLSCEQNLNHDAMYWYRQDPGQGLRLIYYSQIVNDFQKGDIAEGYSVSREKKESFPLTVTSAQKNPTAFYLCASSRAEGGEQYFGPGTRLTVTEDLKNVFPPEVAVFEPSEAEISHTQKATLVCLATGFYPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSESYQQGVLSATILYEILLGKATLYAVLVSALVLMAMVKRKDSRG

SEQ ID NO: 43 - protein, 273 aa, synthetic construct
  REGION 1..273: note = R35P3A4 alpha chain
MTSIRAVFIFLWLQLDLVNGENVEQHPSTLSVQEGDSAVIKCTYSDSASNYFPWYKQELGKRPQLIIDIRSNVGEKKDQRIAVTLNKTAKHFSLHITETQPEDSAVYFCAASPTGGYNKLIFGAGTRLAVHPYIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 44 - protein, 311 aa, synthetic construct
  REGION 1..311: note = R35P3A4 beta chain
MSIGLLCCAALSLLWAGPVNAGVTQTPKFQVLKTGQSMTLQCAQDMNHEYMSWYRQDPGMGLRLIHYSVGAGITDQGEVPNGYNVSRSTTEDFPLRLLSAAPSQTSVYFCASSLGGASQEQYFGPGTRLTVTEDLKNVFPPEVAVFEPSEAEISHTQKATLVCLATGFYPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSESYQQGVLSATILYEILLGKATLYAVLVSALVLMAMVKRKDSRG

SEQ ID NO: 45 - protein, 266 aa, synthetic construct
  REGION 1..266: note = R37P1C9 alpha chain
MKLVTSITVLLSLGIMGDAKTTQPNSMESNEEEPVHLPCNHSTISGTDYIHWYRQLPSQGPEYVIHGLTSNVNNRMASLAIAEDRKSSTLILHRATLRDAAVYYCILFNFNKFYFGSGTKLNVKPNIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 46 - protein, 309 aa, synthetic construct
  REGION 1..309: note = R37P1C9 beta chain
MGPGLLHWMALCLLGTGHGDAMVIQNPRYQVTQFGKPVTLSCSQTLNHNVMYWYQQKSSQAPKLLFHYYDKDFNNEADTPDNFQSRRPNTSFCFLDIRSPGLGDAAMYLCATSSGETNEKLFFGSGTQLSVLEDLNKVFPPEVAVFEPSEAEISHTQKATLVCLATGFFPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSVSYQQGVLSATILYEILLGKATLYAVLVSALVLMAMVKRKDF

SEQ ID NO: 47 - protein, 277 aa, synthetic construct
  REGION 1..277: note = R37P1H1 alpha chain
MTRVSLLWAVVVSTCLESGMAQTVTQSQPEMSVQEAETVTLSCTYDTSESNYYLFWYKQPPSRQMILVIRQEAYKQQNATENRFSVNFQKAAKSFSLKISDSQLGDTAMYFCAFGYSGGGADGLTFGKGTHLIIQPYIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 48 - protein, 311 aa, synthetic construct
  REGION 1..311: note = R37P1H1 beta chain
MGPGLLCWALLCLLGAGLVDAGVTQSPTHLIKTRGQQVTLRCSPKSGHDTVSWYQQALGQGPQFIFQYYEEEERQRGNFPDRFSGHQFPNYSSELNVNALLLGDSALYLCASSNEGQGWEAEAFFGQGTRLTVVEDLNKVFPPEVAVFEPSEAEISHTQKATLVCLATGFFPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSVSYQQGVLSATILYEILLGKATLYAVLVSALVLMAMVKRKDF

SEQ ID NO: 49 - protein, 268 aa, synthetic construct
  REGION 1..268: note = R42P3A9 alpha chain
MKRILGALLGLLSAQVCCVRGIQVEQSPPDLILQEGANSTLRCNFSDSVNNLQWFHQNPWGQLINLFYIPSGTKQNGRLSATTVATERYSLLYISSSQTTDSGVYFCAVHNFNKFYFGSGTKLNVKPNIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 50 - protein, 322 aa, synthetic construct
  REGION 1..322: note = R42P3A9 beta chain
MLSPDLPDSAWNTRLLCHVMLCLLGAVSVAAGVIQSPRHLIKEKRETATLKCYPIPRHDTVYWYQQGPGQDPQFLISFYEKMQSDKGSIPDRFSAQQFSDYHSELNMSSLELGDSALYFCASSLLGQGYNEQFFGPGTRLTVLEDLKNVFPPEVAVFEPSEAEISHTQKATLVCLATGFYPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSESYQQGVLSATILYEILLGKATLYAVLVSALVLMAMVKRKDSRG

SEQ ID NO: 51 - protein, 276 aa, synthetic construct
  REGION 1..276: note = R43P3F2 alpha chain
MLTASLLRAVIASICVVSSMAQKVTQAQTEISVVEKEDVTLDCVYETRDTTYYLFWYKQPPSGELVFLIRRNSFDEQNEISGRYSWNFQKSTSSFNFTITASQVVDSAVYFCALSNNNAGNMLTFGGGTRLMVKPHIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 52 - protein, 323 aa, synthetic construct
  REGION 1..323: note = R43P3F2 beta chain
MLSPDLPDSAWNTRLLCHVMLCLLGAVSVAAGVIQSPRHLIKEKRETATLKCYPIPRHDTVYWYQQGPGQDPQFLISFYEKMQSDKGSIPDRFSAQQFSDYHSELNMSSLELGDSALYFCASSPTGTSGYNEQFFGPGTRLTVLEDLKNVFPPEVAVFEPSEAEISHTQKATLVCLATGFYPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSESYQQGVLSATILYEILLGKATLYAVLVSALVLMAMVKRKDSRG

SEQ ID NO: 53 - protein, 273 aa, synthetic construct
  REGION 1..273: note = R43P3G5 alpha chain
MEKNPLAAPLLILWFHLDCVSSILNVEQSPQSLHVQEGDSTNFTCSFPSSNFYALHWYRWETAKSPEALFVMTLNGDEKKKGRISATLNTKEGYSYLYIKGSQPEDSATYLCALNRDDKIIFGKGTRLHILPNIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 54 - protein, 311 aa, synthetic construct
  REGION 1..311: note = R43P3G5 beta chain
MGIRLLCRVAFCFLAVGLVDVKVTQSSRYLVKRTGEKVFLECVQDMDHENMFWYRQDPGLGLRLIYFSYDVKMKEKGDIPEGYSVSREKKERFSLILESASTNQTSMYLCASRLPSRTYEQYFGPGTRLTVTEDLKNVFPPEVAVFEPSEAEISHTQKATLVCLATGFYPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSESYQQGVLSATILYEILLGKATLYAVLVSALVLMAMVKRKDSRG

SEQ ID NO: 55 - protein, 270 aa, synthetic construct
  REGION 1..270: note = R59P2E7 alpha chain
METLLGLLILWLQLQWVSSKQEVTQIPAALSVPEGENLVLNCSFTDSAIYNLQWFRQDPGKGLTSLLLIQSSQREQTSGRLNASLDKSSGRSTLYIAASQPGDSATYLCAVNSDYKLSFGAGTTVTVRANIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 56 - protein, 321 aa, synthetic construct
  REGION 1..321: note = R59P2E7 beta chain
MLSPDLPDSAWNTRLLCHVMLCLLGAVSVAAGVIQSPRHLIKEKRETATLKCYPIPRHDTVYWYQQGPGQDPQFLISFYEKMQSDKGSIPDRFSAQQFSDYHSELNMSSLELGDSALYFCASSLGLGTGDYGYTFGSGTRLTVVEDLNKVFPPEVAVFEPSEAEISHTQKATLVCLATGFFPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSVSYQQGVLSATILYEILLGKATLYAVLVSALVLMAMVKRKDF

SEQ ID NO: 57 - protein, 273 aa, synthetic construct
  REGION 1..273: note = R11P3D3 alpha chain
MEKNPLAAPLLILWFHLDCVSSILNVEQSPQSLHVQEGDSTNFTCSFPSSNFYALHWYRWETAKSPEALFVMTLNGDEKKKGRISATLNTKEGYSYLYIKGSQPEDSATYLCALYNNNDMRFGAGTRLTVKPNIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 58 - protein, 311 aa, synthetic construct
  REGION 1..311: note = R11P3D3 beta chain
MDSWTFCCVSLCILVAKHTDAGVIQSPRHEVTEMGQEVTLRCKPISGHNSLFWYRQTMMRGLELLIYFNNNVPIDDSGMPEDRFSAKMPNASFSTLKIQPSEPRDSAVYFCASSPGSTDTQYFGPGTRLTVLEDLKNVFPPEVAVFEPSEAEISHTQKATLVCLATGFYPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSESYQQGVLSATLLYEILLGKATLYAVLVSALVLMAMVKRKDSRG

SEQ ID NO: 59 - protein, 275 aa, synthetic construct
  REGION 1..275: note = R16P1C10 alpha chain
MKSLRVLLVILWLQLSWVWSQQKEVEQNSGPLSVPEGAIASLNCTYSDRGSQSFFWYRQYSGKSPELIMFIYSNGDKEDGRFTAQLNKASQYVSLLIRDSQPSDSATYLCAAVISNFGNEKLTFGTGTRLTIIPNIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 60 - protein, 311 aa, synthetic construct
  REGION 1..311: note = R16P1C10 beta chain
MGSRLLCWVLLCLLGAGPVKAGVTQTPRYLIKTRGQQVTLSCSPISGHRSVSWYQQTPGQGLQFLFEYFSETQRNKGNFPGRFSGRQFSNSRSEMNVSTLELGDSALYLCASSPWDSPNEQYFGPGTRLTVTEDLKNVFPPEVAVFEPSEAEISHTQKATLVCLATGFYPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSESYQQGVLSATILYEILLGKATLYAVLVSALVLMAMVKRKDSRG

SEQ ID NO: 61 - protein, 274 aa, synthetic construct
  REGION 1..274: note = R16P1E8 alpha chain
MMKSLRVLLVILWLQLSWVWSQQKEVEQDPGPLSVPEGAIVSLNCTYSNSAFQYFMWYRQYSRKGPELLMYTYSSGNKEDGRFTAQVDKSSKYISLFIRDSQPSDSATYLCAMSEAAGNKLTFGGGTRVLVKPNIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 62 - protein, 309 aa, synthetic construct
  REGION 1..309: note = R16P1E8 beta chain
MGTRLLCWAALCLLGAELTEAGVAQSPRYKIIEKRQSVAFWCNPISGHATLYWYQQILGQGPKLLIQFQNNGVVDDSQLPKDRFSAERLKGVDSTLKIQPAKLEDSAVYLCASSYTNQGEAFFGQGTRLTVVEDLNKVFPPEVAVFEPSEAEISHTQKATLVCLATGFFPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSVSYQQGVLSATILYEILLGKATLYAVLVSALVLMAMVKRKDF

SEQ ID NO: 63 - protein, 273 aa, synthetic construct
  REGION 1..273: note = R17P1A9 alpha chain
MKSLRVLLVILWLQLSWVWSQQKEVEQNSGPLSVPEGAIASLNCTYSDRGSQSFFWYRQYSGKSPELIMSIYSNGDKEDGRFTAQLNKASQYVSLLIRDSQPSDSATYLCAVLNQAGTALIFGKGTTLSVSSNIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 64 - protein, 314 aa, synthetic construct
  REGION 1..314: note = R17P1A9 beta chain
MGFRLLCCVAFCLLGAGPVDSGVTQTPKHLITATGQRVTLRCSPRSGDLSVYWYQQSLDQGLQFLIQYYNGEERAKGNILERFSAQQFPDLHSELNLSSLELGDSALYFCASSAETGPWLGNEQFFGPGTRLTVLEDLKNVFPPEVAVFEPSEAEISHTQKATLVCLATGFYPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSESYQQGVLSATILYEILLGKATLYAVLVSALVLMAMVKRKDSRG

SEQ ID NO: 65 - protein, 277 aa, synthetic construct
  REGION 1..277: note = R17P1D7 alpha chain
MACPGFLWALVISTCLEFSMAQTVTQSQPEMSVQEAETVTLSCTYDTSESDYYLFWYKQPPSRQMILVIRQEAYKQQNATENRFSVNFQKAAKSFSLKISDSQLGDAAMYFCAYRWAQGGSEKLVFGKGTKLTVNPYIQKPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 66 - protein, 313 aa, synthetic construct
  REGION 1..313: note = R17P1D7 beta chain
MTIRLLCYMGFYFLGAGLMEADIYQTPRYLVIGTGKKITLECSQTMGHDKMYWYQQDPGMELHLIHYSYGVNSTEKGDLSSESTVSRIRTEHFPLTLESARPSHTSQYLCATELWSSGGTGELFFGEGSRLTVLEDLKNVFPPEVAVFEPSEAEISHTQKATLVCLATGFYPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSESYQQGVLSATILYEILLGKATLYAVLVSALVLMAMVKRKDSRG

SEQ ID NO: 67 - protein, 273 aa, synthetic construct
  REGION 1..273: note = R17P1G3 alpha chain
MKSLRVLLVILWLQLSWVWSQQKEVEQNSGPLSVPEGAIASLNCTYSDRGSQSFFWYRQYSGKSPELIMSIYSNGDKEDGRFTAQLNKASQYVSLLIRDSQPSDSATYLCAVGPSGTYKYIFGTGTRLKVLANIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 68 - protein, 311 aa, synthetic construct
  REGION 1..311: note = R17P1G3 beta chain
MGPQLLGYVVLCLLGAGPLEAQVTQNPRYLITVTGKKLTVTCSQNMNHEYMSWYRQDPGLGLRQIYYSMNVEVTDKGDVPEGYKVSRKEKRNFPLILESPSPNQTSLYFCASSPGGSGNEQFFGPGTRLTVLEDLKNVFPPEVAVFEPSEAEISHTQKATLVCLATGFYPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSESYQQGVLSATILYEILLGKATLYAVLVSALVLMAMVKRKDSRG

SEQ ID NO: 69 - protein, 274 aa, synthetic construct
  REGION 1..274: note = R17P2B6 alpha chain
MKSLRVLLVILWLQLSWVWSQQKEVEQNSGPLSVPEGAIASLNCTYSDRGSQSFFWYRQYSGKSPELIMFIYSNGDKEDGRFTAQLNKASQYVSLLIRDSQPSDSATYLCAVVSGGGADGLTFGKGTHLIIQPYIQKPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 70 - protein, 319 aa, synthetic construct
  REGION 1..319: note = R17P2B6 beta chain
MLSPDLPDSAWNTRLLCHVMLCLLGAVSVAAGVIQSPRHLIKEKRETATLKCYPIPRHDTVYWYQQGPGQDPQFLISFYEKMQSDKGSIPDRFSAQQFSDYHSELNMSSLELGDSALYFCASSLGRGGQPQHFGDGTRLSILEDLNKVFPPEVAVFEPSEAEISHTQKATLVCLATGFFPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSVSYQQGVLSATILYEILLGKATLYAVLVSALVLMAMVKRKDF

SEQ ID NO: 71 - protein, 273 aa, synthetic construct; identical to SEQ ID NO: 15
  REGION 1..273: note = R11P3D3KE alpha chain

SEQ ID NO: 72 - protein, 243 aa, synthetic construct
  REGION 1..243: note = R11P3D3KE beta chain
NNNVPIDDSGMPEDRFSAKMPNASFSTLKIQPSEPRDSAVYFCASSPGSTDTQYFGPGTRLTVLEDLKNVFPPEVAVFEPSEAEISHTQKATLVCLATGFYPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSESYQQGVLSATLLYEILLGKATLYAVLVSALVLMAMVKRKDSRG

SEQ ID NO: 73 - protein, 223 aa, synthetic construct
  REGION 1..223: note = R39P1C12 alpha chain
TYLYWYKQEPGAGLQLLTYIFSNMDMKQDQRLTVLLNKKDKHLSLRIADTQTGDSAIYFCAEIDNQGGKLIFGQGTELSVKPNIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 74 - protein, 307 aa, synthetic construct
  REGION 1..307: note = R39P1C12 beta chain
MGPGLLCWALLCLLGAGLVDAGVTQSPTHLIKTRGQQVTLRCSPKSGHDTVSWYQQALGQGPQFIFQYYEEEERQRGNFPDRFSGHQFPNYSSELNVNALLLGDSALYLCASSQLNTEAFFGQGTRLTVVEDLNKVFPPEVAVFEPSEAEISHTQKATLVCLATGFFPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSVSYQQGVLSATILYEILLGKATLYAVLVSALVLMAMVKRKDF

SEQ ID NO: 75 - protein, 270 aa, synthetic construct
  REGION 1..270: note = R39P1F5 alpha chain
MKSLRVLLVILWLQLSWVWSQQKEVEQNSGPLSVPEGAIASLNCTYSDRGSQSFFWYRQYSGKSPELIMFIYSNGDKEDGRFTAQLNKASQYVSLLIRDSQPSDSATYLCAVNNARLMFGDGTQLVVKPNIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 76 - protein, 311 aa, synthetic construct
  REGION 1..311: note = R39P1F5 beta chain
MDTWLVCWAIFSLLKAGLTEPEVTQTPSHQVTQMGQEVILRCVPISNHLYFYWYRQILGQKVEFLVSFYNNEISEKSEIFDDQFSVERPDGSNFTLKIRSTKLEDSAMYFCASSGQGANEQYFGPGTRLTVTEDLKNVFPPEVAVFEPSEAEISHTQKATLVCLATGFYPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSESYQQGVLSATILYEILLGKATLYAVLVSALVLMAMVKRKDSRG

SEQ ID NO: 77 - protein, 272 aa, synthetic construct
  REGION 1..272: note = R40P1C2 alpha chain
MACPGFLWALVISTCLEFSMAQTVTQSQPEMSVQEAETVTLSCTYDTSESDYYLFWYKQPPSRQMILVIRQEAYKQQNATENRFSVNFQKAAKSFSLKISDSQLGDAAMYFCAYLNYQLIWGAGTKLIIKPDIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 78 - protein, 311 aa, synthetic construct
  REGION 1..311: note = R40P1C2 beta chain
MDTWLVCWAIFSLLKAGLTEPEVTQTPSHQVTQMGQEVILRCVPISNHLYFYWYRQILGQKVEFLVSFYNNEISEKSEIFDDQFSVERPDGSNFTLKIRSTKLEDSAMYFCASSEMTAVGQYFGPGTRLTVTEDLKNVFPPEVAVFEPSEAEISHTQKATLVCLATGFYPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSESYQQGVLSATILYEILLGKATLYAVLVSALVLMAMVKRKDSRG

SEQ ID NO: 79 - protein, 271 aa, synthetic construct
  REGION 1..271: note = R41P3E6 alpha chain
MKSLRVLLVILWLQLSWVWSQQKEVEQNSGPLSVPEGAIASLNCTYSDRGSQSFFWYRQYSGKSPELIMFIYSNGDKEDGRFTAQLNKASQYVSLLIRDSQPSDSATYLCAAFSGYALNFGKGTSLLVTPHIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 80 - protein, 310 aa, synthetic construct
  REGION 1..310: note = R41P3E6 beta chain
MDTWLVCWAIFSLLKAGLTEPEVTQTPSHQVTQMGQEVILRCVPISNHLYFYWYRQILGQKVEFLVSFYNNEISEKSEIFDDQFSVERPDGSNFTLKIRSTKLEDSAMYFCASSQYTGELFFGEGSRLTVLEDLKNVFPPEVAVFEPSEAEISHTQKATLVCLATGFYPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSESYQQGVLSATILYEILLGKATLYAVLVSALVLMAMVKRKDSRG

SEQ ID NO: 81 - protein, 270 aa, synthetic construct
  REGION 1..270: note = R43P3G4 alpha chain
MKSLRVLLVILWLQLSWVWSQQKEVEQNSGPLSVPEGAIASLNCTYSDRGSQSFFWYRQYSGKSPELIMFIYSNGDKEDGRFTAQLNKASQYVSLLIRDSQPSDSATYLCAVNGGDMRFGAGTRLTVKPNIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 82 - protein, 311 aa, synthetic construct
  REGION 1..311: note = R43P3G4 beta chain
MDTWLVCWAIFSLLKAGLTEPEVTQTPSHQVTQMGQEVILRCVPISNHLYFYWYRQILGQKVEFLVSFYNNEISEKSEIFDDQFSVERPDGSNFTLKIRSTKLEDSAMYFCASSGQGALEQYFGPGTRLTVTEDLKNVFPPEVAVFEPSEAEISHTQKATLVCLATGFYPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSESYQQGVLSATILYEILLGKATLYAVLVSALVLMAMVKRKDSRG

SEQ ID NO: 83 - protein, 281 aa, synthetic construct
  REGION 1..281: note = R44P3B3 alpha chain
MAMLLGASVLILWLQPDWVNSQQKNDDQQVKQNSPSLSVQEGRISILNCDYTNSMFDYFLWYKKYPAEGPTFLISISSIKDKNEDGRFTVFLNKSAKHLSLHIVPSQPGDSAVYFCAASGLYNQGGKLIFGQGTELSVKPNIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 84 - protein, 311 aa, synthetic construct
  REGION 1..311: note = R44P3B3 beta chain
MGCRLLCCVVFCLLQAGPLDTAVSQTPKYLVTQMGNDKSIKCEQNLGHDTMYWYKQDSKKFLKIMFSYNNKELIINETVPNRFSPKSPDKAHLNLHINSLELGDSAVYFCASSLGDRGYEQYFGPGTRLTVTEDLKNVFPPEVAVFEPSEAEISHTQKATLVCLATGFYPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSESYQQGVLSATILYEILLGKATLYAVLVSALVLMAMVKRKDSRG

SEQ ID NO: 85 - protein, 272 aa, synthetic construct
  REGION 1..272: note = R44P3E7 alpha chain
MKTFAGFSFLFLWLQLDCMSRGEDVEQSLFLSVREGDSSVINCTYTDSSSTYLYWYKQEPGAGLQLLTYIFSNMDMKQDQRLTVLLNKKDKHLSLRIADTQTGDSAIYFCAEINNNARLMFGDGTQLVVKPNIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 86 - protein, 320 aa, synthetic construct
  REGION 1..320: note = R44P3E7 beta chain
MLSPDLPDSAWNTRLLCHVMLCLLGAVSVAAGVIQSPRHLIKEKRETATLKCYPIPRHDTVYWYQQGPGQDPQFLISFYEKMQSDKGSIPDRFSAQQFSDYHSELNMSSLELGDSALYFCASSPPDQNTQYFGPGTRLTVLEDLKNVFPPEVAVFEPSEAEISHTQKATLVCLATGFYPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSESYQQGVLSATILYEILLGKATLYAVLVSALVLMAMVKRKDSRG

SEQ ID NO: 87 - protein, 273 aa, synthetic construct
  REGION 1..273: note = R49P2B7 alpha chain
MLLLLVPVLEVIFTLGGTRAQSVTQLGSHVSVSEGALVLLRCNYSSSVPPYLFWYVQYPNQGLQLLLKYTTGATLVKGINGFEAEFKKSETSFHLTKPSAHMSDAAEYFCAVRIFGNEKLTFGTGTRLTIIPNIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 88 - protein, 312 aa, synthetic construct
  REGION 1..312: note = R49P2B7 beta chain
MGIRLLCRVAFCFLAVGLVDVKVTQSSRYLVKRTGEKVFLECVQDMDHENMFWYRQDPGLGLRLIYFSYDVKMKEKGDIPEGYSVSREKKERFSLILESASTNQTSMYLCASSLMGELTGELFFGEGSRLTVLEDLKNVFPPEVAVFEPSEAEISHTQKATLVCLATGFYPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSESYQQGVLSATILYEILLGKATLYAVLVSALVLMAMVKRKDSRG

SEQ ID NO: 89 - protein, 276 aa, synthetic construct
  REGION 1..276: note = R55P1G7 alpha chain
MMKSLRVLLVILWLQLSWVWSQQKEVEQDPGPLSVPEGAIVSLNCTYSNSAFQYFMWYRQYSRKGPELLMYTYSSGNKEDGRFTAQVDKSSKYISLFIRDSQPSDSATYLCAMMGDTGTASKLTFGTGTRLQVTLDIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 90 - protein, 309 aa, synthetic construct
  REGION 1..309: note = R55P1G7 beta chain
MGIRLLCRVAFCFLAVGLVDVKVTQSSRYLVKRTGEKVFLECVQDMDHENMFWYRQDPGLGLRLIYFSYDVKMKEKGDIPEGYSVSREKKERFSLILESASTNQTSMYLCASSFGGYEQYFGPGTRLTVTEDLKNVFPPEVAVFEPSEAEISHTQKATLVCLATGFYPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSESYQQGVLSATILYEILLGKATLYAVLVSALVLMAMVKRKDSRG

SEQ ID NO: 91 - protein, 270 aa, synthetic construct
  REGION 1..270: note = R59P2A7 alpha chain
MKSLRVLLVILWLQLSWVWSQQKEVEQNSGPLSVPEGAIASLNCTYSDRGSQSFFWYRQYSGKSPELIMSIYSNGDKEDGRFTAQLNKASQYVSLLIRDSQPSDSATYLCAVQPHDMRFGAGTRLTVKPNIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 92 - protein, 307 aa, synthetic construct
  REGION 1..307: note = R59P2A7 beta chain
MLCSLLALLLGTFFGVRSQTIHQWPATLVQPVGSPLSLECTVEGTSNPNLYWYRQAAGRGLQLLFYSVGIGQISSEVPQNLSASRPQDRQFILSSKKLLLSDSGFYLCAWSGLVAEQFFGPGTRLTVLEDLKNVFPPEVAVFEPSEAEISHTQKATLVCLATGFYPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSESYQQGVLSATILYEILLGKATLYAVLVSALVLMAMVKRKDSRG

SEQ ID NO: 93 - protein, 19 aa, synthetic construct
  REGION 1..19: note = P2A
ATNFSLLKQAGDVEENPGP

SEQ ID NO: 94 - protein, 18 aa, synthetic construct
  REGION 1..18: note = T2A
EGRGSLLTCGDVEENPGP

SEQ ID NO: 95 - protein, 20 aa, synthetic construct
  REGION 1..20: note = E2A
QCTNYALLKLAGDVESNPGP

SEQ ID NO: 96 - protein, 22 aa, synthetic construct
  REGION 1..22: note = F2A
VKQTLNFDLLKLAGDVESNPGP

SEQ ID NO: 97 - protein, 559 aa, synthetic construct
  REGION 1..559: note = RD114TR
MKLPTGMVILCSLIIVRAGFDDPRKAIALVQKQHGKPCECSGGQVSEAPPNSIQQVTCPGKTAYLMTNQKWKCRVTPKISPSGGELQNCPCNTFQDSMHSSCYTEYRQCRRINKTYYTATLLKIRSGSLNEVQILQNPNQLLQSPCRGSINQPVCWSATAPIHISDGGGPLDTKRVWTVQKRLEQIHKAMTPELQYHPLALPKVRDDLSLDARTFDILNTTFRLLQMSNFSLAQDCWLCLKLGTPTPLAIPTPSLTYSLADSLANASCQIIPPLLVQPMQFSNSSCLSSPFINDTEQIDLGAVTFTNCTSVANVSSPLCALNGSVFLCGNNMAYTYLPQNWTRLCVQASLLPDIDINPGDEPVPIPAIDHYIHRPKRAVQFIPLLAGLGITAAFTTGATGLGVSVTQYTKLSHQLISDVQVLSGTIQDLQDQVDSLAEVVLQNRRGLDLLTAEQGGICLALQEKCCFYANKSGIVRNKIRTLQEELQKRRESLASNPLWTGLQGFLPYLLPLLGPLLTLLLILTIGPCVFNRLVQFVKDRISVVQALVLTQQYHQLKPL

SEQ ID NO: 98 - protein, 10 aa, Homo sapiens
YLYDSETKNA

SEQ ID NO: 99 - protein, 9 aa, Homo sapiens
HLMDQPLSV

SEQ ID NO: 100 - protein, 9 aa, Homo sapiens
GLLKKINSV

SEQ ID NO: 101 - protein, 9 aa, Homo sapiens
FLVDGSSAL

SEQ ID NO: 102 - protein, 10 aa, Homo sapiens
FLFDGSANLV

SEQ ID NO: 103 - protein, 9 aa, Homo sapiens
FLYKIIDEL

SEQ ID NO: 104 - protein, 11 aa, Homo sapiens
FILDSAETTTL

SEQ ID NO: 105 - protein, 9 aa, Homo sapiens
SVDVSPPKV

SEQ ID NO: 106 - protein, 8 aa, Homo sapiens
VADKIHSV

SEQ ID NO: 107 - protein, 9 aa, Homo sapiens
IVDDLTINL

SEQ ID NO: 108 - protein, 9 aa, Homo sapiens
GLLEELVTV

SEQ ID NO: 109 - protein, 10 aa, Homo sapiens
TLDGAAVNQV

SEQ ID NO: 110 - protein, 10 aa, Homo sapiens
SVLEKEIYSI

SEQ ID NO: 111 - protein, 9 aa, Homo sapiens
LLDPKTIFL

SEQ ID NO: 112 - protein, 9 aa, Homo sapiens
YTFSGDVQL

SEQ ID NO: 113 - protein, 9 aa, Homo sapiens
YLMDDFSSL

SEQ ID NO: 114 - protein, 9 aa, Homo sapiens
KVWSDVTPL

SEQ ID NO: 115 - protein, 11 aa, Homo sapiens
LLWGHPRVALA

SEQ ID NO: 116 - protein, 11 aa, Homo sapiens
KIWEELSVLEV

SEQ ID NO: 117 - protein, 9 aa, Homo sapiens
LLIPFTIFM

SEQ ID NO: 118 - protein, 9 aa, Homo sapiens
FLIENLLAA

SEQ ID NO: 119 - protein, 11 aa, Homo sapiens
LLWGHPRVALA

SEQ ID NO: 120 - protein, 9 aa, Homo sapiens
FLLEREQLL

SEQ ID NO: 121 - protein, 9 aa, Homo sapiens
SLAETIFIV

SEQ ID NO: 122 - protein, 9 aa, Homo sapiens
TLLEGISRA

SEQ ID NO: 123 - protein, 9 aa, Homo sapiens
KIQEILTQV

SEQ ID NO: 124 - protein, 10 aa, Homo sapiens
VIFEGEPMYL

SEQ ID NO: 125 - protein, 9 aa, Homo sapiens
SLFESLEYL

SEQ ID NO: 126 - protein, 9 aa, Homo sapiens
SLLNQPKAV

SEQ ID NO: 127 - protein, 9 aa, Homo sapiens
GLAEFQENV

SEQ ID NO: 128 - protein, 9 aa, Homo sapiens
KLLAVIHEL

SEQ ID NO: 129 - protein, 9 aa, Homo sapiens
TLHDQVHLL

SEQ ID NO: 130 - protein, 11 aa, Homo sapiens
TLYNPERTITV

SEQ ID NO: 131 - protein, 9 aa, Homo sapiens
KLQEKIQEL

SEQ ID NO: 132 - protein, 10 aa, Homo sapiens
SVLEKEIYSI

SEQ ID NO: 133 - protein, 11 aa, Homo sapiens
RVIDDSLVVGV

SEQ ID NO: 134 - protein, 9 aa, Homo sapiens
VLFGELPAL

SEQ ID NO: 135 - protein, 9 aa, Homo sapiens
GLVDIMVHL

SEQ ID NO: 136 - protein, 9 aa, Homo sapiens
FLNAIETAL

SEQ ID NO: 137 - protein, 9 aa, Homo sapiens
ALLQALMEL

SEQ ID NO: 138 - protein, 9 aa, Homo sapiens
ALSSSQAEV

SEQ ID NO: 139 - protein, 11 aa, Homo sapiens
SLITGQDLLSV

SEQ ID NO: 140 - protein, 9 aa, Homo sapiens
QLIEKNWLL

SEQ ID NO: 141 - protein, 9 aa, Homo sapiens
LLDPKTIFL

SEQ ID NO: 142 - protein, 9 aa, Homo sapiens
RLHDENILL

SEQ ID NO: 143 - protein, 9 aa, Homo sapiens
YTFSGDVQL

SEQ ID NO: 144 - protein, 9 aa, Homo sapiens
GLPSATTTV

SEQ ID NO: 145 - protein, 11 aa, Homo sapiens
GLLPSAESIKL

SEQ ID NO: 146 - protein, 9 aa, Homo sapiens
KTASINQNV

SEQ ID NO: 147 - protein, 9 aa, Homo sapiens
SLLQHLIGL

SEQ ID NO: 148 - protein, 9 aa, Homo sapiens
YLMDDFSSL

SEQ ID NO: 149 - protein, 9 aa, Homo sapiens
LMYPYIYHV

SEQ ID NO: 150 - protein, 9 aa, Homo sapiens
KVWSDVTPL

SEQ ID NO: 151 - protein, 11 aa, Homo sapiens
LLWGHPRVALA

SEQ ID NO: 152 - protein, 9 aa, Homo sapiens
VLDGKVAVV

SEQ ID NO: 153 - protein, 9 aa, Homo sapiens
GLLGKVTSV

SEQ ID NO: 154 - protein, 9 aa, Homo sapiens
KMISAIPTL

SEQ ID NO: 155 - protein, 11 aa, Homo sapiens
GLLETTGLLAT

SEQ ID NO: 156 - protein, 9 aa, Homo sapiens
TLNTLDINL

SEQ ID NO: 157 - protein, 9 aa, Homo sapiens
VIIKGLEEI

SEQ ID NO: 158 - protein, 9 aa, Homo sapiens
YLEDGFAYV

SEQ ID NO: 159 - protein, 11 aa, Homo sapiens
KIWEELSVLEV

SEQ ID NO: 160 - protein, 9 aa, Homo sapiens
LLIPFTIFM

SEQ ID NO: 161 - protein, 10 aa, Homo sapiens
ISLDEVAVSL

SEQ ID NO: 162 - protein, 10 aa, Homo sapiens
KISDFGLATV

SEQ ID NO: 163 - protein, 11 aa, Homo sapiens
KLIGNIHGNEV

SEQ ID NO: 164 - protein, 9 aa, Homo sapiens
ILLSVLHQL

SEQ ID NO: 165 - protein, 9 aa, Homo sapiens
LDSEALLTL

SEQ ID NO: 166 - protein, 13 aa, Homo sapiens
VLQENSSDYQSNL

SEQ ID NO: 167 - protein, 11 aa, Homo sapiens
HLLGEGAFAQV

SEQ ID NO: 168 - protein, 9 aa, Homo sapiens
SLVENIHVL

SEQ ID NO: 169 - protein, 9 aa, Homo sapiens
YTFSGDVQL

SEQ ID NO: 170 - protein, 9 aa, Homo sapiens
SLSEKSPEV

SEQ ID NO: 171 - protein, 10 aa, Homo sapiens
AMFPDTIPRV

SEQ ID NO: 172 - protein, 9 aa, Homo sapiens
FLIENLLAA

SEQ ID NO: 173 - protein, 9 aa, Homo sapiens
FTAEFLEKV

SEQ ID NO: 174 - protein, 9 aa, Homo sapiens
ALYGNVQQV

SEQ ID NO: 175 - protein, 9 aa, Homo sapiens
LFQSRIAGV

SEQ ID NO: 176 - protein, 11 aa, Homo sapiens
ILAEEPIYIRV

SEQ ID NO: 177 - protein, 9 aa, Homo sapiens
FLLEREQLL

SEQ ID NO: 178 - protein, 10 aa, Homo sapiens
LLLPLELSLA

SEQ ID NO: 179 - protein, 9 aa, Homo sapiens
SLAETIFIV

SEQ ID NO: 180 - protein, 11 aa, Homo sapiens
AILNVDEKNQV

SEQ ID NO: 181 - protein, 9 aa, Homo sapiens
RLFEEVLGV

SEQ ID NO: 182 - protein, 9 aa, Homo sapiens
YLDEVAFML

SEQ ID NO: 183 - protein, 11 aa, Homo sapiens
KLIDEDEPLFL

SEQ ID NO: 184 - protein, 9 aa, Homo sapiens
KLFEKSTGL

SEQ ID NO: 185 - protein, 11 aa, Homo sapiens
SLLEVNEASSV

SEQ ID NO: 186 - protein, 10 aa, Homo sapiens
GVYDGREHTV

SEQ ID NO: 187 - protein, 10 aa, Homo sapiens
GLYPVTLVGV

SEQ ID NO: 188 - protein, 9 aa, Homo sapiens
ALLSSVAEA

SEQ ID NO: 189 - protein, 9 aa, Homo sapiens
TLLEGISRA

SEQ ID NO: 190 - protein, 9 aa, Homo sapiens
SLIEESEEL

SEQ ID NO: 191 - protein, 9 aa, Homo sapiens
ALYVQAPTV

SEQ ID NO: 192 - protein, 10 aa, Homo sapiens
KLIYKDLVSV

SEQ ID NO: 193 - protein, 9 aa, Homo sapiens
ILQDGQFLV

SEQ ID NO: 194 - protein, 9 aa, Homo sapiens
SLLDYEVSI

SEQ ID NO: 195 - protein, 9 aa, Homo sapiens
LLGDSSFFL

SEQ ID NO: 196 - protein, 10 aa, Homo sapiens
VIFEGEPMYL

SEQ ID NO: 197 - protein, 9 aa, Homo sapiens
ALSYILPYL

SEQ ID NO: 198 - protein, 9 aa, Homo sapiens
FLFVDPELV

SEQ ID NO: 199 - protein, 11 aa, Homo sapiens
SEWGSPHAAVP

SEQ ID NO: 200 - protein, 9 aa, Homo sapiens
ALSELERVL

SEQ ID NO: 201 - protein, 9 aa, Homo sapiens
SLFESLEYL

SEQ ID NO: 202 - protein, 9 aa, Homo sapiens
KVLEYVIKV

SEQ ID NO: 203 - protein, 10 aa, Homo sapiens
VLLNEILEQV

SEQ ID NO: 204 - protein, 9 aa, Homo sapiens
SLLNQPKAV

SEQ ID NO: 205 - protein, 9 aa, Homo sapiens
KMSELQTYV

SEQ ID NO: 206 - protein, 11 aa, Homo sapiens
ALLEQTGDMSL

SEQ ID NO: 207 - protein, 11 aa, Homo sapiens
VIIKGLEEITV

SEQ ID NO: 208 - protein, 9 aa, Homo sapiens
KQFEGTVEI

SEQ ID NO: 209 - protein, 9 aa, Homo sapiens
KLQEEIPVL

SEQ ID NO: 210 - protein, 9 aa, Homo sapiens
GLAEFQENV

SEQ ID NO: 211 - protein, 9 aa, Homo sapiens
NVAEIVIHI

SEQ ID NO: 212 - protein, 9 aa, Homo sapiens
ALAGIVTNV

SEQ ID NO: 213 - protein, 12 aa, Homo sapiens
NLLIDDKGTIKL

SEQ ID NO: 214 - protein, 10 aa, Homo sapiens
VLMQDSRLYL

SEQ ID NO: 215 - protein, 9 aa, Homo sapiens
KVLEHVVRV

SEQ ID NO: 216 - protein, 9 aa, Homo sapiens
LLWGNLPEI

SEQ ID NO: 217 - protein, 9 aa, Homo sapiens
SLMEKNQSL

SEQ ID NO: 218 - protein, 9 aa, Homo sapiens
KLLAVIHEL

SEQ ID NO: 219 - protein, 10 aa, Homo sapiens
ALGDKFLLRV

SEQ ID NO: 220 - protein, 11 aa, Homo sapiens
FLMKNSDLYGA

SEQ ID NO: 221 - protein, 10 aa, Homo sapiens
KLIDHQGLYL

SEQ ID NO: 222 - protein, 12 aa, Homo sapiens
GPGIFPPPPPQP

SEQ ID NO: 223 - protein, 9 aa, Homo sapiens
ALNESLVEC

SEQ ID NO: 224 - protein, 9 aa, Homo sapiens
GLAALAVHL

SEQ ID NO: 225 - protein, 9 aa, Homo sapiens
LLLEAVWHL

SEQ ID NO: 226 - protein, 9 aa, Homo sapiens
SIIEYLPTL

SEQ ID NO: 227 - protein, 9 aa, Homo sapiens
TLHDQVHLL

SEQ ID NO: 228 - protein, 9 aa, Homo sapiens
SLLMWITQC

SEQ ID NO: 229 - protein, 11 aa, Homo sapiens
FLLDKPQDLSI

SEQ ID NO: 230 - protein, 10 aa, Homo sapiens
YLLDMPLWYL

SEQ ID NO: 231 - protein, 9 aa, Homo sapiens
GLLDCPIFL

SEQ ID NO: 232 - protein, 9 aa, Homo sapiens
VLIEYNFSI

SEQ ID NO: 233 - protein, 11 aa, Homo sapiens
TLYNPERTITV

SEQ ID NO: 234 - protein, 9 aa, Homo sapiens
AVPPPPSSV

SEQ ID NO: 235 - protein, 9 aa, Homo sapiens
KLQEELNKV

SEQ ID NO: 236 - protein, 11 aa, Homo sapiens
KLMDPGSLPPL

SEQ ID NO: 237 - protein, 9 aa, Homo sapiens
ALIVSLPYL

SEQ ID NO: 238 - protein, 9 aa, Homo sapiens
FLLDGSANV

SEQ ID NO: 239 - protein, 10 aa, Homo sapiens
ALDPSGNQLI

SEQ ID NO: 240 - protein, 9 aa, Homo sapiens
ILIKHLVKV

SEQ ID NO: 241 - protein, 9 aa, Homo sapiens
VLLDTILQL

SEQ ID NO: 242 - protein, 9 aa, Homo sapiens
HLIAEIHTA

SEQ ID NO: 243 - protein, 9 aa, Homo sapiens
SMNGGVFAV

SEQ ID NO: 244 - protein, 9 aa, Homo sapiens
MLAEKLLQA

SEQ ID NO: 245 - protein, 9 aa, Homo sapiens
YMLDIFHEV

SEQ ID NO: 246 - protein, 11 aa, Homo sapiens
ALWLPTDSATV

SEQ ID NO: 247 - protein, 9 aa, Homo sapiens
GLASRILDA

SEQ ID NO: 248 - protein, 9 aa, Homo sapiens
ALSVLRLAL

SEQ ID NO: 249 - protein, 9 aa, Homo sapiens
SYVKVLHHL

SEQ ID NO: 250 - protein, 9 aa, Homo sapiens
VYLPKIPSW

SEQ ID NO: 251 - protein, 9 aa, Homo sapiens
NYEDHFPLL

SEQ ID NO: 252 - protein, 9 aa, Homo sapiens
VYIAELEKI

SEQ ID NO: 253 - protein, 12 aa, Homo sapiens
VHFEDTGKTLLF

SEQ ID NO: 254 - protein, 9 aa, Homo sapiens
VLSPFILTL

SEQ ID NO: 255 - protein, 9 aa, Homo sapiens
HLLEGSVGV

SEQ ID NO: 256 - DNA, 607 bp, synthetic construct
  misc_feature 1..607: note = WPREmut1
  source 1..607: mol_type = other DNA
cagtctgacgtacgcgtaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcc

SEQ ID NO: 257 - DNA, 581 bp, synthetic construct
  misc_feature 1..581: note = WPREmut2
  source 1..581: mol_type = other DNA
gagcatcttaccgccatttatacccatatttgttctgtttttcttgatttgggtatacatttaaatgttaataaaacaaaatggtggggcaatcatttacattttttgggatatgtaattactagttcaggtgtattgccacaagacaaacttgttaagaaactttcccgttatttacgctctgttcctgttaatcaacctctggattacaaaatttgtgaaagattgactgatattcttaactttgttgctccttttacgctgtgtggatttgctgctttattgcctctgtatcttgctattgcttcccgtacggctttcgttttctcctccttgtataaatcctggttgctgtctctttttgaggagttgtggcccgttgtccgtcaacgtggcgtggtgtgctctgtgtttgctgacgcaacccccactggctggggcattgccaccacctgtcaactcctttctgggactttcgctttccccctcccgatcgccacggcagaactcatcgccgcctgccttgcccgctgctggacaggggctaggttgctgggcactgataattccgtggtgttgtc

SEQ ID NO: 258 - protein, 235 aa, Homo sapiens
MALPVTALLLPLALLLHAARPSQFRVSPLDRTWNLGETVELKCQVLLSNPTSGCSWLFQPRGAAASPTFLLYLSQNKPKAAEGLDTQRFSGKRLGDTFVLTLSDFRRENEGYYFCSALSNSIMYFSHFVPVFLPAKPTTTPAPRPPTPAPTIASQPLSLRPEACRPAAGGAVHTRGLDFACDIYIWAPLAGTCGVLLLSLVITLYCNHRNRRRVCKCPRPVVKSGDKPSLSARYV

SEQ ID NO: 259 - protein, 235 aa, Homo sapiens
MALPVTALLLPLALLLHAARPSQFRVSPLDRTWNLGETVELKCQVLLSNPTSGCSWLFQPRGAAASPTFLLYLSQNKPKAAEGLDTQRFSGKRLGDTFVLTLSDFRRENEGCYFCSALSNSIMYFSHFVPVFLPAKPTTTPAPRPPTPAPTIASQPLSLRPEACRPAAGGAVHTRGLDFACDIYIWAPLAGTCGVLLLSLVITLYCNHRNRRRVCKCPRPVVKSGDKPSLSARYV

SEQ ID NO: 260 - protein, 47 aa, synthetic construct
  REGION 1..47: note = CD8alpha stalk
KPTTTPAPRPPTPAPTIASQPLSLRPEACRPAAGGAVHTRGLDFACD

SEQ ID NO: 261 - protein, 114 aa, synthetic construct
  REGION 1..114: note = CD8alpha Ig-like domain-2
SQFRVSPLDRTWNLGETVELKCQVLLSNPTSGCSWLFQPRGAAASPTFLLYLSQNKPKAAEGLDTQRFSGKRLGDTFVLTLSDFRRENEGCYFCSALSNSIMYFSHFVPVFLPA

SEQ ID NO: 262 - protein, 226 aa, synthetic construct
  REGION 1..226: note = m2CD8alpha
MALPVTALLLPLALLLHAARPSQFRVSPLDRTWNLGETVELKCQVLLSNPTSGCSWLFQPRGAAASPTFLLYLSQNKPKAAEGLDTQRFSGKRLGDTFVLTLSDFRRENEGCYFCSALSNSIMYFSHFVPVFLPASVVDFLPTTAQPTKKSTLKKRVCRLPRPETQKGPLCSPIYIWAPLAGTCGVLLLSLVITLYCNHRNRRRVCKCPRPVVKSGDKPSLSARYV

SEQ ID NO: 263 - DNA, 367 bp, synthetic construct
  misc_feature 1..367: note = MSCV promoter
  source 1..367: mol_type = other DNA
tgaaagaccccacctgtaggtttggcaagctagcttaagtaacgccattttgcaaggcatggaaaatacataactgagaatagagaagttcagatcaaggttaggaacagagagacagcagaatatgggccaaacaggatatctgtggtaagcagttcctgccccggctcagggccaagaacagatggtccccagatgcggtcccgccctcagcagtttctagagaaccatcagatgtttccagggtgccccaaggacctgaaaatgaccctgtgccttatttgaactaaccaatcagttcgcttctcgcttctgttcgcgcgcttctgctccccgagctcaataaaagagcccacaacccctcact

SEQ ID NO: 264 - DNA, 607 bp, synthetic construct
  misc_feature 1..607: note = WPRE
  source 1..607: mol_type = other DNA
cagtctgacgtacgcgtaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccatggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcc

SEQ ID NO: 265 - skipped

SEQ ID NO: 266 - protein, 4 aa, synthetic construct
  REGION 1..4: note = Linker
SGSG

SEQ ID NO: 267 - DNA, 4320 bp, synthetic construct
  misc_feature 1..4320: note = CD8b3.CD8a.TCR.WPREmut2 construct nucleotide sequence
  source 1..4320: mol_type = other DNA
tgaaagaccccacctgtaggtttggcaagctagcttaagtaacgccattttgcaaggcatggaaaatacataactgagaatagagaagttcagatcaaggttaggaacagagagacagcagaatatgggccaaacaggatatctgtggtaagcagttcctgccccggctcagggccaagaacagatggtccccagatgcggtcccgccctcagcagtttctagagaaccatcagatgtttccagggtgccccaaggacctgaaaatgaccctgtgccttatttgaactaaccaatcagttcgcttctcgcttctgttcgcgcgcttctgctccccgagctcaataaaagagcccacaacccctcactcagcggccgccccgggtcgacgctaccaccatgcgcccgagactgtggcttctgctcgccgcgcaactgactgtcctgcacggaaacagcgtgctgcagcagacaccggcctacatcaaagtgcagaccaacaagatggtcatgctgtcctgcgaggccaagatttccctctccaacatgcggatctattggttgcggcagagacaggcgccttcctcggactcccaccatgagttcttggccctgtgggactccgccaagggaactattcacggcgaagaagtggaacaggagaagatcgccgtgtttcgcgatgcctcccgctttatactgaatctgacctccgtgaagcccgaagatagcgggatctacttttgcatgattgtgggctcacccgaactgaccttcgggaagggcactcagctgagcgtggtggacttcctccccactaccgcccaacccactaagaagtcaaccctgaagaagcgggtttgcagactcccacggccggaaacgcagaagggtccgctgtgttccccgatcaccctggggctccttgtggctggagtgctggtccttctggtgtcccttggcgtcgccattcacctctgctgccggagaaggagggccagactgaggttcatgaagcagttctacaagcgggccaagagatctggcagcggcgccaccaatttcagcctgctgaaacaggccggcgacgtggaagagaaccctggccccatggcgcttcccgtgaccgcactcctgttgccccttgccctgctgttgcacgccgcacgaccttcccaattccgggtgtcccctctggatcgcacctggaacctcggggaaacggtggagctcaagtgtcaagtcctcctgtcgaacccgaccagcggatgcagctggctgttccagccgagaggagctgccgcctcacccaccttcctcctgtacttgagccagaacaagccgaaggccgctgagggtctggacacccagcgcttctcgggcaaacggctgggagacacttttgtgctgactctctccgacttccggcgggagaacgagggctactacttctgctctgcgctctccaattcaatcatgtacttctcacacttcgtgccggtgttcctgcctgccaagcccaccactactccggcacccagacctccaactcccgctcccaccatcgcgtcccaacccctttcgctgcgccctgaagcgtgtcggcctgctgctggaggagccgtgcatacccgcggtctggacttcgcgtgcgacatctacatttgggcccctttggctggcacctgtggagtgctgctcctgtcccttgtgatcaccctgtactgcaaccaccggaataggcggagagtctgcaagtgtccgcggcctgtcgtgaagtcaggagataagccgagcctgtccgcacgctacgtgcgggccaagagatctggcagcggcgagggcagaggcagcctgctgacctgcggcgacgtggaggagaaccccggccccatggactcttggaccttctgctgcgtgagcctgtgcatcctggtggccaagcacacagacgccggcgtgatccagtcccctaggcacgaggtgaccgagatgggccaggaggtgacactgcgctgtaagccaatctctggccacaacagcctgttttggtatagggagaccatgatgcgcggcctggagctgctgatctacttcaataacaatgtgcccatcgacgattccggcatgcctgaggatcggttttctgccaagatgcccaatgccagcttctccacactgaagatccagcctagcgagccaagagactccgccgtgtatttttgcgcctctagcccaggcagcaccgatacacagtacttcggaccaggaaccaggctgacagtgctggaggacctgaagaacgtgttcccccctgaggtggccgtgtttgagccctctgaggccgagatcagccacacccagaaggccaccctggtgtgcctggcaaccggcttctatcctgatcacgtggagctgtcctggtgggtgaacggcaaggaggtgcacagcggcgtgtccacagacccacagcccctgaaggagcagccagccctgaatgatagccggtattgcctgtcctctcggctgagagtgtccgccaccttttggcagaacccccggaatcacttcagatgtcaggtgcagttttacggcctgtccgagaacgatgagtggacccaggaccgggccaagcctgtgacacagatcgtgtctgccgaggcatggggaagagcagactgtggcttcacctctgagagctaccagcagggcgtgctgagcgccaccatcctgtatgagatcctgctgggcaaggccacactgtacgccgtcctggtctccgctctggtgctgatggcaatggtcaaaagaaaagatagtcggggacgggccaagagatctggcagcggccagtgcaccaactacgccctgctgaagctggccggcgacgtggagagcaaccccggccccatggagaagaatcccctggctgcccccctgctgatcctgtggtttcacctggactgcgtgtcctctatcctgaatgtggaacagagcccacagagcctgcacgtgcaggagggcgactccaccaacttcacatgctcttttcctagctccaacttctacgccctgcactggtacagaaaggagaccgcaaagtccccagaggccctgttcgtgatgacactgaacggcgatgagaagaagaagggccgcatcagcgccaccctgaatacaaaggagggctactcctatctgtacatcaagggctcccagcctgaggactctgccacctatctgtgcgccctgtacaacaataacgatatgcggtttggcgccggcaccagactgacagtgaagccaaacatccagaatccagaccccgccgtgtatcagctgcgggacagcaagtctagcgataagagcgtgtgcctgttcaccgactttgattctcagacaaacgtgagccagtccaaggacagcgacgtgtacatcaccgacaagacagtgctggatatgagaagcatggacttcaagtctaacagcgccgtggcctggtccaataagtctgatttcgcctgcgccaatgcctttaataactccatcatccccgaggataccttctttccttctccagagtcctcttgtgacgtgaagctggtggagaagtctttcgagaccgatacaaacctgaattttcagaacctgagcgtgatcggcttcaggatcctgctgctgaaggtggccggctttaatctgctgatgaccctgaggctgtggagctcctgaaccggtccggagcatcttaccgccatttatacccatatttgttctgtttttcttgatttgggtatacatttaaatgttaataaaacaaaatggtggggcaatcatttacattttttgggatatgtaattactagttcaggtgtattgccacaagacaaacttgttaagaaactttcccgttatttacgctctgttcctgttaatcaacctctggattacaaaatttgtgaaagattgactgatattcttaactttgttgctccttttacgctgtgtggatttgctgctttattgcctctgtatcttgctattgcttcccgtacggctttcgttttctcctccttgtataaatcctggttgctgtctctttttgaggagttgtggcccgttgtccgtcaacgtggcgtggtgtgctctgtgtttgctgacgcaacccccactggctggggcattgccaccacctgtcaactcctttctgggactttcgctttccccctcccgatcgccacggcagaactcatcgccgcctgccttgcccgctgctggacaggggctaggttgctgggcactgataattccgtggtgttgtc

SEQ ID NO: 268 - protein, 1110 aa, synthetic construct
  REGION 1..1110: note = CD8b3.CD8a.TCR.WPREmut2 amino acid sequence
MRPRLWLLLAAQLTVLHGNSVLQQTPAYIKVQTNKMVMLSCEAKISLSNMRIYWLRQRQAPSSDSHHEFLALWDSAKGTIHGEEVEQEKIAVFRDASRFILNLTSVKPEDSGIYFCMIVGSPELTFGKGTQLSVVDFLPTTAQPTKKSTLKKRVCRLPRPETQKGPLCSPITLGLLVAGVLVLLVSLGVAIHLCCRRRRARLRFMKQFYKRAKRSGSGATNFSLLKQAGDVEENPGPMALPVTALLLPLALLLHAARPSQFRVSPLDRTWNLGETVELKCQVLLSNPTSGCSWLFQPRGAAASPTFLLYLSQNKPKAAEGLDTQRFSGKRLGDTFVLTLSDFRRENEGYYFCSALSNSIMYFSHFVPVFLPAKPTTTPAPRPPTPAPTIASQPLSLRPEACRPAAGGAVHTRGLDFACDIYIWAPLAGTCGVLLLSLVITLYCNHRNRRRVCKCPRPVVKSGDKPSLSARYVRAKRSGSGEGRGSLLTCGDVEENPGPMDSWTFCCVSLCILVAKHTDAGVIQSPRHEVTEMGQEVTLRCKPISGHNSLFWYRETMMRGLELLIYFNNNVPIDDSGMPEDRFSAKMPNASFSTLKIQPSEPRDSAVYFCASSPGSTDTQYFGPGTRLTVLEDLKNVFPPEVAVFEPSEAEISHTQKATLVCLATGFYPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSESYQQGVLSATILYEILLGKATLYAVLVSALVLMAMVKRKDSRGRAKRSGSGQCTNYALLKLAGDVESNPGPMEKNPLAAPLLILWFHLDCVSSILNVEQSPQSLHVQEGDSTNFTCSFPSSNFYALHWYRKETAKSPEALFVMTLNGDEKKKGRISATLNTKEGYSYLYIKGSQPEDSATYLCALYNNNDMRFGAGTRLTVKPNIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 269 - DNA, 4314 bp, synthetic construct
  misc_feature 1..4314: note = CD8b1(S19).CD8a.TCR.WPREmut2 construct nucleotide sequence
  source 1..4314: mol_type = other DNA
tgaaagaccccacctgtaggtttggcaagctagcttaagtaacgccattttgcaaggcatggaaaatacataactgagaatagagaagttcagatcaaggttaggaacagagagacagcagaatatgggccaaacaggatatctgtggtaagcagttcctgccccggctcagggccaagaacagatggtccccagatgcggtcccgccctcagcagtttctagagaaccatcagatgtttccagggtgccccaaggacctgaaaatgaccctgtgccttatttgaactaaccaatcagttcgcttctcgcttctgttcgcgcgcttctgctccccgagctcaataaaagagcccacaacccctcactcagcggccgccccgggtcgacgctaccaccatggaattcggcctgagctggctgttcctggtggccatcctgaagggcgtgcagtgcctgcagcagacaccggcctacatcaaagtgcagaccaacaagatggtcatgctgtcctgcgaggccaagatttccctctccaacatgcggatctattggttgcggcagagacaggcgccttcctcggactcccaccatgagttcttggccctgtgggactccgccaagggaactattcacggcgaagaagtggaacaggagaagatcgccgtgtttcgcgatgcctcccgctttatactgaatctgacctccgtgaagcccgaagatagcgggatctacttttgcatgattgtgggctcacccgaactgaccttcgggaagggcactcagctgagcgtggtggacttcctccccactaccgcccaacccactaagaagtcaaccctgaagaagcgggtttgcagactcccacggccggaaacgcagaagggtccgctgtgttccccgatcaccctggggctccttgtggctggagtgctggtccttctggtgtcccttggcgtcgccattcacctctgctgccggagaaggagggccagactgaggttcatgaagcagttctacaagcgggccaagagatctggcagcggcgccaccaatttcagcctgctgaaacaggccggcgacgtggaagagaaccctggccccatggcgcttcccgtgaccgcactcctgttgccccttgccctgctgttgcacgccgcacgaccttcccaattccgggtgtcccctctggatcgcacctggaacctcggggaaacggtggagctcaagtgtcaagtcctcctgtcgaacccgaccagcggatgcagctggctgttccagccgagaggagctgccgcctcacccaccttcctcctgtacttgagccagaacaagccgaaggccgctgagggtctggacacccagcgcttctcgggcaaacggctgggagacacttttgtgctgactctctccgacttccggcgggagaacgagggctactacttctgctctgcgctctccaattcaatcatgtacttctcacacttcgtgccggtgttcctgcctgccaagcccaccactactccggcacccagacctccaactcccgctcccaccatcgcgtcccaacccctttcgctgcgccctgaagcgtgtcggcctgctgctggaggagccgtgcatacccgcggtctggacttcgcgtgcgacatctacatttgggcccctttggctggcacctgtggagtgctgctcctgtcccttgtgatcaccctgtactgcaaccaccggaataggcggagagtctgcaagtgtccgcggcctgtcgtgaagtcaggagataagccgagcctgtccgcacgctacgtgcgggccaagagatctggcagcggcgagggcagaggcagcctgctgacctgcggcgacgtggaggagaaccccggccccatggactcttggaccttctgctgcgtgagcctgtgcatcctggtggccaagcacacagacgccggcgtgatccagtcccctaggcacgaggtgaccgagatgggccaggaggtgacactgcgctgtaagccaatctctggccacaacagcctgttttggtatagggagaccatgatgcgcggcctggagctgctgatctacttcaataacaatgtgcccatcgacgattccggcatgcctgaggatcggttttctgccaagatgcccaatgccagcttctccacactgaagatccagcctagcgagccaagagactccgccgtgtatttttgcgcctctagcccaggcagcaccgatacacagtacttcggaccaggaaccaggctgacagtgctggaggacctgaagaacgtgttcccccctgaggtggccgtgtttgagccctctgaggccgagatcagccacacccagaaggccaccctggtgtgcctggcaaccggcttctatcctgatcacgtggagctgtcctggtgggtgaacggcaaggaggtgcacagcggcgtgtccacagacccacagcccctgaaggagcagccagccctgaatgatagccggtattgcctgtcctctcggctgagagtgtccgccaccttttggcagaacccccggaatcacttcagatgtcaggtgcagttttacggcctgtccgagaacgatgagtggacccaggaccgggccaagcctgtgacacagatcgtgtctgccgaggcatggggaagagcagactgtggcttcacctctgagagctaccagcagggcgtgctgagcgccaccatcctgtatgagatcctgctgggcaaggccacactgtacgccgtcctggtctccgctctggtgctgatggcaatggtcaaaagaaaagatagtcggggacgggccaagagatctggcagcggccagtgcaccaactacgccctgctgaagctggccggcgacgtggagagcaaccccggccccatggagaagaatcccctggctgcccccctgctgatcctgtggtttcacctggactgcgtgtcctctatcctgaatgtggaacagagcccacagagcctgcacgtgcaggagggcgactccaccaacttcacatgctcttttcctagctccaacttctacgccctgcactggtacagaaaggagaccgcaaagtccccagaggccctgttcgtgatgacactgaacggcgatgagaagaagaagggccgcatcagcgccaccctgaatacaaaggagggctactcctatctgtacatcaagggctcccagcctgaggactctgccacctatctgtgcgccctgtacaacaataacgatatgcggtttggcgccggcaccagactgacagtgaagccaaacatccagaatccagaccccgccgtgtatcagctgcgggacagcaagtctagcgataagagcgtgtgcctgttcaccgactttgattctcagacaaacgtgagccagtccaaggacagcgacgtgtacatcaccgacaagacagtgctggatatgagaagcatggacttcaagtctaacagcgccgtggcctggtccaataagtctgatttcgcctgcgccaatgcctttaataactccatcatccccgaggataccttctttccttctccagagtcctcttgtgacgtgaagctggtggagaagtctttcgagaccgatacaaacctgaattttcagaacctgagcgtgatcggcttcaggatcctgctgctgaaggtggccggctttaatctgctgatgaccctgaggctgtggagctcctgaaccggtccggagcatcttaccgccatttatacccatatttgttctgtttttcttgatttgggtatacatttaaatgttaataaaacaaaatggtggggcaatcatttacattttttgggatatgtaattactagttcaggtgtattgccacaagacaaacttgttaagaaactttcccgttatttacgctctgttcctgttaatcaacctctggattacaaaatttgtgaaagattgactgatattcttaactttgttgctccttttacgctgtgtggatttgctgctttattgcctctgtatcttgctattgcttcccgtacggctttcgttttctcctccttgtataaatcctggttgctgtctctttttgaggagttgtggcccgttgtccgtcaacgtggcgtggtgtgctctgtgtttgctgacgcaacccccactggctggggcattgccaccacctgtcaactcctttctgggactttcgctttccccctcccgatcgccacggcagaactcatcgccgcctgccttgcccgctgctggacaggggctaggttgctgggcactgataattccgtggtgttgtc

SEQ ID NO: 270 - protein, 1108 aa, synthetic construct
  REGION 1..1108: note = CD8b1(S19).CD8a.TCR.WPREmut2 amino acid sequence
MEFGLSWLFLVAILKGVQCLQQTPAYIKVQTNKMVMLSCEAKISLSNMRIYWLRQRQAPSSDSHHEFLALWDSAKGTIHGEEVEQEKIAVFRDASRFILNLTSVKPEDSGIYFCMIVGSPELTFGKGTQLSVVDFLPTTAQPTKKSTLKKRVCRLPRPETQKGPLCSPITLGLLVAGVLVLLVSLGVAIHLCCRRRRARLRFMKQFYKRAKRSGSGATNFSLLKQAGDVEENPGPMALPVTALLLPLALLLHAARPSQFRVSPLDRTWNLGETVELKCQVLLSNPTSGCSWLFQPRGAAASPTFLLYLSQNKPKAAEGLDTQRFSGKRLGDTFVLTLSDFRRENEGYYFCSALSNSIMYFSHFVPVFLPAKPTTTPAPRPPTPAPTIASQPLSLRPEACRPAAGGAVHTRGLDFACDIYIWAPLAGTCGVLLLSLVITLYCNHRNRRRVCKCPRPVVKSGDKPSLSARYVRAKRSGSGEGRGSLLTCGDVEENPGPMDSWTFCCVSLCILVAKHTDAGVIQSPRHEVTEMGQEVTLRCKPISGHNSLFWYRETMMRGLELLIYFNNNVPIDDSGMPEDRFSAKMPNASFSTLKIQPSEPRDSAVYFCASSPGSTDTQYFGPGTRLTVLEDLKNVFPPEVAVFEPSEAEISHTQKATLVCLATGFYPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSESYQQGVLSATILYEILLGKATLYAVLVSALVLMAMVKRKDSRGRAKRSGSGQCTNYALLKLAGDVESNPGPMEKNPLAAPLLILWFHLDCVSSILNVEQSPQSLHVQEGDSTNFTCSFPSSNFYALHWYRKETAKSPEALFVMTLNGDEKKKGRISATLNTKEGYSYLYIKGSQPEDSATYLCALYNNNDMRFGAGTRLTVKPNIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 271 - DNA, 4428 bp, synthetic construct
  misc_feature 1..4428: note = CD8b4.CD8a.TCR.WPREmut2 construct nucleotide sequence
  source 1..4428: mol_type = other DNA
tgaaagaccccacctgtaggtttggcaagctagcttaagtaacgccattttgcaaggcatggaaaatacataactgagaatagagaagttcagatcaaggttaggaacagagagacagcagaatatgggccaaacaggatatctgtggtaagcagttcctgccccggctcagggccaagaacagatggtccccagatgcggtcccgccctcagcagtttctagagaaccatcagatgtttccagggtgccccaaggacctgaaaatgaccctgtgccttatttgaactaaccaatcagttcgcttctcgcttctgttcgcgcgcttctgctccccgagctcaataaaagagcccacaacccctcactcagcggccgccccgggtcgacgctaccaccatgcgcccgagactgtggcttctgctcgccgcgcaactgactgtcctgcacggaaacagcgtgctgcagcagacaccggcctacatcaaagtgcagaccaacaagatggtcatgctgtcctgcgaggccaagatttccctctccaacatgcggatctattggttgcggcagagacaggcgccttcctcggactcccaccatgagttcttggccctgtgggactccgccaagggaactattcacggcgaagaagtggaacaggagaagatcgccgtgtttcgcgatgcctcccgctttatactgaatctgacctccgtgaagcccgaagatagcgggatctacttttgcatgattgtgggctcacccgaactgaccttcgggaagggcactcagctgagcgtggtggacttcctccccactaccgcccaacccactaagaagtcaaccctgaagaagcgggtttgcagactcccacggccggaaacgcagaagggtccgctgtgttccccgatcaccctggggctccttgtggctggagtgctggtccttctggtgtcccttggcgtcgccattcacctctgctgccggagaaggagggccagactgaggttcatgaagcagaagttcaacatagtctgcctgaagatttccgggttcactacttgctgctgcttccaaatactccagatttcacgagaatatgggttcggtgtgttgctccaaaaagacatcggtcaacgggccaagagatctggcagcggcgccaccaatttcagcctgctgaaacaggccggcgacgtggaagagaaccctggccccatggcgcttcccgtgaccgcactcctgttgccccttgccctgctgttgcacgccgcacgaccttcccaattccgggtgtcccctctggatcgcacctggaacctcggggaaacggtggagctcaagtgtcaagtcctcctgtcgaacccgaccagcggatgcagctggctgttccagccgagaggagctgccgcctcacccaccttcctcctgtacttgagccagaacaagccgaaggccgctgagggtctggacacccagcgcttctcgggcaaacggctgggagacacttttgtgctgactctctccgacttccggcgggagaacgagggctactacttctgctctgcgctctccaattcaatcatgtacttctcacacttcgtgccggtgttcctgcctgccaagcccaccactactccggcacccagacctccaactcccgctcccaccatcgcgtcccaacccctttcgctgcgccctgaagcgtgtcggcctgctgctggaggagccgtgcatacccgcggtctggacttcgcgtgcgacatctacatttgggcccctttggctggcacctgtggagtgctgctcctgtcccttgtgatcaccctgtactgcaaccaccggaataggcggagagtctgcaagtgtccgcggcctgtcgtgaagtcaggagataagccgagcctgtccgcacgctacgtgcgggccaagagatctggcagcggcgagggcagaggcagcctgctgacctgcggcgacgtggaggagaaccccggccccatggactcttggaccttctgctgcgtgagcctgtgcatcctggtggccaagcacacagacgccggcgtgatccagtcccctaggcacgaggtgaccgagatgggccaggaggtgacactgcgctgtaagccaatctctggccacaacagcctgttttggtatagggagaccatgatgcgcggcctggagctgctgatctacttcaataacaatgtgcccatcgacgattccggcatgcctgaggatcggttttctgccaagatgcccaatgccagcttctccacactgaagatccagcctagcgagccaagagactccgccgtgtatttttgcgcctctagcccaggcagcaccgatacacagtacttcggaccaggaaccaggctgacagtgctggaggacctgaagaacgtgttcccccctgaggtggccgtgtttgagccctctgaggccgagatcagccacacccagaaggccaccctggtgtgcctggcaaccggcttctatcctgatcacgtggagctgtcctggtgggtgaacggcaaggaggtgcacagcggcgtgtccacagacccacagcccctgaaggagcagccagccctgaatgatagccggtattgcctgtcctctcggctgagagtgtccgccaccttttggcagaacccccggaatcacttcagatgtcaggtgcagttttacggcctgtccgagaacgatgagtggacccaggaccgggccaagcctgtgacacagatcgtgtctgccgaggcatggggaagagcagactgtggcttcacctctgagagctaccagcagggcgtgctgagcgccaccatcctgtatgagatcctgctgggcaaggccacactgtacgccgtcctggtctccgctctggtgctgatggcaatggtcaaaagaaaagatagtcggggacgggccaagagatctggcagcggccagtgcaccaactacgccctgctgaagctggccggcgacgtggagagcaaccccggccccatggagaagaatcccctggctgcccccctgctgatcctgtggtttcacctggactgcgtgtcctctatcctgaatgtggaacagagcccacagagcctgcacgtgcaggagggcgactccaccaacttcacatgctcttttcctagctccaacttctacgccctgcactggtacagaaaggagaccgcaaagtccccagaggccctgttcgtgatgacactgaacggcgatgagaagaagaagggccgcatcagcgccaccctgaatacaaaggagggctactcctatctgtacatcaagggctcccagcctgaggactctgccacctatctgtgcgccctgtacaacaataacgatatgcggtttggcgccggcaccagactgacagtgaagccaaacatccagaatccagaccccgccgtgtatcagctgcgggacagcaagtctagcgataagagcgtgtgcctgttcaccgactttgattctcagacaaacgtgagccagtccaaggacagcgacgtgtacatcaccgacaagacagtgctggatatgagaagcatggacttcaagtctaacagcgccgtggcctggtccaataagtctgatttcgcctgcgccaatgcctttaataactccatcatccccgaggataccttctttccttctccagagtcctcttgtgacgtgaagctggtggagaagtctttcgagaccgatacaaacctgaattttcagaacctgagcgtgatcggcttcaggatcctgctgctgaaggtggccggctttaatctgctgatgaccctgaggctgtggagctcctgaaccggtccggagcatcttaccgccatttatacccatatttgttctgtttttcttgatttgggtatacatttaaatgttaataaaacaaaatggtggggcaatcatttacattttttgggatatgtaattactagttcaggtgtattgccacaagacaaacttgttaagaaactttcccgttatttacgctctgttcctgttaatcaacctctggattacaaaatttgtgaaagattgactgatattcttaactttgttgctccttttacgctgtgtggatttgctgctttattgcctctgtatcttgctattgcttcccgtacggctttcgttttctcctccttgtataaatcctggttgctgtctctttttgaggagttgtggcccgttgtccgtcaacgtggcgtggtgtgctctgtgtttgctgacgcaacccccactggctggggcattgccaccacctgtcaactcctttctgggactttcgctttccccctcccgatcgccacggcagaactcatcgccgcctgccttgcccgctgctggacaggggctaggttgctgggcactgataattccgtggtgttgtc

SEQ ID NO: 272 - protein, 1146 aa, synthetic construct
  REGION 1..1146: note = CD8b4.CD8a.TCR.WPREmut2 amino acid sequence
MRPRLWLLLAAQLTVLHGNSVLQQTPAYIKVQTNKMVMLSCEAKISLSNMRIYWLRQRQAPSSDSHHEFLALWDSAKGTIHGEEVEQEKIAVFRDASRFILNLTSVKPEDSGIYFCMIVGSPELTFGKGTQLSVVDFLPTTAQPTKKSTLKKRVCRLPRPETQKGPLCSPITLGLLVAGVLVLLVSLGVAIHLCCRRRRARLRFMKQKFNIVCLKISGFTTCCCFQILQISREYGFGVLLQKDIGQRAKRSGSGATNFSLLKQAGDVEENPGPMALPVTALLLPLALLLHAARPSQFRVSPLDRTWNLGETVELKCQVLLSNPTSGCSWLFQPRGAAASPTFLLYLSQNKPKAAEGLDTQRFSGKRLGDTFVLTLSDFRRENEGYYFCSALSNSIMYFSHFVPVFLPAKPTTTPAPRPPTPAPTIASQPLSLRPEACRPAAGGAVHTRGLDFACDIYIWAPLAGTCGVLLLSLVITLYCNHRNRRRVCKCPRPVVKSGDKPSLSARYVRAKRSGSGEGRGSLLTCGDVEENPGPMDSWTFCCVSLCILVAKHTDAGVIQSPRHEVTEMGQEVTLRCKPISGHNSLFWYRETMMRGLELLIYFNNNVPIDDSGMPEDRFSAKMPNASFSTLKIQPSEPRDSAVYFCASSPGSTDTQYFGPGTRLTVLEDLKNVFPPEVAVFEPSEAEISHTQKATLVCLATGFYPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSESYQQGVLSATILYEILLGKATLYAVLVSALVLMAMVKRKDSRGRAKRSGSGQCTNYALLKLAGDVESNPGPMEKNPLAAPLLILWFHLDCVSSILNVEQSPQSLHVQEGDSTNFTCSFPSSNFYALHWYRKETAKSPEALFVMTLNGDEKKKGRISATLNTKEGYSYLYIKGSQPEDSATYLCALYNNNDMRFGAGTRLTVKPNIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 273 - DNA, 4308 bp, synthetic construct
  misc_feature 1..4308: note = CD8b1(S19).CD8a(S19).TCR.WPREmut2 construct nucleotide sequence
  source 1..4308: mol_type = other DNA
tgaaagaccccacctgtaggtttggcaagctagcttaagtaacgccattttgcaaggcatggaaaatacataactgagaatagagaagttcagatcaaggttaggaacagagagacagcagaatatgggccaaacaggatatctgtggtaagcagttcctgccccggctcagggccaagaacagatggtccccagatgcggtcccgccctcagcagtttctagagaaccatcagatgtttccagggtgccccaaggacctgaaaatgaccctgtgccttatttgaactaaccaatcagttcgcttctcgcttctgttcgcgcgcttctgctccccgagctcaataaaagagcccacaacccctcactcagcggccgccccgggtcgacgctaccaccatggaattcggcctgagctggctgttcctggtggccatcctgaagggcgtgcagtgcctgcagcagacaccggcctacatcaaagtgcagaccaacaagatggtcatgctgtcctgcgaggccaagatttccctctccaacatgcggatctattggttgcggcagagacaggcgccttcctcggactcccaccatgagttcttggccctgtgggactccgccaagggaactattcacggcgaagaagtggaacaggagaagatcgccgtgtttcgcgatgcctcccgctttatactgaatctgacctccgtgaagcccgaagatagcgggatctacttttgcatgattgtgggctcacccgaactgaccttcgggaagggcactcagctgagcgtggtggacttcctccccactaccgcccaacccactaagaagtcaaccctgaagaagcgggtttgcagactcccacggccggaaacgcagaagggtccgctgtgttccccgatcaccctggggctccttgtggctggagtgctggtccttctggtgtcccttggcgtcgccattcacctctgctgccggagaaggagggccagactgaggttcatgaagcagttctacaagcgggccaagagatctggcagcggcgccaccaatttcagcctgctgaaacaggccggcgacgtggaagagaaccctggccccatggagttcggcctgagctggctgttcctggtggccatcctgaagggcgtgcagtgctcccaattccgggtgtcccctctggatcgcacctggaacctcggggaaacggtggagctcaagtgtcaagtcctcctgtcgaacccgaccagcggatgcagctggctgttccagccgagaggagctgccgcctcacccaccttcctcctgtacttgagccagaacaagccgaaggccgctgagggtctggacacccagcgcttctcgggcaaacggctgggagacacttttgtgctgactctctccgacttccggcgggagaacgagggctactacttctgctctgcgctctccaattcaatcatgtacttctcacacttcgtgccggtgttcctgcctgccaagcccaccactactccggcacccagacctccaactcccgctcccaccatcgcgtcccaacccctttcgctgcgccctgaagcgtgtcggcctgctgctggaggagccgtgcatacccgcggtctggacttcgcgtgcgacatctacatttgggcccctttggctggcacctgtggagtgctgctcctgtcccttgtgatcaccctgtactgcaaccaccggaataggcggagagtctgcaagtgtccgcggcctgtcgtgaagtcaggagataagccgagcctgtccgcacgctacgtgcgggccaagagatctggcagcggcgagggcagaggcagcctgctgacctgcggcgacgtggaggagaaccccggccccatggactcttggaccttctgctgcgtgagcctgtgcatcctggtggccaagcacacagacgccggcgtgatccagtcccctaggcacgaggtgaccgagatgggccaggaggtgacactgcgctgtaagccaatctctggccacaacagcctgttttggtatagggagaccatgatgcgcggcctggagctgctgatctacttcaataacaatgtgcccatcgacgattccggcatgcctgaggatcggttttctgccaagatgcccaatgccagcttctccacactgaagatccagcctagcgagccaagagactccgccgtgtatttttgcgcctctagcccaggcagcaccgatacacagtacttcggaccaggaaccaggctgacagtgctggaggacctgaagaacgtgttcccccctgaggtggccgtgtttgagccctctgaggccgagatcagccacacccagaaggccaccctggtgtgcctggcaaccggcttctatcctgatcacgtggagctgtcctggtgggtgaacggcaaggaggtgcacagcggcgtgtccacagacccacagcccctgaaggagcagccagccctgaatgatagccggtattgcctgtcctctcggctgagagtgtccgccaccttttggcagaacccccggaatcacttcagatgtcaggtgcagttttacggcctgtccgagaacgatgagtggacccaggaccgggccaagcctgtgacacagatcgtgtctgccgaggcatggggaagagcagactgtggcttcacctctgagagctaccagcagggcgtgctgagcgccaccatcctgtatgagatcctgctgggcaaggccacactgtacgccgtcctggtctccgctctggtgctgatggcaatggtcaaaagaaaagatagtcggggacgggccaagagatctggcagcggccagtgcaccaactacgccctgctgaagctggccggcgacgtggagagcaaccccggccccatggagaagaatcccctggctgcccccctgctgatcctgtggtttcacctggactgcgtgtcctctatcctgaatgtggaacagagcccacagagcctgcacgtgcaggagggcgactccaccaacttcacatgctcttttcctagctccaacttctacgccctgcactggtacagaaaggagaccgcaaagtccccagaggccctgttcgtgatgacactgaacggcgatgagaagaagaagggccgcatcagcgccaccctgaatacaaaggagggctactcctatctgtacatcaagggctcccagcctgaggactctgccacctatctgtgcgccctgtacaacaataacgatatgcggtttggcgccggcaccagactgacagtgaagccaaacatccagaatccagaccccgccgtgtatcagctgcgggacagcaagtctagcgataagagcgtgtgcctgttcaccgactttgattctcagacaaacgtgagccagtccaaggacagcgacgtgtacatcaccgacaagacagtgctggatatgagaagcatggacttcaagtctaacagcgccgtggcctggtccaataagtctgatttcgcctgcgccaatgcctttaataactccatcatccccgaggataccttctttccttctccagagtcctcttgtgacgtgaagctggtggagaagtctttcgagaccgatacaaacctgaattttcagaacctgagcgtgatcggcttcaggatcctgctgctgaaggtggccggctttaatctgctgatgaccctgaggctgtggagctcctgaaccggtccggagcatcttaccgccatttatacccatatttgttctgtttttcttgatttgggtatacatttaaatgttaataaaacaaaatggtggggcaatcatttacattttttgggatatgtaattactagttcaggtgtattgccacaagacaaacttgttaagaaactttcccgttatttacgctctgttcctgttaatcaacctctggattacaaaatttgtgaaagattgactgatattcttaactttgttgctccttttacgctgtgtggatttgctgctttattgcctctgtatcttgctattgcttcccgtacggctttcgttttctcctccttgtataaatcctggttgctgtctctttttgaggagttgtggcccgttgtccgtcaacgtggcgtggtgtgctctgtgtttgctgacgcaacccccactggctggggcattgccaccacctgtcaactcctttctgggactttcgctttccccctcccgatcgccacggcagaactcatcgccgcctgccttgcccgctgctggacaggggctaggttgctgggcactgataattccgtggtgttgtc

SEQ ID NO: 274 - protein, 1106 aa, synthetic construct
  REGION 1..1106: note = CD8b1(S19).CD8a(S19).TCR.WPREmut2 amino acid sequence
MEFGLSWLFLVAILKGVQCLQQTPAYIKVQTNKMVMLSCEAKISLSNMRIYWLRQRQAPSSDSHHEFLALWDSAKGTIHGEEVEQEKIAVFRDASRFILNLTSVKPEDSGIYFCMIVGSPELTFGKGTQLSVVDFLPTTAQPTKKSTLKKRVCRLPRPETQKGPLCSPITLGLLVAGVLVLLVSLGVAIHLCCRRRRARLRFMKQFYKRAKRSGSGATNFSLLKQAGDVEENPGPMEFGLSWLFLVAILKGVQCSQFRVSPLDRTWNLGETVELKCQVLLSNPTSGCSWLFQPRGAAASPTFLLYLSQNKPKAAEGLDTQRFSGKRLGDTFVLTLSDFRRENEGYYFCSALSNSIMYFSHFVPVFLPAKPTTTPAPRPPTPAPTIASQPLSLRPEACRPAAGGAVHTRGLDFACDIYIWAPLAGTCGVLLLSLVITLYCNHRNRRRVCKCPRPVVKSGDKPSLSARYVRAKRSGSGEGRGSLLTCGDVEENPGPMDSWTFCCVSLCILVAKHTDAGVIQSPRHEVTEMGQEVTLRCKPISGHNSLFWYRETMMRGLELLIYFNNNVPIDDSGMPEDRFSAKMPNASFSTLKIQPSEPRDSAVYFCASSPGSTDTQYFGPGTRLTVLEDLKNVFPPEVAVFEPSEAEISHTQKATLVCLATGFYPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSESYQQGVLSATILYEILLGKATLYAVLVSALVLMAMVKRKDSRGRAKRSGSGQCTNYALLKLAGDVESNPGPMEKNPLAAPLLILWFHLDCVSSILNVEQSPQSLHVQEGDSTNFTCSFPSSNFYALHWYRKETAKSPEALFVMTLNGDEKKKGRISATLNTKEGYSYLYIKGSQPEDSATYLCALYNNNDMRFGAGTRLTVKPNIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 275 - DNA, 4416 bp, synthetic construct
  misc_feature 1..4416: note = CD8b4(S19).CD8a(S19).TCR.WPREmut2 construct nucleotide sequence
  source 1..4416: mol_type = other DNA
tgaaagaccccacctgtaggtttggcaagctagcttaagtaacgccattttgcaaggcatggaaaatacataactgagaatagagaagttcagatcaaggttaggaacagagagacagcagaatatgggccaaacaggatatctgtggtaagcagttcctgccccggctcagggccaagaacagatggtccccagatgcggtcccgccctcagcagtttctagagaaccatcagatgtttccagggtgccccaaggacctgaaaatgaccctgtgccttatttgaactaaccaatcagttcgcttctcgcttctgttcgcgcgcttctgctccccgagctcaataaaagagcccacaacccctcactcagcggccgccccgggtcgacgctaccaccatggaattcggcctgagctggctgttcctggtggccatcctgaagggcgtgcagtgcctgcagcagacaccggcctacatcaaagtgcagaccaacaagatggtcatgctgtcctgcgaggccaagatttccctctccaacatgcggatctattggttgcggcagagacaggcgccttcctcggactcccaccatgagttcttggccctgtgggactccgccaagggaactattcacggcgaagaagtggaacaggagaagatcgccgtgtttcgcgatgcctcccgctttatactgaatctgacctccgtgaagcccgaagatagcgggatctacttttgcatgattgtgggctcacccgaactgaccttcgggaagggcactcagctgagcgtggtggacttcctccccactaccgcccaacccactaagaagtcaaccctgaagaagcgggtttgcagactcccacggccggaaacgcagaagggtccgctgtgttccccgatcaccctggggctccttgtggctggagtgctggtccttctggtgtcccttggcgtcgccattcacctctgctgccggagaaggagggccagactgaggttcatgaagcagaagttcaacatagtctgcctgaagatttccgggttcactacttgctgctgcttccaaatactccagatttcacgagaatatgggttcggtgtgttgctccaaaaagacatcggtcaacgggccaagagatctggcagcggcgccaccaatttcagcctgctgaaacaggccggcgacgtggaagagaaccctggccccatggagttcggcctgagctggctgttcctggtggccatcctgaagggcgtgcagtgctcccaattccgggtgtcccctctggatcgcacctggaacctcggggaaacggtggagctcaagtgtcaagtcctcctgtcgaacccgaccagcggatgcagctggctgttccagccgagaggagctgccgcctcacccaccttcctcctgtacttgagccagaacaagccgaaggccgctgagggtctggacacccagcgcttctcgggcaaacggctgggagacacttttgtgctgactctctccgacttccggcgggagaacgagggctactacttctgctctgcgctctccaattcaatcatgtacttctcacacttcgtgccggtgttcctgcctgccaagcccaccactactccggcacccagacctccaactcccgctcccaccatcgcgtcccaacccctttcgctgcgccctgaagcgtgtcggcctgctgctggaggagccgtgcatacccgcggtctggacttcgcgtgcgacatctacatttgggcccctttggctggcacctgtggagtgctgctcctgtcccttgtgatcaccctgtactgcaaccaccggaataggcggagagtctgcaagtgtccgcggcctgtcgtgaagtcaggagataagccgagcctgtccgcacgctacgtgcgggccaagagatctggcagcggcgagggcagaggcagcctgctgacctgcggcgacgtggaggagaaccccggccccatggactcttggaccttctgctgcgtgagcctgtgcatcctggtggccaagcacacagacgccggcgtgatccagtcccctaggcacgaggtgaccgagatgggccaggaggtgacactgcgctgtaagccaatctctggccacaacagcctgttttggtatagggagaccatgatgcgcggcctggagctgctgatctacttcaataacaatgtgcccatcgacgattccggcatgcctgaggatcggttttctgccaagatgcccaatgccagcttctccacactgaagatccagcctagcgagccaagagactccgccgtgtatttttgcgcctctagcccaggcagcaccgatacacagtacttcggaccaggaaccaggctgacagtgctggaggacctgaagaacgtgttcccccctgaggtggccgtgtttgagccctctgaggccgagatcagccacacccagaaggccaccctggtgtgcctggcaaccggcttctatcctgatcacgtggagctgtcctggtgggtgaacggcaaggaggtgcacagcggcgtgtccacagacccacagcccctgaaggagcagccagccctgaatgatagccggtattgcctgtcctctcggctgagagtgtccgccaccttttggcagaacccccggaatcacttcagatgtcaggtgcagttttacggcctgtccgagaacgatgagtggacccaggaccgggccaagcctgtgacacagatcgtgtctgccgaggcatggggaagagcagactgtggcttcacctctgagagctaccagcagggcgtgctgagcgccaccatcctgtatgagatcctgctgggcaaggccacactgtacgccgtcctggtctccgctctggtgctgatggcaatggtcaaaagaaaagatagtcggggacgggccaagagatctggcagcggccagtgcaccaactacgccctgctgaagctggccggcgacgtggagagcaaccccggccccatggagaagaatcccctggctgcccccctgctgatcctgtggtttcacctggactgcgtgtcctctatcctgaatgtggaacagagcccacagagcctgcacgtgcaggagggcgactccaccaacttcacatgctcttttcctagctccaacttctacgccctgcactggtacagaaaggagaccgcaaagtccccagaggccctgttcgtgatgacactgaacggcgatgagaagaagaagggccgcatcagcgccaccctgaatacaaaggagggctactcctatctgtacatcaagggctcccagcctgaggactctgccacctatctgtgcgccctgtacaacaataacgatatgcggtttggcgccggcaccagactgacagtgaagccaaacatccagaatccagaccccgccgtgtatcagctgcgggacagcaagtctagcgataagagcgtgtgcctgttcaccgactttgattctcagacaaacgtgagccagtccaaggacagcgacgtgtacatcaccgacaagacagtgctggatatgagaagcatggacttcaagtctaacagcgccgtggcctggtccaataagtctgatttcgcctgcgccaatgcctttaataactccatcatccccgaggataccttctttccttctccagagtcctcttgtgacgtgaagctggtggagaagtctttcgagaccgatacaaacctgaattttcagaacctgagcgtgatcggcttcaggatcctgctgctgaaggtggccggctttaatctgctgatgaccctgaggctgtggagctcctgaaccggtccggagcatcttaccgccatttatacccatatttgttctgtttttcttgatttgggtatacatttaaatgttaataaaacaaaatggtggggcaatcatttacattttttgggatatgtaattactagttcaggtgtattgccacaagacaaacttgttaagaaactttcccgttatttacgctctgttcctgttaatcaacctctggattacaaaatttgtgaaagattgactgatattcttaactttgttgctccttttacgctgtgtggatttgctgctttattgcctctgtatcttgctattgcttcccgtacggctttcgttttctcctccttgtataaatcctggttgctgtctctttttgaggagttgtggcccgttgtccgtcaacgtggcgtggtgtgctctgtgtttgctgacgcaacccccactggctggggcattgccaccacctgtcaactcctttctgggactttcgctttccccctcccgatcgccacggcagaactcatcgccgcctgccttgcccgctgctggacaggggctaggttgctgggcactgataattccgtggtgttgtc

SEQ ID NO: 276 - protein, 1142 aa, synthetic construct
  REGION 1..1142: note = CD8b4(S19).CD8a(S19).TCR.WPREmut2 amino acid sequence
MEFGLSWLFLVAILKGVQCLQQTPAYIKVQTNKMVMLSCEAKISLSNMRIYWLRQRQAPSSDSHHEFLALWDSAKGTIHGEEVEQEKIAVFRDASRFILNLTSVKPEDSGIYFCMIVGSPELTFGKGTQLSVVDFLPTTAQPTKKSTLKKRVCRLPRPETQKGPLCSPITLGLLVAGVLVLLVSLGVAIHLCCRRRRARLRFMKQKFNIVCLKISGFTTCCCFQILQISREYGFGVLLQKDIGQRAKRSGSGATNFSLLKQAGDVEENPGPMEFGLSWLFLVAILKGVQCSQFRVSPLDRTWNLGETVELKCQVLLSNPTSGCSWLFQPRGAAASPTFLLYLSQNKPKAAEGLDTQRFSGKRLGDTFVLTLSDFRRENEGYYFCSALSNSIMYFSHFVPVFLPAKPTTTPAPRPPTPAPTIASQPLSLRPEACRPAAGGAVHTRGLDFACDIYIWAPLAGTCGVLLLSLVITLYCNHRNRRRVCKCPRPVVKSGDKPSLSARYVRAKRSGSGEGRGSLLTCGDVEENPGPMDSWTFCCVSLCILVAKHTDAGVIQSPRHEVTEMGQEVTLRCKPISGHNSLFWYRETMMRGLELLIYFNNNVPIDDSGMPEDRFSAKMPNASFSTLKIQPSEPRDSAVYFCASSPGSTDTQYFGPGTRLTVLEDLKNVFPPEVAVFEPSEAEISHTQKATLVCLATGFYPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSESYQQGVLSATILYEILLGKATLYAVLVSALVLMAMVKRKDSRGRAKRSGSGQCTNYALLKLAGDVESNPGPMEKNPLAAPLLILWFHLDCVSSILNVEQSPQSLHVQEGDSTNFTCSFPSSNFYALHWYRKETAKSPEALFVMTLNGDEKKKGRISATLNTKEGYSYLYIKGSQPEDSATYLCALYNNNDMRFGAGTRLTVKPNIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 277 - DNA, 4407 bp, synthetic construct
  misc_feature 1..4407: note = CD8b5(S19).CD8a(S19).TCR.WPREmut2 construct nucleotide sequence
  source 1..4407: mol_type = other DNA
tgaaagaccccacctgtaggtttggcaagctagcttaagtaacgccattttgcaaggcatggaaaatacataactgagaatagagaagttcagatcaaggttaggaacagagagacagcagaatatgggccaaacaggatatctgtggtaagcagttcctgccccggctcagggccaagaacagatggtccccagatgcggtcccgccctcagcagtttctagagaaccatcagatgtttccagggtgccccaaggacctgaaaatgaccctgtgccttatttgaactaaccaatcagttcgcttctcgcttctgttcgcgcgcttctgctccccgagctcaataaaagagcccacaacccctcactcagcggccgccccgggtcgacgctaccaccatggaattcggcctgagctggctgttcctggtggccatcctgaagggcgtgcagtgcctgcagcagacaccggcctacatcaaagtgcagaccaacaagatggtcatgctgtcctgcgaggccaagatttccctctccaacatgcggatctattggttgcggcagagacaggcgccttcctcggactcccaccatgagttcttggccctgtgggactccgccaagggaactattcacggcgaagaagtggaacaggagaagatcgccgtgtttcgcgatgcctcccgctttatactgaatctgacctccgtgaagcccgaagatagcgggatctacttttgcatgattgtgggctcacccgaactgaccttcgggaagggcactcagctgagcgtggtggacttcctccccactaccgcccaacccactaagaagtcaaccctgaagaagcgggtttgcagactcccacggccggaaacgcagaagggtccgctgtgttccccgatcaccctggggctccttgtggctggagtgctggtccttctggtgtcccttggcgtcgccattcacctctgctgccggagaaggagggccagactgaggttcatgaagcagcctcagggagaggggatcagtggcactttcgtgccacaatgcctccatggctactattccaacaccaccacctcgcaaaagctgctgaacccctggatcctgaaaacccgggccaagagatctggcagcggcgccaccaatttcagcctgctgaaacaggccggcgacgtggaagagaaccctggccccatggagttcggcctgagctggctgttcctggtggccatcctgaagggcgtgcagtgctcccaattccgggtgtcccctctggatcgcacctggaacctcggggaaacggtggagctcaagtgtcaagtcctcctgtcgaacccgaccagcggatgcagctggctgttccagccgagaggagctgccgcctcacccaccttcctcctgtacttgagccagaacaagccgaaggccgctgagggtctggacacccagcgcttctcgggcaaacggctgggagacacttttgtgctgactctctccgacttccggcgggagaacgagggctactacttctgctctgcgctctccaattcaatcatgtacttctcacacttcgtgccggtgttcctgcctgccaagcccaccactactccggcacccagacctccaactcccgctcccaccatcgcgtcccaacccctttcgctgcgccctgaagcgtgtcggcctgctgctggaggagccgtgcatacccgcggtctggacttcgcgtgcgacatctacatttgggcccctttggctggcacctgtggagtgctgctcctgtcccttgtgatcaccctgtactgcaaccaccggaataggcggagagtctgcaagtgtccgcggcctgtcgtgaagtcaggagataagccgagcctgtccgcacgctacgtgcgggccaagagatctggcagcggcgagggcagaggcagcctgctgacctgcggcgacgtggaggagaaccccggccccatggactcttggaccttctgctgcgtgagcctgtgcatcctggtggccaagcacacagacgccggcgtgatccagtcccctaggcacgaggtgaccgagatgggccaggaggtgacactgcgctgtaagccaatctctggccacaacagcctgttttggtatagggagaccatgatgcgcggcctggagctgctgatctacttcaataacaatgtgcccatcgacgattccggcatgcctgaggatcggttttctgccaagatgcccaatgccagcttctccacactgaagatccagcctagcgagccaagagactccgccgtgtatttttgcgcctctagcccaggcagcaccgatacacagtacttcggaccaggaaccaggctgacagtgctggaggacctgaagaacgtgttcccccctgaggtggccgtgtttgagccctctgaggccgagatcagccacacccagaaggccaccctggtgtgcctggcaaccggcttctatcctgatcacgtggagctgtcctggtgggtgaacggcaaggaggtgcacagcggcgtgtccacagacccacagcccctgaaggagcagccagccctgaatgatagccggtattgcctgtcctctcggctgagagtgtccgccaccttttggcagaacccccggaatcacttcagatgtcaggtgcagttttacggcctgtccgagaacgatgagtggacccaggaccgggccaagcctgtgacacagatcgtgtctgccgaggcatggggaagagcagactgtggcttcacctctgagagctaccagcagggcgtgctgagcgccaccatcctgtatgagatcctgctgggcaaggccacactgtacgccgtcctggtctccgctctggtgctgatggcaatggtcaaaagaaaagatagtcggggacgggccaagagatctggcagcggccagtgcaccaactacgccctgctgaagctggccggcgacgtggagagcaaccccggccccatggagaagaatcccctggctgcccccctgctgatcctgtggtttcacctggactgcgtgtcctctatcctgaatgtggaacagagcccacagagcctgcacgtgcaggagggcgactccaccaacttcacatgctcttttcctagctccaacttctacgccctgcactggtacagaaaggagaccgcaaagtccccagaggccctgttcgtgatgacactgaacggcgatgagaagaagaagggccgcatcagcgccaccctgaatacaaaggagggctactcctatctgtacatcaagggctcccagcctgaggactctgccacctatctgtgcgccctgtacaacaataacgatatgcggtttggcgccggcaccagactgacagtgaagccaaacatccagaatccagaccccgccgtgtatcagctgcgggacagcaagtctagcgataagagcgtgtgcctgttcaccgactttgattctcagacaaacgtgagccagtccaaggacagcgacgtgtacatcaccgacaagacagtgctggatatgagaagcatggacttcaagtctaacagcgccgtggcctggtccaataagtctgatttcgcctgcgccaatgcctttaataactccatcatccccgaggataccttctttccttctccagagtcctcttgtgacgtgaagctggtggagaagtctttcgagaccgatacaaacctgaattttcagaacctgagcgtgatcggcttcaggatcctgctgctgaaggtggccggctttaatctgctgatgaccctgaggctgtggagctcctgaaccggtccggagcatcttaccgccatttatacccatatttgttctgtttttcttgatttgggtatacatttaaatgttaataaaacaaaatggtggggcaatcatttacattttttgggatatgtaattactagttcaggtgtattgccacaagacaaacttgttaagaaactttcccgttatttacgctctgttcctgttaatcaacctctggattacaaaatttgtgaaagattgactgatattcttaactttgttgctccttttacgctgtgtggatttgctgctttattgcctctgtatcttgctattgcttcccgtacggctttcgttttctcctccttgtataaatcctggttgctgtctctttttgaggagttgtggcccgttgtccgtcaacgtggcgtggtgtgctctgtgtttgctgacgcaacccccactggctggggcattgccaccacctgtcaactcctttctgggactttcgctttccccctcccgatcgccacggcagaactcatcgccgcctgccttgcccgctgctggacaggggctaggttgctgggcactgataattccgtggtgttgtc

SEQ ID NO: 278 - protein, 1139 aa, synthetic construct
  REGION 1..1139: note = CD8b5(S19).CD8a(S19).TCR.WPREmut2 amino acid sequence
MEFGLSWLFLVAILKGVQCLQQTPAYIKVQTNKMVMLSCEAKISLSNMRIYWLRQRQAPSSDSHHEFLALWDSAKGTIHGEEVEQEKIAVFRDASRFILNLTSVKPEDSGIYFCMIVGSPELTFGKGTQLSVVDFLPTTAQPTKKSTLKKRVCRLPRPETQKGPLCSPITLGLLVAGVLVLLVSLGVAIHLCCRRRRARLRFMKQPQGEGISGTFVPQCLHGYYSNTTTSQKLLNPWILKTRAKRSGSGATNFSLLKQAGDVEENPGPMEFGLSWLFLVAILKGVQCSQFRVSPLDRTWNLGETVELKCQVLLSNPTSGCSWLFQPRGAAASPTFLLYLSQNKPKAAEGLDTQRFSGKRLGDTFVLTLSDFRRENEGYYFCSALSNSIMYFSHFVPVFLPAKPTTTPAPRPPTPAPTIASQPLSLRPEACRPAAGGAVHTRGLDFACDIYIWAPLAGTCGVLLLSLVITLYCNHRNRRRVCKCPRPVVKSGDKPSLSARYVRAKRSGSGEGRGSLLTCGDVEENPGPMDSWTFCCVSLCILVAKHTDAGVIQSPRHEVTEMGQEVTLRCKPISGHNSLFWYRETMMRGLELLIYFNNNVPIDDSGMPEDRFSAKMPNASFSTLKIQPSEPRDSAVYFCASSPGSTDTQYFGPGTRLTVLEDLKNVFPPEVAVFEPSEAEISHTQKATLVCLATGFYPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSESYQQGVLSATILYEILLGKATLYAVLVSALVLMAMVKRKDSRGRAKRSGSGQCTNYALLKLAGDVESNPGPMEKNPLAAPLLILWFHLDCVSSILNVEQSPQSLHVQEGDSTNFTCSFPSSNFYALHWYRKETAKSPEALFVMTLNGDEKKKGRISATLNTKEGYSYLYIKGSQPEDSATYLCALYNNNDMRFGAGTRLTVKPNIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 279 - DNA, 3573 bp, synthetic construct
  misc_feature 1..3573: note = CD8a(S19).CD8bstalk.TCR.WPREmut2 construct nucleotide sequence
  source 1..3573: mol_type = other DNA
tgaaagaccccacctgtaggtttggcaagctagcttaagtaacgccattttgcaaggcatggaaaatacataactgagaatagagaagttcagatcaaggttaggaacagagagacagcagaatatgggccaaacaggatatctgtggtaagcagttcctgccccggctcagggccaagaacagatggtccccagatgcggtcccgccctcagcagtttctagagaaccatcagatgtttccagggtgccccaaggacctgaaaatgaccctgtgccttatttgaactaaccaatcagttcgcttctcgcttctgttcgcgcgcttctgctccccgagctcaataaaagagcccacaacccctcactcagcggccgccccgggtcgacgctaccaccatggagttcggcctgagctggctgttcctggtggccatcctgaagggcgtgcagtgctcccaattccgggtgtcccctctggatcgcacctggaacctcggggaaacggtggagctcaagtgtcaagtcctcctgtcgaacccgaccagcggatgcagctggctgttccagccgagaggagctgccgcctcacccaccttcctcctgtacttgagccagaacaagccgaaggccgctgagggtctggacacccagcgcttctcgggcaaacggctgggagacacttttgtgctgactctctccgacttccggcgggagaacgagggctactacttctgctctgcgctctccaattcaatcatgtacttctcacacttcgtgccggtgttcctgcctgccagcgtggtggacttcctccccactaccgcccaacccactaagaagtcaaccctgaagaagcgggtttgcagactcccacggccggaaacgcagaagggtccgctgtgttccccgatctacatttgggcccctttggctggcacctgtggagtgctgctcctgtcccttgtgatcaccctgtactgcaaccaccggaataggcggagagtctgcaagtgtccgcggcctgtcgtgaagtcaggagataagccgagcctgtccgcacgctacgtgcgggccaagagatctggcagcggcgccaccaatttcagcctgctgaaacaggccggcgacgtggaagagaaccctggccccatggactcttggaccttctgctgcgtgagcctgtgcatcctggtggccaagcacacagacgccggcgtgatccagtcccctaggcacgaggtgaccgagatgggccaggaggtgacactgcgctgtaagccaatctctggccacaacagcctgttttggtatagggagaccatgatgcgcggcctggagctgctgatctacttcaataacaatgtgcccatcgacgattccggcatgcctgaggatcggttttctgccaagatgcccaatgccagcttctccacactgaagatccagcctagcgagccaagagactccgccgtgtatttttgcgcctctagcccaggcagcaccgatacacagtacttcggaccaggaaccaggctgacagtgctggaggacctgaagaacgtgttcccccctgaggtggccgtgtttgagccctctgaggccgagatcagccacacccagaaggccaccctggtgtgcctggcaaccggcttctatcctgatcacgtggagctgtcctggtgggtgaacggcaaggaggtgcacagcggcgtgtccacagacccacagcccctgaaggagcagccagccctgaatgatagccggtattgcctgtcctctcggctgagagtgtccgccaccttttggcagaacccccggaatcacttcagatgtcaggtgcagttttacggcctgtccgagaacgatgagtggacccaggaccgggccaagcctgtgacacagatcgtgtctgccgaggcatggggaagagcagactgtggcttcacctctgagagctaccagcagggcgtgctgagcgccaccatcctgtatgagatcctgctgggcaaggccacactgtacgccgtcctggtctccgctctggtgctgatggcaatggtcaaaagaaaagatagtcggggacgggccaagagatctggcagcggcgagggcagaggcagcctgctgacctgcggcgacgtggaggagaaccccggccccatggagaagaatcccctggctgcccccctgctgatcctgtggtttcacctggactgcgtgtcctctatcctgaatgtggaacagagcccacagagcctgcacgtgcaggagggcgactccaccaacttcacatgctcttttcctagctccaacttctacgccctgcactggtacagaaaggagaccgcaaagtccccagaggccctgttcgtgatgacactgaacggcgatgagaagaagaagggccgcatcagcgccaccctgaatacaaaggagggctactcctatctgtacatcaagggctcccagcctgaggactctgccacctatctgtgcgccctgtacaacaataacgatatgcggtttggcgccggcaccagactgacagtgaagccaaacatccagaatccagaccccgccgtgtatcagctgcgggacagcaagtctagcgataagagcgtgtgcctgttcaccgactttgattctcagacaaacgtgagccagtccaaggacagcgacgtgtacatcaccgacaagacagtgctggatatgagaagcatggacttcaagtctaacagcgccgtggcctggtccaataagtctgatttcgcctgcgccaatgcctttaataactccatcatccccgaggataccttctttccttctccagagtcctcttgtgacgtgaagctggtggagaagtctttcgagaccgatacaaacctgaattttcagaacctgagcgtgatcggcttcaggatcctgctgctgaaggtggccggctttaatctgctgatgaccctgaggctgtggagctcctgaaccggtccggagcatcttaccgccatttatacccatatttgttctgtttttcttgatttgggtatacatttaaatgttaataaaacaaaatggtggggcaatcatttacattttttgggatatgtaattactagttcaggtgtattgccacaagacaaacttgttaagaaactttcccgttatttacgctctgttcctgttaatcaacctctggattacaaaatttgtgaaagattgactgatattcttaactttgttgctccttttacgctgtgtggatttgctgctttattgcctctgtatcttgctattgcttcccgtacggctttcgttttctcctccttgtataaatcctggttgctgtctctttttgaggagttgtggcccgttgtccgtcaacgtggcgtggtgtgctctgtgtttgctgacgcaacccccactggctggggcattgccaccacctgtcaactcctttctgggactttcgctttccccctcccgatcgccacggcagaactcatcgccgcctgccttgcccgctgctggacaggggctaggttgctgggcactgataattccgtggtgttgtc

SEQ ID NO: 280 - protein, 861 aa, synthetic construct
  REGION 1..861: note = CD8a(S19).CD8bstalk.TCR.WPREmut2 amino acid sequence
MEFGLSWLFLVAILKGVQCSQFRVSPLDRTWNLGETVELKCQVLLSNPTSGCSWLFQPRGAAASPTFLLYLSQNKPKAAEGLDTQRFSGKRLGDTFVLTLSDFRRENEGYYFCSALSNSIMYFSHFVPVFLPASVVDFLPTTAQPTKKSTLKKRVCRLPRPETQKGPLCSPIYIWAPLAGTCGVLLLSLVITLYCNHRNRRRVCKCPRPVVKSGDKPSLSARYVRAKRSGSGATNFSLLKQAGDVEENPGPMDSWTFCCVSLCILVAKHTDAGVIQSPRHEVTEMGQEVTLRCKPISGHNSLFWYRETMMRGLELLIYFNNNVPIDDSGMPEDRFSAKMPNASFSTLKIQPSEPRDSAVYFCASSPGSTDTQYFGPGTRLTVLEDLKNVFPPEVAVFEPSEAEISHTQKATLVCLATGFYPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSESYQQGVLSATILYEILLGKATLYAVLVSALVLMAMVKRKDSRGRAKRSGSGEGRGSLLTCGDVEENPGPMEKNPLAAPLLILWFHLDCVSSILNVEQSPQSLHVQEGDSTNFTCSFPSSNFYALHWYRKETAKSPEALFVMTLNGDEKKKGRISATLNTKEGYSYLYIKGSQPEDSATYLCALYNNNDMRFGAGTRLTVKPNIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 281 - DNA, 4284 bp, synthetic construct
  misc_feature 1..4284: note = TCRba.CD8a.CD8b1.2.WPREmut2 construct nucleotide sequence
  source 1..4284: mol_type = other DNA
tgaaagaccccacctgtaggtttggcaagctagcttaagtaacgccattttgcaaggcatggaaaatacataactgagaatagagaagttcagatcaaggttaggaacagagagacagcagaatatgggccaaacaggatatctgtggtaagcagttcctgccccggctcagggccaagaacagatggtccccagatgcggtcccgccctcagcagtttctagagaaccatcagatgtttccagggtgccccaaggacctgaaaatgaccctgtgccttatttgaactaaccaatcagttcgcttctcgcttctgttcgcgcgcttctgctccccgagctcaataaaagagcccacaacccctcactcagcggccgccccgggtcgacgctaccaccatggactcttggaccttctgctgcgtgagcctgtgcatcctggtggccaagcacacagacgccggcgtgatccagtcccctaggcacgaggtgaccgagatgggccaggaggtgacactgcgctgtaagccaatctctggccacaacagcctgttttggtatagggagaccatgatgcgcggcctggagctgctgatctacttcaataacaatgtgcccatcgacgattccggcatgcctgaggatcggttttctgccaagatgcccaatgccagcttctccacactgaagatccagcctagcgagccaagagactccgccgtgtatttttgcgcctctagcccaggcagcaccgatacacagtacttcggaccaggaaccaggctgacagtgctggaggacctgaagaacgtgttcccccctgaggtggccgtgtttgagccctctgaggccgagatcagccacacccagaaggccaccctggtgtgcctggcaaccggcttctatcctgatcacgtggagctgtcctggtgggtgaacggcaaggaggtgcacagcggcgtgtccacagacccacagcccctgaaggagcagccagccctgaatgatagccggtattgcctgtcctctcggctgagagtgtccgccaccttttggcagaacccccggaatcacttcagatgtcaggtgcagttttacggcctgtccgagaacgatgagtggacccaggaccgggccaagcctgtgacacagatcgtgtctgccgaggcatggggaagagcagactgtggcttcacctctgagagctaccagcagggcgtgctgagcgccaccatcctgtatgagatcctgctgggcaaggccacactgtacgccgtcctggtctccgctctggtgctgatggcaatggtcaaaagaaaagatagtcggggacgggccaagagatctggcagcggcgagggcagaggcagcctgctgacctgcggcgacgtggaggagaaccccggccccatggagaagaatcccctggctgcccccctgctgatcctgtggtttcacctggactgcgtgtcctctatcctgaatgtggaacagagcccacagagcctgcacgtgcaggagggcgactccaccaacttcacatgctcttttcctagctccaacttctacgccctgcactggtacagaaaggagaccgcaaagtccccagaggccctgttcgtgatgacactgaacggcgatgagaagaagaagggccgcatcagcgccaccctgaatacaaaggagggctactcctatctgtacatcaagggctcccagcctgaggactctgccacctatctgtgcgccctgtacaacaataacgatatgcggtttggcgccggcaccagactgacagtgaagccaaacatccagaatccagaccccgccgtgtatcagctgcgggacagcaagtctagcgataagagcgtgtgcctgttcaccgactttgattctcagacaaacgtgagccagtccaaggacagcgacgtgtacatcaccgacaagacagtgctggatatgagaagcatggacttcaagtctaacagcgccgtggcctggtccaataagtctgatttcgcctgcgccaatgcctttaataactccatcatccccgaggataccttctttccttctccagagtcctcttgtgacgtgaagctggtggagaagtctttcgagaccgatacaaacctgaattttcagaacctgagcgtgatcggcttcaggatcctgctgctgaaggtggccggctttaatctgctgatgaccctgaggctgtggagctcccgggccaagagatctggcagcggccagtgcaccaactacgccctgctgaagctggccggcgacgtggagagcaaccccggccccatggcgcttcccgtgaccgcactcctgttgccccttgccctgctgttgcacgccgcacgacctagccagttcagagtgtcccccctggaccggacctggaacctgggagagacagtggaactgaagtgccaggtgctgctgagcaaccccaccagcggctgctcttggctgtttcagcctagaggcgccgctgccagccctacctttctgctgtacctgagccagaacaagcccaaggccgccgagggcctggacacccagagattcagcggcaagagactgggcgacaccttcgtgctgaccctgagcgacttcagaagagagaacgagggctactacttctgcagcgccctgagcaacagcatcatgtacttcagccacttcgtgcccgtgtttctgcccgccaagcctaccacaacccctgcccctagacctcctaccccagcccctacaatcgccagccagcctctgtctctgaggcccgaggcttgtagacctgctgctggcggagccgtgcacaccagaggactggatttcgcctgcgacatctacatctgggcccctctggccggcacatgtggcgtgctgctgctgtccctcgtgatcaccctgtactgcaaccaccggaaccggcggagagtgtgcaagtgccctagacccgtcgtgaagtccggcgacaagcctagcctgagcgccagatacgtgcgggccaagagatctggcagcggcgccaccaatttcagcctgctgaaacaggccggcgacgtggaagagaaccctggccccatgcgcccgagactgtggcttctgctcgccgcgcaactgactgtcctgcacggaaacagcgtgctgcagcagacaccggcctacatcaaagtgcagaccaacaagatggtcatgctgtcctgcgaggccaagatttccctctccaacatgcggatctattggttgcggcagagacaggcgccttcctcggactcccaccatgagttcttggccctgtgggactccgccaagggaactattcacggcgaagaagtggaacaggagaagatcgccgtgtttcgcgatgcctcccgctttatactgaatctgacctccgtgaagcccgaagatagcgggatctacttttgcatgattgtgggctcacccgaactgaccttcgggaagggcactcagctgagcgtggtggacttcctccccactaccgcccaacccactaagaagtcaaccctgaagaagcgggtttgcagactcccacggccggaaacgcagaagggtctgaaaggtaaggtttaccaagagcctttgtcaccgaacgcttgtatggacacgaccgctatcctccaaccgcataggtcttgtctcactcacgggtcatgaaccggtccggagcatcttaccgccatttatacccatatttgttctgtttttcttgatttgggtatacatttaaatgttaataaaacaaaatggtggggcaatcatttacattttttgggatatgtaattactagttcaggtgtattgccacaagacaaacttgttaagaaactttcccgttatttacgctctgttcctgttaatcaacctctggattacaaaatttgtgaaagattgactgatattcttaactttgttgctccttttacgctgtgtggatttgctgctttattgcctctgtatcttgctattgcttcccgtacggctttcgttttctcctccttgtataaatcctggttgctgtctctttttgaggagttgtggcccgttgtccgtcaacgtggcgtggtgtgctctgtgtttgctgacgcaacccccactggctggggcattgccaccacctgtcaactcctttctgggactttcgctttccccctcccgatcgccacggcagaactcatcgccgcctgccttgcccgctgctggacaggggctaggttgctgggcactgataattccgtggtgttgtc

SEQ ID NO: 282 - protein, 1098 aa, synthetic construct
  REGION 1..1098: note = TCRba.CD8a.CD8b1.2.WPREmut2 amino acid sequence
MDSWTFCCVSLCILVAKHTDAGVIQSPRHEVTEMGQEVTLRCKPISGHNSLFWYRETMMRGLELLIYFNNNVPIDDSGMPEDRFSAKMPNASFSTLKIQPSEPRDSAVYFCASSPGSTDTQYFGPGTRLTVLEDLKNVFPPEVAVFEPSEAEISHTQKATLVCLATGFYPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSESYQQGVLSATILYEILLGKATLYAVLVSALVLMAMVKRKDSRGRAKRSGSGEGRGSLLTCGDVEENPGPMEKNPLAAPLLILWFHLDCVSSILNVEQSPQSLHVQEGDSTNFTCSFPSSNFYALHWYRKETAKSPEALFVMTLNGDEKKKGRISATLNTKEGYSYLYIKGSQPEDSATYLCALYNNNDMRFGAGTRLTVKPNIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSSRAKRSGSGQCTNYALLKLAGDVESNPGPMALPVTALLLPLALLLHAARPSQFRVSPLDRTWNLGETVELKCQVLLSNPTSGCSWLFQPRGAAASPTFLLYLSQNKPKAAEGLDTQRFSGKRLGDTFVLTLSDFRRENEGYYFCSALSNSIMYFSHFVPVFLPAKPTTTPAPRPPTPAPTIASQPLSLRPEACRPAAGGAVHTRGLDFACDIYIWAPLAGTCGVLLLSLVITLYCNHRNRRRVCKCPRPVVKSGDKPSLSARYVRAKRSGSGATNFSLLKQAGDVEENPGPMRPRLWLLLAAQLTVLHGNSVLQQTPAYIKVQTNKMVMLSCEAKISLSNMRIYWLRQRQAPSSDSHHEFLALWDSAKGTIHGEEVEQEKIAVFRDASRFILNLTSVKPEDSGIYFCMIVGSPELTFGKGTQLSVVDFLPTTAQPTKKSTLKKRVCRLPRPETQKGLKGKVYQEPLSPNACMDTTAILQPHRSCLTHGS

SEQ ID NO: 283 - DNA, 3606 bp, synthetic construct
  misc_feature 1..3606: note = WT-SPa/CD8a-Ig like domain 1/CD8bstalk/CD4 TM+IC/TCRba/WPREmut2 construct nucleotide sequence
  source 1..3606: mol_type = other DNA
tgaaagaccccacctgtaggtttggcaagctagcttaagtaacgccattttgcaaggcatggaaaatacataactgagaatagagaagttcagatcaaggttaggaacagagagacagcagaatatgggccaaacaggatatctgtggtaagcagttcctgccccggctcagggccaagaacagatggtccccagatgcggtcccgccctcagcagtttctagagaaccatcagatgtttccagggtgccccaaggacctgaaaatgaccctgtgccttatttgaactaaccaatcagttcgcttctcgcttctgttcgcgcgcttctgctccccgagctcaataaaagagcccacaacccctcactcagcggccgccccgggtcgacgctaccaccatggcgcttcccgtgaccgcactcctgttgccccttgccctgctgttgcacgccgcacgaccttcccaattccgggtgtcccctctggatcgcacctggaacctcggggaaacggtggagctcaagtgtcaagtcctcctgtcgaacccgaccagcggatgcagctggctgttccagccgagaggagctgccgcctcacccaccttcctcctgtacttgagccagaacaagccgaaggccgctgagggtctggacacccagcgcttctcgggcaaacggctgggagacacttttgtgctgactctctccgacttccggcgggagaacgagggctactacttctgctctgcgctctccaattcaatcatgtacttctcacacttcgtgccggtgttcctgcctgccagcgtggtggacttcctccccactaccgcccaacccactaagaagtcaaccctgaagaagcgggtttgcagactcccacggccggaaacgcagaagggtccgctgtgttccccgatggccctgattgtgctggggggcgtcgccggcctcctgcttttcattgggctaggcatcttcttctgtgtcaggtgccggcaccgaaggcgccaagcagagcggatgtctcagatcaagagactcctcagtgagaagaagacctgccagtgtcctcaccggtttcagaagacatgtagccccattcgggccaagagatctggcagcggcgccaccaatttcagcctgctgaaacaggccggcgacgtggaagagaaccctggccccatggactcttggaccttctgctgcgtgagcctgtgcatcctggtggccaagcacacagacgccggcgtgatccagtcccctaggcacgaggtgaccgagatgggccaggaggtgacactgcgctgtaagccaatctctggccacaacagcctgttttggtatagggagaccatgatgcgcggcctggagctgctgatctacttcaataacaatgtgcccatcgacgattccggcatgcctgaggatcggttttctgccaagatgcccaatgccagcttctccacactgaagatccagcctagcgagccaagagactccgccgtgtatttttgcgcctctagcccaggcagcaccgatacacagtacttcggaccaggaaccaggctgacagtgctggaggacctgaagaacgtgttcccccctgaggtggccgtgtttgagccctctgaggccgagatcagccacacccagaaggccaccctggtgtgcctggcaaccggcttctatcctgatcacgtggagctgtcctggtgggtgaacggcaaggaggtgcacagcggcgtgtccacagacccacagcccctgaaggagcagccagccctgaatgatagccggtattgcctgtcctctcggctgagagtgtccgccaccttttggcagaacccccggaatcacttcagatgtcaggtgcagttttacggcctgtccgagaacgatgagtggacccaggaccgggccaagcctgtgacacagatcgtgtctgccgaggcatggggaagagcagactgtggcttcacctctgagagctaccagcagggcgtgctgagcgccaccatcctgtatgagatcctgctgggcaaggccacactgtacgccgtcctggtctccgctctggtgctgatggcaatggtcaaaagaaaagatagtcggggacgggccaagagatctggcagcggcgagggcagaggcagcctgctgacctgcggcgacgtggaggagaaccccggccccatggagaagaatcccctggctgcccccctgctgatcctgtggtttcacctggactgcgtgtcctctatcctgaatgtggaacagagcccacagagcctgcacgtgcaggagggcgactccaccaacttcacatgctcttttcctagctccaacttctacgccctgcactggtacagaaaggagaccgcaaagtccccagaggccctgttcgtgatgacactgaacggcgatgagaagaagaagggccgcatcagcgccaccctgaatacaaaggagggctactcctatctgtacatcaagggctcccagcctgaggactctgccacctatctgtgcgccctgtacaacaataacgatatgcggtttggcgccggcaccagactgacagtgaagccaaacatccagaatccagaccccgccgtgtatcagctgcgggacagcaagtctagcgataagagcgtgtgcctgttcaccgactttgattctcagacaaacgtgagccagtccaaggacagcgacgtgtacatcaccgacaagacagtgctggatatgagaagcatggacttcaagtctaacagcgccgtggcctggtccaataagtctgatttcgcctgcgccaatgcctttaataactccatcatccccgaggataccttctttccttctccagagtcctcttgtgacgtgaagctggtggagaagtctttcgagaccgatacaaacctgaattttcagaacctgagcgtgatcggcttcaggatcctgctgctgaaggtggccggctttaatctgctgatgaccctgaggctgtggagctcctgaaccggtccggagcatcttaccgccatttatacccatatttgttctgtttttcttgatttgggtatacatttaaatgttaataaaacaaaatggtggggcaatcatttacattttttgggatatgtaattactagttcaggtgtattgccacaagacaaacttgttaagaaactttcccgttatttacgctctgttcctgttaatcaacctctggattacaaaatttgtgaaagattgactgatattcttaactttgttgctccttttacgctgtgtggatttgctgctttattgcctctgtatcttgctattgcttcccgtacggctttcgttttctcctccttgtataaatcctggttgctgtctctttttgaggagttgtggcccgttgtccgtcaacgtggcgtggtgtgctctgtgtttgctgacgcaacccccactggctggggcattgccaccacctgtcaactcctttctgggactttcgctttccccctcccgatcgccacggcagaactcatcgccgcctgccttgcccgctgctggacaggggctaggttgctgggcactgataattccgtggtgttgtc

SEQ ID NO: 284 - protein, 872 aa, synthetic construct
  REGION 1..872: note = WT-SPa/CD8a-Ig like domain 1/CD8bstalk/CD4 TM+IC/TCRba/WPREmut2 amino acid sequence
MALPVTALLLPLALLLHAARPSQFRVSPLDRTWNLGETVELKCQVLLSNPTSGCSWLFQPRGAAASPTFLLYLSQNKPKAAEGLDTQRFSGKRLGDTFVLTLSDFRRENEGYYFCSALSNSIMYFSHFVPVFLPASVVDFLPTTAQPTKKSTLKKRVCRLPRPETQKGPLCSPMALIVLGGVAGLLLFIGLGIFFCVRCRHRRRQAERMSQIKRLLSEKKTCQCPHRFQKTCSPIRAKRSGSGATNFSLLKQAGDVEENPGPMDSWTFCCVSLCILVAKHTDAGVIQSPRHEVTEMGQEVTLRCKPISGHNSLFWYRETMMRGLELLIYFNNNVPIDDSGMPEDRFSAKMPNASFSTLKIQPSEPRDSAVYFCASSPGSTDTQYFGPGTRLTVLEDLKNVFPPEVAVFEPSEAEISHTQKATLVCLATGFYPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSESYQQGVLSATILYEILLGKATLYAVLVSALVLMAMVKRKDSRGRAKRSGSGEGRGSLLTCGDVEENPGPMEKNPLAAPLLILWFHLDCVSSILNVEQSPQSLHVQEGDSTNFTCSFPSSNFYALHWYRKETAKSPEALFVMTLNGDEKKKGRISATLNTKEGYSYLYIKGSQPEDSATYLCALYNNNDMRFGAGTRLTVKPNIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 285 - DNA, 3579 bp, synthetic construct
  misc_feature 1..3579: note = CD8a.CD8bstalk.TCR.WRPEmut2 construct nucleotide sequence
  source 1..3579: mol_type = other DNA
tgaaagaccccacctgtaggtttggcaagctagcttaagtaacgccattttgcaaggcatggaaaatacataactgagaatagagaagttcagatcaaggttaggaacagagagacagcagaatatgggccaaacaggatatctgtggtaagcagttcctgccccggctcagggccaagaacagatggtccccagatgcggtcccgccctcagcagtttctagagaaccatcagatgtttccagggtgccccaaggacctgaaaatgaccctgtgccttatttgaactaaccaatcagttcgcttctcgcttctgttcgcgcgcttctgctccccgagctcaataaaagagcccacaacccctcactcagcggccgccccgggtcgacgctaccaccatggcgcttcccgtgaccgcactcctgttgccccttgccctgctgttgcacgccgcacgaccttcccaattccgggtgtcccctctggatcgcacctggaacctcggggaaacggtggagctcaagtgtcaagtcctcctgtcgaacccgaccagcggatgcagctggctgttccagccgagaggagctgccgcctcacccaccttcctcctgtacttgagccagaacaagccgaaggccgctgagggtctggacacccagcgcttctcgggcaaacggctgggagacacttttgtgctgactctctccgacttccggcgggagaacgagggctactacttctgctctgcgctctccaattcaatcatgtacttctcacacttcgtgccggtgttcctgcctgccagcgtggtggacttcctccccactaccgcccaacccactaagaagtcaaccctgaagaagcgggtttgcagactcccacggccggaaacgcagaagggtccgctgtgttccccgatctacatttgggcccctttggctggcacctgtggagtgctgctcctgtcccttgtgatcaccctgtactgcaaccaccggaataggcggagagtctgcaagtgtccgcggcctgtcgtgaagtcaggagataagccgagcctgtccgcacgctacgtgcgggccaagagatctggcagcggcgccaccaatttcagcctgctgaaacaggccggcgacgtggaagagaaccctggccccatggactcttggaccttctgctgcgtgagcctgtgcatcctggtggccaagcacacagacgccggcgtgatccagtcccctaggcacgaggtgaccgagatgggccaggaggtgacactgcgctgtaagccaatctctggccacaacagcctgttttggtatagggagaccatgatgcgcggcctggagctgctgatctacttcaataacaatgtgcccatcgacgattccggcatgcctgaggatcggttttctgccaagatgcccaatgccagcttctccacactgaagatccagcctagcgagccaagagactccgccgtgtatttttgcgcctctagcccaggcagcaccgatacacagtacttcggaccaggaaccaggctgacagtgctggaggacctgaagaacgtgttcccccctgaggtggccgtgtttgagccctctgaggccgagatcagccacacccagaaggccaccctggtgtgcctggcaaccggcttctatcctgatcacgtggagctgtcctggtgggtgaacggcaaggaggtgcacagcggcgtgtccacagacccacagcccctgaaggagcagccagccctgaatgatagccggtattgcctgtcctctcggctgagagtgtccgccaccttttggcagaacccccggaatcacttcagatgtcaggtgcagttttacggcctgtccgagaacgatgagtggacccaggaccgggccaagcctgtgacacagatcgtgtctgccgaggcatggggaagagcagactgtggcttcacctctgagagctaccagcagggcgtgctgagcgccaccatcctgtatgagatcctgctgggcaaggccacactgtacgccgtcctggtctccgctctggtgctgatggcaatggtcaaaagaaaagatagtcggggacgggccaagagatctggcagcggcgagggcagaggcagcctgctgacctgcggcgacgtggaggagaaccccggccccatggagaagaatcccctggctgcccccctgctgatcctgtggtttcacctggactgcgtgtcctctatcctgaatgtggaacagagcccacagagcctgcacgtgcaggagggcgactccaccaacttcacatgctcttttcctagctccaacttctacgccctgcactggtacagaaaggagaccgcaaagtccccagaggccctgttcgtgatgacactgaacggcgatgagaagaagaagggccgcatcagcgccaccctgaatacaaaggagggctactcctatctgtacatcaagggctcccagcctgaggactctgccacctatctgtgcgccctgtacaacaataacgatatgcggtttggcgccggcaccagactgacagtgaagccaaacatccagaatccagaccccgccgtgtatcagctgcgggacagcaagtctagcgataagagcgtgtgcctgttcaccgactttgattctcagacaaacgtgagccagtccaaggacagcgacgtgtacatcaccgacaagacagtgctggatatgagaagcatggacttcaagtctaacagcgccgtggcctggtccaataagtctgatttcgcctgcgccaatgcctttaataactccatcatccccgaggataccttctttccttctccagagtcctcttgtgacgtgaagctggtggagaagtctttcgagaccgatacaaacctgaattttcagaacctgagcgtgatcggcttcaggatcctgctgctgaaggtggccggctttaatctgctgatgaccctgaggctgtggagctcctgaaccggtccggagcatcttaccgccatttatacccatatttgttctgtttttcttgatttgggtatacatttaaatgttaataaaacaaaatggtggggcaatcatttacattttttgggatatgtaattactagttcaggtgtattgccacaagacaaacttgttaagaaactttcccgttatttacgctctgttcctgttaatcaacctctggattacaaaatttgtgaaagattgactgatattcttaactttgttgctccttttacgctgtgtggatttgctgctttattgcctctgtatcttgctattgcttcccgtacggctttcgttttctcctccttgtataaatcctggttgctgtctctttttgaggagttgtggcccgttgtccgtcaacgtggcgtggtgtgctctgtgtttgctgacgcaacccccactggctggggcattgccaccacctgtcaactcctttctgggactttcgctttccccctcccgatcgccacggcagaactcatcgccgcctgccttgcccgctgctggacaggggctaggttgctgggcactgataattccgtggtgttgtc

SEQ ID NO: 286 - protein, 863 aa, synthetic construct
  REGION 1..863: note = CD8a.CD8bstalk.TCR.WRPEmut2 amino acid sequence
MALPVTALLLPLALLLHAARPSQFRVSPLDRTWNLGETVELKCQVLLSNPTSGCSWLFQPRGAAASPTFLLYLSQNKPKAAEGLDTQRFSGKRLGDTFVLTLSDFRRENEGYYFCSALSNSIMYFSHFVPVFLPASVVDFLPTTAQPTKKSTLKKRVCRLPRPETQKGPLCSPIYIWAPLAGTCGVLLLSLVITLYCNHRNRRRVCKCPRPVVKSGDKPSLSARYVRAKRSGSGATNFSLLKQAGDVEENPGPMDSWTFCCVSLCILVAKHTDAGVIQSPRHEVTEMGQEVTLRCKPISGHNSLFWYRETMMRGLELLIYFNNNVPIDDSGMPEDRFSAKMPNASFSTLKIQPSEPRDSAVYFCASSPGSTDTQYFGPGTRLTVLEDLKNVFPPEVAVFEPSEAEISHTQKATLVCLATGFYPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSESYQQGVLSATILYEILLGKATLYAVLVSALVLMAMVKRKDSRGRAKRSGSGEGRGSLLTCGDVEENPGPMEKNPLAAPLLILWFHLDCVSSILNVEQSPQSLHVQEGDSTNFTCSFPSSNFYALHWYRKETAKSPEALFVMTLNGDEKKKGRISATLNTKEGYSYLYIKGSQPEDSATYLCALYNNNDMRFGAGTRLTVKPNIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 287 - DNA, 3606 bp, synthetic construct
  misc_feature 1..3606: note = CD8a.TCR.WPREmut2 construct nucleotide sequence
  source 1..3606: mol_type = other DNA
tgaaagaccccacctgtaggtttggcaagctagcttaagtaacgccattttgcaaggcatggaaaatacataactgagaatagagaagttcagatcaaggttaggaacagagagacagcagaatatgggccaaacaggatatctgtggtaagcagttcctgccccggctcagggccaagaacagatggtccccagatgcggtcccgccctcagcagtttctagagaaccatcagatgtttccagggtgccccaaggacctgaaaatgaccctgtgccttatttgaactaaccaatcagttcgcttctcgcttctgttcgcgcgcttctgctccccgagctcaataaaagagcccacaacccctcactcagcggccgccccgggtcgacgctaccaccatggcgcttcccgtgaccgcactcctgttgccccttgccctgctgttgcacgccgcacgaccttcccaattccgggtgtcccctctggatcgcacctggaacctcggggaaacggtggagctcaagtgtcaagtcctcctgtcgaacccgaccagcggatgcagctggctgttccagccgagaggagctgccgcctcacccaccttcctcctgtacttgagccagaacaagccgaaggccgctgagggtctggacacccagcgcttctcgggcaaacggctgggagacacttttgtgctgactctctccgacttccggcgggagaacgagggctactacttctgctctgcgctctccaattcaatcatgtacttctcacacttcgtgccggtgttcctgcctgccaagcccaccactactccggcacccagacctccaactcccgctcccaccatcgcgtcccaacccctttcgctgcgccctgaagcgtgtcggcctgctgctggaggagccgtgcatacccgcggtctggacttcgcgtgcgacatctacatttgggcccctttggctggcacctgtggagtgctgctcctgtcccttgtgatcaccctgtactgcaaccaccggaataggcggagagtctgcaagtgtccgcggcctgtcgtgaagtcaggagataagccgagcctgtccgcacgctacgtgcgggccaagagatctggcagcggcgccaccaatttcagcctgctgaaacaggccggcgacgtggaagagaaccctggccccatggactcttggaccttctgctgcgtgagcctgtgcatcctggtggccaagcacacagacgccggcgtgatccagtcccctaggcacgaggtgaccgagatgggccaggaggtgacactgcgctgtaagccaatctctggccacaacagcctgttttggtatagggagaccatgatgcgcggcctggagctgctgatctacttcaataacaatgtgcccatcgacgattccggcatgcctgaggatcggttttctgccaagatgcccaatgccagcttctccacactgaagatccagcctagcgagccaagagactccgccgtgtatttttgcgcctctagcccaggcagcaccgatacacagtacttcggaccaggaaccaggctgacagtgctggaggacctgaagaacgtgttcccccctgaggtggccgtgtttgagccctctgaggccgagatcagccacacccagaaggccaccctggtgtgcctggcaaccggcttctatcctgatcacgtggagctgtcctggtgggtgaacggcaaggaggtgcacagcggcgtgtccacagacccacagcccctgaaggagcagccagccctgaatgatagccggtattgcctgtcctctcggctgagagtgtccgccaccttttggcagaacccccggaatcacttcagatgtcaggtgcagttttacggcctgtccgagaacgatgagtggacccaggaccgggccaagcctgtgacacagatcgtgtctgccgaggcatggggaagagcagactgtggcttcacctctgagagctaccagcagggcgtgctgagcgccaccatcctgtatgagatcctgctgggcaaggccacactgtacgccgtcctggtctccgctctggtgctgatggcaatggtcaaaagaaaagatagtcggggacgggccaagagatctggcagcggcgagggcagaggcagcctgctgacctgcggcgacgtggaggagaaccccggccccatggagaagaatcccctggctgcccccctgctgatcctgtggtttcacctggactgcgtgtcctctatcctgaatgtggaacagagcccacagagcctgcacgtgcaggagggcgactccaccaacttcacatgctcttttcctagctccaacttctacgccctgcactggtacagaaaggagaccgcaaagtccccagaggccctgttcgtgatgacactgaacggcgatgagaagaagaagggccgcatcagcgccaccctgaatacaaaggagggctactcctatctgtacatcaagggctcccagcctgaggactctgccacctatctgtgcgccctgtacaacaataacgatatgcggtttggcgccggcaccagactgacagtgaagccaaacatccagaatccagaccccgccgtgtatcagctgcgggacagcaagtctagcgataagagcgtgtgcctgttcaccgactttgattctcagacaaacgtgagccagtccaaggacagcgacgtgtacatcaccgacaagacagtgctggatatgagaagcatggacttcaagtctaacagcgccgtggcctggtccaataagtctgatttcgcctgcgccaatgcctttaataactccatcatccccgaggataccttctttccttctccagagtcctcttgtgacgtgaagctggtggagaagtctttcgagaccgatacaaacctgaattttcagaacctgagcgtgatcggcttcaggatcctgctgctgaaggtggccggctttaatctgctgatgaccctgaggctgtggagctcctgaaccggtccggagcatcttaccgccatttatacccatatttgttctgtttttcttgatttgggtatacatttaaatgttaataaaacaaaatggtggggcaatcatttacattttttgggatatgtaattactagttcaggtgtattgccacaagacaaacttgttaagaaactttcccgttatttacgctctgttcctgttaatcaacctctggattacaaaatttgtgaaagattgactgatattcttaactttgttgctccttttacgctgtgtggatttgctgctttattgcctctgtatcttgctattgcttcccgtacggctttcgttttctcctccttgtataaatcctggttgctgtctctttttgaggagttgtggcccgttgtccgtcaacgtggcgtggtgtgctctgtgtttgctgacgcaacccccactggctggggcattgccaccacctgtcaactcctttctgggactttcgctttccccctcccgatcgccacggcagaactcatcgccgcctgccttgcccgctgctggacaggggctaggttgctgggcactgataattccgtggtgttgtc

SEQ ID NO: 288 - protein, 872 aa, synthetic construct
  REGION 1..872: note = CD8a.TCR.WPREmut2 amino acid sequence
MALPVTALLLPLALLLHAARPSQFRVSPLDRTWNLGETVELKCQVLLSNPTSGCSWLFQPRGAAASPTFLLYLSQNKPKAAEGLDTQRFSGKRLGDTFVLTLSDFRRENEGYYFCSALSNSIMYFSHFVPVFLPAKPTTTPAPRPPTPAPTIASQPLSLRPEACRPAAGGAVHTRGLDFACDIYIWAPLAGTCGVLLLSLVITLYCNHRNRRRVCKCPRPVVKSGDKPSLSARYVRAKRSGSGATNFSLLKQAGDVEENPGPMDSWTFCCVSLCILVAKHTDAGVIQSPRHEVTEMGQEVTLRCKPISGHNSLFWYRETMMRGLELLIYFNNNVPIDDSGMPEDRFSAKMPNASFSTLKIQPSEPRDSAVYFCASSPGSTDTQYFGPGTRLTVLEDLKNVFPPEVAVFEPSEAEISHTQKATLVCLATGFYPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSESYQQGVLSATILYEILLGKATLYAVLVSALVLMAMVKRKDSRGRAKRSGSGEGRGSLLTCGDVEENPGPMEKNPLAAPLLILWFHLDCVSSILNVEQSPQSLHVQEGDSTNFTCSFPSSNFYALHWYRKETAKSPEALFVMTLNGDEKKKGRISATLNTKEGYSYLYIKGSQPEDSATYLCALYNNNDMRFGAGTRLTVKPNIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 289 - DNA, 3633 bp, synthetic construct
  misc_feature 1..3633: note = WT-SPa/CD8a-Ig like domain 1(co)/CD8astalk(co)/CD4 TM+IC/TCRba/WPREmut2 construct nucleotide sequence
  source 1..3633: mol_type = other DNA
tgaaagaccccacctgtaggtttggcaagctagcttaagtaacgccattttgcaaggcatggaaaatacataactgagaatagagaagttcagatcaaggttaggaacagagagacagcagaatatgggccaaacaggatatctgtggtaagcagttcctgccccggctcagggccaagaacagatggtccccagatgcggtcccgccctcagcagtttctagagaaccatcagatgtttccagggtgccccaaggacctgaaaatgaccctgtgccttatttgaactaaccaatcagttcgcttctcgcttctgttcgcgcgcttctgctccccgagctcaataaaagagcccacaacccctcactcagcggccgccccgggtcgacgctaccaccatggcgcttcccgtgaccgcactcctgttgccccttgccctgctgttgcacgccgcacgaccttcccaattccgggtgtcccctctggatcgcacctggaacctcggggaaacggtggagctcaagtgtcaagtcctcctgtcgaacccgaccagcggatgcagctggctgttccagccgagaggagctgccgcctcacccaccttcctcctgtacttgagccagaacaagccgaaggccgctgagggtctggacacccagcgcttctcgggcaaacggctgggagacacttttgtgctgactctctccgacttccggcgggagaacgagggctactacttctgctctgcgctctccaattcaatcatgtacttctcacacttcgtgccggtgttcctgcctgccaagcccaccactactccggcacccagacctccaactcccgctcccaccatcgcgtcccaacccctttcgctgcgccctgaagcgtgtcggcctgctgctggaggagccgtgcatacccgcggtctggacttcgcgtgcgacatggccctgattgtgctggggggcgtcgccggcctcctgcttttcattgggctaggcatcttcttctgtgtcaggtgccggcaccgaaggcgccaagcagagcggatgtctcagatcaagagactcctcagtgagaagaagacctgccagtgtcctcaccggtttcagaagacatgtagccccattcgggccaagagatctggcagcggcgccaccaatttcagcctgctgaaacaggccggcgacgtggaagagaaccctggccccatggactcttggaccttctgctgcgtgagcctgtgcatcctggtggccaagcacacagacgccggcgtgatccagtcccctaggcacgaggtgaccgagatgggccaggaggtgacactgcgctgtaagccaatctctggccacaacagcctgttttggtatagggagaccatgatgcgcggcctggagctgctgatctacttcaataacaatgtgcccatcgacgattccggcatgcctgaggatcggttttctgccaagatgcccaatgccagcttctccacactgaagatccagcctagcgagccaagagactccgccgtgtatttttgcgcctctagcccaggcagcaccgatacacagtacttcggaccaggaaccaggctgacagtgctggaggacctgaagaacgtgttcccccctgaggtggccgtgtttgagccctctgaggccgagatcagccacacccagaaggccaccctggtgtgcctggcaaccggcttctatcctgatcacgtggagctgtcctggtgggtgaacggcaaggaggtgcacagcggcgtgtccacagacccacagcccctgaaggagcagccagccctgaatgatagccggtattgcctgtcctctcggctgagagtgtccgccaccttttggcagaacccccggaatcacttcagatgtcaggtgcagttttacggcctgtccgagaacgatgagtggacccaggaccgggccaagcctgtgacacagatcgtgtctgccgaggcatggggaagagcagactgtggcttcacctctgagagctaccagcagggcgtgctgagcgccaccatcctgtatgagatcctgctgggcaaggccacactgtacgccgtcctggtctccgctctggtgctgatggcaatggtcaaaagaaaagatagtcggggacgggccaagagatctggcagcggcgagggcagaggcagcctgctgacctgcggcgacgtggaggagaaccccggccccatggagaagaatcccctggctgcccccctgctgatcctgtggtttcacctggactgcgtgtcctctatcctgaatgtggaacagagcccacagagcctgcacgtgcaggagggcgactccaccaacttcacatgctcttttcctagctccaacttctacgccctgcactggtacagaaaggagaccgcaaagtccccagaggccctgttcgtgatgacactgaacggcgatgagaagaagaagggccgcatcagcgccaccctgaatacaaaggagggctactcctatctgtacatcaagggctcccagcctgaggactctgccacctatctgtgcgccctgtacaacaataacgatatgcggtttggcgccggcaccagactgacagtgaagccaaacatccagaatccagaccccgccgtgtatcagctgcgggacagcaagtctagcgataagagcgtgtgcctgttcaccgactttgattctcagacaaacgtgagccagtccaaggacagcgacgtgtacatcaccgacaagacagtgctggatatgagaagcatggacttcaagtctaacagcgccgtggcctggtccaataagtctgatttcgcctgcgccaatgcctttaataactccatcatccccgaggataccttctttccttctccagagtcctcttgtgacgtgaagctggtggagaagtctttcgagaccgatacaaacctgaattttcagaacctgagcgtgatcggcttcaggatcctgctgctgaaggtggccggctttaatctgctgatgaccctgaggctgtggagctcctgaaccggtccggagcatcttaccgccatttatacccatatttgttctgtttttcttgatttgggtatacatttaaatgttaataaaacaaaatggtggggcaatcatttacattttttgggatatgtaattactagttcaggtgtattgccacaagacaaacttgttaagaaactttcccgttatttacgctctgttcctgttaatcaacctctggattacaaaatttgtgaaagattgactgatattcttaactttgttgctccttttacgctgtgtggatttgctgctttattgcctctgtatcttgctattgcttcccgtacggctttcgttttctcctccttgtataaatcctggttgctgtctctttttgaggagttgtggcccgttgtccgtcaacgtggcgtggtgtgctctgtgtttgctgacgcaacccccactggctggggcattgccaccacctgtcaactcctttctgggactttcgctttccccctcccgatcgccacggcagaactcatcgccgcctgccttgcccgctgctggacaggggctaggttgctgggcactgataattccgtggtgttgtc

SEQ ID NO: 290 - protein, 881 aa, synthetic construct
  REGION 1..881: note = WT-SPa/CD8a-Ig like domain 1(co)/CD8astalk(co)/CD4 TM+IC/TCRba/WPREmut2 amino acid sequence
MALPVTALLLPLALLLHAARPSQFRVSPLDRTWNLGETVELKCQVLLSNPTSGCSWLFQPRGAAASPTFLLYLSQNKPKAAEGLDTQRFSGKRLGDTFVLTLSDFRRENEGYYFCSALSNSIMYFSHFVPVFLPAKPTTTPAPRPPTPAPTIASQPLSLRPEACRPAAGGAVHTRGLDFACDMALIVLGGVAGLLLFIGLGIFFCVRCRHRRRQAERMSQIKRLLSEKKTCQCPHRFQKTCSPIRAKRSGSGATNFSLLKQAGDVEENPGPMDSWTFCCVSLCILVAKHTDAGVIQSPRHEVTEMGQEVTLRCKPISGHNSLFWYRETMMRGLELLIYFNNNVPIDDSGMPEDRFSAKMPNASFSTLKIQPSEPRDSAVYFCASSPGSTDTQYFGPGTRLTVLEDLKNVFPPEVAVFEPSEAEISHTQKATLVCLATGFYPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSESYQQGVLSATILYEILLGKATLYAVLVSALVLMAMVKRKDSRGRAKRSGSGEGRGSLLTCGDVEENPGPMEKNPLAAPLLILWFHLDCVSSILNVEQSPQSLHVQEGDSTNFTCSFPSSNFYALHWYRKETAKSPEALFVMTLNGDEKKKGRISATLNTKEGYSYLYIKGSQPEDSATYLCALYNNNDMRFGAGTRLTVKPNIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 291 - DNA, 4419 bp, synthetic construct
  misc_feature 1..4419: note = PTE.CD8ab.TCR.WPREmut2 construct nucleotide sequence
  source 1..4419: mol_type = other DNA
tgaaagaccccacctgtaggtttggcaagctagcttaagtaacgccattttgcaaggcatggaaaatacataactgagaatagagaagttcagatcaaggttaggaacagagagacagcagaatatgggccaaacaggatatctgtggtaagcagttcctgccccggctcagggccaagaacagatggtccccagatgcggtcccgccctcagcagtttctagagaaccatcagatgtttccagggtgccccaaggacctgaaaatgaccctgtgccttatttgaactaaccaatcagttcgcttctcgcttctgttcgcgcgcttctgctccccgagctcaataaaagagcccacaacccctcactcagcggccgccccgggtcgacgctaccaccatgcgcccgagactgtggcttctgctcgccgcgcaactgactgtcctgcacggaaacagcgtgctgcagcagacaccggcctacatcaaagtgcagaccaacaagatggtcatgctgtcctgcgaggccaagatttccctctccaacatgcggatctattggttgcggcagagacaggcgccttcctcggactcccaccatgagttcttggccctgtgggactccgccaagggaactattcacggcgaagaagtggaacaggagaagatcgccgtgtttcgcgatgcctcccgctttatactgaatctgacctccgtgaagcccgaagatagcgggatctacttttgcatgattgtgggctcacccgaactgaccttcgggaagggcactcagctgagcgtggtggacttcctccccactaccgcccaacccactaagaagtcaaccctgaagaagcgggtttgcagactcccacggccggaaacgcagaagggtccgctgtgttccccgatcaccctggggctccttgtggctggagtgctggtccttctggtgtcccttggcgtcgccattcacctctgctgccggagaaggagggccagactgaggttcatgaagcagcctcagggagaggggatcagtggcactttcgtgccacaatgcctccatggctactattccaacaccaccacctcgcaaaagctgctgaacccctggatcctgaaaacccgggccaagagatctggcagcggcgccaccaatttcagcctgctgaaacaggccggcgacgtggaagagaaccctggccccatggcgcttcccgtgaccgcactcctgttgccccttgccctgctgttgcacgccgcacgaccttcccaattccgggtgtcccctctggatcgcacctggaacctcggggaaacggtggagctcaagtgtcaagtcctcctgtcgaacccgaccagcggatgcagctggctgttccagccgagaggagctgccgcctcacccaccttcctcctgtacttgagccagaacaagccgaaggccgctgagggtctggacacccagcgcttctcgggcaaacggctgggagacacttttgtgctgactctctccgacttccggcgggagaacgagggctactacttctgctctgcgctctccaattcaatcatgtacttctcacacttcgtgccggtgttcctgcctgccaagcccaccactactccggcacccagacctccaactcccgctcccaccatcgcgtcccaacccctttcgctgcgccctgaagcgtgtcggcctgctgctggaggagccgtgcatacccgcggtctggacttcgcgtgcgacatctacatttgggcccctttggctggcacctgtggagtgctgctcctgtcccttgtgatcaccctgtactgcaaccaccggaataggcggagagtctgcaagtgtccgcggcctgtcgtgaagtcaggagataagccgagcctgtccgcacgctacgtgcgggccaagagatctggcagcggcgagggcagaggcagcctgctgacctgcggcgacgtggaggagaaccccggccccatggactcttggaccttctgctgcgtgagcctgtgcatcctggtggccaagcacacagacgccggcgtgatccagtcccctaggcacgaggtgaccgagatgggccaggaggtgacactgcgctgtaagccaatctctggccacaacagcctgttttggtatagggagaccatgatgcgcggcctggagctgctgatctacttcaataacaatgtgcccatcgacgattccggcatgcctgaggatcggttttctgccaagatgcccaatgccagcttctccacactgaagatccagcctagcgagccaagagactccgccgtgtatttttgcgcctctagcccaggcagcaccgatacacagtacttcggaccaggaaccaggctgacagtgctggaggacctgaagaacgtgttcccccctgaggtggccgtgtttgagccctctgaggccgagatcagccacacccagaaggccaccctggtgtgcctggcaaccggcttctatcctgatcacgtggagctgtcctggtgggtgaacggcaaggaggtgcacagcggcgtgtccacagacccacagcccctgaaggagcagccagccctgaatgatagccggtattgcctgtcctctcggctgagagtgtccgccaccttttggcagaacccccggaatcacttcagatgtcaggtgcagttttacggcctgtccgagaacgatgagtggacccaggaccgggccaagcctgtgacacagatcgtgtctgccgaggcatggggaagagcagactgtggcttcacctctgagagctaccagcagggcgtgctgagcgccaccatcctgtatgagatcctgctgggcaaggccacactgtacgccgtcctggtctccgctctggtgctgatggcaatggtcaaaagaaaagatagtcggggacgggccaagagatctggcagcggccagtgcaccaactacgccctgctgaagctggccggcgacgtggagagcaaccccggccccatggagaagaatcccctggctgcccccctgctgatcctgtggtttcacctggactgcgtgtcctctatcctgaatgtggaacagagcccacagagcctgcacgtgcaggagggcgactccaccaacttcacatgctcttttcctagctccaacttctacgccctgcactggtacagaaaggagaccgcaaagtccccagaggccctgttcgtgatgacactgaacggcgatgagaagaagaagggccgcatcagcgccaccctgaatacaaaggagggctactcctatctgtacatcaagggctcccagcctgaggactctgccacctatctgtgcgccctgtacaacaataacgatatgcggtttggcgccggcaccagactgacagtgaagccaaacatccagaatccagaccccgccgtgtatcagctgcgggacagcaagtctagcgataagagcgtgtgcctgttcaccgactttgattctcagacaaacgtgagccagtccaaggacagcgacgtgtacatcaccgacaagacagtgctggatatgagaagcatggacttcaagtctaacagcgccgtggcctggtccaataagtctgatttcgcctgcgccaatgcctttaataactccatcatccccgaggataccttctttccttctccagagtcctcttgtgacgtgaagctggtggagaagtctttcgagaccgatacaaacctgaattttcagaacctgagcgtgatcggcttcaggatcctgctgctgaaggtggccggctttaatctgctgatgaccctgaggctgtggagctcctgaaccggtccggagcatcttaccgccatttatacccatatttgttctgtttttcttgatttgggtatacatttaaatgttaataaaacaaaatggtggggcaatcatttacattttttgggatatgtaattactagttcaggtgtattgccacaagacaaacttgttaagaaactttcccgttatttacgctctgttcctgttaatcaacctctggattacaaaatttgtgaaagattgactgatattcttaactttgttgctccttttacgctgtgtggatttgctgctttattgcctctgtatcttgctattgcttcccgtacggctttcgttttctcctccttgtataaatcctggttgctgtctctttttgaggagttgtggcccgttgtccgtcaacgtggcgtggtgtgctctgtgtttgctgacgcaacccccactggctggggcattgccaccacctgtcaactcctttctgggactttcgctttccccctcccgatcgccacggcagaactcatcgccgcctgccttgcccgctgctggacaggggctaggttgctgggcactgataattccgtggtgttgtc

SEQ ID NO: 292 - protein, 1143 aa, synthetic construct
  REGION 1..1143: note = PTE.CD8ab.TCR.WPREmut2 amino acid sequence
MRPRLWLLLAAQLTVLHGNSVLQQTPAYIKVQTNKMVMLSCEAKISLSNMRIYWLRQRQAPSSDSHHEFLALWDSAKGTIHGEEVEQEKIAVFRDASRFILNLTSVKPEDSGIYFCMIVGSPELTFGKGTQLSVVDFLPTTAQPTKKSTLKKRVCRLPRPETQKGPLCSPITLGLLVAGVLVLLVSLGVAIHLCCRRRRARLRFMKQPQGEGISGTFVPQCLHGYYSNTTTSQKLLNPWILKTRAKRSGSGATNFSLLKQAGDVEENPGPMALPVTALLLPLALLLHAARPSQFRVSPLDRTWNLGETVELKCQVLLSNPTSGCSWLFQPRGAAASPTFLLYLSQNKPKAAEGLDTQRFSGKRLGDTFVLTLSDFRRENEGYYFCSALSNSIMYFSHFVPVFLPAKPTTTPAPRPPTPAPTIASQPLSLRPEACRPAAGGAVHTRGLDFACDIYIWAPLAGTCGVLLLSLVITLYCNHRNRRRVCKCPRPVVKSGDKPSLSARYVRAKRSGSGEGRGSLLTCGDVEENPGPMDSWTFCCVSLCILVAKHTDAGVIQSPRHEVTEMGQEVTLRCKPISGHNSLFWYRETMMRGLELLIYFNNNVPIDDSGMPEDRFSAKMPNASFSTLKIQPSEPRDSAVYFCASSPGSTDTQYFGPGTRLTVLEDLKNVFPPEVAVFEPSEAEISHTQKATLVCLATGFYPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSESYQQGVLSATILYEILLGKATLYAVLVSALVLMAMVKRKDSRGRAKRSGSGQCTNYALLKLAGDVESNPGPMEKNPLAAPLLILWFHLDCVSSILNVEQSPQSLHVQEGDSTNFTCSFPSSNFYALHWYRKETAKSPEALFVMTLNGDEKKKGRISATLNTKEGYSYLYIKGSQPEDSATYLCALYNNNDMRFGAGTRLTVKPNIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 293 - protein, 21 aa, synthetic construct
  REGION 1..21: note = Signal peptide
MRPRLWLLLAAQLTVLHGNSV

SEQ ID NO: 294 - protein, 19 aa, synthetic construct
  REGION 1..19: note = Signal peptide
MEFGLSWLFLVAILKGVQC

SEQ ID NO: 295 - DNA, 3631 bp, synthetic construct
  misc_feature 1..3631: note = CD8a.TCR.WPRE construct nucleotide sequence
  source 1..3631: mol_type = other DNA
tgaaagaccccacctgtaggtttggcaagctagcttaagtaacgccattttgcaaggcatggaaaatacataactgagaatagagaagttcagatcaaggttaggaacagagagacagcagaatatgggccaaacaggatatctgtggtaagcagttcctgccccggctcagggccaagaacagatggtccccagatgcggtcccgccctcagcagtttctagagaaccatcagatgtttccagggtgccccaaggacctgaaaatgaccctgtgccttatttgaactaaccaatcagttcgcttctcgcttctgttcgcgcgcttctgctccccgagctcaataaaagagcccacaacccctcactagcggccgccccgggtcgacgctaccaccatggcgcttcccgtgaccgcactcctgttgccccttgccctgctgttgcacgccgcacgaccttcccaattccgggtgtcccctctggatcgcacctggaacctcggggaaacggtggagctcaagtgtcaagtcctcctgtcgaacccgaccagcggatgcagctggctgttccagccgagaggagctgccgcctcacccaccttcctcctgtacttgagccagaacaagccgaaggccgctgagggtctggacacccagcgcttctcgggcaaacggctgggagacacttttgtgctgactctctccgacttccggcgggagaacgagggctactacttctgctctgcgctctccaattcaatcatgtacttctcacacttcgtgccggtgttcctgcctgccaagcccaccactactccggcacccagacctccaactcccgctcccaccatcgcgtcccaacccctttcgctgcgccctgaagcgtgtcggcctgctgctggaggagccgtgcatacccgcggtctggacttcgcgtgcgacatctacatttgggcccctttggctggcacctgtggagtgctgctcctgtcccttgtgatcaccctgtactgcaaccaccggaataggcggagagtctgcaagtgtccgcggcctgtcgtgaagtcaggagataagccgagcctgtccgcacgctacgtgcgggccaagagatctggcagcggcgccaccaatttcagcctgctgaaacaggccggcgacgtggaagagaaccctggccccatggactcttggaccttctgctgcgtgagcctgtgcatcctggtggccaagcacacagacgccggcgtgatccagtcccctaggcacgaggtgaccgagatgggccaggaggtgacactgcgctgtaagccaatctctggccacaacagcctgttttggtatagggagaccatgatgcgcggcctggagctgctgatctacttcaataacaatgtgcccatcgacgattccggcatgcctgaggatcggttttctgccaagatgcccaatgccagcttctccacactgaagatccagcctagcgagccaagagactccgccgtgtatttttgcgcctctagcccaggcagcaccgatacacagtacttcggaccaggaaccaggctgacagtgctggaggacctgaagaacgtgttcccccctgaggtggccgtgtttgagccctctgaggccgagatcagccacacccagaaggccaccctggtgtgcctggcaaccggcttctatcctgatcacgtggagctgtcctggtgggtgaacggcaaggaggtgcacagcggcgtgtccacagacccacagcccctgaaggagcagccagccctgaatgatagccggtattgcctgtcctctcggctgagagtgtccgccaccttttggcagaacccccggaatcacttcagatgtcaggtgcagttttacggcctgtccgagaacgatgagtggacccaggaccgggccaagcctgtgacacagatcgtgtctgccgaggcatggggaagagcagactgtggcttcacctctgagagctaccagcagggcgtgctgagcgccaccatcctgtatgagatcctgctgggcaaggccacactgtacgccgtcctggtctccgctctggtgctgatggcaatggtcaaaagaaaagatagtcggggacgggccaagagatctggcagcggcgagggcagaggcagcctgctgacctgcggcgacgtggaggagaaccccggccccatggagaagaatcccctggctgcccccctgctgatcctgtggtttcacctggactgcgtgtcctctatcctgaatgtggaacagagcccacagagcctgcacgtgcaggagggcgactccaccaacttcacatgctcttttcctagctccaacttctacgccctgcactggtacagaaaggagaccgcaaagtccccagaggccctgttcgtgatgacactgaacggcgatgagaagaagaagggccgcatcagcgccaccctgaatacaaaggagggctactcctatctgtacatcaagggctcccagcctgaggactctgccacctatctgtgcgccctgtacaacaataacgatatgcggtttggcgccggcaccagactgacagtgaagccaaacatccagaatccagaccccgccgtgtatcagctgcgggacagcaagtctagcgataagagcgtgtgcctgttcaccgactttgattctcagacaaacgtgagccagtccaaggacagcgacgtgtacatcaccgacaagacagtgctggatatgagaagcatggacttcaagtctaacagcgccgtggcctggtccaataagtctgatttcgcctgcgccaatgcctttaataactccatcatccccgaggataccttctttccttctccagagtcctcttgtgacgtgaagctggtggagaagtctttcgagaccgatacaaacctgaattttcagaacctgagcgtgatcggcttcaggatcctgctgctgaaggtggccggctttaatctgctgatgaccctgaggctgtggagctcctgaaccggtccgcagtctgacgtacgcgtaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccatggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcc

SEQ ID NO: 296 - protein, 872 aa, synthetic construct; identical to SEQ ID NO: 288
  REGION 1..872: note = CD8a.TCR.WPRE amino acid sequence

SEQ ID NO: 297 - DNA, 4419 bp, synthetic construct; identical to SEQ ID NO: 291
  misc_feature 1..4419: note = CD8ba.TCR.WPRE nucleotide sequence
  source 1..4419: mol_type = other DNA

SEQ ID NO: 298 - protein, 1143 aa, synthetic construct; identical to SEQ ID NO: 292
  REGION 1..1143: note = CD8ba.TCR.WPRE amino acid sequence

SEQ ID NO: 299 - DNA, 7751 bp, synthetic construct
  misc_feature 1..7751: note = TCR.WPRE nucleotide sequence
  source 1..7751: mol_type = other DNA
aaacgccagcaacgcggcctttttacggttcctggccttttgctggccttttgctcacatgttcttgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccggggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagaggcctctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcggttaacttttaaaagaaaaggggggattggggggtacagtgcaggggaaagaatagtagacataatagcaacagacatacaaactaaagaattacaaaaacaaattacaaaaattcaaaattttatcgatcacgagactagcctcgaatctgcagaattcgcccttatcgattgaaagaccccacctgtaggtttggcaagctagcttaagtaacgccattttgcaaggcatggaaaatacataactgagaatagagaagttcagatcaaggttaggaacagagagacagcagaatatgggccaaacaggatatctgtggtaagcagttcctgccccggctcagggccaagaacagatggtccccagatgcggtcccgccctcagcagtttctagagaaccatcagatgtttccagggtgccccaaggacctgaaaatgaccctgtgccttatttgaactaaccaatcagttcgcttctcgcttctgttcgcgcgcttctgctccccgagctcaataaaagagcccacaacccctcactcagcggccgccccgggtcgacgctaccaccatggactcttggaccttctgctgcgtgagcctgtgcatcctggtggccaagcacacagacgccggcgtgatccagtcccctaggcacgaggtgaccgagatgggccaggaggtgacactgcgctgtaagccaatctctggccacaacagcctgttttggtatagggagaccatgatgcgcggcctggagctgctgatctacttcaataacaatgtgcccatcgacgattccggcatgcctgaggatcggttttctgccaagatgcccaatgccagcttctccacactgaagatccagcctagcgagccaagagactccgccgtgtatttttgcgcctctagcccaggcagcaccgatacacagtacttcggaccaggaaccaggctgacagtgctggaggacctgaagaacgtgttcccccctgaggtggccgtgtttgagccctctgaggccgagatcagccacacccagaaggccaccctggtgtgcctggcaaccggcttctatcctgatcacgtggagctgtcctggtgggtgaacggcaaggaggtgcacagcggcgtgtccacagacccacagcccctgaaggagcagccagccctgaatgatagccggtattgcctgtcctctcggctgagagtgtccgccaccttttggcagaacccccggaatcacttcagatgtcaggtgcagttttacggcctgtccgagaacgatgagtggacccaggaccgggccaagcctgtgacacagatcgtgtctgccgaggcatggggaagagcagactgtggcttcacctctgagagctaccagcagggcgtgctgagcgccaccatcctgtatgagatcctgctgggcaaggccacactgtacgccgtcctggtctccgctctggtgctgatggcaatggtcaaaagaaaagatagtcggggatctggcagcggcgccaccaatttcagcctgctgaaacaggccggcgacgtggaagagaaccctggccccatggagaagaatcccctggctgcccccctgctgatcctgtggtttcacctggactgcgtgtcctctatcctgaatgtggaacagagcccacagagcctgcacgtgcaggagggcgactccaccaacttcacatgctcttttcctagctccaacttctacgccctgcactggtacagaaaggagaccgcaaagtccccagaggccctgttcgtgatgacactgaacggcgatgagaagaagaagggccgcatcagcgccaccctgaatacaaaggagggctactcctatctgtacatcaagggctcccagcctgaggactctgccacctatctgtgcgccctgtacaacaataacgatatgcggtttggcgccggcaccagactgacagtgaagccaaacatccagaatccagaccccgccgtgtatcagctgcgggacagcaagtctagcgataagagcgtgtgcctgttcaccgactttgattctcagacaaacgtgagccagtccaaggacagcgacgtgtacatcaccgacaagacagtgctggatatgagaagcatggacttcaagtctaacagcgccgtggcctggtccaataagtctgatttcgcctgcgccaatgcctttaataactccatcatccccgaggataccttctttccttctccagagtcctcttgtgacgtgaagctggtggagaagtctttcgagaccgatacaaacctgaattttcagaacctgagcgtgatcggcttcaggatcctgctgctgaaggtggccggctttaatctgctgatgaccctgaggctgtggagctcctgaaccggtccgcagtctgacgtacgcgtaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccatggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcccgtacgtgtacattacgcgcctaggatttaaatccactcctgacaactactctttaatgcacgaggtgtcagtaggtgaaggagtcgtagttgtcgataagcagcaacatcttactacactggtctcaactccggatccgagctcggtacctttaagaccaatgacttacaaggcagctgtagatcttagccactttttaaaagaaaaggggggactggaagggctaattcactcccaacgaagacaagatctgctttttgcttgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagcatctagagggcccgtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctactgggcggttttatggacagcaagcgaaccggaattgccagctggggcgccctctggtaaggttgggaagccctgcaaagtaaactggatggctttcttgccgccaaggatctgatggcgcaggggatcaagctctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaagacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgagcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgaattattaacgcttacaatttcctgatgcggtattttctccttacgcatctgtgcggtatttcacaccgcatcaggtggcacttttcggggaaatgtgcgcggaacccctatttgtttatttttctaaatacattcaaatatgtatccgctcatgagacaataaccctgataaatgcttcaataatagcacgtgctaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaa

SEQ ID NO: 300 - protein, 607 aa, synthetic construct
  REGION 1..607: note = TCR.WPRE amino acid sequence
MDSWTFCCVSLCILVAKHTDAGVIQSPRHEVTEMGQEVTLRCKPISGHNSLFWYRETMMRGLELLIYFNNNVPIDDSGMPEDRFSAKMPNASFSTLKIQPSEPRDSAVYFCASSPGSTDTQYFGPGTRLTVLEDLKNVFPPEVAVFEPSEAEISHTQKATLVCLATGFYPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSESYQQGVLSATILYEILLGKATLYAVLVSALVLMAMVKRKDSRGSGSGATNFSLLKQAGDVEENPGPMEKNPLAAPLLILWFHLDCVSSILNVEQSPQSLHVQEGDSTNFTCSFPSSNFYALHWYRKETAKSPEALFVMTLNGDEKKKGRISATLNTKEGYSYLYIKGSQPEDSATYLCALYNNNDMRFGAGTRLTVKPNIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 301 - DNA, 3828 bp, synthetic construct
  misc_feature 1..3828: note = CD8aNCAM1Fusion.TCR.WPREmut2 nucleotide sequence
  source 1..3828: mol_type = other DNA
tgaaagaccccacctgtaggtttggcaagctagcttaagtaacgccattttgcaaggcatggaaaatacataactgagaatagagaagttcagatcaaggttaggaacagagagacagcagaatatgggccaaacaggatatctgtggtaagcagttcctgccccggctcagggccaagaacagatggtccccagatgcggtcccgccctcagcagtttctagagaaccatcagatgtttccagggtgccccaaggacctgaaaatgaccctgtgccttatttgaactaaccaatcagttcgcttctcgcttctgttcgcgcgcttctgctccccgagctcaataaaagagcccacaacccctcactcagcggccgccccgggtcgacgctaccaccatggcgcttcccgtgaccgcactcctgttgccccttgccctgctgttgcacgccgcacgaccttcccaattccgggtgtcccctctggatcgcacctggaacctcggggaaacggtggagctcaagtgtcaagtcctcctgtcgaacccgaccagcggatgcagctggctgttccagccgagaggagctgccgcctcacccaccttcctcctgtacttgagccagaacaagccgaaggccgctgagggtctggacacccagcgcttctcgggcaaacggctgggagacacttttgtgctgactctctccgacttccggcgggagaacgagggctactacttctgctctgcgctctccaattcaatcatgtacttctcacacttcgtgccggtgttcctgcctgccagcgtggtggacttcctccccactaccgcccaacccactaagaagtcaaccctgaagaagcgggtttgcagactcccacggccggaaacgcagaagggtccgctgtgttccccggccatcgtgggcatcctgatcgtgatcttcgtgctgctgctggtggtggtggacatcacctgctacttcctgaacaagtgcggcctgttcatgtgcatcgccgtgaacctgtgcggcaaggccggccccggcgccaagggcaaggacatggaggagggcaaggccgccttcagcaaggacgagagcaaggagcccatcgtggaggtgaggaccgaggaggagaggacccccaaccacgacggcggcaagcacaccgagcccaacgagaccacccccctgaccgagcccgagaagggccccgtggaggccaagcccgagtgccaggagaccgagaccaagcccgcccccgccgaggtgaagaccgtgcccaacgacgccacccagaccaaggagaacgagagcaaggcccgggccaagagatctggcagcggcgccaccaatttcagcctgctgaaacaggccggcgacgtggaagagaaccctggccccatggactcttggaccttctgctgcgtgagcctgtgcatcctggtggccaagcacacagacgccggcgtgatccagtcccctaggcacgaggtgaccgagatgggccaggaggtgacactgcgctgtaagccaatctctggccacaacagcctgttttggtatagggagaccatgatgcgcggcctggagctgctgatctacttcaataacaatgtgcccatcgacgattccggcatgcctgaggatcggttttctgccaagatgcccaatgccagcttctccacactgaagatccagcctagcgagccaagagactccgccgtgtatttttgcgcctctagcccaggcagcaccgatacacagtacttcggaccaggaaccaggctgacagtgctggaggacctgaagaacgtgttcccccctgaggtggccgtgtttgagccctctgaggccgagatcagccacacccagaaggccaccctggtgtgcctggcaaccggcttctatcctgatcacgtggagctgtcctggtgggtgaacggcaaggaggtgcacagcggcgtgtccacagacccacagcccctgaaggagcagccagccctgaatgatagccggtattgcctgtcctctcggctgagagtgtccgccaccttttggcagaacccccggaatcacttcagatgtcaggtgcagttttacggcctgtccgagaacgatgagtggacccaggaccgggccaagcctgtgacacagatcgtgtctgccgaggcatggggaagagcagactgtggcttcacctctgagagctaccagcagggcgtgctgagcgccaccatcctgtatgagatcctgctgggcaaggccacactgtacgccgtcctggtctccgctctggtgctgatggcaatggtcaaaagaaaagatagtcggggacgggccaagagatctggcagcggcgagggcagaggcagcctgctgacctgcggcgacgtggaggagaaccccggccccatggagaagaatcccctggctgcccccctgctgatcctgtggtttcacctggactgcgtgtcctctatcctgaatgtggaacagagcccacagagcctgcacgtgcaggagggcgactccaccaacttcacatgctcttttcctagctccaacttctacgccctgcactggtacagaaaggagaccgcaaagtccccagaggccctgttcgtgatgacactgaacggcgatgagaagaagaagggccgcatcagcgccaccctgaatacaaaggagggctactcctatctgtacatcaagggctcccagcctgaggactctgccacctatctgtgcgccctgtacaacaataacgatatgcggtttggcgccggcaccagactgacagtgaagccaaacatccagaatccagaccccgccgtgtatcagctgcgggacagcaagtctagcgataagagcgtgtgcctgttcaccgactttgattctcagacaaacgtgagccagtccaaggacagcgacgtgtacatcaccgacaagacagtgctggatatgagaagcatggacttcaagtctaacagcgccgtggcctggtccaataagtctgatttcgcctgcgccaatgcctttaataactccatcatccccgaggataccttctttccttctccagagtcctcttgtgacgtgaagctggtggagaagtctttcgagaccgatacaaacctgaattttcagaacctgagcgtgatcggcttcaggatcctgctgctgaaggtggccggctttaatctgctgatgaccctgaggctgtggagctcctgaaccggtccggagcatcttaccgccatttatacccatatttgttctgtttttcttgatttgggtatacatttaaatgttaataaaacaaaatggtggggcaatcatttacattttttgggatatgtaattactagttcaggtgtattgccacaagacaaacttgttaagaaactttcccgttatttacgctctgttcctgttaatcaacctctggattacaaaatttgtgaaagattgactgatattcttaactttgttgctccttttacgctgtgtggatttgctgctttattgcctctgtatcttgctattgcttcccgtacggctttcgttttctcctccttgtataaatcctggttgctgtctctttttgaggagttgtggcccgttgtccgtcaacgtggcgtggtgtgctctgtgtttgctgacgcaacccccactggctggggcattgccaccacctgtcaactcctttctgggactttcgctttccccctcccgatcgccacggcagaactcatcgccgcctgccttgcccgctgctggacaggggctaggttgctgggcactgataattccgtggtgttgtc

SEQ ID NO: 302 - protein, 946 aa, synthetic construct
  REGION 1..946: note = CD8aNCAM1Fusion.TCR.WPREmut2 amino acid sequence
MALPVTALLLPLALLLHAARPSQFRVSPLDRTWNLGETVELKCQVLLSNPTSGCSWLFQPRGAAASPTFLLYLSQNKPKAAEGLDTQRFSGKRLGDTFVLTLSDFRRENEGYYFCSALSNSIMYFSHFVPVFLPASVVDFLPTTAQPTKKSTLKKRVCRLPRPETQKGPLCSPAIVGILIVIFVLLLVVVDITCYFLNKCGLFMCIAVNLCGKAGPGAKGKDMEEGKAAFSKDESKEPIVEVRTEEERTPNHDGGKHTEPNETTPLTEPEKGPVEAKPECQETETKPAPAEVKTVPNDATQTKENESKARAKRSGSGATNFSLLKQAGDVEENPGPMDSWTFCCVSLCILVAKHTDAGVIQSPRHEVTEMGQEVTLRCKPISGHNSLFWYRETMMRGLELLIYFNNNVPIDDSGMPEDRFSAKMPNASFSTLKIQPSEPRDSAVYFCASSPGSTDTQYFGPGTRLTVLEDLKNVFPPEVAVFEPSEAEISHTQKATLVCLATGFYPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSESYQQGVLSATILYEILLGKATLYAVLVSALVLMAMVKRKDSRGRAKRSGSGEGRGSLLTCGDVEENPGPMEKNPLAAPLLILWFHLDCVSSILNVEQSPQSLHVQEGDSTNFTCSFPSSNFYALHWYRKETAKSPEALFVMTLNGDEKKKGRISATLNTKEGYSYLYIKGSQPEDSATYLCALYNNNDMRFGAGTRLTVKPNIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 303 - protein, 311 aa, synthetic construct; identical to SEQ ID NO: 16
  REGION 1..311: note = R11P3D3KE beta chain

SEQ ID NO: 304 - protein, 273 aa, synthetic construct
  REGION 1..273: note = R39P1C12 alpha chain
MKTFAGFSFLFLWLQLDCMSRGEDVEQSLFLSVREGDSSVINCTYTDSSSTYLYWYKQEPGAGLQLLTYIFSNMDMKQDQRLTVLLNKKDKHLSLRIADTQTGDSAIYFCAEIDNQGGKLIFGQGTELSVKPNIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

(19) United States
(12) Patent Application Publication
(10) Pub. No.: US 2025/0146019 A1
(43) Pub. Date: May 8, 2025

(54) CD8 POLYPEPTIDES, COMPOSITIONS, AND METHODS OF USING THEREOF

(71) Applicant: Immatics US, Inc., Stafford, TX (US)

(72) Inventors: Gagan BAJWA, Richmond, TX (US); Mamta KALRA, Stafford, TX (US); Melinda MATA, Missouri City, TX (US)

(21) Appl. No.: 19/020,283

(22) Filed: Jan. 14, 2025

Related U.S. Application Data
(63) Continuation of application No. 17/563,599, filed on Dec. 28, 2021, now Pat. No. 12,234,473.
(60) Provisional application No. 63/247,775, filed on Sep. 23, 2021.
(60) Provisional application No. 63/132,824, filed on Dec. 31, 2020.

(30) Foreign Application Priority Data
Jan. 4, 2021 (DE) 102021100038.6

(51) Int. Cl. C12N 15/86; A61K 40/11; A61K 40/32; A61K 40/42; C07K 14/705; C07K 14/725; C12N 5/0783

(52) U.S. Cl. CPC C12N 15/86; A61K 40/11; A61K 40/32; A61K 40/421; A61K 40/427; C07K 14/7051; C07K 14/70517; C12N 5/0636; C12N 2810/55

(57) ABSTRACT

The present disclosure relates to T cells capable of co-expressing T cell receptors (“TCR”) together with CD8 polypeptides and the use thereof in adoptive cellular therapy. The present disclosure further provides for modified CD8 sequences, vectors, and associated methods thereof.

### CROSS-REFERENCE TO RELATED APPLICATIONS

This application is a Continuation Application of U.S. application Ser. No. 17/563,599, filed 28 Dec. 2021, which claims priority to U.S. Provisional Patent Application No. 63/132,824, filed 31 Dec. 2020, U.S. Provisional Patent Application No. 63/247,775, filed 23 Sep. 2021 and German Patent Application No. 10 2021 100 038.6, filed 4 Jan. 2021, the entire contents of each of which are incorporated herein by reference.

### REFERENCE TO SEQUENCE LISTING SUBMITTED AS AN XML FILE

Pursuant to the EFS-Web legal framework and 37 C.F.R. § 1.831-1.834 (see M.P.E.P. § 2412), a Sequence Listing in the form of an XML file. entitled “3000011-022003_Sequence_Listing_ST26” created on 13 Jan. 2025, and 432,976 bytes in size, is submitted concurrently with the instant application, and the entire contents of the Sequence Listing are incorporated herein by reference.

### BACKGROUND

### Field

The present disclosure relates to T cells capable of co-expressing T cell receptors (“TCR”) together with CD8 polypeptides and the use thereof in adoptive cellular therapy. The present disclosure further provides for modified CD8 sequences, vectors, compositions, transformed T cells, and associated methods thereof.

CD8 and CD4 are transmembrane glycoproteins characteristic of distinct populations of T lymphocytes whose antigen responses are restricted by class I and class II MHC molecules, respectively. They play major roles both in the differentiation and selection of T cells during thymic development and in the activation of mature T lymphocytes in response to antigen presenting cells. Both CD8 and CD4 are immunoglobulin superfamily proteins. They determine antigen restriction by binding to MHC molecules at an interface distinct from the region presenting the antigenic peptide, but the structural basis for their similar functions appears to be very different. Their sequence similarity is low and, whereas CD4 is expressed on the cell surface as a monomer, CD8 is expressed as an aa homodimer (e.g., FIG. 55C) or an αβ heterodimer (e.g., FIG. 55A). In humans, this CD8αα homodimer may functionally substitute for the CD8αβ heterodimer. CD8 contacts an acidic loop in the α3 domain of Class I MHC, thereby increasing the avidity of the T cell for its target. CD8 is also involved in the phosphorylation events leading to CTL activation through the association of its α chain cytoplasmic tail with the tyrosine kinase p56^{lck}.

It is desirable to develop methods of manufacturing T cells with enhanced, specific cytotoxic activity for immunotherapy.

### SUMMARY OF THE INVENTION

In an embodiment, CD8 polypeptides described herein may comprise a CD8α immunoglobulin (Ig)-like domain, a CD8β region, a CD8α transmembrane domain, and a CD8α cytoplasmic domain. In another embodiment, the CD8β region is a CD8β stalk region or domain.

In an embodiment, CD8 polypeptides described herein may comprise (a) an immunoglobulin (Ig)-like domain comprising at least about 80%, 85%, 90%, 91%, 92%, 93%, 94%, 95%, 96%, 97%, 98%, 99%, or 100% sequence identity to the amino acid sequence of SEQ ID NO: 1, (b) a CD8β region comprising at least about 80%, 85%, 90%, 91%, 92%, 93%, 94%, 95%, 96%, 97%, 98%, 99%, or 100% identity sequence identity to the amino acid sequence of SEQ ID NO: 2, (c) a transmembrane domain comprising at least about 80%, 85%, 90%, 91%, 92%, 93%, 94%, 95%, 96%, 97%, 98%, 99%, or 100% sequence identity to the amino acid sequence of SEQ ID NO: 3, and (d) a cytoplasmic domain comprising at least about 80%, 85%, 90%, 91%, 92%, 93%, 94%, 95%, 96%, 97%, 98%, 99%, or 100% sequence identity to the amino acid sequence of SEQ ID NO: 4.

In an embodiment, CD8 polypeptides described herein have at least about 80%, 85%, 90%, 91%, 92%, 93%, 94%, 95%, 96%, 97%, 98%, 99%, or 100% sequence identity to the amino acid sequence of SEQ ID NO: 5.

In an embodiment, CD8 polypeptides described herein have at least about 80%, 85%, 90%, 91%, 92%, 93%, 94%, 95%, 96%, 97%, 98%, 99%, or 100% sequence identity to the amino acid sequence of SEQ ID NO: 7.

In an embodiment, the CD8 polypeptides described herein may comprise a signal peptide with at least about 80%, 85%, 90%, 91%, 92%, 93%, 94%, 95%, 96%, 97%, 98%, 99%, or 100% sequence identity to the amino acid sequence of any one of SEQ ID NO: 6, SEQ ID NO: 293, or SEQ ID NO: 294 fused to the N-terminus or to the C-terminus of CD8 polypeptides described herein.

In an embodiment, CD8 polypeptides described herein may comprise (a) SEQ ID NO: 1 comprising one, two, three, four, or five amino acid substitutions; (b) SEQ ID NO: 2 comprising one, two, three, four, or five amino acid substitutions; (c) SEQ ID NO: 3 comprising one, two, three, four, or five amino acid substitutions, and (d) SEQ ID NO: 4 comprising one, two, three, four, or five amino acid substitutions.

In an embodiment, CD8 polypeptides described herein may be CD8α or modified CD8α polypeptides.

In an embodiment, the disclosure provides for nucleic acids encode polypeptides described herein.

In an embodiment, a vector may comprise a nucleic acid encoding CD8 polypeptides described herein.

In an embodiment, the vector may comprise a nucleic acid encoding T cell receptor (TCR) comprising an α chain and a β chain. In another embodiment, the vector may comprise a nucleic acid encoding a CAR-T.

In an embodiment, TCR α chain and TCR β chain may be selected from SEQ ID NO: 15 and 16; 17 and 18; 19 and 20; 21 and 22; 23 and 24; 25 and 26; 27 and 28; 29 and 30; 31 and 32; 33 and 34; 35 and 36; 37 and 38; 39 and 40; 41 and 42; 43 and 44; 45 and 46; 47 and 48; 49 and 50; 51 and 52; 53 and 54; 55 and 56; 57 and 58; 59 and 60; 61 and 62; 63 and 64; 65 and 66; 67 and 68; 69 and 70; 71 and 303; 304 and 74; 75 and 76; 77 and 78; 79 and 80; 81 and 82; 83 and 84; 85 and 86; 87 and 88; 89 and 90; and 91 and 92.

In an embodiment, the vector may comprise a nucleic acid encoding a CD8β polypeptide.

In an embodiment, CD8β polypeptide may comprise the amino acid sequence of any one of SEQ ID NO: 8, 9, 10, 11, 12, 13, or 14.

In an embodiment, the vector may comprise nucleic acid encoding a 2A peptide or an internal ribosome entry site (IRES) positioned between the nucleic acid encoding the modified CD8α polypeptide and the nucleic acid encoding a CD8β polypeptide.

In an embodiment, the vector may comprise nucleic acid encoding a 2A peptide positioned between the nucleic acid encoding a TCR α chain and the nucleic acid encoding a TCR β chain.

In an embodiment, the 2A peptide may be selected from P2A (SEQ ID NO: 93), T2A (SEQ ID NO: 94), E2A (SEQ ID NO: 95), or F2A (SEQ ID NO: 96).

In an embodiment, the IRES may be selected from the group consisting of IRES from picornavirus, IRES from flavivirus, IRES from pestivirus, IRES from retrovirus, IRES from lentivirus, IRES from insect RNA virus, and IRES from cellular mRNA.

In an embodiment, the vector may further comprise a post-transcriptional regulatory element (PRE) sequence selected from a Woodchuck PRE (WPRE) and variants thereof, a hepatitis B virus (HBV) PRE (HPRE), or a combination thereof.

In an embodiment, the vector may further comprise a promoter selected from cytomegalovirus (CMV) promoter, phosphoglycerate kinase (PGK) promoter, myelin basic protein (MBP) promoter, glial fibrillary acidic protein (GFAP) promoter, modified MoMuLV LTR comprising myeloproliferative sarcoma virus enhancer (MNDU3), Ubiqitin C promoter, EF-1 alpha promoter, Murine Stem Cell Virus (MSCV) promoter, or a combination thereof.

In an embodiment, the vector may be a viral vector or a non-viral vector.

In an embodiment, the vector may be selected from adenoviruses, poxviruses, alphaviruses, arenaviruses, flaviruses, rhabdoviruses, retroviruses, lentiviruses, herpesviruses, paramyxoviruses, picornaviruses, or a combination thereof.

In an embodiment, the vector may be pseudotyped with an envelope protein of a virus selected from the native feline endogenous virus (RD114), a chimeric version of RD114 (RD114TR), gibbon ape leukemia virus (GALV), a chimeric version of GALV (GALV-TR), amphotropic murine leukemia virus (MLV 4070A), baculovirus (GP64), vesicular stomatitis virus (VSV-G), fowl plague virus (FPV), Ebola virus (EboV), or baboon retroviral envelope glycoprotein (BaEV), lymphocytic choriomeningitis virus (LCMV), or a combination thereof.

In an embodiment, the vector may further comprise a nucleic acid encoding a T cell receptor (TCR).

In another embodiment, the vector may further comprise a nucleic acid encoding a chimeric antigen receptor (CAR).

In an embodiment, an isolated nucleic acid may comprise a nucleic acid sequence encoding a T-cell receptor comprising an α chain and a β chain and a CD8 polypeptide comprising an α chain and a β chain. The isolated nucleic acid may comprise a nucleic acid at least 80% identical to the nucleic acid sequence of SEQ ID NO: 267, 269, 271, 273, 275, 277, 279, 281, 283, 285, 287, 289, 291, 295, 297, 299, or 301. The isolated nucleic acid may be at least 90%, 91%, 92%, 93%, 94%, 95%, 96%, 97%, 98%, 99%, or 100% identical to the nucleic acid sequence of SEQ ID NO: 267, 269, 271, 273, 275, 277, 279, 281, 283, 285, 287, 289, 291, 295, 297, 299, or 301. In an aspect, sequences described herein may be isolated or recombinant sequences.

In an embodiment, the isolated nucleic acid comprises the nucleic acid sequence of SEQ ID NO: 267.

In an embodiment, the isolated nucleic acid comprises the nucleic acid sequence of SEQ ID NO: 279.

In an embodiment, the isolated polypeptide(s) may be encoded by the nucleic acids described herein.

In an embodiment, the isolated polypeptide may comprise the amino acid sequence at least about 80% identical to the amino acid sequence of SEQ ID NO: 268, 270, 272, 274, 276, 278, 280, 282, 284, 286, 288, 290, 292, 296, 298, 300, or 302. The amino acid sequence may be at least 90%, 91%, 92%, 93%, 94%, 95%, 96%, 97%, 98%, 99%, or 100% identical to the amino acid sequence of SEQ ID NO: 268, 270, 272, 274, 276, 278, 280, 282, 284, 286, 288, 290, 292, 296, 298, 300, or 302. In another aspect, SEQ ID NO: 268, 270, 272, 274, 276, 278, 280, 282, 284, 286, 288, 290, 292, 296, 298, 300, or 302 comprise 1, 2, 3, 4, 5, 10, 15, or 20 or more amino acid substitutions or deletions. In yet another aspect, SEQ ID NO: 268, 270, 272, 274, 276, 278, 280, 282, 284, 286, 288, 290, 292, 296, 298, 300, or 302 comprise at most 1, 2, 3, 4, 5, 10, 15, or 20 amino acid substitutions or deletions.

In an embodiment, the isolated polypeptide may comprise the amino acid sequence of SEQ ID NO: 268.

In an embodiment, the isolated polypeptide may comprise the amino acid sequence of SEQ ID NO: 280. In an embodiment, a cell may be transduced with the vector.

In an embodiment, the cell may comprise αβ T cell, γδ T cell, natural killer cell, CD4+/CD8+ cell, or combinations thereof.

In an embodiment, αβ T cell may comprise CD4+ T cell and CD8+ T cell.

In an embodiment, a method of preparing T cells for immunotherapy may comprise isolating T cells from a blood sample of a human subject, activating the isolated T cells, transducing the activated T cells with the vector, and expanding the transduced T cells.

In an embodiment, the T cell may be CD4+ T cell.

In an embodiment, the T cell may be CD8+ T cell.

In an embodiment, the T cell may be γδ T cell.

In an embodiment, the T cells may be a αβ T cell and express a CD8 polypeptide described herein.

In an embodiment, the T cells may be a γδ T cell and express a modified CD8 polypeptide described herein, for example, a modified CD8α polypeptide or a modified CD8α polypeptide with a CD8β stalk region, e.g., m1CD8α in Constructs #11 and #12 (FIG. 4) and CD8α* (FIG. 55B).

In an embodiment, a method of treating a patient who has cancer may comprise administering to the patient a composition comprising the population of expanded T cells, wherein the T cells kill cancer cells that present a peptide in a complex with an MHC molecule on the surface, wherein the peptide is selected from SEQ ID NO: 98-255, wherein the cancer is selected from the group consisting of non-small cell lung cancer, small cell lung cancer, melanoma, liver cancer, breast cancer, uterine cancer, Merkel cell carcinoma, pancreatic cancer, gallbladder cancer, bile duct cancer, colorectal cancer, urinary bladder cancer, kidney cancer, leukemia, ovarian cancer, esophageal cancer, brain cancer, gastric cancer, prostate cancer, or a combination thereof.

In an embodiment, the composition may further comprise an adjuvant.

In an embodiment, the adjuvant may be selected from anti-CD40 antibody, imiquimod, resiquimod, GM-CSF, cyclophosphamide, sunitinib, bevacizumab, atezolizumab, interferon-alpha, interferon-beta, CpG oligonucleotides and derivatives, poly(I:C) and derivatives, RNA, sildenafil, particulate formulations with poly(lactide co-glycolide) (PLG), virosomes, interleukin (IL)-1, IL-2, IL-4, IL-7, IL-12, IL-13, IL-15, IL-21, IL-23, or combinations thereof.

In an embodiment, a method of eliciting an immune response in a patient who has cancer may comprise administering to the patient a composition comprising the population of expanded T cells, wherein the T cells kill cancer cells that present a peptide in a complex with an MHC molecule on the surface, wherein the peptide is selected from SEQ ID NO: 98-255, wherein the cancer is selected from the group consisting of non-small cell lung cancer, small cell lung cancer, melanoma, liver cancer, breast cancer, uterine cancer, Merkel cell carcinoma, pancreatic cancer, gallbladder cancer, bile duct cancer, colorectal cancer, urinary bladder cancer, kidney cancer, leukemia, ovarian cancer, esophageal cancer, brain cancer, gastric cancer, prostate cancer, or a combination thereof.

The disclosure further provides for a population of modified T cells that present an exogenous CD8 co-receptor comprising a polypeptide described herein, for example, amino acid sequences at least 80%, at least 85%, at least 90%, or at least 95%, at least 99%, or 100% to SEQ ID NO: 5, 7, 258, 259, 8, 9, 10, 11, 12, 13, or 14 and a T cell receptor.

### BRIEF DESCRIPTION OF THE DRAWINGS

FIG. 1 shows a representative CD8α subunit, e.g., SEQ ID NO: 258 (CD8α1). In this embodiment, CD8α1 includes five domains: (1) signal peptide, (2) Ig-like domain-1, (3) a stalk region, (4) transmembrane (TM) domain, and (5) a cytoplasmic tail (Cyto) comprising a lck-binding motif.

FIG. 2 shows a sequence alignment between CD8α1 (SEQ ID NO: 258) and m1CD8α (SEQ ID NO: 7).

FIG. 3 shows a sequence alignment between CD8α2 (SEQ ID NO: 259) and m2CD8α (SEQ ID NO: 262), in which the cysteine substitution at position 112 is indicated by an arrow.

FIG. 4 shows vectors according to an aspect of the disclosure.

FIG. 5A shows titers of viral vectors shown in FIG. 4.

FIG. 5B shows titers of further viral vectors in accordance with an embodiment of the present disclosure. Construct #13; Construct #14; Construct #15; Construct #16; Construct #17; Construct #18; Construct #19; Construct #21; Construct #10n; Construct #11n; and TCR: R11KEA (SEQ ID NO: 15 and SEQ ID NO: 16) (Construct #8), which binds PRAME-004 (SLLQHLIGL) (SEQ ID NO: 147). Note that Constructs #10 and #10n are different batches of the same construct (SEQ ID NO: 291 and 292) and Constructs #11 and #11n are different batches of the same construct (SEQ ID NO: 285 and 286).

FIG. 6 shows T cell manufacturing.

FIG. 7A shows expression of activation markers before and after activation in CD3+ CD8+ cells.

FIG. 7B shows expression of activation markers before and after activation in CD3+ CD4+ cells.

FIG. 8A shows fold expansion of cells transduced with various constructs from Donor #1. The constructs are as follows: Construct #9b; Construct #10; Construct #11; Construct #12; Construct #1; Construct #2; TCR=R11KEA.WPRE^{wt} (TCR with wild type WPRE); NT=Non-transduced T cells (as a negative control). Note that Constructs #9 and #9b are different batches of the same construct (SEQ ID NO: 287 and 288).

FIG. 8B shows fold expansion of cells transduced with various constructs from Donor #2. The constructs are as follows: Construct #9b; Construct #10; Construct #11; Construct #12; Construct #1; Construct #2; TCR=R11KEA.WPRE^{wt} (TCR with wild type WPRE) (Construct #8); NT=Non-transduced T cells (as a negative control).

FIG. 9A shows flow plots of cells transduced with Construct #9.

FIG. 9B shows flow plots of cells transduced with Construct #10 in accordance with one embodiment of the present disclosure.

FIG. 9C shows flow plots of cells transduced with Construct #11.

FIG. 9D shows flow plots of cells transduced with Construct #12.

FIG. 10 shows % CD8+CD4+ of cells transduced with various constructs for Donor #1 and Donor #2. The constructs are as follows: Construct #9b; Construct #10; Construct #11; Construct #12; Construct #1; Construct #2; TCR=R11KEA.WPRE^{wt} TCR with wild type WPRE); NT=Non-transduced T cells (as a negative control).

FIG. 11 shows % Tet of CD8+CD4+ of cells transduced with various constructs. The constructs are as follows: Construct #9b; Construct #10; Construct #11; Construct #12; Construct #1; Construct #2; TCR=R11KEA.WPRE^{wt} (TCR with wild type WPRE); NT=Non-transduced T cells (as a negative control).

FIG. 12 shows Tet MFI (CD8+CD4+Tet+) of cells transduced with various constructs. The constructs are as follows: Construct #9b; Construct #10; Construct #11; Construct #12; Construct #1; Construct #2; TCR=R11KEA.WPRE^{wt} (TCR with wild type WPRE); NT=Non-transduced T cells (as a negative control).

FIG. 13 shows CD8α MFI (CD8+CD4+Tet+) of cells transduced with various constructs. The constructs are as follows: Construct #9b; Construct #10; Construct #11; Construct #12; Construct #1; Construct #2; TCR=R11KEA.WPRE^{wt} (TCR with wild type WPRE); NT=Non-transduced T cells (as a negative control).

FIG. 14 shows % CD8+CD4 (of CD3+) of cells transduced with various constructs. The constructs are as follows: Construct #9b; Construct #10; Construct #11; Construct #12; Construct #1; Construct #2; TCR=R11KEA.WPRE^{wt} (TCR with wild type WPRE); NT=Non-transduced T cells (as a negative control).

FIG. 15 shows % CD8+Tet+(of CD3+) of cells transduced with various constructs. The constructs are as follows: Construct #9b; Construct #10; Construct #11; Construct #12; Construct #1; Construct #2; TCR=R11KEA.WPRE^{wt} TCR with wild type WPRE); NT=Non-transduced T cells (as a negative control).

FIG. 16 shows Tet MFI (CD8+Tet+) of cells transduced with various constructs. The constructs are as follows: Construct #9b; Construct #10; Construct #11; Construct #12; Construct #1; Construct #2; TCR=R11KEA.WPRE^{wt} (TCR with wild type WPRE); NT=Non-transduced T cells (as a negative control).

FIG. 17 shows CD8α MFI (CD8+Tet+) of cells transduced with various constructs. The constructs are as follows: Construct #9b; Construct #10; Construct #11; Construct #12; Construct #1; Construct #2; TCR=R11KEA.WPRE^{wt} (TCR with wild type WPRE); NT=Non-transduced T cells (as a negative control).

FIG. 18 shows % Tet+(of CD3+) of cells transduced with various constructs. The constructs are as follows: Construct #9b; Construct #10; Construct #11; Construct #12; Construct #1; Construct #2; TCR=R11KEA.WPRE^{wt} (TCR with wild type WPRE); NT=Non-transduced T cells (as a negative control).

FIG. 19 shows VCN (upper panel) and CD3+Tet+/VCN (lower panel) of cells transduced with various constructs. The constructs are as follows: Construct #9b; Construct #10; Construct #11; Construct #12; Construct #1; Construct #2; TCR=R11KEA.WPRE^{wt} (TCR with wild type WPRE); NT=Non-transduced T cells (as a negative control).

FIG. 20A-20C depicts data showing that constructs (#10, #11, & #12) are comparable to TCR-only in mediating cytotoxicity against target positive cells lines expressing antigen at different levels (UACC257 at 1081 copies per cell and A375 at 50 copies per cell).

FIG. 21A-21B depict data showing that IFNγ secretion in response to UACC257 is comparable among constructs, however with A375, #10 expressing is the highest among all constructs. However, comparing #9 with #11 expressing wild type and modified CD8 coreceptor sequences respectively, T cells transduced with #11 induced stronger cytokine response measured as IFNγ quantified in the supernatants from Incucyte plates. Construct #9; Construct #10; Construct #11; Construct #12; Construct #1; Construct #2; Construct #8=R11KEA TCR only.

FIG. 22 depicts an exemplary experiment design to assess DC maturation and cytokine secretion by PBMC-derived product in response to UACC257 and A375 targets. N=2.

FIG. 23A-23B depicts data showing that the IFNγ secretion in response to A375 increases in the presence of iDCs. In the tri-cocultures with iDCs, IFNγ secretion is higher in Construct #10 compared to the other constructs. However, comparing Construct #9 with Construct #11 expressing wild type and modified CD8 coreceptor sequences respectively, T cells transduced with #11 induced stronger cytokine response measured as IFNγ quantified in the culture supernatants of three-way cocultures using donor D600115, E:T:iDC::1:1/10:1/4. Construct #9; Construct #10; Construct #11; Construct #12; Construct #1; Construct #2; Construct #8=R11KEA TCR only.

FIG. 24A-24B depicts data showing that IFNγ secretion in response to A375 increases in the presence of iDCs. In the tri-cocultures with iDCs, IFNγ secretion was higher in Construct #10 compared to the other constructs. IFNγ quantified in the culture supernatants of three-way cocultures using donor D150081, E:T:iDC:: 1:1/10:1/4. Construct #9; Construct #10; Construct #11; Construct #12; Construct #1; Construct #2; Construct #8=R11KEA TCR only.

FIG. 25A-25B depicts data showing that IFNγ secretion in response to UACC257 increases in the presence of iDCs. In the tri-cocultures with iDCs, IFNγ secretion is higher in Construct #10 compared to the other constructs. However, comparing Construct #9 with Construct #11 expressing wild type and modified CD8 coreceptor sequences respectively, T cells transduced with Construct #11 induced stronger cytokine response measured as IFNγ quantified in the culture supernatants of three-way cocultures using donor D600115, E:T:iDC:: 1:1/10:1/4. Construct #9; Construct #10; Construct #11; Construct #12; Construct #1; Construct #2; Construct #8=R11KEA TCR only.

FIG. 26 shows T cell manufacturing in accordance with one embodiment of the present disclosure.

FIG. 27A shows expression of activation markers before and after activation in CD3+CD8+ cells.

FIG. 27B shows expression of activation markers before and after activation in CD3+CD4+ cells in accordance with one embodiment of the present disclosure.

FIG. 28 shows fold expansion of cells transduced with various constructs.

FIGS. 29A & 29B show % CD8+CD4+ of cells transduced with various constructs in accordance with one embodiment of the present disclosure.

FIGS. 30A & 30B show % Tet of CD8+CD4+ of cells transduced with various constructs in accordance with one embodiment of the present disclosure.

FIGS. 31A & 31B show Tet MFI (CD8+CD4+Tet+) of cells transduced with various constructs in accordance with one embodiment of the present disclosure.

FIGS. 32A & 32B show % CD8+CD4−(of CD3+) of cells transduced with various constructs in accordance with one embodiment of the present disclosure.

FIGS. 33A & 33B show % CD8+Tet+(of CD3+) of cells transduced with various constructs in accordance with one embodiment of the present disclosure.

FIGS. 34A & 34B show Tet MFI (CD8+Tet+) of cells transduced with various constructs in accordance with one embodiment of the present disclosure.

FIGS. 35A & 35B show % Tet+(of CD3+) of cells transduced with various constructs in accordance with one embodiment of the present disclosure.

FIGS. 36A & 36B show VCN of cells transduced with various constructs in accordance with one embodiment of the present disclosure.

FIG. 37 shows T cell manufacturing in accordance with one embodiment of the present disclosure.

FIG. 38 shows % Tet of CD8+CD4+ of cells transduced with various constructs.

FIG. 39 shows Tet MFI of CD8+CD4+Tet+ of cells transduced with various constructs.

FIG. 40 shows Tet MFI of CD8+Tet+ of cells transduced with various constructs.

FIG. 41 shows % Tet+ of CD3+ cells transduced with various constructs.

FIG. 42 shows vector copy number (VCN) of cells transduced with various constructs.

FIG. 43 shows the % T cell subsets in cells transduced with various constructs. FACS analysis was gated on CD3+TCR+.

FIG. 44A and FIG. 44B shows % T cell subsets in cells transduced with various constructs. FACS analysis was gated on CD4+CD8+ for FIG. 44A and on CD4−CD8+TCR+ for FIG. 44B.

FIGS. 45A and 45B depicts data showing that Constructs #13 and #10 are comparable to TCR-only in mediating cytotoxicity against UACC257 target positive cells lines expressing high levels of antigen (1081 copies per cell). Construct #15 was also effective but slower in killing compared to Constructs #13 and #10. The effector:target ratio used to generate these results was 4:1.

FIG. 46 shows IFNγ secretion in response in UACC257 cell line was higher with Construct #13 compared to Construct #10. IFNγ quantified in the supernatants from Incucyte plates. The effector:target ratio used to generate these results was 4:1.

FIG. 47 shows ICI marker frequency (2B4, 41BB, LAG3, PD-1, TIGIT, TIM3, CD39+CD69+, and CD39−CD69−).

FIG. 48A-48G show increased expression of IFNγ, IL-2, and TNFα with CD4+CD8+ cells transduced with Construct #10 (WT signal peptide, CD8β1) compared to other constructs. FACS analysis was gated on CD3+CD4+CD8+ cells against UACC257, 4:1 E:T.

FIG. 49A-49G show increased expression of IFNγ, IL-2, MIP-1β, and TNFα with CD4-CD8+ cells transduced with Construct #10 (WT signal peptide, CD8β1) compared to other constructs. FACS analysis was gated on CD3+CD4−CD8+ cells against UACC257, 4:1 E:T.

FIG. 50A-50G show increased expression of IL-2 and TNFα with CD3+TCR+ cells transduced with Construct #10 (WT signal peptide, CD8β1) compared to other constructs. FACS analysis was gated on CD3+TCR+ cells against UACC257, 4:1 E:T.

FIG. 51A-51C show results from FACS analysis gated on CD4+CD8+ cells against A375, 4:1 E:T.

FIG. 52A-52C show results from FACS analysis gated on CD4−CD8+ cells against A375, 4:1 E:T.

FIG. 53A-53C show results from FACS analysis gated on CD3+TCR+ cells against A375, 4:1 E:T.

FIG. 54 shows T cell manufacturing in accordance with one embodiment of the present disclosure.

FIG. 55A-55C show interaction between peptide/MHC complex of antigen-presenting cell (APC) with T cell by binding a complex of TCR and CD8αβ heterodimer (FIG. 55A, e.g., produced by transducing T cells with Constructs #2, #3, #4, #10, #13, #14, #15, #16, #17, #18, or #21), a complex of TCR and homodimer CD8α having its stalk region replaced with CD8β stalk region (CD8αα*) (FIG. 55B, e.g., produced by transducing T cells with Construct #11, #12, or #19), and a complex of TCR and CD8α homodimer (FIG. 55C, e.g., produced by transducing T cells with Constructs #1, #5, #6, #7, or #9).

FIG. 56 shows the levels of IL-12 secretion by dendritic cells (DC) in the presence of CD4+ T cells transduced with Construct #10 or #11 and immature dendritic cells (iDCs) in accordance with one embodiment of the present disclosure.

FIG. 57 shows the levels of TNF-α secretion by dendritic cells (DC) in the presence of CD4+ T cells transduced with Construct #10 or #11 and immature dendritic cells (iDCs) in accordance with one embodiment of the present disclosure.

FIG. 58 shows the levels of IL-6 secretion by dendritic cells (DC) in the presence of CD4+ T cells transduced with Construct #10 or #11 and immature dendritic cells (iDCs) in accordance with one embodiment of the present disclosure.

FIG. 59 shows a scheme of determining the levels of cytokine secretion by dendritic cells (DC) in the presence of PBMCs transduced with various constructs and target cells in accordance with one embodiment of the present disclosure.

FIG. 60 shows the levels of IL-12 secretion by dendritic cells (DC) in the presence of PBMCs transduced with various constructs and target cells in accordance with one embodiment of the present disclosure.

FIG. 61 shows the levels of TNF-α secretion by dendritic cells (DC) in the presence of PBMCs transduced with various constructs and target cells in accordance with one embodiment of the present disclosure

FIG. 62 shows the levels of IL-6 secretion by dendritic cells (DC) in the presence of PBMCs transduced with various constructs and target cells in accordance with one embodiment of the present disclosure.

FIG. 63A-63C show IFNγ production from the transduced CD4+ selected T cells obtained from Donor #1 (FIG. 63A), Donor #2 (FIG. 63B), and Donor #3 (FIG. 63C) in accordance to one embodiment of the present disclosure.

FIG. 63D shows EC50 values (ng/ml) in FIG. 63A-63C.

FIG. 64A-64C show IFNγ production from the transduced PBMC obtained from Donor #4 (FIG. 64A), Donor #1 (FIG. 64B), and Donor #3 (FIG. 64C) and their respective EC50 values (ng/ml) in accordance to one embodiment of the present disclosure.

FIG. 64D shows comparison of EC50 values (ng/ml) among different donors in FIG. 64A-64C.

FIG. 65A-65C show IFNγ production from the transduced PBMC (FIG. 65A), CD8+ selected T cells (FIG. 65B), and CD4+ selected T cells (FIG. 65C) and their respective EC50 values (ng/ml) from a single donor in accordance to one embodiment of the present disclosure.

### DETAILED DESCRIPTION OF A PREFERRED EMBODIMENT

### Modified CD8 Polypeptides

CD8 polypeptides described herein may comprise the general structure of a N-terminal signal peptide (optional), CD8α immunoglobulin (Ig)-like domain, CD8 [symbol] region (domain), CD8α transmembrane domain, and a CD8α cytoplasmic domain. The modified CD8 polypeptides described herein shown an unexpected improvement in functionality of T cells co-transduced with a vector expressing a TCR and CD8 polypeptide.

CD8 polypeptides described herein may comprise the general structure of a N-terminal signal peptide (optional), CD8α immunoglobulin (Ig)-like domain, a stalk domain or region, CD8α transmembrane domain, and a CD8α cytoplasmic domain.

In an embodiment, CD8 polypeptides described herein may comprise (a) an immunoglobulin (Ig)-like domain comprising at least about 80%, at least 85%, at least 90%, at least 95%, at least 98%, at least 99%, or 100% sequence identity to the amino acid sequence of SEQ ID NO: 1; (b) a region comprising at least about 80%, at least 85%, at least 90%, at least 95%, at least 98%, at least 99%, or 100% sequence identity to the amino acid sequence of SEQ ID NO: 2; (c) a transmembrane domain comprising at least about 80%, at least 85%, at least 90%, at least 95%, at least 98%, at least 99%, or 100% sequence identity to the amino acid sequence of SEQ ID NO: 3, and (d) a cytoplasmic domain comprising at least about 80%, at least 85%, at least 90%, at least 95%, at least 98%, at least 99%, or 100% sequence identity to the amino acid sequence of SEQ ID NO: 4. The CD8 polypeptides described herein may be co-expressed with a T-cell receptor or CAR-T in a T-cell and used in methods of adoptive cell therapy (ACT). The T-cell may be an αβ T-cell or a γδ T-cell.

In another embodiment, CD8 polypeptides described herein may comprise (a) at least about 80%, at least 85%, at least 90%, at least 95%, at least 98%, at least 99%, or 100% sequence identity to the amino acid sequence of SEQ ID NO: 1; (b) at least about 80%, at least 85%, at least 90%, at least 95%, at least 98%, at least 99%, or 100% sequence identity to the amino acid sequence of SEQ ID NO: 2; (c) at least about 80%, at least 85%, at least 90%, at least 95%, at least 98%, at least 99%, or 100% sequence identity to the amino acid sequence of SEQ ID NO: 3, and (d) a at least about 80%, at least 85%, at least 90%, at least 95%, at least 98%, at least 99%, or 100% sequence identity to the amino acid sequence of SEQ ID NO: 4. The CD8 polypeptides described herein may be co-expressed with a T-cell receptor or CAR-T in a T-cell and used in methods of adoptive cell therapy (ACT). The T-cell may be an α T-cell or a γδ T-cell.

In another embodiment, CD8 polypeptides described herein may comprise (a) SEQ ID NO: 1 comprising one, two, three, four, or five amino acid substitutions; (b) SEQ ID NO: 2 comprising one, two, three, four, or five amino acid substitutions; (c) SEQ ID NO: 3 comprising one, two, three, four, or five amino acid substitutions, and (d) SEQ ID NO: 4 comprising one, two, three, four, or five amino acid substitutions. In an embodiment, the substitutions are conservative amino acid substitutions. The CD8 polypeptides described herein may be co-expressed with a T-cell receptor or CAR-T in a T-cell and used in methods of adoptive cell therapy (ACT). The T-cell may be an γδ T-cell or a γδ T-cell.

CD8 is a membrane-anchored glycoprotein that functions as a coreceptor for antigen recognition of the peptide/MHC class I complexes by T cell receptors (TCR) and plays an important role in T cell development in the thymus and T cell activation in the periphery. Functional CD8 is a dimeric protein made of either two a chains (CD8αα) or an αchain and a β chain (CD8αβ), and the surface expression of the β chain may require its association with the coexpressed αchain to form the CD8αβheterodimer. CD8αα and CD8αβ may be differentially expressed on a variety of lymphocytes. CD8αβ is expressed predominantly on the surface of αβTCR⁺ T cells and thymocytes, and CD8αα on a subset of αβTCR⁺, γδTCR⁺ intestinal intraepithelial lymphocytes, NK cells, dendritic cells, and a small fraction of CD4⁺ T cells.

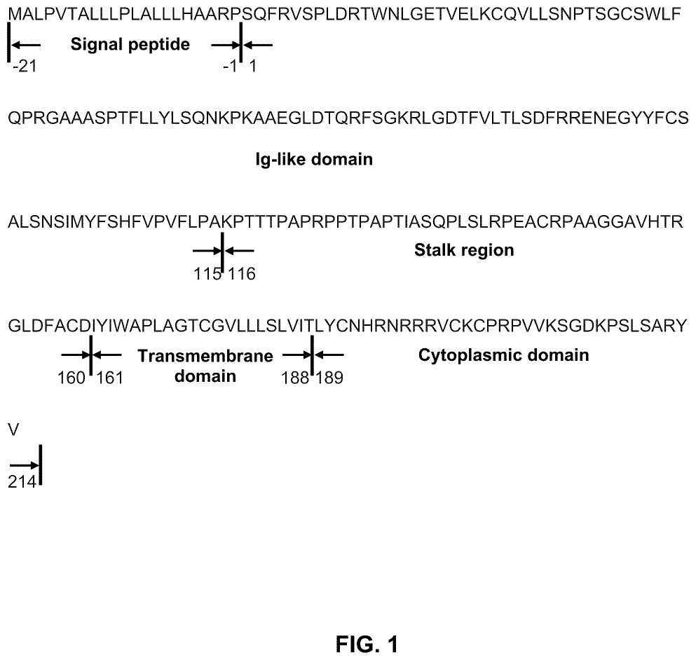

For example, human CD8 gene may express a protein of 235 amino acids. FIG. 1 shows a CD8α protein (CD8α1-SEQ ID NO: 258), which in an aspect is divided into the following domains (starting at the amino terminal and ending at the carboxy terminal of the polypeptide): (1) signal peptide (amino acids −21 to −1), which may be cleaved off in human cells during the transport of the receptor to the cell surface and thus may not constitute part of the mature, active receptor; (2) immunoglobulin (Ig)-like domain (in this embodiment, amino acids 1-115), which may assume a structure, referred to as the immunoglobulin fold, which is similar to those of many other molecules involved in regulating the immune system, the immunoglobulin family of proteins. The crystal structure of the CD8ααreceptor in complex with the human MHC molecule HLA-A2 has demonstrated how the Ig domain of CD8ααreceptor binds the ligand; (3) membrane proximal region (in this embodiment, amino acids 116-160), which may be an extended linker region allowing the CD8ααreceptor to “reach” from the surface of the T-cell over the top of the MHC to the α3 domain of the MHC where it binds. The stalk region may be glycosylated and may be inflexible; (4) transmembrane domain (in this embodiment, amino acids 161-188), which may anchor the CD8ααreceptor in the cell membrane and is therefore not part of the soluble recombinant protein; and (5) cytoplasmic domain (in this embodiment, amino acids 189-214), which can mediate a signaling function in T-cells through its association with p56^{lck}, which may be involved in the T cell activation cascade of phosphorylation events.

CD8α sequences may generally have a sufficient portion of the immunoglobulin domain to be able to bind to MHC. Generally, CD8α molecules may contain all or a substantial part of immunoglobulin domain of CD8α, e.g., SEQ ID NO: 258, but in an aspect may contain at least 10, 15, 20, 25, 30, 35, 40, 45, 50, 55, 60, 65, 70, 75, 80, 85, 90, 95, 100, 105, 110 or 115 amino acids of the immunoglobulin domain. The CD8α molecules of the present disclosure may be preferably dimers (e.g., CD8αα or CD8αβ), although CD8α monomer may be included within the scope of the present disclosure. In an aspect, CD8α of the present disclosure may comprise CD8α1 (SEQ ID NO: 258) and CD8α2 (SEQ ID NO: 259).

CD8α and β subunits may have similar structural motifs, including an Ig-like domain, a stalk region of 30-40 amino acids, a transmembrane region, and a short cytoplasmic domain of about 20 amino acids. CD8α and β chains have two and one N-linked glycosylation sites, respectively, in the Ig-like domains where they share<20% identity in their amino acid sequences. The CD8β stalk region is 10-13 amino acids shorter than the CD8α stalk and is highly glycosylated with O-linked carbohydrates. These carbohydrates on the β, but not the α, stalk region appear to be quite heterogeneous due to complex sialylations, which may be differentially regulated during the developmental stages of thymocytes and upon activation of T cells. Glycan adducts have been shown to play regulatory roles in the functions of glycoproteins and in immune responses. Glycans proximal to transmembrane domains can affect the orientation of adjacent motifs. The unique biochemical properties of the CD8β chain stalk region may present a plausible candidate for modulating the coreceptor function.

The CD8 polypeptide may be modified, in which CD8α region, for example a stalk region, may be replaced by CD8β region. In another aspect, to create a CD8 [symbol] -CD8 [symbol] polypeptide. In an embodiment, the modified CD8 polypeptides described herein may have a region comprising at least 80%, 81%, 82%, 83%, 84%, 85%, 86%, 87%, 88%, 89%, 90%, 91%, 92%, 93%, 94%, 95%, 96%, 97%, 98%, 99%, or 100% sequence identity to the amino acid sequence of SEQ ID NO: 2. The modified CD8α polypeptides described herein may have an immunoglobulin (Ig)-like domain having at least 80%, 81%, 82%, 83%, 84%, 85%, 86%, 87%, 88%, 89%, 90%, 91%, 92%, 93%, 94%, 95%, 96%, 97%, 98%, 99%, or 100% sequence identity to the amino acid sequence of SEQ ID NO: 1. Modified CD8 polypeptides may have a transmembrane domain comprising at least at least 80%, 81%, 82%, 83%, 84%, 85%, 86%, 87%, 88%, 89%, 90%, 91%, 92%, 93%, 94%, 95%, 96%, 97%, 98%, 99%, or 100% sequence identity to the amino acid sequence of SEQ ID NO: 3. Modified CD8 polypeptides described herein may have a cytoplasmic tail comprising at least at least 80%, 81%, 82%, 83%, 84%, 85%, 86%, 87%, 88%, 89%, 90%, 91%, 92%, 93%, 94%, 95%, 96%, 97%, 98%, 99%, or 100% sequence identity to the amino acid sequence of SEQ ID NO: 4. The CD8 polypeptides described herein may have at least 80%, 81%, 82%, 83%, 84%, 85%, 86%, 87%, 88%, 89%, 90%, 91%, 92%, 93%, 94%, 95%, 96%, 97%, 98%, 99%, or 100% sequence identity to the amino acid sequence of SEQ ID NO: 5. The CD8 polypeptides described herein may comprise a signal peptide comprising at least 80%, at least 85%, at least 90%, at least 95%, at least 99%, or 100% sequence identity to the amino acid sequence of SEQ ID NO: 6 or SEQ ID NO: 294 fused to the N-terminus or fused to the C-terminus of mCD8α polypeptide. The CD8 polypeptides described herein may have at least 80%, 81%, 82%, 83%, 84%, 85%, 86%, 87%, 88%, 89%, 90%, 91%, 92%, 93%, 94%, 95%, 96%, 97%, 98%, 99%, or 100% sequence identity to the amino acid sequence of SEQ ID NO: 7.

### T-Cells

T-cells may express the modified CD8 polypeptides described herein. For example, a T-cell may co-express a T-cell Receptor (TCR) and modified CD8 polypeptides described herein. T-cells may also express a chimeric antigen receptor (CAR), CAR-analogues, or CAR derivatives.

The T-cell may be a αβ T cell, γδ T cell, natural killer T cell, or a combination thereof if in a population. The T cell may be a CD4+ T cell, CD8+ T cell, or a CD4+/CD8+ T cell.

### T-Cell Receptors

A T-cell may co-express a T-cell receptor (TCR), antigen binding protein, or both, with modified CD8 polypeptides described herein, including, but are not limited to, those listed in Table 3 (SEQ ID NOs: 15-92). Further, a T-cell may express a TCRs and antigen binding proteins described in U.S. Patent Application Publication No. 2017/0267738; U.S. Patent Application Publication No. 2017/0312350; U.S. Patent Application Publication No. 2018/0051080; U.S. Patent Application Publication No. 2018/0164315; U.S. Patent Application Publication No. 2018/0161396; U.S. Patent Application Publication No. 2018/0162922; U.S. Patent Application Publication No. 2018/0273602; U.S. Patent Application Publication No. 2019/0016801; U.S. Patent Application Publication No. 2019/0002556; U.S. Patent Application Publication No. 2019/0135914; U.S. Pat. Nos. 10,538,573; 10,626,160; U.S. Patent Application Publication No. 2019/0321478; U.S. Patent Application Publication No. 2019/0256572; U.S. Pat. Nos. 10,550,182; 10,526,407; U.S. Patent Application Publication No. 2019/0284276; U.S. Patent Application Publication No. 2019/0016802; U.S. Patent Application Publication No. 2019/0016803; U.S. Patent Application Publication No. 2019/0016804; U.S. Pat. No. 10,583,573; U.S. Patent Application Publication No. 2020/0339652; U.S. Pat. Nos. 10,537,624; 10,596,242; U.S. Patent Application Publication No. 2020/0188497; U.S. Pat. No. 10,800,845; U.S. Patent Application Publication No. 2020/0385468; U.S. Pat. Nos. 10,527,623; 10,725,044; U.S. Patent Application Publication No. 2020/0249233; U.S. Pat. No. 10,702,609; U.S. Patent Application Publication No. 2020/0254106; U.S. Pat. No. 10,800,832; U.S. Patent Application Publication No. 2020/0123221; U.S. Pat. Nos. 10,590,194; 10,723,796; U.S. Patent Application Publication No. 2020/0140540; U.S. Pat. No. 10,618,956; U.S. Patent Application Publication No. 2020/0207849; U.S. Patent Application Publication No. 2020/0088726; and U.S. Patent Application Publication No. 2020/0384028; the contents of each of these publications and sequence listings described therein are herein incorporated by reference in their entireties. The T-cell may be a αβ T cell, γδ T cell, natural killer T cell. Natural killer cell. In an embodiment, TCRs described herein are single-chain TCRs or soluble TCRs.

Further, the TCRs that may be co-expressed with the modified CD8 polypeptides described herein in a T-cell may be TCRs comprised of an alpha chain (TCR [symbol] ) and a beta chain (TCR [symbol] ). The TCRα chains and TCRβ chains that may be used in TCRs may be selected from R11KEA (SEQ ID NO: 15 and 16), R20P1H7 (SEQ ID NO: 17 and 18), R7P1D5 (SEQ ID NO: 19 and 20), R10P2G12 (SEQ ID NO: 21 and 22), R10P1A7 (SEQ ID NO: 23 and 24), R4P1D10 (SEQ ID NO: 25 and 26), R4P3F9 (SEQ ID NO: 27 and 28), R4P3H3 (SEQ ID NO: 29 and 30), R36P3F9 (SEQ ID NO: 31 and 32), R52P2G11 (SEQ ID NO: 33 and 34), R53P2A9 (SEQ ID NO: 35 and 36), R26P1A9 (SEQ ID NO: 37 and 38), R26P2A6 (SEQ ID NO: 39 and 40), R26P3H1 (SEQ ID NO: 41 and 42), R35P3A4 (SEQ ID NO: 43 and 44), R37P1C9 (SEQ ID NO: 45 and 46), R37P1H1 (SEQ ID NO: 47 and 48), R42P3A9 (SEQ ID NO: 49 and 50), R43P3F2 (SEQ ID NO: 51 and 52), R43P3G5 (SEQ ID NO: 53 and 54), R59P2E7 (SEQ ID NO: 55 and 56), R11P3D3 (SEQ ID NO: 57 and 58), R16P1C10 (SEQ ID NO: 59 and 60), R16P1E8 (SEQ ID NO: 61 and 62), R17P1A9 (SEQ ID NO: 63 and 64), R17P1D7 (SEQ ID NO: 65 and 66), R17P1G3 (SEQ ID NO: 67 and 68), R17P2B6 (SEQ ID NO: 69 and 70), R11P3D3KE (SEQ ID NO: 71 and 303), R39P1C12 (SEQ ID NO: 304 and 74), R39P1F5 (SEQ ID NO: 75 and 76), R40P1C2 (SEQ ID NO: 77 and 78), R41P3E6 (SEQ ID NO: 79 and 80), R43P3G4 (SEQ ID NO: 81 and 82), R44P3B3 (SEQ ID NO: 83 and 84), R44P3E7 (SEQ ID NO: 85 and 86), R49P2B7 (SEQ ID NO: 87 and 88), R55P1G7 (SEQ ID NO: 89 and 90), or R59P2A7 (SEQ ID NO: 91 and 92). The T-cell may be a αβ T cell, γδ T cell, or a natural killer T cell.

Table 1 shows examples of the peptides to which TCRs bind when the peptide is in a complex with an MHC molecule. (MHC molecules in humans may be referred to as HLA, human leukocyte-antigens).

TABLE 1
T-Cell Receptor and Peptides

| TCR name | Peptide (SEQ ID NO:) |
|---|---|
| R20P1H7, R7P1D5, R10P2G12 | KVLEHVVRV (SEQ ID NO: 215) |
| R10P1A7 | KIQEILTQV (SEQ ID NO: 123) |
| R4P1D10, R4P3F9, R4P3H3 | FLLDGSANV (SEQ ID NO: 238) |
| R36P3F9, R52P2G11, R53P2A9 | ILQDGQFLV (SEQ ID NO: 193) |
| R26P1A9, R26P2A6, R26P3H1, R35P3A4, | KVLEYVIKV (SEQ ID NO: 202) |
| R37P1C9, R37P1H1, R42P3A9, R43P3F2, | |
| R43P3G5, R59P2E7 | |
| R11KEA, R11P3D3, R16P1C10, R16P1E8, | SLLQHLIGL (SEQ ID NO: 147) |
| R17P1A9, R17P1D7, R17P1G3, R17P2B6, | |
| R11P3D3KE | |
| R39P1C12, R39P1F5, R40P1C2, R41P3E6, | ALSVLRLAL (SEQ ID NO: 248) |
| R43P3G4, R44P3B3, R44P3E7, R49P2B7, | |
| R55P1G7, R59P2A7 | |

### Tumor Associated Antigens (TAA)

Tumor associated antigen (TAA) peptides may be used with the CD8 polypeptides constructs, methods and embodiments described herein. For example, the T-cell receptors (TCRs) described herein may specifically bind to the TAA peptide when bound to a human leukocyte antigen (HLA). This is also known as a major histocompatibility complex (MHC) molecule. The MHC-molecules of the human are also designated as human leukocyte-antigens (HLA).

Tumor associated antigen (TAA) peptides that may be used with the CD8 polypeptides described herein include, but are not limited to, those listed in Table 3 and those TAA peptides described in U.S. Patent Application Publication No. 2016/0187351; U.S. Patent Application Publication No. 2017/0165335; U.S. Patent Application Publication No. 2017/0035807; U.S. Patent Application Publication No. 2016/0280759; U.S. Patent Application Publication No. 2016/0287687; U.S. Patent Application Publication No. 2016/0346371; U.S. Patent Application Publication No. 2016/0368965; U.S. Patent Application Publication No. 2017/0022251; U.S. Patent Application Publication No. 2017/0002055; U.S. Patent Application Publication No. 2017/0029486; U.S. Patent Application Publication No. 2017/0037089; U.S. Patent Application Publication No. 2017/0136108; U.S. Patent Application Publication No. 2017/0101473; U.S. Patent Application Publication No. 2017/0096461; U.S. Patent Application Publication No. 2017/0165337; U.S. Patent Application Publication No. 2017/0189505; U.S. Patent Application Publication No. 2017/0173132; U.S. Patent Application Publication No. 2017/0296640; U.S. Patent Application Publication No. 2017/0253633; U.S. Patent Application Publication No. 2017/0260249; U.S. Patent Application Publication No. 2018/0051080; U.S. Patent Application Publication No. 2018/0164315; U.S. Patent Application Publication No. 2018/0291082; U.S. Patent Application Publication No. 2018/0291083; U.S. Patent Application Publication No. 2019/0255110; U.S. Pat. Nos. 9,717,774; 9,895,415; U.S. Patent Application Publication No. 2019/0247433; U.S. Patent Application Publication No. 2019/0292520; U.S. Patent Application Publication No. 2020/0085930; U.S. Pat. Nos. 10,336,809; 10,131,703; 10,081,664; 10,081,664; 10,093,715; 10,583,573; and U.S. Patent Application Publication No. 2020/00085930; the contents of each of these publications, sequences, and sequence listings described therein are herein incorporated by reference in their entireties. The Tumor associated antigen (TAA) peptides described herein may be bound to an HLA (MHC molecule). The Tumor associated antigen (TAA) peptides bound to an HLA may be recognized by a TCR described herein, optionally co-expressed with CD8 polypeptides described herein.

T cells may be engineered to express a chimeric antigen receptor (CAR) comprising a ligand binding domain derived from NKG2D, NKG2A, NKG2C, NKG2F, LLT1, AICL, CD26, NKRP1, NKp30, NKp44, NKp46, CD244 (2B4), DNAM-1, and NKp80, or an anti-tumor antibody such as anti-Her2neu or anti-EGFR and a signaling domain obtained from CD3-ζ, Dap 10, CD28, 4-IBB, and CD40L. In some examples, the chimeric receptor binds MICA, MICB, Her2neu, EGFR, mesothelin, CD38, CD20, CD 19, PSA, RON, CD30, CD22, CD37, CD38, CD56, CD33, CD30, CD138, CD123, CD79b, CD70, CD75, CA6, GD2, alpha-fetoprotein (AFP), carcinoembryonic antigen (CEA), CEACAM5, CA-125, MUC-16, 5T4, NaPi2b, ROR1, ROR2, 5T4, PLIF, Her2/Neu, EGFRvIII, GPMNB, LIV-1, glycolipidF77, fibroblast activating protein, PSMA, STEAP-1, STEAP-2, c-met, CSPG4, Nectin-4, VEGFR2, PSCA, folate binding protein/receptor, SLC44A4, Cripto, CTAG1B, AXL, IL-13R, IL-3R, SLTRK6, gp100, MART1, Tyrosinase, SSX2, SSX4, NYESO-1, epithelial tumor antigen (ETA), MAGEA family genes (such as MAGE3A. MAGE4A), KKLC1, mutated ras, βraf, p53, MHC class I chain-related molecule A (MICA), or MHC class I chain-related molecule B (MICB), HPV, or CMV. The T-cell may be a αβ T cell, γδ T cell, or a natural killer T cell.

### Culturing T-Cells

Methods for the activation, transduction, and/or expansion of T cells, e.g., tumor-infiltrating lymphocytes, CD8+ T cells, CD4+ T cells, and T cells, that may be used for transgene expression are described herein. T cells may be activated, transduced, and expanded, while depleting α- and/or β-TCR positive cells. The T-cell may be a αβ T cell, γδ T cell, or a natural killer T cell.

Methods for the ex vivo expansion of a population of engineered γδ T-cells for adoptive transfer therapy are described herein. Engineered γδ T cells of the disclosure may be expanded ex vivo. Engineered T cells described herein can be expanded in vitro without activation by APCs, or without co-culture with APCs, and aminophosphates. Methods for transducing T cells are described in U.S. Patent Application No. Patent Application No. 2019/0175650, published on Jun. 13, 2019, the contents of which are incorporated by reference in their entirety. Other methods for transduction and culturing of T-cells may be used.

T cells, including γδ T cells, may be isolated from a complex sample that is cultured in vitro. In an embodiment, whole PBMC population, without prior depletion of specific cell populations, such as monocytes, αβ T-cells, B-cells, and NK cells, can be activated and expanded. In an embodiment, enriched T cell populations can be generated prior to their specific activation and expansion. In an embodiment, activation and expansion of γδ T cells may be performed with or without the presence of native or engineered antigen presenting cells (APCs). In an embodiments, isolation and expansion of T cells from tumor specimens can be performed using immobilized T cell mitogens, including antibodies specific to γδ TCR, and other γδ TCR activating agents, including lectins. In an embodiment, isolation and expansion of γδ T cells from tumor specimens can be performed in the absence of γδ T cell mitogens, including antibodies specific to γδ TCR, and other γδ TCR activating agents, including lectins.

T cells, including γδ T cells, may be isolated from leukapheresis of a subject, for example, a human subject. In an embodiment, γδ T cells are not isolated from peripheral blood mononuclear cells (PBMC). The T cells may be isolated using anti-CD3 and anti-CD28 antibodies, optionally with recombinant human Interleukin-2 (rhlL-2), e.g., between about 50 and 150 U/mL rhlL-2.

The isolated T cells can rapidly expand in response to contact with one or more antigens. Some γδ T cells, such as Vγ9Vδ2+ T cells, can rapidly expand in vitro in response to contact with some antigens, like prenyl-pyrophosphates, alkyl amines, and metabolites or microbial extracts during tissue culture. Stimulated T-cells can exhibit numerous antigen-presentation, co-stimulation, and adhesion molecules that can facilitate the isolation of T-cells from a complex sample. T cells within a complex sample can be stimulated in vitro with at least one antigen for 1 day, 2 days, 3 days, 4 days, 5 days, 6 days, 7 days, or another suitable period of time. Stimulation of T cells with a suitable antigen can expand T cell population in vitro.

Activation and expansion of γδ T cells can be performed using activation and co-stimulatory agents described herein to trigger specific γδ T cell proliferation and persistence populations. In an embodiment, activation and expansion of γδ T-cells from different cultures can achieve distinct clonal or mixed polyclonal population subsets. In an embodiment, different agonist agents can be used to identify agents that provide specific γδ activating signals. In an embodiment, agents that provide specific γδ activating signals can be different monoclonal antibodies (MAbs) directed against the γδ TCRs. In an embodiment, companion co-stimulatory agents to assist in triggering specific γδ T cell proliferation without induction of cell energy and apoptosis can be used. These co-stimulatory agents can include ligands binding to receptors expressed on γδ cells, such as NKG2D, CD161, CD70, JAML, DNAX accessory molecule-1 (DNAM-1), ICOS, CD27, CD137, CD30, HVEM, SLAM, CD122, DAP, and CD28. In an embodiment, co-stimulatory agents can be antibodies specific to unique epitopes on CD2 and CD3 molecules. CD2 and CD3 can have different conformation structures when expressed on αβ or γδ T-cells. In an embodiment, specific antibodies to CD3 and CD2 can lead to distinct activation of γδ T cells.

Non-limiting examples of antigens that may be used to stimulate the expansion of T cells, including γδ T cells, from a complex sample in vitro may comprise, prenyl-pyrophosphates, such as isopentenyl pyrophosphate (IPP), alkyl-amines, metabolites of human microbial pathogens, metabolites of commensal bacteria, methyl-3-butenyl-1-pyrophosphate (2M3B1PP), (E)-4-hydroxy-3-methyl-but-2-enyl pyrophosphate (HMB-PP), ethyl pyrophosphate (EPP), farnesyl pyrophosphate (FPP), dimethylallyl phosphate (DMAP), dimethylallyl pyrophosphate (DMAPP), ethyl-adenosine triphosphate (EPPPA), geranyl pyrophosphate (GPP), geranylgeranyl pyrophosphate (GGPP), isopentenyl-adenosine triphosphate (IPPPA), monoethyl phosphate (MEP), monoethyl pyrophosphate (MEPP), 3-formyl-1-butyl-pyrophosphate (TUBAg 1), X-pyrophosphate (TUBAg 2), 3-formyl-1-butyl-uridine triphosphate (TUBAg 3), 3-formyl-1-butyl-deoxythymidine triphosphate (TUBAg 4), monoethyl alkylamines, allyl pyrophosphate, crotoyl pyrophosphate, dimethylallyl-γ-uridine triphosphate, crotoyl-γ-uridine triphosphate, allyl-γ-uridine triphosphate, ethylamine, isobutylamine, sec-butylamine, iso-amylamine and nitrogen containing bisphosphonates.

A population of T-cells, including γδ T cells, may be expanded ex vivo prior to engineering of the T-cells. Non-limiting example of reagents that can be used to facilitate the expansion of a T-cell population in vitro may comprise anti-CD3 or anti-CD2, anti-CD27, anti-CD30, anti-CD70, anti-OX40 antibodies, IL-2, IL-15, IL-12, IL-9, IL-33, IL-18, or IL-21, CD70 (CD27 ligand), phytohaemagglutinin (PHA), concavalin A (ConA), pokeweed (PWM), protein peanut agglutinin (PNA), soybean agglutinin (SBA), Les Culinaris Agglutinin (LCA), Pisum Sativum Agglutinin (PSA), Helix pomatia agglutinin (HPA), Vicia graminea Lectin (VGA), or another suitable mitogen capable of stimulating T-cell proliferation. Further, the T-cells may be expanded using MCSF, IL-6, eotaxin, IFN-alpha, IL-7, gamma-induced protein 10, IFN-gamma, IL-1RA, IL-12, MIP-1alpha, IL-2, IL-13, MIP-1beta, IL-2R, IL-15, and combinations thereof.

The ability of γδ T cells to recognize a broad spectrum of antigens can be enhanced by genetic engineering of the γδ T cells. The γδ T cells can be engineered to provide a universal allogeneic therapy that recognizes an antigen of choice in vivo. Genetic engineering of the γδ T-cells may comprise stably integrating a construct expressing a tumor recognition moiety, such as αβ TCR, γδ TCR, chimeric antigen receptor (CAR), which combines both antigen-binding and T-cell activating functions into a single receptor, an antigen binding fragment thereof, or a lymphocyte activation domain into the genome of the isolated γδ T-cell(s), a cytokine (for example, IL-15, IL-12, IL-2. IL-7. IL-21, IL-18, IL-19, IL-33, IL-4, IL-9, IL-23, or IL1(3) to enhance T-cell proliferation, survival, and function ex vivo and in vivo. Genetic engineering of the isolated γδ T-cell may also include deleting or disrupting gene expression from one or more endogenous genes in the genome of the isolated γδ T-cells, such as the MHC locus (loci).

Engineered (or transduced) T cells, including γδ T cells, can be expanded ex vivo without stimulation by an antigen presenting cell or aminobisphosphonate. Antigen reactive engineered T cells of the present disclosure may be expanded ex vivo and in vivo. In an embodiment, an active population of engineered T cells may be expanded ex vivo without antigen stimulation by an antigen presenting cell, an antigenic peptide, a non-peptide molecule, or a small molecule compound, such as an aminobisphosphonate but using certain antibodies, cytokines, mitogens, or fusion proteins, such as IL-17 Fc fusion, MICA Fc fusion, and CD70 Fc fusion. Examples of antibodies that can be used in the expansion of a γδ T-cell population include anti-CD3, anti-CD27, anti-CD30, anti-CD70, anti-OX40, anti-NKG2D, or anti-CD2 antibodies, examples of cytokines may comprise IL-2, IL-15, IL-12, IL-21, IL-18, IL-9, IL-7, and/or IL-33, and examples of mitogens may comprise CD70 the ligand for human CD27, phytohaemagglutinin (PHA), concavalin A (ConA), pokeweed mitogen (PWM), protein peanut agglutinin (PNA), soybean agglutinin (SBA), Les culinaris agglutinin (LCA), Pisum sativum agglutinin (PSA), Helix pomatia agglutinin (HPA), Vicia graminea Lectin (VGA) or another suitable mitogen capable of stimulating T-cell proliferation.

A population of engineered T cells, including γδ T cells, can be expanded in less than 60 days, less than 48 days, less than 36 days, less than 24 days, less than 12 days, or less than 6 days. In an embodiment, a population of engineered T cells can be expanded from about 7 days to about 49 days, about 7 days to about 42 days, from about 7 days to about 35 days, from about 7 days to about 28 days, from about 7 days to about 21 days, or from about 7 days to about 14 days. The T-cells may be expanded for between about 1 and 21 days. For example, the T-cells may be expanded for about at least 1, 2, 3, 4, 5, 6, 7, 8, 9, 10, 11, 12, 13, 14, 15, 16, 17, 18, 19, or 20 days.

In an embodiment, the same methodology may be used to isolate, activate, and expand αβ T cells.

In an embodiment, the same methodology may be used to isolate, activate, and expand γδ T cells.

### Vectors

Engineered T-cells may be generated using various methods, including those recognized in the literature. For example, a polynucleotide encoding an expression cassette that comprises a tumor recognition, or another type of recognition moiety, can be stably introduced into the T-cell by a transposon/transposase system or a viral-based gene transfer system, such as a lentiviral or a retroviral system, or another suitable method, such as transfection, electroporation, transduction, lipofection, calcium phosphate (CaPO₄), nanoengineered substances, such as Ormosil, viral delivery methods, including adenoviruses, retroviruses, lentiviruses, adeno-associated viruses, or another suitable method. A number of viral methods have been used for human gene therapy, such as the methods described in WO 1993/020221, the content of which is incorporated herein in its entirety. Non-limiting examples of viral methods that can be used to engineer T cells may comprise γ-retroviral, adenoviral, lentiviral, herpes simplex virus, vaccinia virus, pox virus, or adeno-virus associated viral methods. The T cells may be αβ T cells or γδ T cells.

Viruses used for transfection of T-cells include naturally occurring viruses as well as artificial viruses. Viruses may be either an enveloped or non-enveloped virus. Parvoviruses (such as AAVs) are examples of non-enveloped viruses. The viruses may be enveloped viruses. The viruses used for transfection of T-cells may be retroviruses and in particular lentiviruses. Viral envelope proteins that can promote viral infection of eukaryotic cells may comprise HIV-1 derived lentiviral vectors (LVs) pseudotyped with envelope glycoproteins (GPs) from the vesicular stomatitis virus (VSV-G), the modified feline endogenous retrovirus (RD114TR) (SEQ ID NO: 97), and the modified gibbon ape leukemia virus (GALVTR). These envelope proteins can efficiently promote entry of other viruses, such as parvoviruses, including adeno-associated viruses (AAV), thereby demonstrating their broad efficiency. For example, other viral envelop proteins may be used including Moloney murine leukemia virus (MLV) 4070 env (such as described in Merten et al., J. Virol. 79:834-840, 2005; the content of which is incorporated herein by reference), RD114 env, chimeric envelope protein RD114pro or RDpro (which is an RD114-HIV chimera that was constructed by replacing the R peptide cleavage sequence of RD114 with the HIV-1 matrix/capsid (MA/CA) cleavage sequence, such as described in Bell et al. Experimental Biology and Medicine 2010; 235: 1269-1276; the content of which is incorporated herein by reference), baculovirus GP64 env (such as described in Wang et al. J. Virol. 81:10869-10878, 2007; the content of which is incorporated herein by reference), or GALV env (such as described in Merten et al., J. Virol. 79:834-840, 2005; the content of which is incorporated herein by reference), or derivatives thereof.

A single lentiviral cassette can be used to create a single lentiviral vector, expressing at least four individual monomer proteins of two distinct dimers from a single multi-cistronic mRNA so as to co-express the dimers on the cell surface. For example, the integration of a single copy of the lentiviral vector was sufficient to transform T cells to co-express TCRαβ and CD8αβ, optionally αβ T cells or γδ T cells.

Vectors may comprise a multi-cistronic cassette within a single vector capable of expressing more than one, more than two, more than three, more than four genes, more than five genes, or more than six genes, in which the polypeptides encoded by these genes may interact with one another or may form dimers. The dimers may be homodimers, e.g., two identical proteins forming a dimer, or heterodimers, e.g., two structurally different proteins forming a dimer.

Additionally, multiple vectors may be used to transfect cells with the constructs and sequences described herein. For example, the TCR transgene may be on one vector and the CD8 transgene encoding a polypeptide described herein may be on a second that are transfected either simultaneously or sequentially using recognized methods. A T-cell line may be stably transfected with a CD8 transgene encoding a CD8 polypeptide described herein and then sequentially transfected with a TCR transgene or visa verse.

In some embodiments, the transgene may further include one or more multicistronic element(s) and the multicistronic element(s) may be positioned, for example, between the nucleic acid sequence encoding the TCRα or a portion thereof and the nucleic acid sequence encoding the TCRβ or a portion thereof; between the nucleic acid sequence encoding the CD8α or a portion thereof and the nucleic acid sequence encoding the CD8p or a portion thereof, or between any two nucleic acid sequences encoding of TCRα, TCRβ, CD8α, and CD8β. In some embodiments, the multicistronic element(s) may include a sequence encoding a ribosome skip element selected from among a T2A, a P2A, a E2A or a F2A or an internal ribosome entry site (IRES).

As used herein, the term “self-cleaving 2A peptide” refers to relatively short peptides (of the order of 20 amino acids long, depending on the virus of origin) acting co-translationally, by preventing the formation of a normal peptide bond between the glycine and last proline, resulting in the ribosome skipping to the next codon, and the nascent peptide cleaving between the Gly and Pro. After cleavage, the short 2A peptide remains fused to the C-terminus of the ‘upstream’ protein, while the proline is added to the N-terminus of the ‘downstream’ protein. Self-cleaving 2A peptide may be selected from porcine teschovirus-1 (P2A), equine rhinitis A virus (E2A), Those a assign a virus (T2A), foot-and-mouth disease virus (F2A), or any combination thereof (see, e.g., Kim et al., PLOS One 6:e18556, 2011, the content of which including 2A nucleic acid and amino acid sequences are incorporated herein by reference in their entireties). By adding the linker sequences (GSG or SGSG (SEQ ID NO: 266)) before the self-cleaving 2A sequence, this may enable efficient synthesis of biologically active proteins, e.g., TCRs.

As used herein, the term “internal ribosome entry site (IRES)” refers to a nucleotide sequence located in a messenger RNA (mRNA) sequence, which can initiate translation without relying on the 5′ cap structure. IRES is usually located in the 5′ untranslated region (5′UTR) but may also be located in other positions of the mRNA. In one embodiment IRES may be selected from IRES from viruses, IRES from cellular mRNAs, in particular IRES from picornavirus, such as polio, EMCV and FMDV, flavivirus, such as hepatitis C virus (HCV), pestivirus, such as classical swine fever virus (CSFV), retrovirus, such as murine leukaemia virus (MLV), lentivirus, such as simian immunodeficiency virus (SIV), and insect RNA virus, such as cricket paralysis virus (CRPV), and IRES from cellular mRNAs, e.g. translation initiation factors, such as eIF4G, and DAP5, transcription factors, such as c-Myc, and NF-κB-repressing factor (NRF), growth factors, such as vascular endothelial growth factor (VEGF), fibroblast growth factor 2 (FGF-2), platelet-derived growth factor B (PDGF-B), homeotic genes, such as antennapedia, survival proteins, such as X-linked inhibitor of apoptosis (XIAP), and Apaf-1, and other cellular mRNA, such as BiP.

Constructs and vectors described herein are used with the methodology described in U.S. Patent Application Publication No. 2019/0175650, published on Jun. 13, 2019, the contents of which are incorporated by reference in their entirety.

Non-viral vectors may also be used with the sequences, constructs, and cells described herein.

The cells may be transfected by other means known in the art including lipofection (liposome-based transfection), electroporation, calcium phosphate transfection, biolistic particle delivery (e.g., gene guns), microinjection, or combinations thereof. Various methods of transfecting cells are known in the art. See, e.g., Sambrook & Russell (Eds.) Molecular Cloning: A Laboratory Manual (3^{rd} Ed.) Volumes 1-3 (2001) Cold Spring Harbor Laboratory Press; Ramamoorth & Narvekar “Non Viral Vectors in Gene Therapy-An Overview.” J Clin Diagn Res. (2015) 9(1): GE01-GE06.

### Compositions

Compositions may comprise the modified CD8 polypeptides described herein. Further, compositions described herein may comprise a T-cell expressing CD8 polypeptides described herein. The compositions described herein may comprise a T-cell expressing CD8 polypeptides described herein and a T-cell receptor (TCR), optionally a TCR that specifically binds one of the TAA described herein complexed with an antigen presenting protein, e.g., MHC, referred to as HLA in humans, for human leukocyte antigen.

To facilitate administration, the T cells described herein can be made into a pharmaceutical composition or made into an implant appropriate for administration in vivo, with pharmaceutically acceptable carriers or diluents. The means of making such a composition or an implant are described in the art. See, e.g., Remington's Pharmaceutical Sciences, 16th Ed., Mack, ed. (1980).

The T cells described herein can be formulated into a preparation in semisolid or liquid form, such as a capsule, solution, infusion, or injection. Means known in the art can be utilized to prevent or minimize release and absorption of the composition until it reaches the target tissue or organ, or to ensure timed-release of the composition. Desirably, however, a pharmaceutically acceptable form is employed that does not hinder the cells from expressing the CARs or TCRs. Thus, desirably the T cells described herein can be made into a pharmaceutical composition comprising a carrier. The T cells described herein can be formulated with a physiologically acceptable carrier or excipient to prepare a pharmaceutical composition. The carrier and composition can be sterile. Preferred carriers include, for example, a balanced salt solution, preferably Hanks' balanced salt solution, or normal saline. The formulation should suit the mode of administration. Suitable pharmaceutically acceptable carriers include but are not limited to water, salt solutions (e.g., NaCl), saline, buffered saline, as well as combinations thereof. The pharmaceutical preparations can, if desired, be mixed with auxiliary agents, e.g., lubricants, preservatives, stabilizers, wetting agents, emulsifiers, salts for influencing osmotic pressure, buffers, that do not deleteriously react with the T-cells. The T-cells may be αβ T cells or γδ T cells that express CD8 polypeptides described herein, optionally a TCR described herein.

A composition of the present invention can be provided in unit dosage form wherein each dosage unit, e.g., an injection, contains a predetermined amount of the composition, alone or in appropriate combination with other active agents.

The compositions described herein may be a pharmaceutical composition. Pharmaceutical composition described herein may further comprise an adjuvant selected from the group consisting of colony-stimulating factors, including but not limited to Granulocyte Macrophage Colony Stimulating Factor (GM-CSF, sargramostim), cyclophosphamide, imiquimod, resiquimod, interferon-alpha, or a combination thereof.

Pharmaceutical composition described herein may comprise an adjuvant selected from the group consisting of colony-stimulating factors, e.g., Granulocyte Macrophage Colony Stimulating Factor (GM-CSF, sargramostim), cyclophosphamide, imiquimod and resiquimod.

Preferred adjuvants include but are not limited to cyclophosphamide, imiquimod or resiquimod. Even more preferred adjuvants are Montanide IMS 1312, Montanide ISA 206, Montanide ISA 50V, Montanide ISA-51, poly-ICLC (Hiltonol®) and anti-CD40 mAB, or combinations thereof.

Other examples for useful adjuvants include, but are not limited to chemically modified CpGs (e.g. CpR, Idera), dsRNA analogues such as Poly(I:C) and derivates thereof (e.g. AmpliGen®, Hiltonol®, poly-(ICLC), poly(IC-R), poly(I:C12U), non-CpG bacterial DNA or RNA as well as immunoactive small molecules and antibodies such as cyclophosphamide, sunitinib, immune checkpoint inhibitors including ipilimumab, nivolumab, pembrolizumab, atezolizumab, avelumab, durvalumab, and cemiplimab, Bevacizumab®, celebrex, NCX-4016, sildenafil, tadalafil, vardenafil, sorafenib, temozolomide, temsirolimus, XL-999, CP-547632, pazopanib, VEGF Trap, ZD2171, AZD2171, anti-CTLA4, other antibodies targeting key structures of the immune system (e.g. anti-CD40, anti-TGFbeta, anti-TNFalpha receptor) and SC58175, which may act therapeutically and/or as an adjuvant. The amounts and concentrations of adjuvants and additives useful in the context of the present invention can readily be determined by the skilled artisan without undue experimentation.

Other adjuvants include but are not limited to anti-CD40, imiquimod, resiquimod, GM-CSF, cyclophosphamide, sunitinib, bevacizumab, atezolizumab, interferon-alpha, interferon-beta, CpG oligonucleotides and derivatives, poly-(I:C) and derivatives, RNA, sildenafil, and particulate formulations with poly(lactide co-glycolide) (PLG), Polyinosinic-polycytidylic acid-poly-l-lysine carboxymethylcellulose (poly-ICLC), virosomes, and/or interleukin-1 (IL-1), IL-2, IL-4, IL-7, IL-12, IL-13, IL-15, IL-18, IL-21, and IL-23. See, e.g., Narayanan et al. J. Med. Chem. (2003) 46(23): 5031-5044; Pohar et al. Scientific Reports 7 14598 (2017); Grajkowski et al. Nucleic Acids Research (2005) 33(11): 3550-3560; Martins et al. Expert Rev Vaccines (2015) 14(3): 447-59.

The composition described herein may also include one or more adjuvants. Adjuvants are substances that non-specifically enhance or potentiate the immune response (e.g., immune responses mediated by CD8-positive T cells and helper-T (TH) cells to an antigen and would thus be considered useful in the medicament of the present invention. Suitable adjuvants include, but are not limited to, 1018 ISS, aluminium salts, AMPLIVAX®, AS15, BCG, CP-870,893, CpG7909, CyaA, dSLIM, flagellin or TLR5 ligands derived from flagellin, FLT3 ligand, GM-CSF, IC30, IC31, Imiquimod (ALDARA®), resiquimod, ImuFact IMP321, Interleukins as IL-2, IL-13, IL-21, Interferon-alpha or -beta, or pegylated derivatives thereof, IS Patch, ISS, ISCOMATRIX, ISCOMs, Juvlmmune®, LipoVac, MALP2, MF59, monophosphoryl lipid A, Montanide IMS 1312, Montanide ISA 206, Montanide ISA 50V, Montanide ISA-51, water-in-oil and oil-in-water emulsions, OK-432, OM-174, OM-197-MP-EC, ONTAK, OspA, PepTel® vector system, poly(lactide co-glycolide) [PLG]-based and dextran microparticles, talactoferrin SRL172, Virosomes and other Virus-like particles, YF-17D, VEGF trap, R848, beta-glucan, Pam3Cys, Aquila's QS21 stimulon, which is derived from saponin, mycobacterial extracts and synthetic bacterial cell wall mimics, and other proprietary adjuvants such as Ribi's Detox, Quil, or Superfos. Adjuvants such as Freund's or GM-CSF are preferred. Several immunological adjuvants (e.g., MF59) specific for dendritic cells and their preparation have been described previously. Also, cytokines may be used. Several cytokines have been directly linked to influencing dendritic cell migration to lymphoid tissues (e.g., TNF−), accelerating the maturation of dendritic cells into efficient antigen-presenting cells for T-lymphocytes (e.g., GM-CSF, IL-1 and IL-4) (U.S. Pat. No. 5,849,589, incorporated herein by reference in its entirety) and acting as immunoadjuvants (e.g., IL-12, IL-15, IL-23, IL-7, IFN-alpha. IFN-beta).

CpG immunostimulatory oligonucleotides have also been reported to enhance the effects of adjuvants in a vaccine setting. Without being bound by theory, CpG oligonucleotides act by activating the innate (non-adaptive) immune system via Toll-like receptors (TLR), mainly TLR9. CpG triggered TLR9 activation enhances antigen-specific humoral and cellular responses to a wide variety of antigens, including peptide or protein antigens, live or killed viruses, dendritic cell vaccines, autologous cellular vaccines and polysaccharide conjugates in both prophylactic and therapeutic vaccines. More importantly it enhances dendritic cell maturation and differentiation, resulting in enhanced activation of TH1 cells and strong cytotoxic T-lymphocyte (CTL) generation, even in the absence of CD4 T cell help. The TH1 bias induced by TLR9 stimulation is maintained even in the presence of vaccine adjuvants such as alum or incomplete Freund's adjuvant (IFA) that normally promote a TH2 bias. CpG oligonucleotides show even greater adjuvant activity when formulated or co-administered with other adjuvants or in formulations such as microparticles, nanoparticles, lipid emulsions or similar formulations, which are especially necessary for inducing a strong response when the antigen is relatively weak. They also accelerate the immune response and enable the antigen doses to be reduced by approximately two orders of magnitude, with comparable antibody responses to the full-dose vaccine without CpG in some experiments (Krieg, 2006). U.S. Pat. No. 6,406,705 B1 describes the combined use of CpG oligonucleotides, non-nucleic acid adjuvants and an antigen to induce an antigen-specific immune response. A CpG TLR9 antagonist is dSLIM (double Stem Loop Immunomodulator) by Mologen (Berlin, Germany) which is a preferred component of the pharmaceutical composition of the present invention. Other TLR binding molecules such as RNA binding TLR 7, TLR 8 and/or TLR 9 may also be used.

### Methods of Treatment and Preparing

Engineered T cells may express modified CD8 polypeptides described herein. Further, the Engineered T cells may express a TCR described herein. The TCR expressed by the engineered T cells may recognize a TAA bound to an HLA as described herein. Engineered T cells of the present disclosure can be used to treat a subject in need of treatment for a condition, for example, a cancer described herein. The T cells may be αβ T cells or γδ T cells that express a modified CD8 polypeptide, optionally a TCR described herein.

A method of treating a condition (e.g., ailment) in a subject with T cells described herein may comprise administering to the subject a therapeutically effective amount of engineered T cells described herein, optionally γδ T cells. T cells described herein may be administered at various regimens (e.g., timing, concentration, dosage, spacing between treatment, and/or formulation). A subject can also be preconditioned with, for example, chemotherapy, radiation, or a combination of both, prior to receiving engineered T cells of the present disclosure. A population of engineered T cells may also be frozen or cryopreserved prior to being administered to a subject. A population of engineered T cells can include two or more cells that express identical, different, or a combination of identical and different tumor recognition moieties. For instance, a population of engineered T-cells can include several distinct engineered T cells that are designed to recognize different antigens, or different epitopes of the same antigen. The T cells may be αβ T cells or γδ T cells that express a CD8 polypeptide described herein, optionally a TCR described herein.

T cells described herein, including αβ T-cells and γδ T cells, may be used to treat various conditions. The T cells may be αβ T cells or γδ T cells that express a CD8 polypeptide, optionally a TCR described herein. T cells described herein may be used to treat a cancer, including solid tumors and hematologic malignancies. Non-limiting examples of cancers include: acute lymphoblastic leukemia, acute myeloid leukemia, adrenocortical carcinoma, AIDS-related cancers, AIDS-related lymphoma, anal cancer, appendix cancer, astrocytomas, neuroblastoma, basal cell carcinoma, bile duct cancer, bladder cancer, bone cancers, brain tumors, such as cerebellar astrocytoma, cerebral astrocytoma/malignant glioma, ependymoma, medulloblastoma, supratentorial primitive neuroectodermal tumors, visual pathway and hypothalamic glioma, breast cancer, bronchial adenomas, Burkitt lymphoma, carcinoma of unknown primary origin, central nervous system lymphoma, cerebellar astrocytoma, cervical cancer, childhood cancers, chronic lymphocytic leukemia, chronic myelogenous leukemia, chronic myeloproliferative disorders, colon cancer, cutaneous T-cell lymphoma, desmoplastic small round cell tumor, endometrial cancer, ependymoma, esophageal cancer, Ewing's sarcoma, germ cell tumors, gallbladder cancer, gastric cancer, gastrointestinal carcinoid tumor, gastrointestinal stromal tumor, gliomas, hairy cell leukemia, head and neck cancer, heart cancer, hepatocellular (liver) cancer, Hodgkin lymphoma, Hypopharyngeal cancer, intraocular melanoma, islet cell carcinoma, Kaposi sarcoma, kidney cancer, laryngeal cancer, lip and oral cavity cancer, liposarcoma, liver cancer, lung cancers, such as non-small cell and small cell lung cancer, lymphomas, leukemias, macroglobulinemia, malignant fibrous histiocytoma of bone/osteosarcoma, medulloblastoma, melanomas, mesothelioma, metastatic squamous neck cancer with occult primary, mouth cancer, multiple endocrine neoplasia syndrome, myelodysplastic syndromes, myeloid leukemia, nasal cavity and paranasal sinus cancer, nasopharyngeal carcinoma, neuroblastoma, non-Hodgkin lymphoma, non-small cell lung cancer, oral cancer, oropharyngeal cancer, osteosarcoma/malignant fibrous histiocytoma of bone, ovarian cancer, ovarian epithelial cancer, ovarian germ cell tumor, pancreatic cancer, pancreatic cancer islet cell, paranasal sinus and nasal cavity cancer, parathyroid cancer, penile cancer, pharyngeal cancer, pheochromocytoma, pineal astrocytoma, pineal germinoma, pituitary adenoma, pleuropulmonary blastoma, plasma cell neoplasia, primary central nervous system lymphoma, prostate cancer, rectal cancer, renal cell carcinoma, renal pelvis and ureter transitional cell cancer, retinoblastoma, rhabdomyosarcoma, salivary gland cancer, sarcomas, skin cancers, skin carcinoma merkel cell, small intestine cancer, soft tissue sarcoma, squamous cell carcinoma, stomach cancer, T-cell lymphoma, throat cancer, thymoma, thymic carcinoma, thyroid cancer, trophoblastic tumor (gestational), cancers of unknown primary site, urethral cancer, uterine sarcoma, vaginal cancer, vulvar cancer, Waldenstrm macroglobulinemia, and Wilms tumor.

The T cells described herein may be used to treat an infectious disease. The T cells described herein may be used to treat an infectious disease, an infectious disease may be caused a virus. The T cells described herein may be used to treat an immune disease, such as an autoimmune disease. The T cells may be αβ T cells or γδ T cells that express a CD8 polypeptide, optionally a TCR described herein.

Treatment with T cells described herein, optionally γδ T cells, may be provided to the subject before, during, and after the clinical onset of the condition. Treatment may be provided to the subject after 1 day, 1 week, 6 months, 12 months, or 2 years after clinical onset of the disease. Treatment may be provided to the subject for more than 1 day, 1 week, 1 month, 6 months, 12 months, 2 years, 3 years, 4 years, 5 years, 6 years, 7 years, 8 years, 9 years, 10 years or more after clinical onset of disease. Treatment may be provided to the subject for less than 1 day, 1 week, 1 month, 6 months, 12 months, or 2 years after clinical onset of the disease. Treatment may also include treating a human in a clinical trial. A treatment can include administering to a subject a pharmaceutical composition comprising engineered T cells described herein. The T cells may be αβ T cells or γδ T cells that express a CD8 polypeptide, optionally a TCR described herein.

In an embodiment, administration of engineered T cells of the present disclosure to a subject may modulate the activity of endogenous lymphocytes in a subject's body. In an embodiment, administration of engineered T cells to a subject may provide an antigen to an endogenous T-cell and may boost an immune response. In an embodiment, the memory T cell may be a CD4+ T-cell. In an embodiment, the memory T cell may be a CD8+ T-cell. In an embodiment, administration of engineered T cells of the present disclosure to a subject may activate the cytotoxicity of another immune cell. In an embodiment, the other immune cell may be a CD8+ T-cell. In an embodiment, the other immune cell may be a Natural Killer T-cell. In an embodiment, administration of engineered γδ T-cells of the present disclosure to a subject may suppress a regulatory T-cell. In an embodiment, the regulatory T-cell may be a FOX3+ Treg cell. In an embodiment, the regulatory T-cell may be a FOX3− Treg cell. Non-limiting examples of cells whose activity can be modulated by engineered T cells of the disclosure may comprise: hematopioietic stem cells; B cells; CD4; CD8; red blood cells; white blood cells; dendritic cells, including dendritic antigen presenting cells; leukocytes; macrophages; memory B cells; memory T-cells; monocytes; natural killer cells; neutrophil granulocytes; T-helper cells; and T-killer cells. The T cells may be αβ T cells or γδ T cells that express a CD8 polypeptide, optionally a TCR described herein.

During most bone marrow transplants, a combination of cyclophosphamide with total body irradiation may be conventionally employed to prevent rejection of the hematopietic stem cells (HSC) in the transplant by the subject's immune system. In an embodiment, incubation of donor bone marrow with interleukin-2 (IL-2) ex vivo may be performed to enhance the generation of killer lymphocytes in the donor marrow. Interleukin-2 (IL-2) is a cytokine that may be necessary for the growth, proliferation, and differentiation of wild-type lymphocytes. Current studies of the adoptive transfer of γδ T-cells into humans may require the co-administration of γδ T-cells and interleukin-2. However, both low- and high-dosages of IL-2 can have highly toxic side effects. IL-2 toxicity can manifest in multiple organs/systems, most significantly the heart, lungs, kidneys, and central nervous system. In an embodiment, the disclosure provides a method for administrating engineered T cells to a subject without the co-administration of a native cytokine or modified versions thereof, such as IL-2, IL-15, IL-12, IL-21. In an embodiment, engineered T cells can be administered to a subject without co-administration with IL-2. In an embodiment, engineered T cells may be administered to a subject during a procedure, such as a bone marrow transplant without the co-administration of IL-2.

In an embodiment, the methods may further comprise administering a chemotherapy agent. The dosage of the chemotherapy agent may be sufficient to deplete the patient's T-cell population. The chemotherapy may be administered about 5-7 days prior to T-cell administration. The chemotherapy agent may be cyclophosphamide, fludarabine, or a combination thereof. The chemotherapy agent may comprise dosing at about 400-600 mg/m²/day of cyclophosphamide. The chemotherapy agent may comprise dosing at about 10-30 mg/m²/day of fludarabine.

In an embodiment, the methods may further comprise pre-treatment of the patient with low-dose radiation prior to administration of the composition comprising T-cells. The low dose radiation may comprise about 1.4 Gy for 1-6 days, preferably about 5 days, prior to administration of the composition comprising T-cells.

In an embodiment, the patient may be HLA-A*02.

In an embodiment, the patient may be HLA-A*06.

In an embodiment, the methods may further comprise administering an anti-PD1 antibody. The anti-PD1 antibody may be a humanized antibody. The anti-PD1 antibody may be pembrolizumab. The dosage of the anti-PD1 antibody may be about 200 mg. The anti-PD1 antibody may be administered every 3 weeks following T-cell administration.

In an embodiment, the dosage of T-cells may be between about 0.8-1.2×10⁹ T cells. The dosage of the T cells may be about 0.5×10⁸ to about 10×10⁹ T cells. The dosage of T-cells may be about 1.2-3×10⁹ T cells, about 3-6×10⁹ T cells, about 10×10⁹ T cells, about 5×10⁹ T cells, about 0.1×10⁹ T cells, about 1×10⁸ T cells, about 5×10⁸ T cells, about 1.2-6×10⁹ T cells, about 1-6×10⁹ T cells, or about 1-8×10⁹ T cells.

In an embodiment, the T cells may be administered in 3 doses. The T-cell doses may escalate with each dose. The T-cells may be administered by intravenous infusion.

In an embodiment, the CD8 sequences described herein and associated products and compositions may be used autologous or allogenic methods of adoptive cellular therapy. In another embodiment, CD8 sequences, T cells thereof, and compositions may be used in, for example, methods described in U.S. Patent Application Publication 2019/0175650; U.S. Patent Application Publication 2019/0216852; U.S. Patent Application Publication 2019/024743; and U.S. Provisional Patent Application 62/980,844, each of which are incorporated by reference in their entireties.

The disclosure also provides for a population of modified T cells that present an exogenous CD8 polypeptide described herein and a T cell receptor wherein the population of modified T cells is activated and expanded with a combination of IL-2 and IL-15. In another embodiment, the population of modified T cells are expanded and/or activated with a combination of IL-2, IL-15, and zoledronate. In yet another embodiment, the population of modified T cells are activated with a combination of IL-2, IL-15, and zoledronate while expanded with a combination of IL-2, IL-15, and without zoledronate. The disclosure further provides for use of other interleukins during activation and/or expansion, such as IL-12, IL-18, IL-21, and combinations thereof.

In an aspect, IL-21, a histone deacetylase inhibitor (HDACi), or combinations thereof may be utilized in the field of cancer treatment, with methods described herein, and/or with ACT processes described herein. In an embodiment, the present disclosure provides methods for re-programming effector T cells to a central memory phenotype comprising culturing the effector T cells with at least one HDACi together with IL-21. Representative HDACi include, for example, trichostatin A, trapoxin B, phenylbutyrate, valproic acid, vorinostat (suberanilohydroxamic acid), belinostat, panobinostat, dacinostat, entinostat, tacedinaline, and mocetinostat.

Compositions comprising engineered T cells described herein may be administered for prophylactic and/or therapeutic treatments. In therapeutic applications, pharmaceutical compositions can be administered to a subject already suffering from a disease or condition in an amount sufficient to cure or at least partially arrest the symptoms of the disease or condition. An engineered T-cell can also be administered to lessen a likelihood of developing, contracting, or worsening a condition. Effective amounts of a population of engineered T-cells for therapeutic use can vary based on the severity and course of the disease or condition, previous therapy, the subject's health status, weight, and/or response to the drugs, and/or the judgment of the treating physician. The T cells may be a T cells or γδ T cells engineered to express modified CD8 polypeptides described herein and optionally a TCR described herein. T-cell therapy has been successful in treating various cancers. Li et al. Signal Transduction and Targeted Therapy 4(35): (2019), the content of which is incorporated by reference in its entirety.

### Methods of Administration

One or multiple engineered T cell populations described herein may be administered to a subject in any order or simultaneously. If simultaneously, the multiple engineered T cell can be provided in a single, unified form, such as an intravenous injection, or in multiple forms, for example, as multiple intravenous infusions, subcutaneous injections or pills. Engineered T-cells can be packed together or separately, in a single package or in a plurality of packages. One or all of the engineered T cells can be given in multiple doses. If not simultaneous, the timing between the multiple doses may vary to as much as about a week, a month, two months, three months, four months, five months, six months, or about a year. In an embodiment, engineered T cells can expand within a subject's body, in vivo, after administration to a subject. Engineered T cells can be frozen to provide cells for multiple treatments with the same cell preparation. Engineered T cells of the present disclosure, and pharmaceutical compositions comprising the same, can be packaged as a kit. A kit may comprise instructions (e.g., written instructions) on the use of engineered T cells and compositions comprising the same.

A method of treating a cancer may comprise administering to a subject a therapeutically-effective amount of engineered T cells, in which the administration treats the cancer. In an embodiments, the therapeutically-effective amount of engineered γδ T cells may be administered for at least about 10 seconds, 30 seconds, 1 minute, 10 minutes, 30 minutes, 1 hour, 2 hours, 3 hours, 4 hours, 5 hours, 6 hours, 12 hours, 24 hours, 2 days, 3 days, 4 days, 5 days, 6 days, 1 week, 2 weeks, 3 weeks, 1 month, 2 months, 3 months, 4 months, 5 months, 6 months, or 1 year. In an embodiment, the therapeutically-effective amount of the engineered T cells may be administered for at least one week. In an embodiment, the therapeutically-effective amount of engineered T cells may be administered for at least two weeks.

Engineered T-cells described herein, optionally γδ T cells, can be administered before, during, or after the occurrence of a disease or condition, and the timing of administering a pharmaceutical composition comprising an engineered T-cell can vary. For example, engineered T cells can be used as a prophylactic and can be administered continuously to subjects with a propensity to conditions or diseases in order to lessen the likelihood of occurrence of the disease or condition. Engineered T-cells can be administered to a subject during or as soon as possible after the onset of the symptoms. The administration of engineered T cells can be initiated immediately within the onset of symptoms, within the first 3 hours of the onset of the symptoms, within the first 6 hours of the onset of the symptoms, within the first 24 hours of the onset of the symptoms, within 48 hours of the onset of the symptoms, or within any period of time from the onset of symptoms. The initial administration can be via any route practical, such as by any route described herein using any formulation described herein. In an embodiment, the administration of engineered T cells of the present disclosure may be an intravenous administration. One or multiple dosages of engineered T cells can be administered as soon as is practicable after the onset of a cancer, an infectious disease, an immune disease, sepsis, or with a bone marrow transplant, and for a length of time necessary for the treatment of the immune disease, such as, for example, from about 24 hours to about 48 hours, from about 48 hours to about 1 week, from about 1 week to about 2 weeks, from about 2 weeks to about 1 month, from about 1 month to about 3 months. For the treatment of cancer, one or multiple dosages of engineered T cells can be administered years after onset of the cancer and before or after other treatments. In an embodiment, engineered γδ T cells can be administered for at least about 10 minutes, 30 minutes, 1 hour, 2 hours, 3 hours, 4 hours, 5 hours, 6 hours, 12 hours, 24 hours, at least 48 hours, at least 72 hours, at least 96 hours, at least 1 week, at least 2 weeks, at least 3 weeks, at least 4 weeks, at least 1 month, at least 2 months, at least 3 months, at least 4 months, at least 5 months, at least 6 months, at least 7 months, at least 8 months, at least 9 months, at least 10 months, at least 11 months, at least 12 months, at least 1 year, at least 2 years at least 3 years, at least 4 years, or at least 5 years. The length of treatment can vary for each subject. The T cells may be αβ T cells or γδ T cells that express a CD8 polypeptide described herein, optionally a TCR described herein.

Engineered T-cell expressing a CD8 polypeptides described herein, optionally a T cells or γδ T cells, may be present in a composition in an amount of at least 1×10³ cells/ml, at least 2×10³ cells/ml, at least 3×10³ cells/ml, at least 4×10³ cells/ml, at least 5×10³ cells/ml, at least 6×10³ cells/ml, at least 7×10³ cells/ml, at least 8×10³ cells/ml, at least 9×10³ cells/ml, at least 1×10⁴ cells/ml, at least 2×10⁴ cells/ml, at least 3×10⁴ cells/ml, at least 4×10⁴ cells/ml, at least 5×10⁴ cells/ml, at least 6×10⁴ cells/ml, at least 7×10⁴ cells/ml, at least 8×10⁴ cells/ml, at least 9×10⁴ cells/ml, at least 1×10⁵ cells/ml, at least 2×10⁵ cells/ml, at least 3×10⁵ cells/ml, at least 4×10⁵ cells/ml, at least 5×10⁵ cells/ml, at least 6×10⁵ cells/ml, at least 7×10⁵ cells/ml, at least 8×10⁵ cells/ml, at least 9×10⁵ cells/ml, at least 1×10⁶ cells/ml, at least 2×10⁶ cells/ml, at least 3×10⁶ cells/ml, at least 4×10⁶ cells/ml, at least 5×10⁶ cells/ml, at least 6×10⁶ cells/ml, at least 7×10⁶ cells/ml, at least 8×10⁶ cells/ml, at least 9×10⁶ cells/ml, at least 1×10⁷ cells/ml, at least 2×10⁷ cells/ml, at least 3×10⁷ cells/ml, at least 4×10⁷ cells/ml, at least 5×10⁷ cells/ml, at least 6×10⁷ cells/ml, at least 7×10⁷ cells/ml, at least 8×10⁷ cells/ml, at least 9×10⁷ cells/ml, at least 1×10⁸ cells/ml, at least 2×10⁸ cells/ml, at least 3×10⁸ cells/ml, at least 4×10⁸ cells/ml, at least 5×10⁸ cells/ml, at least 6×10⁸ cells/ml, at least 7×10⁸ cells/ml, at least 8×10⁸ cells/ml, at least 9×10⁸ cells/ml, at least 1×10⁹ cells/ml, or more, from about 1×10³ cells/ml to about at least 1×10⁸ cells/ml, from about 1×10⁵ cells/ml to about at least 1×10⁸ cells/ml, or from about 1×10⁶ cells/ml to about at least 1×10⁸ cells/ml.

### Sequences

The sequences described herein may comprise about 80%, about 85%, about 90%, about 85%, about 96%, about 97%, about 98%, or about 99% or 100% identity to the sequence of any of SEQ ID NO: 1-97, 256-266, 293 and 294. The sequences described herein may comprise at least 80%, at least 85%, at least 90%, at least 85%, at least 96%, at least 97%, at least 98%, at least 99% or 100% identity to the sequence of any of SEQ ID NO: 1-97 and 256-266. A sequence “at least 85% identical to a reference sequence” is a sequence having, on its entire length, 85%, or more, in particular 90%, 91%, 92%, 93%, 94%, 95%, 96%, 97%, 98%, 99%, or 100% sequence identity with the entire length of the reference sequence.

In another embodiment, the disclosure provides for sequences at least 80%, at least 85%, at least 90%, at least 85%, at least 96%, at least 97%, at least 98%, at least 99% or 100% identity to WPREmut1 (SEQ ID NO: 256), or WPRE version 2, e.g., WPREmut2 (SEQ ID NO: 257). In another aspect, the disclosure provides for sequences at least 1, 2, 3, 4, 5, 10, 15, or 20 amino acid substitutions in WPREmut1 (SEQ ID NO: 256), or WPRE version 2, e.g., WPREmut2 (SEQ ID NO: 257). In yet another aspect, the disclosure provides for sequences at most 1, 2, 3, 4, 5, 10, 15, or 20 amino acid substitutions in WPREmut1 (SEQ ID NO: 256), or WPRE version 2, e.g., WPREmut2 (SEQ ID NO: 257). In another aspect, the sequence substitutions are conservative substitutions.

Percentage of identity may be calculated using a global pairwise alignment (e.g., the two sequences are compared over their entire length). Methods for comparing the identity of two or more sequences are well known in the art. The needle program, which uses the Needleman-Wunsch global alignment algorithm (Needleman and Wunsch, 1970 J. Mol. Biol. 48:443-453) to find the optimum alignment (including gaps) of two sequences when considering their entire length, may for example be used. The needle program is for example available on the ebi.ac.uk World Wide Web site and is further described in the following publication (EMBOSS: The European Molecular Biology Open Software Suite (2000) Rice, P. Longden, I. and Bleasby, A. Trends in Genetics 16, (6) pp. 276-277). The percentage of identity between two polypeptides, in accordance with the invention, is calculated using the EMBOSS: needle (global) program with a “Gap Open” parameter equal to 10.0, a “Gap Extend” parameter equal to 0.5, and a Blosum62 matrix.

Proteins consisting of an amino acid sequence “at least 80%, 85%, 90%, 95%, 96%, 97%, 98% or 99% identical” to a reference sequence may comprise mutations such as deletions, insertions and/or substitutions compared to the reference sequence. In case of substitutions, the protein consisting of an amino acid sequence at least 80%, 85%, 90%, 95%, 96%, 97%, 98% or 99% identical to a reference sequence may correspond to a homologous sequence derived from another species than the reference sequence.

Amino acid substitutions may be conservative or non-conservative. Preferably, substitutions are conservative substitutions, in which one amino acid is substituted for another amino acid with similar structural and/or chemical properties.

Conservative substitutions may comprise those, which are described by Dayhoff in “The Atlas of Protein Sequence and Structure. Vol. 5”, Natl. Biomedical Research, the contents of which are incorporated by reference in their entirety. For example, in an embodiment, amino acids, which belong to one of the following groups, can be exchanged for one another, thus, constituting a conservative exchange: Group 1: alanine (A), proline (P), glycine (G), asparagine (N), serine (S), threonine (T); Group 2: cysteine (C), serine (S), tyrosine (Y), threonine (T); Group 3: valine (V), isoleucine (I), leucine (L), methionine (M), alanine (A), phenylalanine (F); Group 4: lysine (K), arginine (R), histidine (H); Group 5: phenylalanine (F), tyrosine (Y), tryptophan (W), histidine (H); and Group 6: aspartic acid (D), glutamic acid (E). In an embodiment, a conservative amino acid substitution may be selected from the following of T→A, G→A, A→1, T→V, A→M, T→1, A→V, T→G, and/or T→S.

A conservative amino acid substitution may comprise the substitution of an amino acid by another amino acid of the same class, for example, (1) nonpolar: Ala, Val, Leu, lie, Pro, Met, Phe, Trp; (2) uncharged polar: Gly, Ser, Thr, Cys, Tyr, Asn, Gln; (3) acidic: Asp, Glu; and (4) basic: Lys, Arg, His. Other conservative amino acid substitutions may also be made as follows: (1) aromatic: Phe, Tyr, His; (2) proton donor: Asn, Gln, Lys, Arg, His, Trp; and (3) proton acceptor: Glu, Asp, Thr, Ser, Tyr, Asn, Gln (see, for example, U.S. Pat. No. 10,106,805, the contents of which are incorporated by reference in their entirety).

Conservative substitutions may be made in accordance with Table A. Methods for predicting tolerance to protein modification may be found in, for example, Guo et al., Proc. Natl. Acad. Sci., USA, 101(25):9205-9210 (2004), the contents of which are incorporated by reference in their entirety.

TABLE A
Conservative Amino Acid substitution
Conservative Amino Acid Substitutions

| Amino Acid | Substitutions (others are known in the art) |
|---|---|
| Ala | Ser, Gly, Cys |
| Arg | Lys, Gln, His |
| Asn | Gln, His, Glu, Asp |
| Asp | Glu, Asn, Gln |
| Cys | Ser, Met, Thr |
| Gln | Asn, Lys, Glu, Asp, Arg |
| Glu | Asp, Asn, Gln |
| Gly | Pro, Ala, Ser |
| His | Asn, Gln, Lys |
| Ile | Leu, Val, Met, Ala |
| Leu | Ile, Val, Met, Ala |
| Lys | Arg, Gln, His |
| Met | Leu, Ile, Val, Ala, Phe |
| Phe | Met, Leu, Tyr, Trp, His |
| Ser | Thr, Cys, Ala |
| Thr | Ser, Val, Ala |
| Trp | Tyr, Phe |
| Tyr | Tip, Phe, His |
| Val | Ile, Leu, Met, Ala, Thr |

The sequences described herein may comprise 1, 2, 3, 4, 5, 10, 15, 20, 25, or 30 amino acid or nucleotide mutations, substitutions, deletions. Any one of SEQ ID NO: 1-97, 256-266, 293, and 294 may comprise 1, 2, 3, 4, 5, 10, 15, 20, 25, or 30 mutations, substitutions, or deletions. In another aspect, any one of SEQ ID NO: 1-97, 256-266, 293, and 294 may comprise at most 1, 2, 3, 4, 5, 10, 15, 20, 25, or 30 mutations, substitutions, or deletions. In an aspect, the mutations or substitutions may be conservative amino acid substitutions.

Conservative substitutions in the polypeptides described herein may be those shown in Table B under the heading of “conservative substitutions.” If such substitutions result in a change in biological activity, then more substantial changes, denominated “exemplary substitutions” in Table B, may be introduced and the products screened if needed.

TABLE B
Amino Acid substitution
Amino Acid Substitutions

| Original Residue | | |
|---|---|---|
| (naturally | | |
| occurring amino | Conservative | |
| acid) | Substitutions | Exemplary Substitutions |
| Ala (A) | Val | Val.; Leu; Ile |
| Arg (R) | Lys | Lys; Gln; Asn |
| Asn (N) | Gln | Gln; His; Asp, Lys; Arg |
| Asp (D) | Glu | Glu; Asn |
| Cys (C) | Ser | Ser; Ala |
| Gln (Q) | Asn | Asn; Glu |
| Glu (E) | Asp | Asp; Gln |
| Gly (G) | Ala | Ala |
| His (H) | Arg | Asn; Gln; Lys; Arg |
| Ile (I) | Leu | Leu; Val; Met; Ala; Phe; |
| | | Norleucine |
| Leu (L) | Ile | Norleucine; Ile; Val; Met; |
| | | Ala; Phe |
| Lys (K) | Arg | Arg; Gln; Asn |
| Met (M) | Leu | Leu; Phe; Ile |
| Phe (F) | Tyr | Leu; Val; Ile; Ala; Tyr |
| Pro (P) | Ala | Ala |
| Ser (S) | Thr | Thr |
| Thr (T) | Ser | Ser |
| Trp (W) | Tyr | Tyr; Phe |
| Tyr (Y) | Phe | Trp; Phe; Thr; Ser |
| Val (V) | Leu | Ile; Leu; Met; Phe; Ala; |
| | | Norleucine |

Unless otherwise indicated, all terms used herein have the same meaning as they would to one skilled in the art.

In this specification and the appended claims, the singular forms “a,” “an,” and “the” include plural reference unless the context clearly dictates otherwise. Unless defined otherwise, all technical and scientific terms used herein have the same meaning as commonly understood to one of ordinary skill in the art to which this disclosure belongs.

Activation” as used herein refers broadly to the state of a T cell that has been sufficiently stimulated to induce detectable cellular proliferation. Activation can also be associated with induced cytokine production, and detectable effector functions. The term “activated T cells” refers to, among other things, T cells that are proliferating.

“Antibodies” as used herein refer broadly to antibodies or immunoglobulins of any isotype, fragments of antibodies, which retain specific binding to antigen, including, but not limited to, Fab, Fab′, Fab′-SH, (Fab′)₂ Fv, scFv, divalent scFv, and Fd fragments, chimeric antibodies, humanized antibodies, single-chain antibodies, and fusion proteins including an antigen-specific targeting region of an antibody and a non-antibody protein. Antibodies are organized into five classes—IgG, IgE, IgA, IgD, and IgM.

“Antigen” or “Antigenic,” as used herein, refers broadly to a peptide or a portion of a peptide capable of being bound by an antibody which is additionally capable of inducing an animal to produce an antibody capable of binding to an epitope of that antigen. An antigen may have one epitope or have more than one epitope. The specific reaction referred to herein indicates that the antigen will react, in a highly selective manner, with its corresponding antibody and not with the multitude of other antibodies which may be evoked by other antigens.

“Chimeric antigen receptor” or “CAR” or “CARs” as used herein refers broadly to genetically modified receptors, which graft an antigen specificity onto cells, for example T cells, NK cells, macrophages, and stem cells. CARs can include at least one antigen-specific targeting region (ASTR), a hinge or stalk domain, a transmembrane domain (TM), one or more co-stimulatory domains (CSDs), and an intracellular activating domain (IAD). In certain embodiments, the CSD is optional. In another embodiment, the CAR is a bispecific CAR, which is specific to two different antigens or epitopes. After the ASTR binds specifically to a target antigen, the IAD activates intracellular signaling. For example, the IAD can redirect T cell specificity and reactivity toward a selected target in a non-MHC-restricted manner, exploiting the antigen-binding properties of antibodies. The non-MHC-restricted antigen recognition gives T cells expressing the CAR the ability to recognize an antigen independent of antigen processing, thus bypassing a major mechanism of tumor escape. Moreover, when expressed in T cells, CARs advantageously do not dimerize with endogenous T cell receptor (TCR) alpha and beta chains.

“Cytotoxic T lymphocyte” (CTL) as used herein refers broadly to a T lymphocyte that expresses CD8 on the surface thereof (e.g., a CD8+ T cell). Such cells may be preferably “memory” T cells (T_{M} cells) that are antigen-experienced.

“Effective amount”, “therapeutically effective amount”, or “efficacious amount” as used herein refers broadly to the amount of an agent, or combined amounts of two agents, that, when administered to a mammal or other subject for treating a disease, is sufficient to affect such treatment for the disease. The “therapeutically effective amount” will vary depending on the agent(s), the disease and its severity and the age, weight, etc., of the subject to be treated.

“Genetically modified” as used herein refers broadly to methods to introduce exogenous nucleic acids into a cell, whether or not the exogenous nucleic acids are integrated into the genome of the cell. “Genetically modified cell” as used herein refers broadly to cells that contain exogenous nucleic acids whether or not the exogenous nucleic acids are integrated into the genome of the cell.

“Immune cells” as used herein refers broadly to white blood cells (leukocytes) derived from hematopoietic stem cells (HSC) produced in the bone marrow “Immune cells” include, without limitation, lymphocytes (T cells, B cells, natural killer (NK) (CD3−CD56+) cells) and myeloid-derived cells (neutrophil, eosinophil, basophil, monocyte, macrophage, dendritic cells). “T cells” include all types of immune cells expressing CD3 including T-helper cells (CD4+ cells), cytotoxic T-cells (CD8+ cells), T-regulatory cells (Treg) and gamma-delta T cells, and NK T cells (CD3+ and CD56+). A skilled artisan will understand T cells and/or NK cells, as used throughout the disclosure, can include only T cells, only NK cells, or both T cells and NK cells. In certain illustrative embodiments and aspects provided herein, T cells are activated and transduced. Furthermore, T cells are provided in certain illustrative composition embodiments and aspects provided herein. A “cytotoxic cell” includes CD8+ T cells, natural-killer (NK) cells, NK-T cells, γδ T cells, and neutrophils, which are cells capable of mediating cytotoxicity responses.

“Individual,” “subject,” “host,” and “patient,” as used interchangeably herein, refer broadly to a mammal, including, but not limited to, humans, murines (e.g., rats, mice), lagomorphs (e.g., rabbits), non-human primates, canines, felines, and ungulates (e.g., equines, bovines, ovines, porcines, caprines).

“Peripheral blood mononuclear cells” or “PBMCs” as used herein refers broadly to any peripheral blood cell having a round nucleus. PBMCs include lymphocytes, such as T cells, B cells, and NK cells, and monocytes.

“Polynucleotide” and “nucleic acid”, as used interchangeably herein, refer broadly to a polymeric form of nucleotides of any length, either ribonucleotides or deoxyribonucleotides. Thus, this term includes, but is not limited to, single-, double-, or multi-stranded DNA or RNA, genomic DNA, cDNA, DNA-RNA hybrids, or a polymer including purine and pyrimidine bases or other natural, chemically or biochemically modified, non-natural, or derivatized nucleotide bases.

“T cell” or “T lymphocyte,” as used herein, refer broadly to thymocytes, naïve T lymphocytes, immature T lymphocytes, mature T lymphocytes, resting T lymphocytes, or activated T lymphocytes. Illustrative populations of T cells suitable for use in particular embodiments include, but are not limited to, helper T cells (HTL; CD4+ T cell), a cytotoxic T cell (CTL; CD8+ T cell), CD4+CD8+ T cell, CD4−CD8− T cell, natural killer T cell, T cells expressing a TCR (αβ T cells), T cells expressing γδ TCR (γδ T cells), or any other subset of T cells. Other illustrative populations of T cells suitable for use in particular embodiments include, but are not limited to, T cells expressing one or more of the following markers: CD3, CD4, CD8, CD27, CD28, CD45RA, CD45RO, CD62L, CD127, CD197, and HLA-DR and if desired, can be further isolated by positive or negative selection techniques.

In the present invention, the term “homologous” refers to the degree of identity (see percent identity above) between sequences of two amino acid sequences, e.g., peptide or polypeptide sequences. The aforementioned “homology” is determined by comparing two sequences aligned under optimal conditions over the sequences to be compared. Such a sequence homology can be calculated by creating an alignment using, for example, the ClustalW algorithm. Commonly available sequence analysis software, more specifically, Vector NTI, GENETYX or other tools are provided by public databases.

The terms “sequence homology” or “sequence identity” are used interchangeably herein. For the purpose of this invention, it is defined here that in order to determine the percentage of sequence homology or sequence identity of two amino acid sequences or of two nucleotide sequences, the sequences are aligned for optimal comparison purposes. In order to optimize the alignment between the two sequences, gaps may be introduced in any of the two sequences that are compared. Such alignment can be carried out over the full-length of the sequences being compared. Alternatively, the alignment may be carried out over a shorter length, for example over about 5, about 10, about 20, about 50, about 100 or more nucleotides or amino acids. The sequence identity is the percentage of identical matches between the two sequences over the reported aligned region.

A comparison of sequences and determination of percentage of sequence identity between two sequences can be accomplished using a mathematical algorithm. The skilled person will be aware of the fact that several different computer programs are available to align two sequences and determine the identity between two sequences (Kruskal, J. B. (1983) An overview of sequence comparison. In D. Sankoff and J. B. Kruskal, (ed.), Time warps, string edits and macromolecules: the theory and practice of sequence comparison, Addison Wesley). The percent sequence identity between two amino acid sequences or between two nucleotide sequences may be determined using the Needleman and Wunsch algorithm for the alignment of two sequences. (Needleman, S. B. and Wunsch, C. D. (1970) J. Mal. Biol. 48, 443-453). Both amino acid sequences and nucleotide sequences can be aligned by the algorithm. The Needleman-Wunsch algorithm has been implemented in the computer program NEEDLE. For the purpose of this invention, the NEEDLE program from the EMBOSS package was used (version 2.8.0 or higher, EMBOSS: The European Molecular Biology Open Software Suite (2000) Rice, Longden, and Bleasby, Trends in Genetics 16, (6) 276-277, emboss.bioinformatics.nl/). For amino acid sequences, EBLOSUM62 is used for the substitution matrix. For nucleotide sequence, EDNAFULL is used. The optional parameters used are a gap-open penalty of 10 and a gap extension penalty of 0.5. The skilled person will appreciate that all these different parameters will yield slightly different results but that the overall percentage identity of two sequences is not significantly altered when using different algorithms.

After alignment by the program NEEDLE as described above the percentage of sequence identity between a query sequence and a sequence of the invention is calculated as follows: Number of corresponding positions in the alignment showing an identical amino acid or identical nucleotide in both sequences divided by the total length of the alignment after subtraction of the total number of gaps in the alignment. The identity defined as herein can be obtained from NEEDLE by using the NOBRIEF option and is labelled in the output of the program as “longest-identity”. The nucleotide and amino acid sequences of the present invention can further be used as a “query sequence” to perform a search against sequence databases to, for example, identify other family members or related sequences. Such searches can be performed using the NBLAST and XBLAST programs (version 2.0) of Altschul et al. (1990) J. Mal. Biol. 215:403-10. BLAST nucleotide searches can be performed with the NBLAST program, score=100, word length=12 to obtain nucleotide sequences homologous to polynucleotides of the invention. BLAST protein searches can be performed with the XBLAST program, score=50, word length=3 to obtain amino acid sequences homologous to polypeptides of the invention. To obtain gapped alignments for comparison purposes, Gapped BLAST can be utilized as described in Altschul et al. (1997) Nucleic Acids Res. 25(17): 3389-3402. When utilizing BLAST and Gapped BLAST programs, the default parameters of the respective programs (e.g., XBLAST and NBLAST) can be used.

“T-cell receptor (TCR)” as used herein refers broadly to a protein receptor on T cells that is composed of a heterodimer of an alpha (a) and beta (P) chain, although in some cells the TCR consists of gamma and delta (γ/δ) chains. The TCR may be modified on any cell comprising a TCR, including a helper T cell, a cytotoxic T cell, a memory T cell, regulatory T cell, natural killer T cell, or a gamma delta T cell.

The TCR is generally found on the surface of T lymphocytes (or T cells) that is generally responsible for recognizing antigens bound to major histocompatibility complex (MHC) molecules. It is a heterodimer consisting of an alpha and beta chain in 95% of T cells, while 5% of T cells have TCRs consisting of gamma and delta chains. Engagement of the TCR with antigen and MHC results in activation of its T lymphocyte through a series of biochemical events mediated by associated enzymes, co-receptors, and specialized accessory molecules. In immunology, the CD3 antigen (CD stands for cluster of differentiation) is a protein complex composed of four distinct chains (CD3-γ, CD3δ, and two times CD3ε) in mammals, that associate with molecules known as the T-cell receptor (TCR) and the (-chain to generate an activation signal in T lymphocytes. The TCR, ζ-chain, and CD3 molecules together comprise the TCR complex. The CD3-γ, CD3δ, and CD3ε chains are highly related cell surface proteins of the immunoglobulin superfamily containing a single extracellular immunoglobulin domain. The transmembrane region of the CD3 chains is negatively charged, a characteristic that allows these chains to associate with the positively charged TCR chains (TCRα and TCRβ). The intracellular tails of the CD3 molecules contain a single conserved motif known as an immunoreceptor tyrosine-based activation motif or ITAM for short, which is essential for the signaling capacity of the TCR.

“Treatment,” “treating,” and the like, as used herein refer broadly to obtaining a desired pharmacologic and/or physiologic effect. The effect may be prophylactic in terms of completely or partially preventing a disease or symptom thereof and/or may be therapeutic in terms of a partial or complete cure for a disease and/or adverse effect attributable to the disease. “Treatment,” as used herein, covers any treatment of a disease in a mammal, e.g., in a human, and includes: (a) preventing the disease from occurring in a subject which may be predisposed to the disease but has not yet been diagnosed as having it; (b) inhibiting the disease, e.g., arresting its development; and (c) relieving the disease, e.g., causing regression of the disease.

The ability of dendritic cells (DC) to activate and expand antigen-specific CD8+ T cells may depend on the DC maturation stage and that DCs may need to receive a “licensing” signal, associated with IL-12 production, in order to elicit cytolytic immune response. In particular, the provision of signals through CD40 Ligand (CD40L)-CD40 interactions on CD4+ T cells and DCs, respectively, may be considered important for the DC licensing and induction of cytotoxic CD8+ T cells. DC licensing may result in the upregulation of co-stimulatory molecules, increased survival and better cross-presenting capabilities of DCs. This process may be mediated via CD40/CD40L interaction [S. R. Bennet et al., “Help for cytotoxic T-cell responses is mediated by CD40 signalling,” Nature 393(6684):478-480 (1998); S. P. Schoenberger et al., “T-cell help for cytotoxic T-cell help is mediated by CD40-CD40L interactions,” Nature 393(6684):480-483 (1998)], but CD40/CD40L-independent mechanisms also exist (CD70, LTβR). In addition, a direct interaction between CD40L expressed on DCs and CD40 on expressed on CD8+ T-cells has also been suggested, providing a possible explanation for the generation of helper-independent CTL responses [S. Johnson et al., “Selected Toll-like receptor ligands and viruses promote helper-independent cytotoxic T-cell priming by upregulating CD40L on dendritic cells,” Immunity 30(2):218-227 (2009)].

### Example 1

### Exemplary Nucleic Acid and Amino Acid Sequences

TABLE 2
CD8-TCR Constructs

| | Nucleic Acid | Amino Acid |
|---|---|---|
| Construct | (SEQ ID | (SEQ ID |
| # | NO) | NO) |
| 1 | 295 | 296 |
| 2 | 297 | 298 |
| 8 | 299 | 300 |
| 9 | 287 | 288 |
| 9b | 287 | 288 |
| 10 | 291 | 292 |
| 10n | 291 | 292 |
| 11 | 285 | 286 |
| 11n | 285 | 286 |
| 12 | 301 | 302 |
| 13 | 267 | 268 |
| 14 | 269 | 270 |
| 15 | 271 | 272 |
| 16 | 273 | 274 |
| 17 | 275 | 276 |
| 18 | 277 | 278 |
| 19 | 279 | 280 |
| 21 | 281 | 282 |
| 22 | 283 | 284 |
| 25 | 289 | 290 |

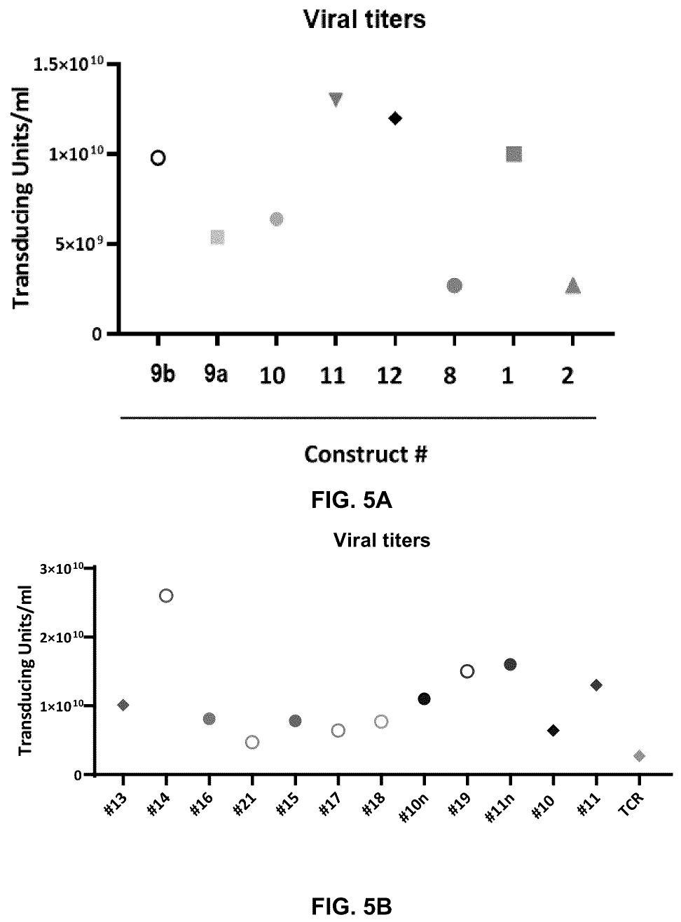

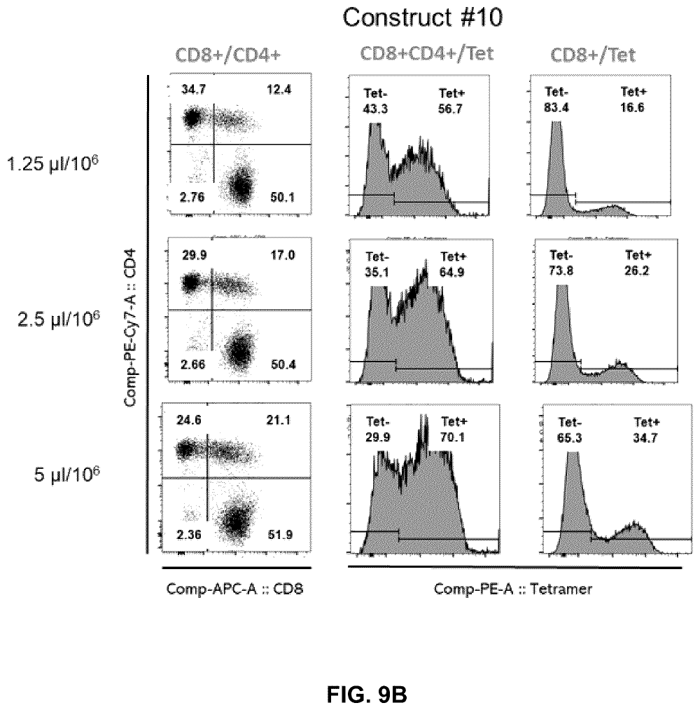

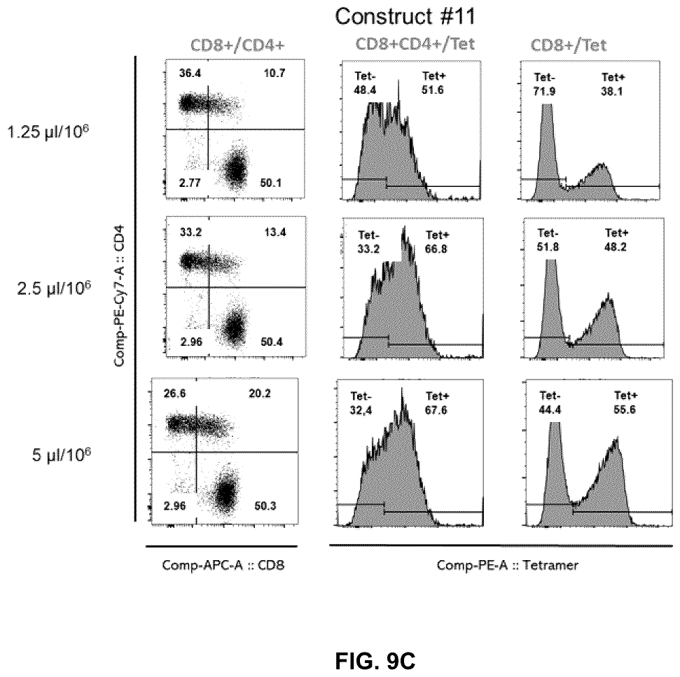

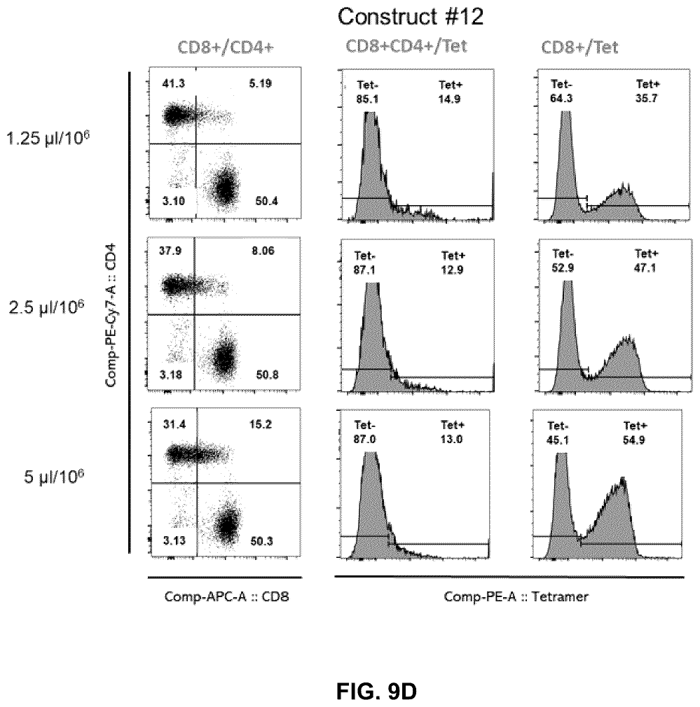

The inventors found that the various CD8 elements in the vector lead to a surprising increase in expression and activity. For example, despite the observation that Construct #10 has lower viral titers than Constructs #9b, #11, and #12 (FIG. 5A), T cells transduced with Construct #10 expressing CD8αβ heterodimer and TCR at the lowest viral volumetric concentration, e.g., 1.25 μl/10⁶ cells, generated higher CD8+CD4+TCR+ cells (56.7%, FIG. 9B) than that of transduced with Construct #9b expressing CD8α and TCR (42.3%, FIG. 9A), Construct #11 expressing CD8αCD8βstalk with CD8α transmembrane and intracellular domain and TCR (51.6%, FIG. 9C), and Construct #12 expressing CD8αCD8βstalk with Neural Cell Adhesion Molecule 1 (NCAM1) transmembrane and intracellular domain and TCR (14.9%, FIG. 9D).

A vector may comprise any one of nucleic acid sequences of SEQ ID NO: 267, 269, 271, 273, 275, 277, 279, 281, 283, 285, 287, 289, 291, 295, 297, 299, or 301.

A T-cell may be transduced to express the nucleic acid of SEQ ID NO: 267, 269, 271, 273, 275, 277, 279, 281, 283, 285, 287, 289, 291, 295, 297, 299, or 301.

Several of the elements of the constructs in Table 2 are described in Table 3.

TABLE 3
Representative Protein and DNA sequences

| SEQ ID | | |
|---|---|---|
| NO: | Description | Sequence |
| 1 | CD8α Ig-like | SQFRVSPLDRTWNLGETVELKCQVLLSNPTSGCSWLFQ |
| | domain-1 | PRGAAASPTFLLYLSQNKPKAAEGLDTQRFSGKRLGDT |
| | | FVLTLSDFRRENEGYYFCSALSNSIMYFSHFVPVFLPA |
| 2 | CD8β region | SVVDFLPTTAQPTKKSTLKKRVCRLPRPETQKGPLCSP |
| 3 | CD8α | IYIWAPLAGTCGVLLLSLVIT |
| | transmembrane | |
| | domain | |
| 4 | CD8α | LYCNHRNRRRVCKCPRPVVKSGDKPSLSARYV |
| | cytoplasmic tail | |
| 5 | m1CD8α (signal- | SQFRVSPLDRTWNLGETVELKCQVLLSNPTSGCSWLFQ |
| | less) | PRGAAASPTFLLYLSQNKPKAAEGLDTQRFSGKRLGDT |
| | | FVLTLSDFRRENEGYYFCSALSNSIMYFSHFVPVFLPAS |
| | | VVDFLPTTAQPTKKSTLKKRVCRLPRPETQKGPLCSPIYI |
| | | WAPLAGTCGVLLLSLVITLYCNHRNRRRVCKCPRPVVK |
| | | SGDKPSLSARYV |
| 6 | CD8α Signal | MALPVTALLLPLALLLHAARP |
| | peptide | |
| 7 | m1CD8α | MALPVTALLLPLALLLHAARPSQFRVSPLDRTWNLGET |
| | | VELKCQVLLSNPTSGCSWLFQPRGAAASPTFLLYLSQN |
| | | KPKAAEGLDTQRFSGKRLGDTFVLTLSDFRRENEGYYF |
| | | CSALSNSIMYFSHFVPVFLPASVVDFLPTTAQPTKKSTL |
| | | KKRVCRLPRPETQKGPLCSPIYIWAPLAGTCGVLLLSLVI |
| | | TLYCNHRNRRRVCKCPRPVVKSGDKPSLSARYV |
| 8 | CD8β1 | MRPRLWLLLAAQLTVLHGNSVLQQTPAYIKVQTNKMV |
| | | MLSCEAKISLSNMRIYWLRQRQAPSSDSHHEFLALWDS |
| | | AKGTIHGEEVEQEKIAVFRDASRFILNLTSVKPEDSGIYF |
| | | CMIVGSPELTFGKGTQLSVVDFLPTTAQPTKKSTLKKRV |
| | | CRLPRPETQKGPLCSPITLGLLVAGVLVLLVSLGVAIHL |
| | | CCRRRRARLRFMKQPQGEGISGTFVPQCLHGYYSNTTT |
| | | SQKLLNPWILKT |
| 9 | CD8β2 | MRPRLWLLLAAQLTVLHGNSVLQQTPAYIKVQTNKMV |
| | | MLSCEAKISLSNMRIYWLRQRQAPSSDSHHEFLALWDS |
| | | AKGTIHGEEVEQEKIAVFRDASRFILNLTSVKPEDSGIYF |
| | | CMIVGSPELTFGKGTQLSVVDFLPTTAQPTKKSTLKKRV |
| | | CRLPRPETQKGLKGKVYQEPLSPNACMDTTAILQPHRS |
| | | CLTHGS |
| 10 | CD8β3 | LQQTPAYIKVQTNKMVMLSCEAKISLSNMRIYWLRQRQ |
| | | APSSDSHHEFLALWDSAKGTIHGEEVEQEKIAVFRDASR |
| | | FILNLTSVKPEDSGIYFCMIVGSPELTFGKGTQLSVVDFL |
| | | PTTAQPTKKSTLKKRVCRLPRPETQKGPLCSPITLGLLV |
| | | AGVLVLLVSLGVAIHLCCRRRRARLRFMKQFYK |
| 11 | CD8β4 | LQQTPAYIKVQTNKMVMLSCEAKISLSNMRIYWLRQRQ |
| | | APSSDSHHEFLALWDSAKGTIHGEEVEQEKIAVFRDASR |
| | | FILNLTSVKPEDSGIYFCMIVGSPELTFGKGTQLSVVDFL |
| | | PTTAQPTKKSTLKKRVCRLPRPETQKGPLCSPITLGLLV |
| | | AGVLVLLVSLGVAIHLCCRRRRARLRFMKQLRLHPLEK |
| | | CSRMDY |
| 12 | CD8β5 | LQQTPAYIKVQTNKMVMLSCEAKISLSNMRIYWLRQRQ |
| | | APSSDSHHEFLALWDSAKGTIHGEEVEQEKIAVFRDASR |
| | | FILNLTSVKPEDSGIYFCMIVGSPELTFGKGTQLSVVDFL |
| | | PTTAQPTKKSTLKKRVCRLPRPETQKGPLCSPITLGLLV |
| | | AGVLVLLVSLGVAIHLCCRRRRARLRFMKQKFNIVCLK |
| | | ISGFTTCCCFQILQISREYGFGVLLQKDIGQ |
| 13 | CD8β6 | LQQTPAYIKVQTNKMVMLSCEAKISLSNMRIYWLRQRQ |
| | | APSSDSHHEFLALWDSAKGTIHGEEVEQEKIAVFRDASR |
| | | FILNLTSVKPEDSGIYFCMIVGSPELTFGKGTQLSVVDFL |
| | | PTTAQPTKKSTLKKRVCRLPRPETQKGPLCSPITLGLLV |
| | | AGVLVLLVSLGVAIHLCCRRRRARLRFMKQKFNIVCLK |
| | | ISGFTTCCCFQILQISREYGFGVLLQKDIGQ |
| 14 | CD8β7 | LQQTPAYIKVQTNKMVMLSCEAKISLSNMRIYWLRQRQ |
| | | APSSDSHHEFLALWDSAKGTIHGEEVEQEKIAVFRDASR |
| | | FILNLTSVKPEDSGIYFCMIVGSPELTFGKGTQLSVVDFL |
| | | PTTAQPTKKSTLKKRVCRLPRPETQKGPLCSPITLGLLV |
| | | AGVLVLLVSLGVAIHLCCRRRRARLRFMKQPQGEGISG |
| | | TFVPQCLHGYYSNTTTSQKLLNPWILKT |
| 15 | R11KEA alpha | MEKNPLAAPLLILWFHLDCVSSILNVEQSPQSLHVQEGD |
| | chain | STNFTCSFPSSNFYALHWYRKETAKSPEALFVMTLNGD |
| | | EKKKGRISATLNTKEGYSYLYIKGSQPEDSATYLCALYN |
| | | NNDMRFGAGTRLTVKPNIQNPDPAVYQLRDSKSSDKSV |
| | | CLFTDFDSQTNVSQSKDSDVYITDKTVLDMRSMDFKSN |
| | | SAVAWSNKSDFACANAFNNSIIPEDTFFPSPESSCDVKL |
| | | VEKSFETDTNLNFQNLSVIGFRILLLKVAGFNLLMTLRL |
| | | WSS |
| 16 | R11KE beta | MDSWTFCCVSLCILVAKHTDAGVIQSPRHEVTEMGQEV |
| | chain | TLRCKPISGHNLFWYRETMMRGLELLIYFNNNVPIDDS |
| | | GMPEDRFSAKMPNASFSTLKIQPSEPRDSAVYFCASSPG |
| | | STDTQYFGPGTRLTVLEDLKNVFPPEVAVFEPSEAEISHT |
| | | QKATLVCLATGFYPDHVELSWWVNGKEVHSGVSTDPQ |
| | | PLKEQPALNDSRYCLSSRLRVSATFWQNPRNHFRCQVQ |
| | | FYGLSENDEWTQDRAKPVTQIVSAEAWGRADCGFTSES |
| | | YQQGVLSATILYEILLGKATLYAVLVSALVLMAMVKR |
| | | KDSRG |
| 17 | R20P1H7 alpha | MEKMLECAFIVLWLQLGWLSGEDQVTQSPEALRLQEG |
| | chain | ESSSLNCSYTVSGLRGLFWYRQDPGKGPEFLFTLYSAGE |
| | | EKEKERLKATLTKKESFLHITAPKPEDSATYLCAVQGEN |
| | | SGYSTLTFGKGTMLLVSPDIQNPDPAVYQLRDSKSSDKS |
| | | VCLFTDFDSQTNVSQSKDSDVYITDKTVLDMRSMDFKS |
| | | NSAVAWSNKSDFACANAFNNSIIPEDTFFPSPESSCDVK |
| | | LVEKSFETDTNLNFQNLSVIGFRILLLKVAGFNLLMTLR |
| | | LWSS |
| 18 | R20P1H7 beta | MGPQLLGYVVLCLLGAGPLEAQVTQNPRYLITVTGKKL |
| | chain | TVTCSQNMNHEYMSWYRQDPGLGLRQIYYSMNVEVT |
| | | DKGDVPEGYKVSRKEKRNFPLILESPSPNQTSLYFCASS |
| | | LGPGLAAYNEQFFGPGTRLTVLEDLKNVFPPEVAVFEPS |
| | | EAEISHTQKATLVCLATGFYPDHVELSWWVNGKEVHS |
| | | GVSTDPQPLKEQPALNDSRYCLSSRLRVSATFWQNPRN |
| | | HFRCQVQFYGLSENDEWTQDRAKPVTQIVSAEAWGRA |
| | | DCGFTSESYQQGVLSATILYEILLGKATLYAVLVSALVL |
| | | MAMVKRKDSRG |
| 19 | R7P1D5 alpha | MKTFAGFSFLFLWLQLDCMSRGEDVEQSLFLSVREGDS |
| | chain | SVINCTYTDSSSTYLYWYKQEPGAGLQLLTYIFSNMDM |
| | | KQDQRLTVLLNKKDKHLSLRIADTQTGDSAIYFCAEYS |
| | | SASKIIFGSGTRLSIRPNIQNPDPAVYQLRDSKSSDKSVC |
| | | LFTDFDSQTNVSQSKDSDVYITDKTVLDMRSMDFKSNS |
| | | AVAWSNKSDFACANAFNNSIIPEDTFFPSPESSCDVKLV |
| | | EKSFETDTNLNFQNLSVIGFRILLLKVAGFNLLMTLRLW |
| | | SS |
| 20 | R7P1D5 beta | MGSWTLCCVSLCILVAKHTDAGVIQSPRHEVTEMGQEV |
| | chain | TLRCKPISGHDYLFWYRQTMMRGLELLIYFNNNVPIDD |
| | | SGMPEDRFSAKMPNASFSTLKIQPSEPRDSAVYFCASRA |
| | | NTGELFFGEGSRLTVLEDLKNVFPPEVAVFEPSEAEISHT |
| | | QKATLVCLATGFYPDHVELSWWVNGKEVHSGVSTDPQ |
| | | PLKEQPALNDSRYCLSSRLRVSATFWQNPRNHFRCQVQ |
| | | FYGLSENDEWTQDRAKPVTQIVSAEAWGRADCGFTSES |
| | | YQQGVLSATILYEILLGKATLYAVLVSALVLMAMVKR |
| | | KDSRG |
| 21 | R10P2G12 alpha | MLTASLLRAVIASICVVSSMAQKVTQAQTEISVVEKED |
| | chain | VTLDCVYETRDTTYYLFWYKQPPSGELVFLIRRNSFDE |
| | | QNEISGRYSWNFQKSTSSFNFTITASQVVDSAVYFCALS |
| | | EGNSGNTPLVFGKGTRLSVIANIQNPDPAVYQLRDSKSS |
| | | DKSVCLFTDFDSQTNVSQSKDSDVYITDKTVLDMRSMD |
| | | FKSNSAVAWSNKSDFACANAFNNSIIPEDTFFPSPESSCD |
| | | VKLVEKSFETDTNLNFQNLSVIGFRILLLKVAGFNLLMT |
| | | LRLWSS |
| 22 | R10P2G12 beta | MGIRLLCRVAFCFLAVGLVDVKVTQSSRYLVKRTGEKV |
| | chain | FLECVQDMDHENMFWYRQDPGLGLRLIYFSYDVKMKE |
| | | KGDIPEGYSVSREKKERFSLILESASTNQTSMYLCASSLS |
| | | SGSHQETQYFGPGTRLLVLEDLKNVFPPEVAVFEPSEAE |
| | | ISHTQKATLVCLATGFYPDHVELSWWVNGKEVHSGVS |
| | | TDPQPLKEQPALNDSRYCLSSRLRVSATFWQNPRNHFR |
| | | CQVQFYGLSENDEWTQDRAKPVTQIVSAEAWGRADCG |
| | | FTSESYQQGVLSATILYEILLGKATLYAVLVSALVLMA |
| | | MVKRKDSRG |
| 23 | R10P1A7 alpha | MKTFAGFSFLFLWLQLDCMSRGEDVEQSLFLSVREGDS |
| | chain | SVINCTYTDSSSTYLYWYKQEPGAGLQLLTYIFSNMDM |
| | | KQDQRLTVLLNKKDKHLSLRIADTQTGDSAIYFCAESK |
| | | ETRLMFGDGTQLVVKPNIQNPDPAVYQLRDSKSSDKSV |
| | | CLFTDFDSQTNVSQSKDSDVYITDKTVLDMRSMDFKSN |
| | | SAVAWSNKSDFACANAFNNSIIPEDTFFPSPESSCDVKL |
| | | VEKSFETDTNLNFQNLSVIGFRILLLKVAGFNLLMTLRL |
| | | WSS |
| 24 | R10P1A7 beta | MLLLLLLLGPGISLLLPGSLAGSGLGAWSQHPSVWICKS |
| | chain | GTSVKIECRSLDFQATTMFWYRQFPKQSLMLMATSNEG |
| | | SKATYEQGVEKDKFLINHASLTLSTLTVTSAHPEDSSFYI |
| | | CSARAGGHEQFFGPGTRLTVLEDLKNVFPPEVAVFEPSE |
| | | AEISHTQKATLVCLATGFYPDHVELSWVWNGKEVHSG |
| | | VSTDPQPLKEQPALNDSRYCLSSRLRVSATFWQNPRNH |
| | | FRCQVQFYGLSENDEWTQDRAKPVTQIVSAEAWGRAD |
| | | CGFTSESYQQGVLSATILYEILLGKATLYAVLVSALVLM |
| | | AMVKRKDSRG |
| 25 | R4P1D10 alpha | MKSLRVLLVILWLQLSWVWSQQKEVEQNSGPLSVPEG |
| | chain | AIASLNCTYSDRGSQSFFWYRQYSGKSPELIMFIYSNGD |
| | | KEDGRFTAQLNKASQYVSLLIRDSQPSDSATYLCAVNF |
| | | HDKIIFGKGTRLHILPNIQNPDPAVYQLRDSKSSDKSVCL |
| | | FTDFDSQTNVSQSKDSDVYITDKTVLDMRSMDFKSNSA |
| | | VAWSNKSDFACANAFNNSIIPEDTFFPSPESSCDVKLVE |
| | | KSFETDTNLNFQNLSVIGFRILLLKVAGFNLLMTLRLWS |
| | | S |
| 26 | R4P1D10 beta | MGFRLLCCVAFCLLGAGPVDSGVTQTPKHLITATGQRV |
| | chain | TLRCSPRSGDLSVYWYQQSLDQGLQFLIHYYNGEERAK |
| | | GNILERFSAQQFPDLHSELNLSSLELGDSALYFCASSVAS |
| | | AYGYTFGSGTRLTVVEDLNKVFPPEVAVFEPSEAEISHT |
| | | QKATLVCLATGFFPDHVELSWWVNGKEVHSGVSTDPQ |
| | | PLKEQPALNDSRYCLSSRLRVSATFWQNPRNHFRCQVQ |
| | | FYGLSENDEWTQDRAKPVTQIVSAEAWGRADCGFTSV |
| | | SYQQGVLSATILYEILLGKATLYAVLVSALVLMAMVKR |
| | | KDF |
| 27 | R4P3F9 alpha | MKSLRVLLVILWLQLSWVWSQQKEVEQNSGPLSVPEG |
| | chain | AIASLNCTYSDRGSQSFFWYRQYSGKSPELIMFIYSNGD |
| | | KEDGRFTAQLNKASQYVSLLIRDSQPSDSATYLCAAYS |
| | | GAGSYQLTFGKGTKLSVIPNIQNPDPAVYQLRDSKSSDK |
| | | SVCLFTDFDSQTNVSQSKDSDVYITDKTVLDMRSMDFK |
| | | SNSAVAWSNKSDFACANAFNNSIIPEDTFFPSPESSCDV |
| | | KLVEKSFETDTNLNFQNLSVIGFRILLLKVAGFNLLMTL |
| | | RLWSS |
| 28 | R4P3F9 beta | MGFRLLCCVAFCLLGAGPVDSGVTQTPKHLITATGQRV |
| | chain | TLRCSPRSGDLSVYWYQQSLDQGLQFLIQYYNGEERAK |
| | | GNILERFSAQQFPDLHSELNLSSLELGDSALYFCASSVES |
| | | SYGYTFGSGTRLTVVEDLNKVFPPEVAVFEPSEAEISHT |
| | | QKATLVCLATGFFPDHVELSWWVNGKEVHSGVSTDPQ |
| | | PLKEQPALNDSRYCLSSRLRVSATFWQNPRNHFRCQVQ |
| | | FYGLSENDEWTQDRAKPVTQIVSAEAWGRADCGFTSV |
| | | SYQQGVLSATILYEILLGKATLYAVLVSALVLMAMVKR |
| | | KDF |
| 29 | R4P3H3 alpha | MKSLRVLLVILWLQLSWVWSQQKEVEQNSGPLSVPEG |
| | chain | AIASLNCTYSDRGSQSFFWYRQYSGKSPELIMFIYSNGD |
| | | KEDGRFTAQLNKASQYVSLLIRDSQPSDSATYLCAVKA |
| | | GNQFYFGTGTSLTVIPNIQNPDPAVYQLRDSKSSDKSVC |
| | | LFTDFDSQTNVSQSKDSDVYITDKTVLDMRSMDFKSNS |
| | | AVAWSNKSDFACANAFNNSIIPEDTFFPSPESSCDVKLV |
| | | EKSFETDTNLNFQNLSVIGFRILLLKVAGFNLLMTLRLW |
| | | SS |
| 30 | R4P3H3 beta | MGTRLLCWVVLGFLGTDHTGAGVSQSPRYKVAKRGQ |
| | chain | DVALRCDPISGHVSLFWYQQALGQGPEFLTYFQNEAQL |
| | | DKSGLPSDRFFAERPEGSVSTLKIQRTQQEDSAVYLCAS |
| | | SLLTSGGDNEQFFGPGTRLTVLEDLKNVFPPEVAVFEPS |
| | | EAEISHTQKATLVCLATGFYPDHVELSWWVNGKEVHS |
| | | GVSTDPQPLKEQPALNDSRYCLSSRLRVSATFWQNPRN |
| | | HFRCQVQFYGLSENDEWTQDRAKPVTQIVSAEAWGRA |
| | | DCGFTSESYQQGVLSATILYEILLGKATLYAVLVSALVL |
| | | MAMVKRKDSRG |
| 31 | R36P3F9 alpha | METLLGVSLVILWLQLARVNSQQGEEDPQALSIQEGEN |
| | chain | ATMNCSYKTSINNLQWYRQNSGRGLVHLILIRSNEREK |
| | | HSGRLRVTLDTSKKSSSLLITASRAADTASYFCATVSNY |
| | | QLIWGAGTKLIIKPDIQNPDPAVYQLRDSKSSDKSVCLF |
| | | TDFDSQTNVSQSKDSDVYITDKTVLDMRSMDFKSNSAV |
| | | AWSNKSDFACANAFNNSIIPEDTFFPSPESSCDVKLVEKS |
| | | FETDTNLNFQNLSVIGFRILLLKVAGFNLLMTLRLWSS |
| 32 | R36P3F9 beta | MGPQLLGYVVLCLLGAGPLEAQVTQNPRYLITVTGKKL |
| | chain | TVTCSQNMNHEYMSWYRQDPGLGLRQIYYSMNVEVT |
| | | DKGDVPEGYKVSRKEKRNFPLILESPSPNQTSLYFCASS |
| | | STSGGLSGETQYFGPGTRLLVLEDLKNVFPPEVAVFEPS |
| | | EAEISHTQKATLVCLATGFYPDHVELSWWVNGKEVHS |
| | | GVSTDPQPLKEQPALNDSRYCLSSRLRVSATFWQNPRN |
| | | HFRCQVQFYGLSENDEWTQDRAKPVTQIVSAEAWGRA |
| | | DCGFTSESYQQGVLSATILYEILLGKATLYAVLVSALVL |
| | | MAMVKRKDSRG |
| 33 | R52P2G11 alpha | MKKHLTTFLVILWLYFYRGNGKNQVEQSPQSLIILEGK |
| | chain | NCTLQCNYTVSPFSNLRWYKQDTGRGPVSLTIMTFSEN |
| | | TKSNGRYTATLDADTKQSSLHITASQLSDSASYICVVSA |
| | | YGKLQFGAGTQVVVTPDIQNPDPAVYQLRDSKSSDKSV |
| | | CLFTDFDSQTNVSQSKDSDVYITDKTVLDMRSMDFKSN |
| | | SAVAWSNKSDFACANAFNNSIIPEDTFFPSPESSCDVKL |
| | | VEKSFETDTNLNFQNLSVIGFRILLLKVAGFNLLMTLRL |
| | | WSS |
| 34 | R52P2G11 beta | MDSWTFCCVSLCILVAKHTDAGVIQSPRHEVTEMGQEV |
| | chain | TLRCKPISGHNSLFWYRQTMMRGLELLIYFNNNVPIDDS |
| | | GMPEDRFSAKMPNASFSTLKIQPSEPRDSAVYFCASSLG |
| | | SPDGNQPQHFGDGTRLSILEDLNKVFPPEVAVFEPSEAEI |
| | | SHTQKATLVCLATGFFPDHVELSWWVNGKEVHSGVST |
| | | DPQPLKEQPALNDSRYCLSSRLRVSATFWQNPRNHFRC |
| | | QVQFYGLSENDEWTQDRAKPVTQIVSAEAWGRADCGF |
| | | TSVSYQQGVLSATILYEILLGKATLYAVLVSALVLMAM |
| | | VKRKDF |
| 35 | R53P2A9 alpha | MACPGFLWALVISTCLEFSMAQTVTQSQPEMSVQEAET |
| | chain | VTLSCTYDTSESDYYLFWYKQPPSRQMILVIRQEAYKQ |
| | | QNATENRFSVNFQKAAKSFSLKISDSQLGDAAMYFCAY |
| | | NSYAGGTSYGKLTFGQGTILTVHPNIQNPDPAVYQLRD |
| | | SKSSDKSVCLFTDFDSQTNVSQSKDSDVYITDKTVLDM |
| | | RSMDFKSNSAVAWSNKSDFACANAFNNSIIPEDTFFPSP |
| | | ESSCDVKLVEKSFETDTNLNFQNLSVIGFRILLLKVAGF |
| | | NLLMTLRLWSS |
| 36 | R53P2A9 beta | MGPGLLCWVLLCLLGAGPVDAGVTQSPTHLIKTRGQQ |
| | chain | VTLRCSPISGHKSVSWYQQVLGQGPQFIFQYYEKEERG |
| | | RGNFPDRFSARQFPNYSSELNVNALLLGDSALYLCASSL |
| | | DGTSEQYFGPGTRLTVTEDLKNVFPPEVAVFEPSEAEIS |
| | | HTQKATLVCLATGFYPDHVELSWWVNGKEVHSGVSTD |
| | | PQPLKEQPALNDSRYCLSSRLRVSATFWQNPRNHFRCQ |
| | | VQFYGLSENDEWTQDRAKPVTQIVSAEAWGRADCGFT |
| | | SESYQQGVLSATILYEILLGKATLYAVLVSALVLMAMV |
| | | KRKDSRG |
| 37 | R26P1A9 alpha | METLLGVSLVILWLQLARVNSQQGEEDPQALSIQEGEN |
| | chain | ATMNCSYKTSINNLQWYRQNSGRGLVHLILIRSNEREK |
| | | HSGRLRVTLDTSKKSSSLLITASRAADTASYFCLIGASGS |
| | | RLTFGEGTQLTVNPDIQNPDPAVYQLRDSKSSDKSVCLF |
| | | TDFDSQTNVSQSKDSDVYITDKTVLDMRSMDFKSNSAV |
| | | AWSNKSDFACANAFNNSIIPEDTFFPSPESSCDVKLVEKS |
| | | FETDTNLNFQNLSVIGFRILLLKVAGFNLLMTLRLWSS |
| 38 | R26P1A9 beta | MGSWTLCCVSLCILVAKHTDAGVIQSPRHEVTEMGQEV |
| | chain | TLRCKPISGHDYLFWYRQTMMRGLELLIYFNNNVPIDD |
| | | SGMPEDRFSAKMPNASFSTLKIQPSEPRDSAVYFCASSY |
| | | FGWNEKLFFGSGTQLSVLEDLNKVFPPEVAVFEPSEAEI |
| | | SHTQKATLVCLATGFFPDHVELSWWVNGKEVHSGVST |
| | | DPQPLKEQPALNDSRYCLSSRLRVSATFWQNPRNHFRC |
| | | QVQFYGLSENDEWTQDRAKPVTQIVSAEAWGRADCGF |
| | | TSVSYQQGVLSATILYEILLGKATLYAVLVSALVLMAM |
| | | VKRKDF |
| 39 | R26P2A6 alpha | MMKSLRVLLVILWLQLSWVWSQQKEVEQDPGPLSVPE |
| | chain | GAIVSLNCTYSNSAFQYFMWYRQYSRKGPELLMYTYSS |
| | | GNKEDGRFTAQVDKSSKYISLFIRDSQPSDSATYLCAMS |
| | | DVSGGYNKLIFGAGTRLAVHPYIQNPDPAVYQLRDSKS |
| | | SDKSVCLFTDFDSQTNVSQSKDSDVYITDKTVLDMRSM |
| | | DFKSNSAVAWSNKSDFACANAFNNSIIPEDTFFPSPESSC |
| | | DVKLVEKSFETDTNLNFQNLSVIGFRILLLKVAGFNLLM |
| | | TLRLWSS |
| 40 | R26P2A6 beta | MGPQLLGYVVLCLLGAGPLEAQVTQNPRYLITVTGKKL |
| | chain | TVTCSQNMNHEYMSWYRQDPGLGLRQIYYSMNVEVT |
| | | DKGDVPEGYKVSRKEKRNFPLILESPSPNQTSLYFCAST |
| | | TPDGTDEQFFGPGTRLTVLEDLKNVFPPEVAVFEPSEAEI |
| | | SHTQKATLVCLATGFYPDHVELSWWVNGKEVHSGVST |
| | | DPQPLKEQPALNDSRYCLSSRLRVSATFWQNPRNHFRC |
| | | QVQFYGLSENDEWTQDRAKPVTQIVSAEAWGRADCGF |
| | | TSESYQQGVLSATILYEILLGKATLYAVLVSALVLMAM |
| | | VKRKDSRG |
| 41 | R26P3H1 alpha | MASAPISMLAMLFTLSGLRAQSVAQPEDQVNVAEGNPL |
| | chain | TVKCTYSVSGNPYLFWYVQYPNRGLQFLLKYITGDNLV |
| | | KGSYGFEAEFNKSQTSFHLKKPSALVSDSALYFCAVRD |
| | | MNRDDKIIFGKGTRLHILPNIQNPDPAVYQLRDSKSSDK |
| | | SVCLFTDFDSQTNVSQSKDSDVYITDKTVLDMRSMDFK |
| | | SNSAVAWSNKSDFACANAFNNSIIPEDTFFPSPESSCDV |
| | | KLVEKSFETDTNLNFQNLSVIGFRILLLKVAGFNLLMTL |
| | | RLWSS |
| 42 | R26P3H1 beta | MSNQVLCCVVLCFLGANTVDGGITQSPKYLFRKEGQN |
| | chain | VTLSCEQNLNHDAMYWYRQDPGQGLRLIYYSQIVNDF |
| | | QKGDIAEGYSVSREKKESFPLTVTSAQKNPTAFYLCASS |
| | | RAEGGEQYFGPGTRLTVTEDLKNVFPPEVAVFEPSEAEI |
| | | SHTQKATLVCLATGFYPDHVELSWWVNGKEVHSGVST |
| | | DPQPLKEQPALNDSRYCLSSRLRVSATFWQNPRNHFRC |
| | | QVQFYGLSENDEWTQDRAKPVTQIVSAEAWGRADCGF |
| | | TSESYQQGVLSATILYEILLGKATLYAVLVSALVLMAM |
| | | VKRKDSRG |
| 43 | R35P3A4 alpha | MTSIRAVFIFLWLQLDLVNGENVEQHPSTLSVQEGDSA |
| | chain | VIKCTYSDSASNYFPWYKQELGKRPQLIIDIRSNVGEKK |
| | | DQRIAVTLNKTAKHFSLHITETQPEDSAVYFCAASPTGG |
| | | YNKLIFGAGTRLAVHPYIQNPDPAVYQLRDSKSSDKSV |
| | | CLFTDFDSQTNVSQSKDSDVYITDKTVLDMRSMDFKSN |
| | | SAVAWSNKSDFACANAFNNSIIPEDTFFPSPESSCDVKL |
| | | VEKSFETDTNLNFQNLSVIGFRILLLKVAGFNLLMTLRL |
| | | WSS |
| 44 | R35P3A4 beta | MSIGLLCCAALSLLWAGPVNAGVTQTPKFQVLKTGQS |
| | chain | MTLQCAQDMNHEYMSWYRQDPGMGLRLIHYSVGAGI |
| | | TDQGEVPNGYNVSRSTTEDFPLRLLSAAPSQTSVYFCAS |
| | | SLGGASQEQYFGPGTRLTVTEDLKNVFPPEVAVFEPSEA |
| | | EISHTQKATLVCLATGFYPDHVELSWWVNGKEVHSGV |
| | | STDPQPLKEQPALNDSRYCLSSRLRVSATFWQNPRNHF |
| | | RCQVQFYGLSENDEWTQDRAKPVTQIVSAEAWGRADC |
| | | GFTSESYQQGVLSATILYEILLGKATLYAVLVSALVLMA |
| | | MVKRKDSRG |
| 45 | R37P1C9 alpha | MKLVTSITVLLSLGIMGDAKTTQPNSMESNEEEPVHLPC |
| | chain | NHSTISGTDYIHWYRQLPSQGPEYVIHGLTSNVNNRMA |
| | | SLAIAEDRKSSTLILHRATLRDAAVYYCILFNFNKFYFGS |
| | | GTKLNVKPNIQNPDPAVYQLRDSKSSDKSVCLFTDFDS |
| | | QTNVSQSKDSDVYITDKTVLDMRSMDFKSNSAVAWSN |
| | | KSDFACANAFNNSIIPEDTFFPSPESSCDVKLVEKSFETD |
| | | TNLNFQNLSVIGFRILLLKVAGFNLLMTLRLWSS |
| 46 | R37P1C9 beta | MGPGLLHWMALCLLGTGHGDAMVIQNPRYQVTQFGK |
| | chain | PVTLSCSQTLNHNVMYWYQQKSSQAPKLLFHYYDKDF |
| | | NNEADTPDNFQSRRPNTSFCFLDIRSPGLGDAAMYLCA |
| | | TSSGETNEKLFFGSGTQLSVLEDLNKVFPPEVAVFEPSE |
| | | AEISHTQKATLVCLATGFFPDHVELSWWVNGKEVHSG |
| | | VSTDPQPLKEQPALNDSRYCLSSRLRVSATFWQNPRNH |
| | | FRCQVQFYGLSENDEWTQDRAKPVTQIVSAEAWGRAD |
| | | CGFTSVSYQQGVLSATILYEILLGKATLYAVLVSALVLM |
| | | AMVKRKDF |
| 47 | R37P1H1 alpha | MTRVSLLWAVVVSTCLESGMAQTVTQSQPEMSVQEAE |
| | chain | TVTLSCTYDTSESNYYLFWYKQPPSRQMILVIRQEAYK |
| | | QQNATENRFSVNFQKAAKSFSLKISDSQLGDTAMYFCA |
| | | FGYSGGGADGLTFGKGTHLIIQPYIQNPDPAVYQLRDSK |
| | | SSDKSVCLFTDFDSQTNVSQSKDSDVYITDKTVLDMRS |
| | | MDFKSNSAVAWSNKSDFACANAFNNSIIPEDTFFPSPES |
| | | SCDVKLVEKSFETDTNLNFQNLSVIGFRILLLKVAGFNL |
| | | LMTLRLWSS |
| 48 | R37P1H1 beta | MGPGLLCWALLCLLGAGLVDAGVTQSPTHLIKTRGQQ |
| | chain | VTLRCSPKSGHDTVSWYQQALGQGPQFIFQYYEEEERQ |
| | | RGNFPDRFSGHQFPNYSSELNVNALLLGDSALYLCASS |
| | | NEGQGWEAEAFFGQGTRLTVVEDLNKVFPPEVAVFEPS |
| | | EAEISHTQKATLVCLATGFFPDHVELSWWVNGKEVHSG |
| | | VSTDPQPLKEQPALNDSRYCLSSRLRVSATFWQNPRNH |
| | | FRCQVQFYGLSENDEWTQDRAKPVTQIVSAEAWGRAD |
| | | CGFTSVSYQQGVLSATILYEILLGKATLYAVLVSALVLM |
| | | AMVKRKDF |
| 49 | R42P3A9 alpha | MKRILGALLGLLSAQVCCVRGIQVEQSPPDLILQEGANS |
| | chain | TLRCNFSDSVNNLQWFHQNPWGQLINLFYIPSGTKQNG |
| | | RLSATTVATERYSLLYISSSQTTDSGVYFCAVHNFNKFY |
| | | FGSGTKLNVKPNIQNPDPAVYQLRDSKSSDKSVCLFTDF |
| | | DSQTNVSQSKDSDVYITDKTVLDMRSMDFKSNSAVAW |
| | | SNKSDFACANAFNNSIIPEDTFFPSPESSCDVKLVEKSFE |
| | | TDTNLNFQNLSVIGFRILLLKVAGFNLLMTLRLWSS |
| 50 | R42P3A9 beta | MLSPDLPDSAWNTRLLCHVMLCLLGAVSVAAGVIQSPR |
| | chain | HLIKEKRETATLKCYPIPRHDTVYWYQQGPGQDPQFLIS |
| | | FYEKMQSDKGSIPDRFSAQQFSDYHSELNMSSLELGDS |
| | | ALYFCASSLLGQGYNEQFFGPGTRLTVLEDLKNVFPPEV |
| | | AVFEPSEAEISHTQKATLVCLATGFYPDHVELSWWVNG |
| | | KEVHSGVSTDPQPLKEQPALNDSRYCLSSRLRVSATFW |
| | | QNPRNHFRCQVQFYGLSENDEWTQDRAKPVTQIVSAE |
| | | AWGRADCGFTSESYQQGVLSATILYEILLGKATLYAVL |
| | | VSALVLMAMVKRKDSRG |
| 51 | R43P3F2 alpha | MLTASLLRAVIASICVVSSMAQKVTQAQTEISVVEKED |
| | chain | VTLDCVYETRDTTYYLFWYKQPPSGELVFLIRRNSFDE |
| | | QNEISGRYSWNFQKSTSSFNFTITASQVVDSAVYFCALS |
| | | NNNAGNMLTFGGGTRLMVKPHIQNPDPAVYQLRDSKS |
| | | SDKSVCLFTDFDSQTNVSQSKDSDVYITDKTVLDMRSM |
| | | DFKSNSAVAWSNKSDFACANAFNNSIIPEDTFFPSPESSC |
| | | DVKLVEKSFETDTNLNFQNLSVIGFRILLLKVAGFNLLM |
| | | TLRLWSS |
| 52 | R43P3F2 beta | MLSPDLPDSAWNTRLLCHVMLCLLGAVSVAAGVIQSPR |
| | chain | HLIKEKRETATLKCYPIPRHDTVYWYQQGPGQDPQFLIS |
| | | FYEKMQSDKGSIPDRFSAQQFSDYHSELNMSSLELGDS |
| | | ALYFCASSPTGTSGYNEQFFGPGTRLTVLEDLKNVFPPE |
| | | VAVFEPSEAEISHTQKATLVCLATGFYPDHVELSWWVN |
| | | GKEVHSGVSTDPQPLKEQPALNDSRYCLSSRLRVSATF |
| | | WQNPRNHFRCQVQFYGLSENDEWTQDRAKPVTQIVSA |
| | | EAWGRADCGFTSESYQQGVLSATILYEILLGKATLYAV |
| | | LVSALVLMAMVKRKDSRG |
| 53 | R43P3G5 alpha | MEKNPLAAPLLILWFHLDCVSSILNVEQSPQSLHVQEGD |
| | chain | STNFTCSFPSSNFYALHWYRWETAKSPEALFVMTLNGD |
| | | EKKKGRISATLNTKEGYSYLYIKGSQPEDSATYLCALNR |
| | | DDKIIFGKGTRLHILPNIQNPDPAVYQLRDSKSSDKSVCL |
| | | FTDFDSQTNVSQSKDSDVYITDKTVLDMRSMDFKSNSA |
| | | VAWSNKSDFACANAFNNSIIPEDTFFPSPESSCDVKLVE |
| | | KSFETDTNLNFQNLSVIGFRILLLKVAGFNLLMTLRLWS |
| | | S |
| 54 | R43P3G5 beta | MGIRLLCRVAFCFLAVGLVDVKVTQSSRYLVKRTGEKV |
| | chain | FLECVQDMDHENMFWYRQDPGLGLRLIYFSYDVKMKE |
| | | KGDIPEGYSVSREKKERFSLILESASTNQTSMYLCASRLP |
| | | SRTYEQYFGPGTRLTVTEDLKNVFPPEVAVFEPSEAEIS |
| | | HTQKATLVCLATGFYPDHVELSWWVNGKEVHSGVSTD |
| | | PQPLKEQPALNDSRYCLSSRLRVSATFWQNPRNHFRCQ |
| | | VQFYGLSENDEWTQDRAKPVTQIVSAEAWGRADCGFT |
| | | SESYQQGVLSATILYEILLGKATLYAVLVSALVLMAMV |
| | | KRKDSRG |
| 55 | R59P2E7 alpha | METLLGLLILWLQLQWVSSKQEVTQIPAALSVPEGENL |
| | chain | VLNCSFTDSAIYNLQWFRQDPGKGLTSLLLIQSSQREQT |
| | | SGRLNASLDKSSGRSTLYIAASQPGDSATYLCAVNSDY |
| | | KLSFGAGTTVTVRANIQNPDPAVYQLRDSKSSDKSVCL |
| | | FTDFDSQTNVSQSKDSDVYITDKTVLDMRSMDFKSNSA |
| | | VAWSNKSDFACANAFNNSIIPEDTFFPSPESSCDVKLVE |
| | | KSFETDTNLNFQNLSVIGFRILLLKVAGFNLLMTLRLWS |
| | | S |
| 56 | R59P2E7 beta | MLSPDLPDSAWNTRLLCHVMLCLLGAVSVAAGVIQSPR |
| | chain | HLIKEKRETATLKCYPIPRHDTVYWYQQGPGQDPQFLIS |
| | | FYEKMQSDKGSIPDRFSAQQFSDYHSELNMSSLELGDS |
| | | ALYFCASSLGLGTGDYGYTFGSGTRLTVVEDLNKVFPP |
| | | EVAVFEPSEAEISHTQKATLVCLATGFFPDHVELSWWV |
| | | NGKEVHSGVSTDPQPLKEQPALNDSRYCLSSRLRVSAT |
| | | FWQNPRNHFRCQVQFYGLSENDEWTQDRAKPVTQIVS |
| | | AEAWGRADCGFTSVSYQQGVLSATILYEILLGKATLYA |
| | | VLVSALVLMAMVKRKDF |
| 57 | R11P3D3 alpha | MEKNPLAAPLLILWFHLDCVSSILNVEQSPQSLHVQEGD |
| | chain | STNFTCSFPSSNFYALHWYRWETAKSPEALFVMTLNGD |
| | | EKKKGRISATLNTKEGYSYLYIKGSQPEDSATYLCALYN |
| | | NNDMRFGAGTRLTVKPNIQNPDPAVYQLRDSKSSDKSV |
| | | CLFTDFDSQTNVSQSKDSDVYITDKTVLDMRSMDFKSN |
| | | SAVAWSNKSDFACANAFNNSIIPEDTFFPSPESSCDVKL |
| | | VEKSFETDTNLNFQNLSVIGFRILLLKVAGFNLLMTLRL |
| | | WSS |
| 58 | R11P3D3 beta | MDSWTFCCVSLCILVAKHTDAGVIQSPRHEVTEMGQEV |
| | chain | TLRCKPISGHNSLFWYRQTMMRGLELLIYFNNNVPIDDS |
| | | GMPEDRFSAKMPNASFSTLKIQPSEPRDSAVYFCASSPG |
| | | STDTQYFGPGTRLTVLEDLKNVFPPEVAVFEPSEAEISHT |
| | | QKATLVCLATGFYPDHVELSWWVNGKEVHSGVSTDPQ |
| | | PLKEQPALNDSRYCLSSRLRVSATFWQNPRNHFRCQVQ |
| | | FYGLSENDEWTQDRAKPVTQIVSAEAWGRADCGFTSES |
| | | YQQGVLSATILYEILLGKATLYAVLVSALVLMAMVKRK |
| | | DSRG |
| 59 | R16P1C10 alpha | MKSLRVLLVILWLQLSWVWSQQKEVEQNSGPLSVPEG |
| | chain | AIASLNCTYSDRGSQSFFWYRQYSGKSPELIMFIYSNGD |
| | | KEDGRFTAQLNKASQYVSLLIRDSQPSDSATYLCAAVIS |
| | | NFGNEKLTFGTGTRLTIIPNIQNPDPAVYQLRDSKSSDKS |
| | | VCLFTDFDSQTNVSQSKDSDVYITDKTVLDMRSMDFKS |
| | | NSAVAWSNKSDFACANAFNNSIIPEDTFFPSPESSCDVK |
| | | LVEKSFETDTNLNFQNLSVIGFRILLLKVAGFNLLMTLR |
| | | LWSS |
| 60 | R16P1C10 beta | MGSRLLCWVLLCLLGAGPVKAGVTQTPRYLIKTRGQQ |
| | chain | VTLSCSPISGHRSVSWYQQTPGQGLQFLFEYFSETQRNK |
| | | GNFPGRFSGRQFSNSRSEMNVSTLELGDSALYLCASSP |
| | | WDSPNEQYFGPGTRLTVTEDLKNVFPPEVAVFEPSEAEI |
| | | SHTQKATLVCLATGFYPDHVELSWWVNGKEVHSGVST |
| | | DPQPLKEQPALNDSRYCLSSRLRVSATFWQNPRNHFRC |
| | | QVQFYGLSENDEWTQDRAKPVTQIVSAEAWGRADCGF |
| | | TSESYQQGVLSATILYEILLGKATLYAVLVSALVLMAM |
| | | VKRKDSRG |
| 61 | R16P1E8 alpha | MMKSLRVLLVILWLQLSWVWSQQKEVEQDPGPLSVPE |
| | chain | GAIVSLNCTYSNSAFQYFMWYRQYSRKGPELLMYTYSS |
| | | GNKEDGRFTAQVDKSSKYISLFIRDSQPSDSATYLCAMS |
| | | EAAGNKLTFGGGTRVLVKPNIQNPDPAVYQLRDSKSSD |
| | | KSVCLFTDFDSQTNVSQSKDSDVYITDKTVLDMRSMDF |
| | | KSNSAVAWSNKSDFACANAFNNSIIPEDTFFPSPESSCD |
| | | VKLVEKSFETDTNLNFQNLSVIGFRILLLKVAGFNLLMT |
| | | LRLWSS |
| 62 | R16P1E8 beta | MGTRLLCWAALCLLGAELTEAGVAQSPRYKIIEKRQSV |
| | chain | AFWCNPISGHATLYWYQQILGQGPKLLIQFQNNGVVDD |
| | | SQLPKDRFSAERLKGVDSTLKIQPAKLEDSAVYLCASSY |
| | | TNQGEAFFGQGTRLTVVEDLNKVFPPEVAVFEPSEAEIS |
| | | HTQKATLVCLATGFFPDHVELSWWVNGKEVHSGVSTD |
| | | PQPLKEQPALNDSRYCLSSRLRVSATFWQNPRNHFRCQ |
| | | VQFYGLSENDEWTQDRAKPVTQIVSAEAWGRADCGFT |
| | | SVSYQQGVLSATILYEILLGKATLYAVLVSALVLMAMV |
| | | KRKDF |
| 63 | R17P1A9 alpha | MKSLRVLLVILWLQLSWVWSQQKEVEQNSGPLSVPEG |
| | chain | AIASLNCTYSDRGSQSFFWYRQYSGKSPELIMSIYSNGD |
| | | KEDGRFTAQLNKASQYVSLLIRDSQPSDSATYLCAVLN |
| | | QAGTALIFGKGTTLSVSSNIQNPDPAVYQLRDSKSSDKS |
| | | VCLFTDFDSQTNVSQSKDSDVYITDKTVLDMRSMDFKS |
| | | NSAVAWSNKSDFACANAFNNSIIPEDTFFPSPESSCDVK |
| | | LVEKSFETDTNLNFQNLSVIGFRILLLKVAGFNLLMTLR |
| | | LWSS |
| 64 | R17P1A9 beta | MGFRLLCCVAFCLLGAGPVDSGVTQTPKHLITATGQRV |
| | chain | TLRCSPRSGDLSVYWYQQSLDQGLQFLIQYYNGEERAK |
| | | GNILERFSAQQFPDLHSELNLSSLELGDSALYFCASSAET |
| | | GPWLGNEQFFGPGTRLTVLEDLKNVFPPEVAVFEPSEAE |
| | | ISHTQKATLVCLATGFYPDHVELSWWVNGKEVHSGVS |
| | | TDPQPLKEQPALNDSRYCLSSRLRVSATFWQNPRNHFR |
| | | CQVQFYGLSENDEWTQDRAKPVTQIVSAEAWGRADCG |
| | | FTSESYQQGVLSATILYEILLGKATLYAVLVSALVLMA |
| | | MVKRKDSRG |
| 65 | R17P1D7 alpha | MACPGFLWALVISTCLEFSMAQTVTQSQPEMSVQEAET |
| | chain | VTLSCTYDTSESDYYLFWYKQPPSRQMILVIRQEAYKQ |
| | | QNATENRFSVNFQKAAKSFSLKISDSQLGDAAMYFCAY |
| | | RWAQGGSEKLVFGKGTKLTVNPYIQKPDPAVYQLRDS |
| | | KSSDKSVCLFTDFDSQTNVSQSKDSDVYITDKTVLDMR |
| | | SMDFKSNSAVAWSNKSDFACANAFNNSIIPEDTFFPSPE |
| | | SSCDVKLVEKSFETDTNLNFQNLSVIGFRILLLKVAGFN |
| | | LLMTLRLWSS |
| 66 | R17P1D7 beta | MTIRLLCYMGFYFLGAGLMEADIYQTPRYLVIGTGKKIT |
| | chain | LECSQTMGHDKMYWYQQDPGMELHLIHYSYGVNSTE |
| | | KGDLSSESTVSRIRTEHFPLTLESARPSHTSQYLCATELW |
| | | SSGGTGELFFGEGSRLTVLEDLKNVFPPEVAVFEPSEAEI |
| | | SHTQKATLVCLATGFYPDHVELSWWVNGKEVHSGVST |
| | | DPQPLKEQPALNDSRYCLSSRLRVSATFWQNPRNHFRC |
| | | QVQFYGLSENDEWTQDRAKPVTQIVSAEAWGRADCGF |
| | | TSESYQQGVLSATILYEILLGKATLYAVLVSALVLMAM |
| | | VKRKDSRG |
| 67 | R17P1G3 alpha | MKSLRVLLVILWLQLSWVWSQQKEVEQNSGPLSVPEG |
| | chain | AIASLNCTYSDRGSQSFFWYRQYSGKSPELIMSIYSNGD |
| | | KEDGRFTAQLNKASQYVSLLIRDSQPSDSATYLCAVGPS |
| | | GTYKYIFGTGTRLKVLANIQNPDPAVYQLRDSKSSDKS |
| | | VCLFTDFDSQTNVSQSKDSDVYITDKTVLDMRSMDFKS |
| | | NSAVAWSNKSDFACANAFNNSIIPEDTFFPSPESSCDVK |
| | | LVEKSFETDTNLNFQNLSVIGFRILLLKVAGFNLLMTLR |
| | | LWSS |
| 68 | R17P1G3 beta | MGPQLLGYVVLCLLGAGPLEAQVTQNPRYLITVTGKKL |
| | chain | TVTCSQNMNHEYMSWYRQDPGLGLRQIYYSMNVEVT |
| | | DKGDVPEGYKVSRKEKRNFPLILESPSPNQTSLYFCASS |
| | | PGGSGNEQFFGPGTRLTVLEDLKNVFPPEVAVFEPSEAE |
| | | ISHTQKATLVCLATGFYPDHVELSWWVNGKEVHSGVS |
| | | TDPQPLKEQPALNDSRYCLSSRLRVSATFWQNPRNHFR |
| | | CQVQFYGLSENDEWTQDRAKPVTQIVSAEAWGRADCG |
| | | FTSESYQQGVLSATILYEILLGKATLYAVLVSALVLMA |
| | | MVKRKDSRG |
| 69 | R17P2B6 alpha | MKSLRVLLVILWLQLSWVWSQQKEVEQNSGPLSVPEG |
| | chain | AIASLNCTYSDRGSQSFFWYRQYSGKSPELIMFIYSNGD |
| | | KEDGRFTAQLNKASQYVSLLIRDSQPSDSATYLCAVVS |
| | | GGGADGLTFGKGTHLIIQPYIQKPDPAVYQLRDSKSSDK |
| | | SVCLFTDFDSQTNVSQSKDSDVYITDKTVLDMRSMDFK |
| | | SNSAVAWSNKSDFACANAFNNSIIPEDTFFPSPESSCDV |
| | | KLVEKSFETDTNLNFQNLSVIGFRILLLKVAGFNLLMTL |
| | | RLWSS |
| 70 | R17P2B6 beta | MLSPDLPDSAWNTRLLCHVMLCLLGAVSVAAGVIQSPR |
| | chain | HLIKEKRETATLKCYPIPRHDTVYWYQQGPGQDPQFLIS |
| | | FYEKMQSDKGSIPDRFSAQQFSDYHSELNMSSLELGDS |
| | | ALYFCASSLGRGGQPQHFGDGTRLSILEDLNKVFPPEVA |
| | | VFEPSEAEISHTQKATLVCLATGFFPDHVELSWWVNGK |
| | | EVHSGVSTDPQPLKEQPALNDSRYCLSSRLRVSATFWQ |
| | | NPRNHFRCQVQFYGLSENDEWTQDRAKPVTQIVSAEA |
| | | WGRADCGFTSVSYQQGVLSATILYEILLGKATLYAVLV |
| | | SALVLMAMVKRKDF |
| 71 | R11P3D3KE | MEKNPLAAPLLILWFHLDCVSSILNVEQSPQSLHVQEGD |
| | alpha chain | STNFTCSFPSSNFYALHWYRKETAKSPEALFVMTLNGD |
| | | EKKKGRISATLNTKEGYSYLYIKGSQPEDSATYLCALYN |
| | | NNDMRFGAGTRLTVKPNIQNPDPAVYQLRDSKSSDKSV |
| | | CLFTDFDSQTNVSQSKDSDVYITDKTVLDMRSMDFKSN |
| | | SAVAWSNKSDFACANAFNNSIIPEDTFFPSPESSCDVKL |
| | | VEKSFETDTNLNFQNLSVIGFRILLLKVAGFNLLMTLRL |
| | | WSS |
| 72 | R11P3D3KE beta | NNNVPIDDSGMPEDRFSAKMPNASFSTLKIQPSEPRDSA |
| | chain | VYFCASSPGSTDTQYFGPGTRLTVLEDLKNVFPPEVAVF |
| | | EPSEAEISHTQKATLVCLATGFYPDHVELSWWVNGKEV |
| | | HSGVSTDPQPLKEQPALNDSRYCLSSRLRVSATFWQNP |
| | | RNHFRCQVQFYGLSENDEWTQDRAKPVTQIVSAEAWG |
| | | RADCGFTSESYQQGVLSATILYEILLGKATLYAVLVSAL |
| | | VLMAMVKRKDSRG |
| 73 | R39P1C12 alpha | TYLYWYKQEPGAGLQLLTYIFSNMDMKQDQRLTVLLN |
| | chain | KKDKHLSLRIADTQTGDSAIYFCAEIDNQGGKLIFGQGT |
| | | ELSVKPNIQNPDPAVYQLRDSKSSDKSVCLFTDFDSQTN |
| | | VSQSKDSDVYITDKTVLDMRSMDFKSNSAVAWSNKSD |
| | | FACANAFNNSIIPEDTFFPSPESSCDVKLVEKSFETDTNL |
| | | NFQNLSVIGFRILLLKVAGFNLLMTLRLWSS |
| 74 | R39P1C12 beta | MGPGLLCWALLCLLGAGLVDAGVTQSPTHLIKTRGQQ |
| | chain | VTLRCSPKSGHDTVSWYQQALGQGPQFIFQYYEEEERQ |
| | | RGNFPDRFSGHQFPNYSSELNVNALLLGDSALYLCASS |
| | | QLNTEAFFGQGTRLTVVEDLNKVFPPEVAVFEPSEAEIS |
| | | HTQKATLVCLATGFFPDHVELSWWVNGKEVHSGVSTD |
| | | PQPLKEQPALNDSRYCLSSRLRVSATFWQNPRNHFRCQ |
| | | VQFYGLSENDEWTQDRAKPVTQIVSAEAWGRADCGFT |
| | | SVSYQQGVLSATILYEILLGKATLYAVLVSALVLMAMV |
| | | KRKDF |
| 75 | R39P1F5 alpha | MKSLRVLLVILWLQLSWVWSQQKEVEQNSGPLSVPEG |
| | chain | AIASLNCTYSDRGSQSFFWYRQYSGKSPELIMFIYSNGD |
| | | KEDGRFTAQLNKASQYVSLLIRDSQPSDSATYLCAVNN |
| | | ARLMFGDGTQLVVKPNIQNPDPAVYQLRDSKSSDKSVC |
| | | LFTDFDSQTNVSQSKDSDVYITDKTVLDMRSMDFKSNS |
| | | AVAWSNKSDFACANAFNNSIIPEDTFFPSPESSCDVKLV |
| | | EKSFETDTNLNFQNLSVIGFRILLLKVAGFNLLMTLRLW |
| | | SS |
| 76 | R39P1F5 beta | MDTWLVCWAIFSLLKAGLTEPEVTQTPSHQVTQMGQE |
| | chain | VILRCVPISNHLYFYWYRQILGQKVEFLVSFYNNEISEKS |
| | | EIFDDQFSVERPDGSNFTLKIRSTKLEDSAMYFCASSGQ |
| | | GANEQYFGPGTRLTVTEDLKNVFPPEVAVFEPSEAEISH |
| | | TQKATLVCLATGFYPDHVELSWWVNGKEVHSGVSTDP |
| | | QPLKEQPALNDSRYCLSSRLRVSATFWQNPRNHFRCQV |
| | | QFYGLSENDEWTQDRAKPVTQIVSAEAWGRADCGFTS |
| | | ESYQQGVLSATILYEILLGKATLYAVLVSALVLMAMVK |
| | | RKDSRG |
| 77 | R40P1C2 alpha | MACPGFLWALVISTCLEFSMAQTVTQSQPEMSVQEAET |
| | chain | VTLSCTYDTSESDYYLFWYKQPPSRQMILVIRQEAYKQ |
| | | QNATENRFSVNFQKAAKSFSLKISDSQLGDAAMYFCAY |
| | | LNYQLIWGAGTKLIIKPDIQNPDPAVYQLRDSKSSDKSV |
| | | CLFTDFDSQTNVSQSKDSDVYITDKTVLDMRSMDFKSN |
| | | SAVAWSNKSDFACANAFNNSIIPEDTFFPSPESSCDVKL |
| | | VEKSFETDTNLNFQNLSVIGFRILLLKVAGFNLLMTLRL |
| | | WSS |
| 78 | R40P1C2 beta | MDTWLVCWAIFSLLKAGLTEPEVTQTPSHQVTQMGQE |
| | chain | VILRCVPISNHLYFYWYRQILGQKVEFLVSFYNNEISEKS |
| | | EIFDDQFSVERPDGSNFTLKIRSTKLEDSAMYFCASSEM |
| | | TAVGQYFGPGTRLTVTEDLKNVFPPEVAVFEPSEAEISH |
| | | TQKATLVCLATGFYPDHVELSWWVNGKEVHSGVSTDP |
| | | QPLKEQPALNDSRYCLSSRLRVSATFWQNPRNHFRCQV |
| | | QFYGLSENDEWTQDRAKPVTQIVSAEAWGRADCGFTS |
| | | ESYQQGVLSATILYEILLGKATLYAVLVSALVLMAMVK |
| | | RKDSRG |
| 79 | R41P3E6 alpha | MKSLRVLLVILWLQLSWVWSQQKEVEQNSGPLSVPEG |
| | chain | AIASLNCTYSDRGSQSFFWYRQYSGKSPELIMFIYSNGD |
| | | KEDGRFT |
| | | AQLNKASQYVSLLIRDSQPSDSATYLCAAFSGYALNFG |
| | | KGTSLLVTPHIQNPDPAVYQLRDSKSSDKSVCLFTDFDS |
| | | QTNVSQSKDSDVYITDKTVLDMRSMDFKSNSAVAWSN |
| | | KSDFACANAFNNSIIPEDTFFPSPESSCDVKLVEKSFETD |
| | | TNLNFQNLSVIGFRILLLKVAGFNLLMTLRLWSS |
| 80 | R41P3E6 beta | MDTWLVCWAIFSLLKAGLTEPEVTQTPSHQVTQMGQE |
| | chain | VILRCVPISNHLYFYWYRQILGQKVEFLVSFYNNEISEKS |
| | | EIFDDQFSVERPDGSNFTLKIRSTKLEDSAMYFCASSQY |
| | | TGELFFGEGSRLTVLEDLKNVFPPEVAVFEPSEAEISHTQ |
| | | KATLVCLATGFYPDHVELSWWVNGKEVHSGVSTDPQP |
| | | LKEQPALNDSRYCLSSRLRVSATFWQNPRNHFRCQVQF |
| | | YGLSENDEWTQDRAKPVTQIVSAEAWGRADCGFTSES |
| | | YQQGVLSATILYEILLGKATLYAVLVSALVLMAMVKR |
| | | KDSRG |
| 81 | R43P3G4 alpha | MKSLRVLLVILWLQLSWVWSQQKEVEQNSGPLSVPEG |
| | chain | AIASLNCTYSDRGSQSFFWYRQYSGKSPELIMFIYSNGD |
| | | KEDGRFTAQLNKASQYVSLLIRDSQPSDSATYLCAVNG |
| | | GDMRFGAGTRLTVKPNIQNPDPAVYQLRDSKSSDKSVC |
| | | LFTDFDSQTNVSQSKDSDVYITDKTVLDMRSMDFKSNS |
| | | AVAWSNKSDFACANAFNNSIIPEDTFFPSPESSCDVKLV |
| | | EKSFETDTNLNFQNLSVIGFRILLLKVAGFNLLMTLRLW |
| | | SS |
| 82 | R43P3G4 beta | MDTWLVCWAIFSLLKAGLTEPEVTQTPSHQVTQMGQE |
| | chain | VILRCVPISNHLYFYWYRQILGQKVEFLVSFYNNEISEKS |
| | | EIFDDQFSVERPDGSNFTLKIRSTKLEDSAMYFCASSGQ |
| | | GALEQYFGPGTRLTVTEDLKNVFPPEVAVFEPSEAEISH |
| | | TQKATLVCLATGFYPDHVELSWWVNGKEVHSGVSTDP |
| | | QPLKEQPALNDSRYCLSSRLRVSATFWQNPRNHFRCQV |
| | | QFYGLSENDEWTQDRAKPVTQIVSAEAWGRADCGFTS |
| | | ESYQQGVLSATILYEILLGKATLYAVLVSALVLMAMVK |
| | | RKDSRG |
| 83 | R44P3B3 alpha | MAMLLGASVLILWLQPDWVNSQQKNDDQQVKQNSPS |
| | chain | LSVQEGRISILNCDYTNSMFDYFLWYKKYPAEGPTFLISI |
| | | SSIKDKNEDGRFTVFLNKSAKHLSLHIVPSQPGDSAVYF |
| | | CAASGLYNQGGKLIFGQGTELSVKPNIQNPDPAVYQLR |
| | | DSKSSDKSVCLFTDFDSQTNVSQSKDSDVYITDKTVLD |
| | | MRSMDFKSNSAVAWSNKSDFACANAFNNSIIPEDTFFPS |
| | | PESSCDVKLVEKSFETDTNLNFQNLSVIGFRILLLKVAGF |
| | | NLLMTLRLWSS |
| 84 | R44P3B3 beta | MGCRLLCCVVFCLLQAGPLDTAVSQTPKYLVTQMGND |
| | chain | KSIKCEQNLGHDTMYWYKQDSKKFLKIMFSYNNKELII |
| | | NETVPNRFSPKSPDKAHLNLHINSLELGDSAVYFCASSL |
| | | GDRGYEQYFGPGTRLTVTEDLKNVFPPEVAVFEPSEAEI |
| | | SHTQKATLVCLATGFYPDHVELSWWVNGKEVHSGVST |
| | | DPQPLKEQPALNDSRYCLSSRLRVSATFWQNPRNHFRC |
| | | QVQFYGLSENDEWTQDRAKPVTQIVSAEAWGRADCGF |
| | | TSESYQQGVLSATILYEILLGKATLYAVLVSALVLMAM |
| | | VKRKDSRG |
| 85 | R44P3E7 alpha | MKTFAGFSFLFLWLQLDCMSRGEDVEQSLFLSVREGDS |
| | chain | SVINCTYTDSSSTYLYWYKQEPGAGLQLLTYIFSNMDM |
| | | KQDQRLTVLLNKKDKHLSLRIADTQTGDSAIYFCAEINN |
| | | NARLMFGDGTQLVVKPNIQNPDPAVYQLRDSKSSDKSV |
| | | CLFTDFDSQTNVSQSKDSDVYITDKTVLDMRSMDFKSN |
| | | SAVAWSNKSDFACANAFNNSIIPEDTFFPSPESSCDVKL |
| | | VEKSFETDTNLNFQNLSVIGFRILLLKVAGFNLLMTLRL |
| | | WSS |
| 86 | R44P3E7 beta | MLSPDLPDSAWNTRLLCHVMLCLLGAVSVAAGVIQSPR |
| | chain | HLIKEKRETATLKCYPIPRHDTVYWYQQGPGQDPQFLIS |
| | | FYEKMQSDKGSIPDRFSAQQFSDYHSELNMSSLELGDS |
| | | ALYFCASSPPDQNTQYFGPGTRLTVLEDLKNVFPPEVA |
| | | VFEPSEAEISHTQKATLVCLATGFYPDHVELSWWVNGK |
| | | EVHSGVSTDPQPLKEQPALNDSRYCLSSRLRVSATFWQ |
| | | NPRNHFRCQVQFYGLSENDEWTQDRAKPVTQIVSAEA |
| | | WGRADCGFTSESYQQGVLSATILYEILLGKATLYAVLV |
| | | SALVLMAMVKRKDSRG |
| 87 | R49P2B7 alpha | MLLLLVPVLEVIFTLGGTRAQSVTQLGSHVSVSEGALVL |
| | chain | LRCNYSSSVPPYLFWYVQYPNQGLQLLLKYTTGATLVK |
| | | GINGFEAEFKKSETSFHLTKPSAHMSDAAEYFCAVRIFG |
| | | NEKLTFGTGTRLTIIPNIQNPDPAVYQLRDSKSSDKSVCL |
| | | FTDFDSQTNVSQSKDSDVYITDKTVLDMRSMDFKSNSA |
| | | VAWSNKSDFACANAFNNSIIPEDTFFPSPESSCDVKLVE |
| | | KSFETDTNLNFQNLSVIGFRILLLKVAGFNLLMTLRLWS |
| | | S |
| 88 | R49P2B7 beta | MGIRLLCRVAFCFLAVGLVDVKVTQSSRYLVKRTGEKV |
| | chain | FLECVQDMDHENMFWYRQDPGLGLRLIYFSYDVKMKE |
| | | KGDIPEGYSVSREKKERFSLILESASTNQTSMYLCASSL |
| | | MGELTGELFFGEGSRLTVLEDLKNVFPPEVAVFEPSEAE |
| | | ISHTQKATLVCLATGFYPDHVELSWWVNGKEVHSGVS |
| | | TDPQPLKEQPALNDSRYCLSSRLRVSATFWQNPRNHFR |
| | | CQVQFYGLSENDEWTQDRAKPVTQIVSAEAWGRADCG |
| | | FTSESYQQGVLSATILYEILLGKATLYAVLVSALVLMA |
| | | MVKRKDSRG |
| 89 | R55P1G7 alpha | MMKSLRVLLVILWLQLSWVWSQQKEVEQDPGPLSVPE |
| | chain | GAIVSLNCTYSNSAFQYFMWYRQYSRKGPELLMYTYSS |
| | | GNKEDGRFTAQVDKSSKYISLFIRDSQPSDSATYLCAM |
| | | MGDTGTASKLTFGTGTRLQVTLDIQNPDPAVYQLRDSK |
| | | SSDKSVCLFTDFDSQTNVSQSKDSDVYITDKTVLDMRS |
| | | MDFKSNSAVAWSNKSDFACANAFNNSIIPEDTFFPSPES |
| | | SCDVKLVEKSFETDTNLNFQNLSVIGFRILLLKVAGFNL |
| | | LMTLRLWSS |
| 90 | R55P1G7 beta | MGIRLLCRVAFCFLAVGLVDVKVTQSSRYLVKRTGEKV |
| | chain | FLECVQDMDHENMFWYRQDPGLGLRLIYFSYDVKMKE |
| | | KGDIPEGYSVSREKKERFSLILESASTNQTSMYLCASSFG |
| | | GYEQYFGPGTRLTVTEDLKNVFPPEVAVFEPSEAEISHT |
| | | QKATLVCLATGFYPDHVELSWWVNGKEVHSGVSTDPQ |
| | | PLKEQPALNDSRYCLSSRLRVSATFWQNPRNHFRCQVQ |
| | | FYGLSENDEWTQDRAKPVTQIVSAEAWGRADCGFTSES |
| | | YQQGVLSATILYEILLGKATLYAVLVSALVLMAMVKR |
| | | KDSRG |
| 91 | R59P2A7 alpha | MKSLRVLLVILWLQLSWVWSQQKEVEQNSGPLSVPEG |
| | chain | AIASLNCTYSDRGSQSFFWYRQYSGKSPELIMSIYSNGD |
| | | KEDGRFTAQLNKASQYVSLLIRDSQPSDSATYLCAVQP |
| | | HDMRFGAGTRLTVKPNIQNPDPAVYQLRDSKSSDKSVC |
| | | LFTDFDSQTNVSQSKDSDVYITDKTVLDMRSMDFKSNS |
| | | AVAWSNKSDFACANAFNNSIIPEDTFFPSPESSCDVKLV |
| | | EKSFETDTNLNFQNLSVIGFRILLLKVAGFNLLMTLRLW |
| | | SS |
| 92 | R59P2A7 beta | MLCSLLALLLGTFFGVRSQTIHQWPATLVQPVGSPLSLE |
| | chain | CTVEGTSNPNLYWYRQAAGRGLQLLFYSVGIGQISSEV |
| | | PQNLSASRPQDRQFILSSKKLLLSDSGFYLCAWSGLVAE |
| | | QFFGPGTRLTVLEDLKNVFPPEVAVFEPSEAEISHTQKA |
| | | TLVCLATGFYPDHVELSWWVNGKEVHSGVSTDPQPLK |
| | | EQPALNDSRYCLSSRLRVSATFWQNPRNHFRCQVQFYG |
| | | LSENDEWTQDRAKPVTQIVSAEAWGRADCGFTSESYQ |
| | | QGVLSATILYEILLGKATLYAVLVSALVLMAMVKRKDS |
| | | RG |
| 93 | P2A | ATNFSLLKQAGDVEENPGP |
| 94 | T2A | EGRGSLLTCGDVEENPGP |
| 95 | E2A | QCTNYALLKLAGDVESNPGP |
| 96 | F2A | VKQTLNFDLLKLAGDVESNPGP |
| 97 | RD114TR | MKLPTGMVILCSLIIVRAGFDDPRKAIALVQKQHGKPCE |
| | | CSGGQVSEAPPNSIQQVTCPGKTAYLMTNQKWKCRVT |
| | | PKISPSGGELQNCPCNTFQDSMHSSCYTEYRQCRRINKT |
| | | YYTATLLKIRSGSLNEVQILQNPNQLLQSPCRGSINQPVC |
| | | WSATAPIHISDGGGPLDTKRVWTVQKRLEQIHKAMTPE |
| | | LQYHPLALPKVRDDLSLDARTFDILNTTFRLLQMSNFSL |
| | | AQDCWLCLKLGTPTPLAIPTPSLTYSLADSLANASCQIIP |
| | | PLLVQPMQFSNSSCLSSPFINDTEQIDLGAVTFTNCTSVA |
| | | NVSSPLCALNGSVFLCGNNMAYTYLPQNWTRLCVQAS |
| | | LLPDIDINPGDEPVPIPAIDHYIHRPKRAVQFIPLLAGLGI |
| | | TAAFTTGATGLGVSVTQYTKLSHQLISDVQVLSGTIQDL |
| | | QDQVDSLAEVVLQNRRGLDLLTAEQGGICLALQEKCCF |
| | | YANKSGIVRNKIRTLQEELQKRRESLASNPLWTGLQGFL |
| | | PYLLPLLGPLLTLLLILTIGPCVFNRLVQFVKDRISVVQA |
| | | LVLTQQYHQLKPL |
| 256 | WPREmut1 | cagtctgacgtacgcgtaatcaacctctggattacaaaatttgtgaaagattgactggtatt |
| | | cttaactatgttgctccttttacgctatgtggatacgctgctttaatgcctttgtatcatgctatt |
| | | gcttcccgtatggctttcattttctcctccttgtataaatcctggttgctgtctctttatgagga |
| | | gttgtggcccgttgtcaggcaacgtggcgtggtgtgcactgtgtttgctgacgcaacccc |
| | | cactggttggggcattgccaccacctgtcagctcctttccgggactttcgctttccccctcc |
| | | ctattgccacggcggaactcatcgccgcctgccttgcccgctgctggacaggggctcg |
| | | gctgttgggcactgacaattccgtggtgttgtcggggaaatcatcgtcctttccttggctgc |
| | | tcgcctgtgttgccacctggattctgcgcgggacgtccttctgctacgtcccttcggccct |
| | | caatccagcggaccttccttcccgcggcctgctgccggctctgcggcctcttccgcgtct |
| | | tcgccttcgccctcagacgagtcggatctccctttgggccgcctccccgcc |
| 257 | WPREmut2 | Gagcatcttaccgccatttatacccatatttgactgtttttcttgatttgggtatacatttaaat |
| | | gttaataaaacaaaatggtggggcaatcatttacattttttgggatatgtaattactagttcag |
| | | gtgtattgccacaagacaaacttgttaagaaactttcccgttatttacgctctgttcctgttaa |
| | | tcaacctctggattacaaaatttgtgaaagattgactgatattcttaactttgttgctccttttac |
| | | gctgtgtggatttgctgctttattgcctctgtatcttgctattgcttcccgtacggctttcgtttt |
| | | ctcctccttgtataaatcctggttgctgtctctttttgaggagttgtggcccgttgtccgtcaa |
| | | cgtggcgtggtgtgctctgtgtttgctgacgcaacccccactggctggggcattgccacc |
| | | acctgtcaactcctttctgggactttcgctttccccctcccgatcgccacggcagaactcat |
| | | cgccgcctgccttgcccgctgctggacaggggctaggttgctgggcactgataattccg |
| | | tggtgttgtc |
| 258 | CD8α1 | MALPVTALLLPLALLLHAARPSQFRVSPLDRTWNLGET |
| | | VELKCQVLLSNPTSGCSWLFQPRGAAASPTFLLYLSQN |
| | | KPKAAEGLDTQRFSGKRLGDTFVLTLSDFRRENEGYYF |
| | | CSALSNSIMYFSHFVPVFLPAKPTTTPAPRPPTPAPTIASQ |
| | | PLSLRPEACRPAAGGAVHTRGLDFACDIYIWAPLAGTC |
| | | GVLLLSLVITLYCNHRNRRRVCKCPRPVVKSGDKPSLS |
| | | ARYV |
| 259 | CD8α2 | MALPVTALLLPLALLLHAARPSQFRVSPLDRTWNLGET |
| | | VELKCQVLLSNPTSGCSWLFQPRGAAASPTFLLYLSQN |
| | | KPKAAEGLDTQRFSGKRLGDTFVLTLSDFRRENEGCYF |
| | | CSALSNSIMYFSHFVPVFLPAKPTTTPAPRPPTPAPTIASQ |
| | | PLSLRPEACRPAAGGAVHTRGLDFACDIYIWAPLAGTC |
| | | GVLLLSLVITLYCNHRNRRRVCKCPRPVVKSGDKPSLS |
| | | ARYV |
| 260 | CD8α stalk | KPTTTPAPRPPTPAPTIASQPLSLRPEACRPAAGGAVHTR |
| | | GLDFACD |
| 261 | CD8α Ig-like | SQFRVSPLDRTWNLGETVELKCQVLLSNPTSGCSWLFQ |
| | domain-2 | PRGAAASPTFLLYLSQNKPKAAEGLDTQRFSGKRLGDT |
| | | FVLTLSDFRRENEGCYFCS2ALSNSIMYFSHFVPVFLPA |
| 262 | m2CD8α | MALPVTALLLPLALLLHAARPSQFRVSPLDRTWNLGET |
| | | VELKCQVLLSNPTSGCSWLFQPRGAAASPTFLLYLSQN |
| | | KPKAAEGLDTQRFSGKRLGDTFVLTLSDFRRENEGCYF |
| | | CSALSNSIMYFSHFVPVFLPASVVDFLPTTAQPTKKSTL |
| | | KKRVCRLPRPETQKGPLCSPIYIWAPLAGTCGVLLLSLVI |
| | | TLYCNHRNRRRVCKCPRPVVKSGDKPSLSARYV |
| 263 | MSCV promoter | Tgaaagaccccacctgtaggtttggcaagctagcttaagtaacgccattttgcaaggcat |
| | | ggaaaatacataactgagaatagagaagttcagatcaaggttaggaacagagagacag |
| | | cagaatatgggccaaacaggatatctgtggtaagcagttcctgccccggctcagggcca |
| | | agaacagatggtccccagatgcggtcccgccctcagcagtttctagagaaccatcagat |
| | | gtttccagggtgccccaaggacctgaaaatgaccctgtgccttatttgaactaaccaatca |
| | | gttcgcttctcgcttctgttcgcgcgcttctgctccccgagctcaataaaagagcccacaa |
| | | cccctcact |
| 264 | WPRE | cagtctgacgtacgcgtaatcaacctctggattacaaaatttgtgaaagattgactggtatt |
| | | cttaactatgttgctccttttacgctatgtggatacgctgctttaatgcctttgtatcatgctatt |
| | | gcttcccgtatggctttcattttctcctccttgtataaatcctggttgctgtctctttatgagga |
| | | gttgtggcccgttgtcaggcaacgtggcgtggtgtgcactgtgtttgctgacgcaacccc |
| | | cactggttggggcattgccaccacctgtcagctcctttccgggactttcgctttccccctcc |
| | | ctattgccacggcggaactcatcgccgcctgccttgcccgctgctggacaggggctcg |
| | | gctgttgggcactgacaattccgtggtgttgtcggggaagctgacgtcctttccatggctg |
| | | ctcgcctgtgttgccacctggattctgcgcgggacgtccttctgctacgtcccttcggccc |
| | | tcaatccagcggaccttccttcccgcggcctgctgccggctctgcggcctcttccgcgtc |
| | | ttcgccttcgccctcagacgagtcggatctccctttgggccgcctccccgcc |
| 265 | Furin consensus | RXXR |
| 266 | Linker | SGSG |
| 293 | CD8β Signal | MRPRLWLLLAAQLTVLHGNSV |
| | peptide | |
| 294 | S19 Signal | MEFGLSWLFLVAILKGVQC |
| | peptide | |
| 303 | R11P3D3KE beta | MDSWTFCCVSLCILVAKHTDAGVIQSPRHEVTEMGQEV |
| | chain | TLRCKPISGHNSLFWYRETMMRGLELLIYFNNNVPIDDS |
| | | GMPEDRFSAKMPNASFSTLKIQPSEPRDSAVYFCASSPG |
| | | STDTQYFGPGTRLTVLEDLKNVFPPEVAVFEPSEAEISHT |
| | | QKATLVCLATGFYPDHVELSWWVNGKEVHSGVSTDPQ |
| | | PLKEQPALNDSRYCLSSRLRVSATFWQNPRNHFRCQVQ |
| | | FYGLSENDEWTQDRAKPVTQIVSAEAWGRADCGFTSES |
| | | YQQGVLSATILYEILLGKATLYAVLVSALVLMAMVKR |
| | | KDSRG |
| 304 | R39P1C12 alpha | MKTFAGFSFLFLWLQLDCMSRGEDVEQSLFLSVREGDS |
| | chain | SVINCTYTDSSSTYLYWYKQEPGAGLQLLTYIFSNMDM |
| | | KQDQRLTVLLNKKDKHLSLRIADTQTGDSAIYFCAEIDN |
| | | QGGKLIFGQGTELSVKPNIQNPDPAVYQLRDSKSSDKSV |
| | | CLFTDFDSQTNVSQSKDSDVYITDKTVLDMRSMDFKSN |
| | | SAVAWSNKSDFACANAFNNSIIPEDTFFPSPESSCDVKL |
| | | VEKSFETDTNLNFQNLSVIGFRILLLKVAGFNLLMTLRL |
| | | WSS |

The constructs in Table 2 may be assemblages of the individual components described in Table 3. The inventors found that the combination, order, and inclusion of transcription enhancers from Table 3 as described in Table 2 provided unexpected improvements in transfection efficiency, expression levels, and induction of cytotoxic T-cell activities, e.g., IL-12 secretion, IFN-γ secretion, TNF-α secretion, granzyme A secretion, MIP-1a secretion, IP-10 secretion, granzyme B secretion, and combinations thereof.

### Tumor Associated Antigens (TAA)

In the MHC class I dependent immune reaction, peptides not only have to be able to bind to certain MHC class I molecules expressed by tumor cells, they subsequently also have to be recognized by T cells bearing specific T cell receptors (TCR).

For proteins to be recognized by T-lymphocytes as tumor-specific or -associated antigens, and to be used in a therapy, particular prerequisites must be fulfilled. The antigen should be expressed mainly by tumor cells and not, or in comparably small amounts, by normal healthy tissues. In a preferred embodiment, the peptide should be over-presented by tumor cells as compared to normal healthy tissues. It is furthermore desirable that the respective antigen is not only present in a type of tumor, but also in high concentrations (e.g., copy numbers of the respective peptide per cell). Tumor-specific and tumor-associated antigens are often derived from proteins directly involved in transformation of a normal cell to a tumor cell due to their function, e.g., in cell cycle control or suppression of apoptosis. Additionally, downstream targets of the proteins directly causative for a transformation may be up-regulated and thus may be indirectly tumor-associated. Such indirect tumor-associated antigens may also be targets of a vaccination approach. Singh-Jasuja et al. Cancer Immunol. Immunother. 53 (2004): 187-195. Epitopes are present in the amino acid sequence of the antigen, making the peptide an “immunogenic peptide”, and being derived from a tumor associated antigen, leads to a T-cell-response, both in vitro and in vivo.

Any peptide able to bind an MHC molecule may function as a T-cell epitope. For the induction of a T-cell-response, the TAA must be presented a T cell having a corresponding TCR and the host must not have immunological tolerance for this particular epitope. Exemplary Tumor Associated Antigens (TAA) that may be used with the CD8 polypeptides described herein are disclosed herein.

TABLE 4
TAA Peptide sequences

| SEQ | Amino Acid |
|---|---|
| ID NO: | Sequence |
| 98 | YLYDSETKNA |
| 99 | HLMDQPLSV |
| 100 | GLLKKINSV |
| 101 | FLVDGSSAL |
| 102 | FLFDGSANLV |
| 103 | FLYKIIDEL |
| 104 | FILDSAETTTL |
| 105 | SVDVSPPKV |
| 106 | VADKIHSV |
| 107 | IVDDLTINL |
| 108 | GLLEELVTV |
| 109 | TLDGAAVNQV |
| 110 | SVLEKEIYSI |
| 111 | LLDPKTIFL |
| 112 | YTFSGDVQL |
| 113 | YLMDDFSSL |
| 114 | KVWSDVTPL |
| 115 | LLWGHPRVALA |
| 116 | KIWEELSVLEV |
| 117 | LLIPFTIFM |
| 118 | FLIENLLAA |
| 119 | LLWGHPRVALA |
| 120 | FLLEREQLL |
| 121 | SLAETIFIV |
| 122 | TLLEGISRA |
| 123 | KIQEILTQV |
| 124 | VIFEGEPMYL |
| 125 | SLFESLEYL |
| 126 | SLLNQPKAV |
| 127 | GLAEFQENV |
| 128 | KLLAVIHEL |
| 129 | TLHDQVHLL |
| 130 | TLYNPERTITV |
| 131 | KLQEKIQEL |
| 132 | SVLEKEIYSI |
| 133 | RVIDDSLVVGV |
| 134 | VLFGELPAL |
| 135 | GLVDIMVHL |
| 136 | FLNAIETAL |
| 137 | ALLQALMEL |
| 138 | ALSSSQAEV |
| 139 | SLITGQDLLSV |
| 140 | QLIEKNWLL |
| 141 | LLDPKTIFL |
| 142 | RLHDENILL |
| 143 | YTFSGDVQL |
| 144 | GLPSATTTV |
| 145 | GLLPSAESIKL |
| 146 | KTASINQNV |
| 147 | SLLQHLIGL |
| 148 | YLMDDFSSL |
| 149 | LMYPYIYHV |
| 150 | KVWSDVTPL |
| 151 | LLWGHPRVALA |
| 152 | VLDGKVAVV |
| 153 | GLLGKVTSV |
| 154 | KMISAIPTL |
| 155 | GLLETTGLLAT |
| 156 | TLNTLDINL |
| 157 | VIIKGLEEI |
| 158 | YLEDGFAYV |
| 159 | KIWEELSVLEV |
| 160 | LLIPFTIFM |
| 161 | ISLDEVAVSL |
| 162 | KISDFGLATV |
| 163 | KLIGNIHGNEV |
| 164 | ILLSVLHQL |
| 165 | LDSEALLTL |
| 166 | VLQENSSDYQSNL |
| 167 | HLLGEGAFAQV |
| 168 | SLVENIHVL |
| 169 | YTFSGDVQL |
| 170 | SLSEKSPEV |
| 171 | AMFPDTIPRV |
| 172 | FLIENLLAA |
| 173 | FTAEFLEKV |
| 174 | ALYGNVQQV |
| 175 | LFQSRIAGV |
| 176 | ILAEEPIYIRV |
| 177 | FLLEREQLL |
| 178 | LLLPLELSLA |
| 179 | SLAETIFIV |
| 180 | AILNVDEKNQV |
| 181 | RLFEEVLGV |
| 182 | YLDEVAFML |
| 183 | KLIDEDEPLFL |
| 184 | KLFEKSTGL |
| 185 | SLLEVNEASSV |
| 186 | GVYDGREHTV |
| 187 | GLYPVTLVGV |
| 188 | ALLSSVAEA |
| 189 | TLLEGISRA |
| 190 | SLIEESEEL |
| 191 | ALYVQAPTV |
| 192 | KLIYKDLVSV |
| 193 | ILQDGQFLV |
| 194 | SLLDYEVSI |
| 195 | LLGDSSFFL |
| 196 | VIFEGEPMYL |
| 197 | ALSYILPYL |
| 198 | FLFVDPELV |
| 199 | SEWGSPHAAVP |
| 200 | ALSELERVL |
| 201 | SLFESLEYL |
| 202 | KVLEYVIKV |
| 203 | VLLNEILEQV |
| 204 | SLLNQPKAV |
| 205 | KMSELQTYV |
| 206 | ALLEQTGDMSL |
| 207 | VIIKGLEEITV |
| 208 | KQFEGTVEI |
| 209 | KLQEEIPVL |
| 210 | GLAEFQENV |
| 211 | NVAEIVIHI |
| 212 | ALAGIVTNV |
| 213 | NLLIDDKGTIKL |
| 214 | VLMQDSRLYL |
| 215 | KVLEHVVRV |
| 216 | LLWGNLPEI |
| 217 | SLMEKNQSL |
| 218 | KLLAVIHEL |
| 219 | ALGDKFLLRV |
| 220 | FLMKNSDLYGA |
| 221 | KLIDHQGLYL |
| 222 | GPGIFPPPPPQP |
| 223 | ALNESLVEC |
| 224 | GLAALAVHL |
| 225 | LLLEAVWHL |
| 226 | SIIEYLPTL |
| 227 | TLHDQVHLL |
| 228 | SLLMWITQC |
| 229 | FLLDKPQDLSI |
| 230 | YLLDMPLWYL |
| 231 | GLLDCPIFL |
| 232 | VLIEYNFSI |
| 233 | TLYNPERTITV |
| 234 | AVPPPPSSV |
| 235 | KLQEELNKV |
| 236 | KLMDPGSLPPL |
| 237 | ALIVSLPYL |
| 238 | FLLDGSANV |
| 239 | ALDPSGNQLI |
| 240 | ILIKHLVKV |
| 241 | VLLDTILQL |
| 242 | HLIAEIHTA |
| 243 | SMNGGVFAV |
| 244 | MLAEKLLQA |
| 245 | YMLDIFHEV |
| 246 | ALWLPTDSATV |
| 247 | GLASRILDA |
| 248 | ALSVLRLAL |
| 249 | SYVKVLHHL |
| 250 | VYLPKIPSW |
| 251 | NYEDHFPLL |
| 252 | VYIAELEKI |
| 253 | VHFEDTGKTLLF |
| 254 | VLSPFILTL |
| 255 | HLLEGSVGV |

### Example 2

### CD8α Molecules

CD8α homodimer (CD8αα) may be composed of two a subunits held together by two disulfide bonds at the stalk regions. FIG. 1 shows a CD8α polypeptide, e.g., SEQ ID NO: 258 (CD8α1), that includes five domains: (1) one signal peptide (from −21 to −1), e.g., SEQ ID NO: 6, (2) one Ig-like domain-1 (from 1 to 115), e.g., SEQ ID NO: 1, (3) one stalk region (from 116 to 160), e.g., SEQ ID NO: 260, (4) one transmembrane (TM) domain (from 161-188), e.g., SEQ ID NO: 3, and (5) one cytoplasmic tail (Cyto) comprising a lck-binding motif (from 189 to 214), e.g., SEQ ID NO: 4. Another example of CD8α subunit, e.g., CD8α2 (SEQ ID NO: 259), differs from CD8α1 at position 112, at which CD8α2 contains a cysteine (C), whereas CD8α1 contains a tyrosine (Y).

### Modified CD8 Polypeptides

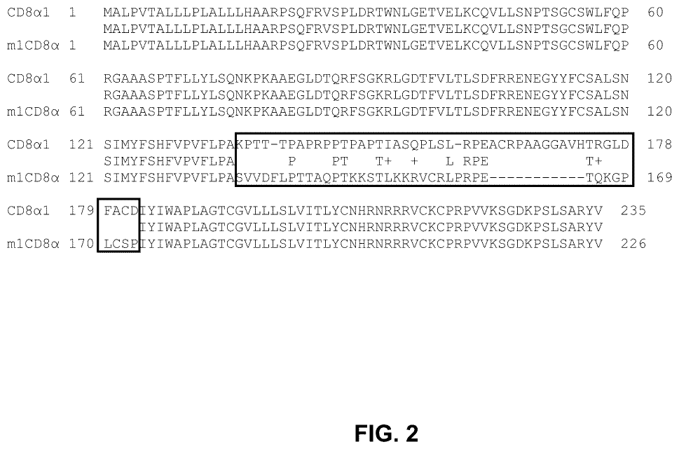

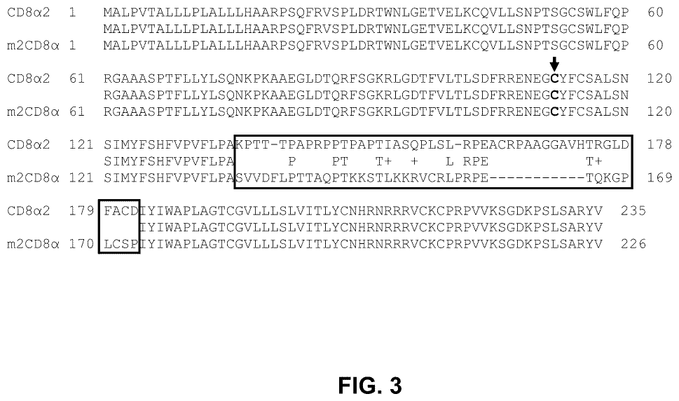

Different from CD8α polypeptide, e.g., CD8α1 (SEQ ID NO: 258) and CD8α2 (SEQ ID NO: 259), a modified CD8α polypeptide, e.g., m1CD8α (SEQ ID NO: 7) and m2CD8α (SEQ ID NO: 262), may contain additional regions, such as sequence stretches from a CD8β polypeptide. In an embodiment, SEQ ID NO: 2 or variants thereof are used with a CD8α polypeptide. In other embodiments, a portion of a CD8α polypeptide, e.g., SEQ ID NO: 260, is removed or not included in modified CD8 polypeptides described herein. FIG. 2 shows a sequence alignment between CD8α1 (SEQ ID NO: 258) and m1CD8α (SEQ ID NO: 7). FIG. 3 shows a sequence alignment between CD8α2 (SEQ ID NO: 259) and m2CD8α (SEQ ID NO: 262), in which the cysteine substitution is indicated by an arrow. The stalk regions are shown within the boxes.

Modified CD8 expressing cells showed improved functionality in terms of cytotoxicity and cytokine response as compared to original CD8 expressing T cells transduced with the TCR.

### Example 3

### Lentiviral Viral Vectors

The lentiviral vectors used herein contain several elements that enhance vector function, including a central polypurine tract (cPPT) for improved replication and nuclear import, a promoter from the murine stem cell virus (MSCV) (SEQ ID NO: 263), which lessens vector silencing in some cell types, a woodchuck hepatitis virus posttranscriptional responsive element (WPRE) (SEQ ID NO: 264) for improved transcriptional termination, and the backbone was a deleted 3′-LTR self-inactivating (SIN) vector design that improves safety, sustained gene expression and anti-silencing properties. Yang et al. Gene Therapy (2008) 15, 1411-1423.

In an embodiment, vectors, constructs, or sequences described herein comprise mutated forms of WPRE. In an embodiment, sequences or vectors described herein comprise mutations in WPRE version 1, e.g., WPREmut1 (SEQ ID NO: 256), or WPRE version 2, e.g., WPREmut2 (SEQ ID NO: 257). Construct #9 and Construct #9b represent two LV production batches with the same construct containing SEQ ID NO: 257 as WPREmut2, with the difference between Construct #9 and Construct #9b being the titer consistent with Table 4. In an embodiment, WPRE mutants comprise at most one mutation, at most two mutations, at most three mutations, at least four mutations, or at most five mutations. In an embodiment, vectors, constructs, or sequences described herein do not comprise WPRE. In an aspect, WPRE sequences described in U.S. 2021/0285011, the content of which is incorporated by reference in its entirety, may be used together with vectors, sequences, or constructs described herein.

In an embodiment, vectors, constructs, or sequences described herein do not include an X protein promoter. The WPRE mutants described herein do not express an X protein. WPRE promotes accumulation of mRNA, theorized to promote export of mRNA from nucleosome to cytoplasm to promote translation of the transgene mRNA.

To obtain optimal co-expression levels of TCRαβ, mCD8α (e.g., m1CD8α (SEQ ID NO: 7) and m2CD8α (SEQ ID NO: 262)) and CD8β (e.g., any one of CD8β1-7 (SEQ ID NO: 8-14)) in the transduced CD4+ T cells, CD8+ T cells, and/or γδ T cells, lentiviral vectors with various designs were generated. T cells may be transduced with two separate lentiviral vectors (2-in-1), e.g., one expressing TCRα and TCRβ and the other expressing mCD8α and CD8β, for co-expression of TCRαβ and CD8αβ heterodimer, or one expressing TCRα and TCRβ and the other expressing mCD8α for co-expression of TCRαβ and mCD8α homodimer. Alternatively, T cells may be transduced with a single lentiviral vector (4-in-1) co-expressing TCRα, TCRβ, mCD8α, and CD8β for co-expression of TCRαβ and CD8αβ heterodimer. In the 4-in-1 vector, the nucleotides encoding TCRα chain, TCRβ chain, mCD8α chain, and CD8β chain may be shuffled in various orders, e.g., from 5′ to 3′ direction, TCRα-TCRβ-mCD8α-CD8P, TCRα-TCRβ-CD8β-mCD8α, TCRβ-TCRα-mCD8α-CD8P, TCRβ-TCRα-CD8β-mCD8α, mCD8α-CD8β-TCRα-TCRβ, mCD8α-CD8β-TCRβ-TCRα, CD8β-mCD8α-TCRα-TCRβ, and CD8β-mCD8α-TCRβ-TCRα. Various 4-in-1 vectors, thus generated, may be used to transduce CD4+ T cells, CD8+ T cells, and/or γδ T cells, followed by measuring TCRαβ/mCD8α/CD8β co-expression levels of the transduced cells using techniques known in the art, e.g., flow cytometry. Similarly, T cells may be transduced with a single lentiviral vector (3-in-1) co-expressing TCRα, TCRβ, and mCD8α (e.g., m1CD8α and m2CD8α) for co-expression of TCRαβ and mCD8α homodimer. In the 3-in-1 vector, the nucleotides encoding TCRα chain, TCRβ chain, mCD8α chain may be shuffled in various orders, e.g., TCRα-TCRβ-mCD8α, TCRβ-TCRα-mCD8α, mCD8α-TCRα-TCRβ, and mCD8α-TCRβ-TCRα. Various 3-in-1 vectors, thus generated, may be used to transduce CD4+ T cells, CD8+ T cells, and/or γδ T cells, followed by measuring TCRαp/mCD8α co-expression levels of the transduced cells using techniques known in the art.

To generate lentiviral vectors co-expressing TCRαβ and mCD8α and/or CD8P, a nucleotide encoding furin-linker (GSG or SGSG (SEQ ID NO: 266))-2A peptide may be positioned between TCRα chain and TCRβ chain, between mCD8α chain and CD8β chain, and between a TCR chain and a CD8 chain to enable highly efficient gene expression. The 2A peptide may be selected from P2A (SEQ ID NO: 93), T2A (SEQ ID NO: 94), E2A (SEQ ID NO: 95), or F2A (SEQ ID NO: 96).

Lentiviral viral vectors may also contain post-transcriptional regulatory element (PRE), such as WPRE (SEQ ID NO: 264), WPREmut1 (SEQ ID NO: 256), or WPREmut2 (SEQ ID NO: 257), to enhance the expression of the transgene by increasing both nuclear and cytoplasmic mRNA levels. One or more regulatory elements including mouse RNA transport element (RTE), the constitutive transport element (CTE) of the simian retrovirus type 1 (SRV-1), and the 5′ untranslated region of the human heat shock protein 70 (Hsp70 5′UTR) may also be used and/or in combination with WPRE to increase transgene expression. The WPREmut1 and WPREmut2 do not express an X protein, but still act to enhance translation of the transgene mRNA.

Lentiviral vectors may be pseudotyped with RD114TR (for example, SEQ ID NO: 97), which is a chimeric glycoprotein comprising an extracellular and transmembrane domain of feline endogenous virus (RD114) fused to cytoplasmic tail (TR) of murine leukemia virus. Other viral envelop proteins, such as VSV-G env, MLV 4070A env, RD114 env, chimeric envelope protein RD114pro, baculovirus GP64 env, or GALV env, or derivatives thereof, may also be used. RD114TR variants comprising at least 85%, at least 90%, at least 95%, at least 98%, at least 99%, or 100% to SEQ ID NO: 97 also provided for.

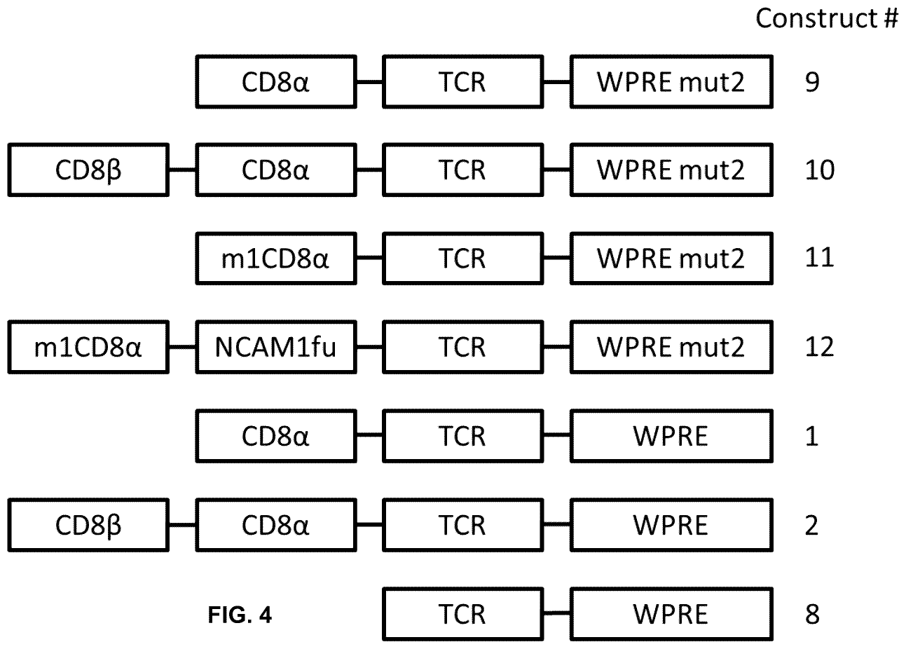

For example, FIG. 4 shows exemplary vectors, which include two 4-in-1 vectors, e.g., Constructs #10 and #2, co-expressing TCR (TCRα chain and TCRβ chain), CD8α, and CD83; three 3-in-1 vectors expressing TCR and CD8α, e.g., Constructs #1 and #9, two 3-in-1 vectors expressing TCR and m1CD8α (SEQ ID NO: 7), e.g., Constructs #11 and #12, and Construct #8 expressing TCR only. To improve transcriptional termination, wild type WPRE (WPRE) (SEQ ID NO: 264) is included in Constructs #1, #2, and #8; WPREmut (SEQ ID NO: 257) is included in Constructs #9, #10, #11, and #12.

Further exemplary constructs (Constructs #13-#19 and #21-#26) are described in Table 2 above. In particular, Constructs #13, #14, and #16 are 4-in-1 constructs co-expressing TCR, CD8α, and CD8β3 with various combinations of signal peptides (SEQ ID NO: 6 [WT CD8α signal peptide]; SEQ ID NO: 293 [WT CD8β signal peptide]; and SEQ ID NO: 294 [S19 signal peptide]) and differing element order. Constructs #15 and #17 are 4-in-1 constructs co-expressing TCR, CD8α, and CD8β5. Construct #15 comprises the WT CD8α signal peptide (SEQ ID NO: 6) and WT CD8β signal peptide (SEQ ID NO: 293), whereas Construct #17 comprises the S19 signal peptide (SEQ ID NO: 294) at the N-terminal end of both CD8α and CD8β5. Construct #21 is a 4-in-1 constructs co-expressing TCR, CD8α, and CD8β2 comprising WT CD8α signal peptide (SEQ ID NO: 6) and WT CD8β signal peptide (SEQ ID NO: 293). Construct #18 is a variant of Construct #10 in which the WT signal peptides for CD8α and CD8β1 (SEQ ID NOs: 6 and 293, respectively) were replaced with S19 signal peptide (SEQ ID NO: 294). Construct #19 is a variant of Construct #11 in which the WT CD8α signal peptide (SEQ ID NO: 6) was replaced with the S19 signal peptide (SEQ ID NO: 294). Construct #22 is a variant of Construct #11 in which the CD4 transmembrane and intracellular domains are fused to the C-terminus of the CD8β stalk sequence in place of the CD8α transmembrane and intracellular domains. Construct #25 is a variant of Construct #22 in which the CD8β stalk sequence (SEQ ID NO: 2) is replaced with the CD8α stalk sequence (SEQ ID NO: 260).

### Example 4

### Vector Screening (Constructs #1, #2, #8, #9, #10, #11, and #12)

### Viral Titers

FIG. 5A shows viral titer of Constructs #1, #2, #8, #9, #10, #11, and #12. Table 5 shows viral titers and lentiviral P24 ELISA data for Constructs #9, #10, #11, and #12.

TABLE 5

| Constructs | | Lentiviral |
|---|---|---|
| # | Titer | P24 |
| 9 | 5.40 × 10⁹ | 6556 |
| 9b | 9.80 × 10⁹ | 16196 |
| 10 | 6.40 × 10⁹ | 9525 |
| 11 | 1.30 × 10¹⁰ | 16797 |
| 12 | 1.20 × 10¹⁰ | 17996 |

For construct 12, NCAMfu refers to NCAMFusion protein expressing modified CD8α extracellular and Neural cell adhesion molecule 1 (CD56) intracellular domain.

For Table 5, the WPREmut2 portion refers to SEQ ID NO: 257.

### T Cell Manufacturing

### Activation

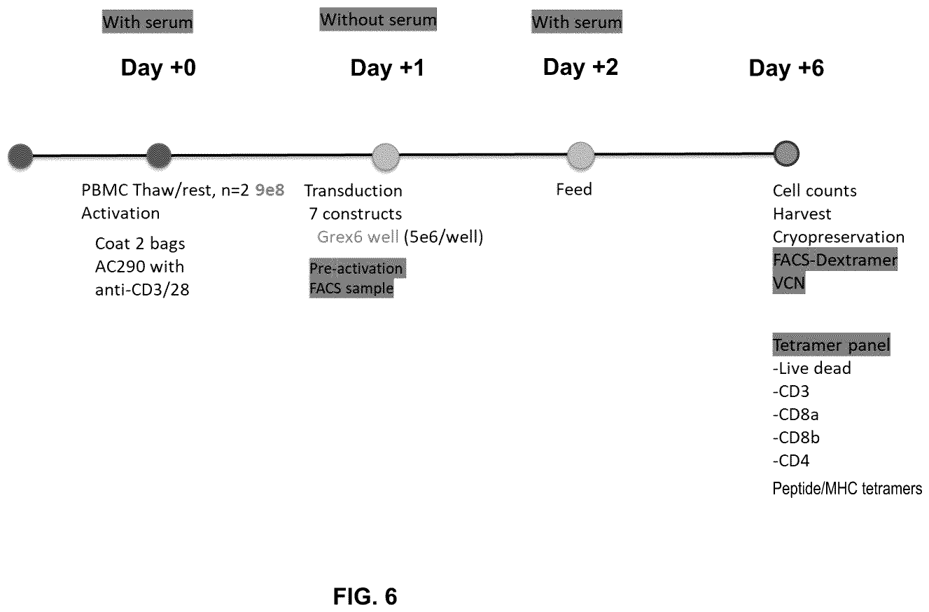

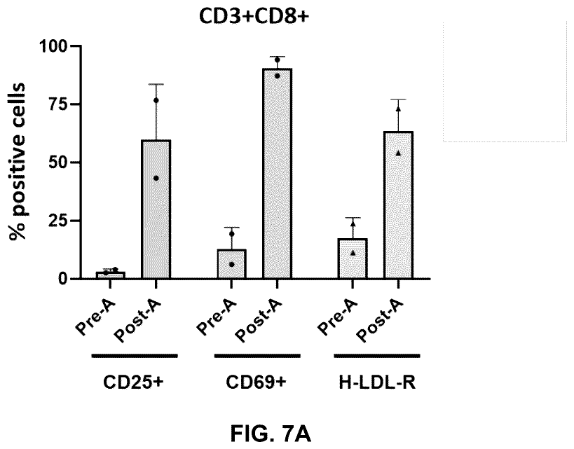

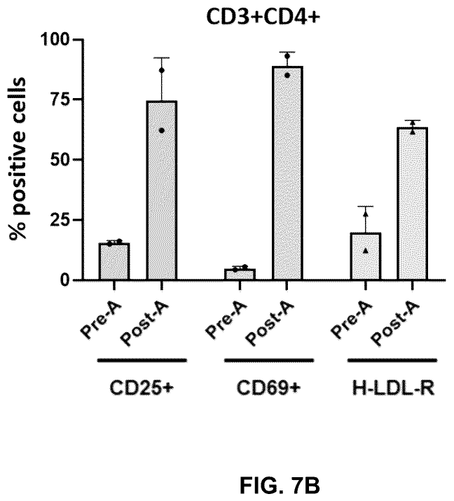

FIG. 6 shows that, on Day +0, PBMCs (about 9×10⁸ cells) obtained from two donors (Donor #1 and Donor #2) were thawed and rested. Cells were activated in bags (AC290) coated with anti-CD3 and anti-CD28 antibodies in the presence of serum. Activation markers, e.g., CD25, CD69, and human low density lipoprotein receptor (H-LDL-R) are in CD8+ and CD4+ cells, were subsequently measured. FIG. 7A shows that % CD3+CD8+CD25+ cells, % CD3+CD8+CD69+ cells, and % CD3+CD8+H-LDL-R+ cells increase after activation (Post-A) as compared with that before activation (Pre-A). Similarly, FIG. 7B shows that % CD3+CD4+CD25+ cells, % CD3+CD4+CD69+ cells, and % CD3+CD4+H-LDL-R+ cells increase after activation (Post-A) as compared with that before activation (Pre-A). These results support the activation of PBMCs.

### Transduction

FIG. 6 shows that, on Day +1, activated PBMCs were transduced with viral vectors, e.g., Constructs #1, #2, #8, #9, #10, #11, and #12, in G-Rex® 6 well plates at about 5×10⁶ cells/well in the absence of serum. The amounts of virus used for transduction are shown in Table 6.

TABLE 6

| Constructs | Virus Volume/1 × 10⁶ cells |
|---|---|
| #9, #10, #11, #12 | 1.25 μl, 2.5 μl, 5 μl |
| #1 | 1.25 μl |
| #2 | 5 μl |
| #8 (TCR) | 2.5 μl |

### Expansion

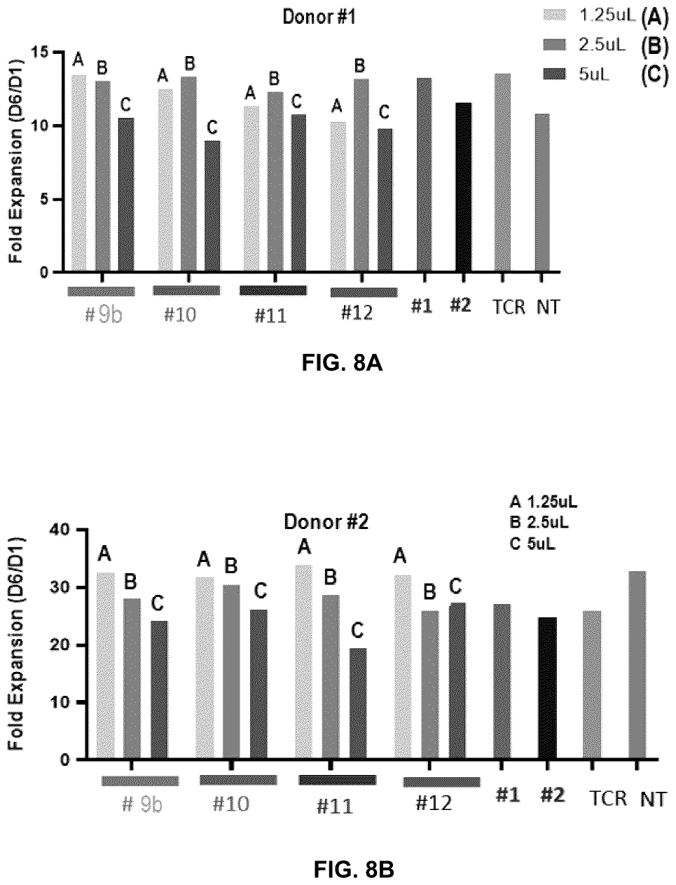

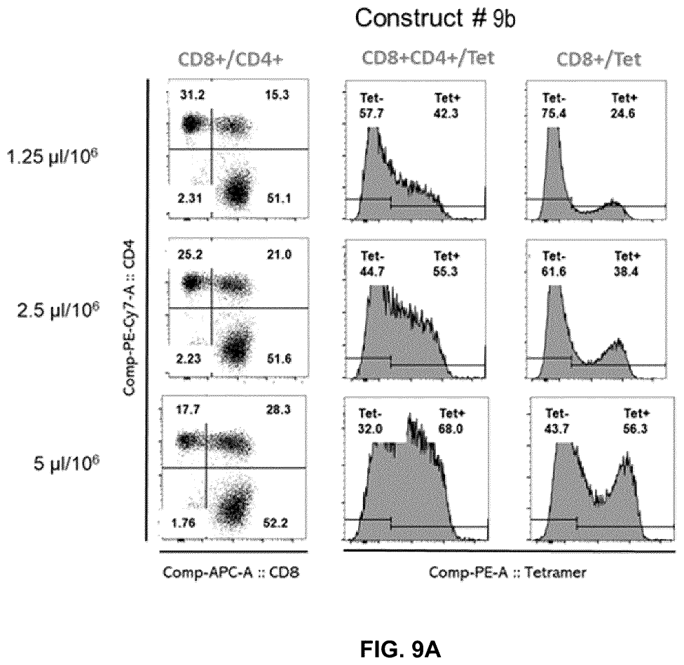

FIG. 6 shows that, on Day +2, transduced PBMCs were expanded in the presence of serum. On Day +6, cells were harvested for subsequent analysis, e.g., FACS-Dextramer and vector copy number (VCN) and were cryopreserved. FIGS. 8A and 8B show fold expansion on Day +6 of transduced T cell products obtained from Donor #1 and donor #2, respectively. Viabilities of cells is greater than 90% on Day +6.

### Characterization of T Cell Products

Cell counts, FACS-dextramers, and vector copy numbers (VCN) were determined. Tetramer panels may comprise live/dead cells, CD3, CD8α, CD8β, CD4, and peptide/MHC tetramers, e.g., PRAME-004 (SLLQHLIGL) (SEQ ID NO: 147)/MHC tetramers. FACS analysis was gated on live singlets, followed by CD3+, followed by CD4+CD8+, followed by CD4+CD8+Tetramer(Tet)+ and CD8+Tet+.

FIGS. 9A, 9B, 9C, and 9D show representative flow plots of cells obtained from Donor #1 indicating % CD8, CD4, and PRAME-004/MHC tetramer (Tet) of cells transduced with Construct #9b, #10, #11, or #12, respectively.

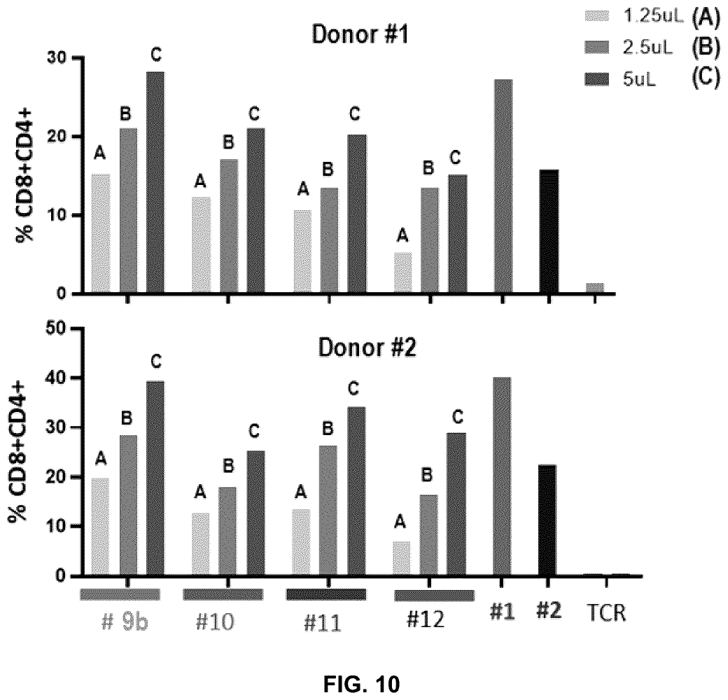

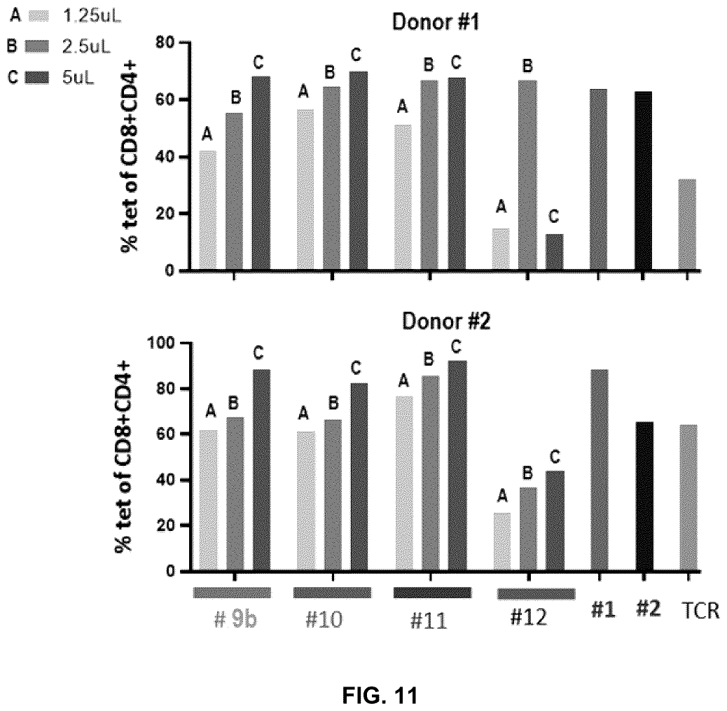

FIG. 10 shows % CD8+CD4+ cells from Donor #1 (upper panel) and Donor #2 (lower panel) transduced with Construct #1, #2, #8 (TCR), #9, #10, #11, or #12 at 1.25 μl, 2.5 μl, or 5 μl per 1×10⁶ cells. These results show that higher % CD8+CD4+ cells were obtained by transduction with vectors expressing CD8α and TCR with wild type WPRE (Construct #1) and WPREmut2 (Construct #9) than that transduced with Constructs #10, #11, or #12. Construct #8 (TCR only) serves as negative control. FIG. 11 shows % Tet of CD8+CD4+ cells from Donor #1 (upper panel) and Donor #2 (lower panel) transduced with Constructs #1, #2, #8 (TCR), #9, #10, #11, and #12 at 1.25 μl, 2.5 μl, or 5 μl per 1×10⁶ cells. These results show that % Tet of CD8+CD4+ cells appear comparable among cells transduced with Constructs #9, #10, and #11, and seems greater than that transduced with Construct #12. FACS analysis was gated on live singlets, followed by CD3+, followed by CD4+CD8+, and followed by CD4+CD8+Tet+.

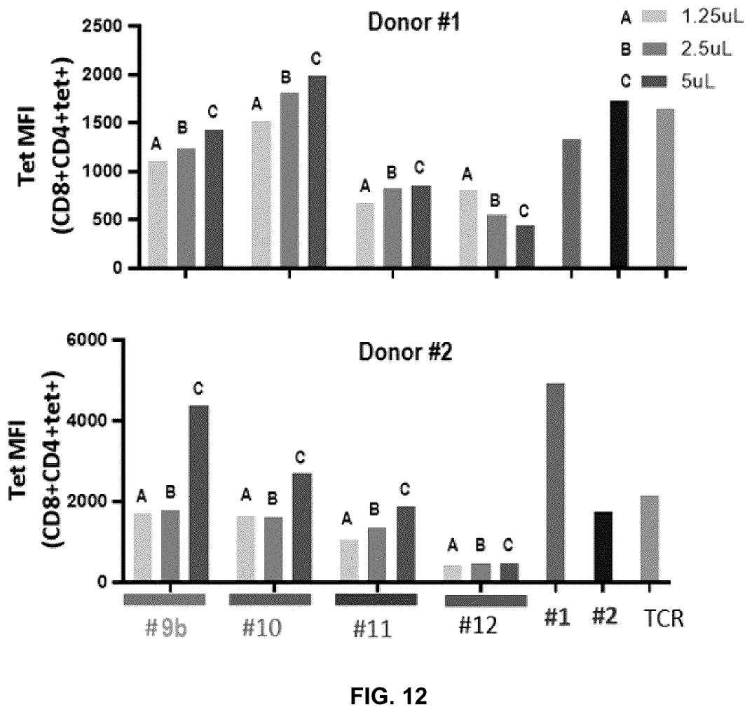

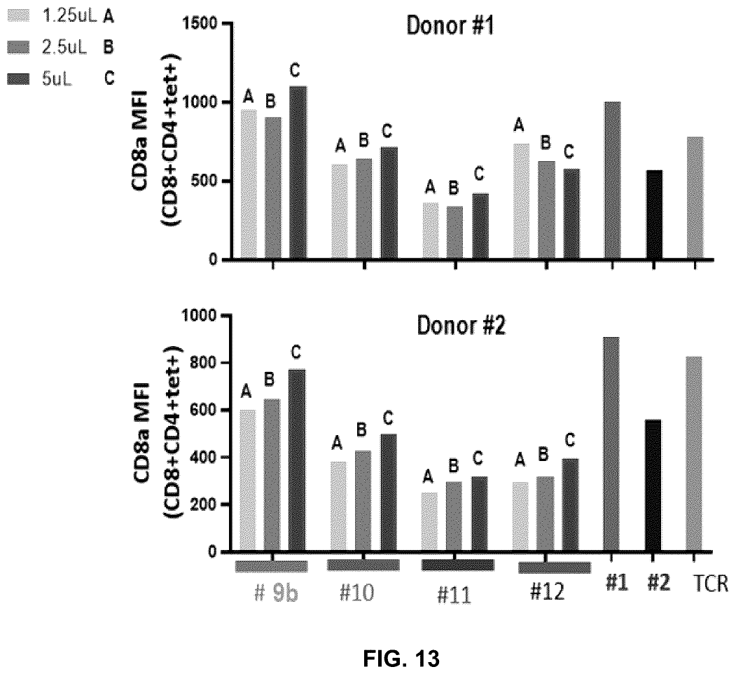

FIG. 12 shows Tet MFI of CD8+CD4+Tet+ cells from Donor #1 (upper panel) and Donor #2 (lower panel) transduced with Construct #1, #2, #8 (TCR), #9, #10, #11, or #12 at 1.25 μl, 2.5 μl, or 5 μl per 1×10⁶ cells. These results show that tetramer MFI on CD4+CD8+Tet+ varies among donors. FIG. 13 shows CD8α MFI of CD8+CD4+Tet+ cells from Donor #1 (upper panel) and Donor #2 (lower panel) transduced with Construct #1, #2, #8 (TCR), #9, #10, #11, or #12 at 1.25 μl, 2.5 μl, or 5 μl per 1×10⁶ cells. These results show higher CD8α MFI in cells transduced with vectors expressing CD8α and TCR with wild type WPRE (Construct #1) and WPREmut2 (Construct #9) than that transduced with the other constructs. Transduction volume of 5 μl/10⁶ appears to yield better results than 1.25 μl/10⁶ and 2.5 μl/10⁶. FACS analysis was gated on live singlets, followed by CD3+, followed by CD4+CD8+, followed by CD4+CD8+Tet+, and followed by Tet MFI/CD8α MFI.

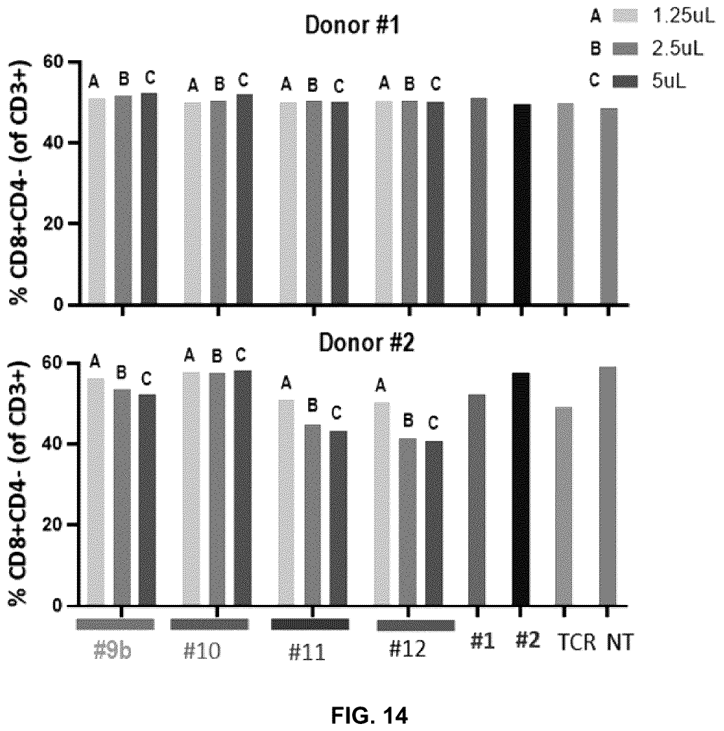

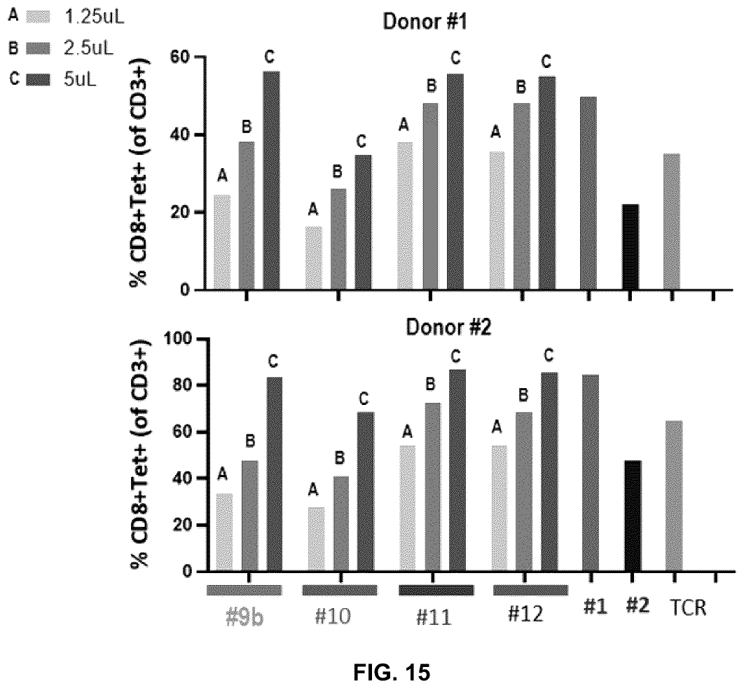

FIG. 14 shows CD8 frequencies (% CD8+CD4− of CD3+) in cells from Donor #1 (upper panel) and Donor #2 (lower panel) transduced with Construct #1, #2, #8 (TCR), #9, #10, #11, or #12 at 1.25 μl, 2.5 μl, or 5 μl per 1×10⁶ cells. These results show no difference in the CD8 frequencies among the constructs. Non-transduction (NT) serves as negative control. FIG. 15 shows % CD8+Tet+(of CD3+) cells from Donor #1 (upper panel) and Donor #2 (lower panel) transduced with Construct #1, #2, #8 (TCR), #9, #10, #11, or #12 at 1.25 μl, 2.5 μl, or 5 μl per 1×10⁶ cells. These results show higher frequencies of CD8+Tet+(of CD3+) in cells transduced with Constructs #9, #11, and #12 than that transduced with Construct #10. FACS analysis was gated on live singlets, followed by CD3+, followed by CD8+CD4−, and followed by CD8+Tet+.

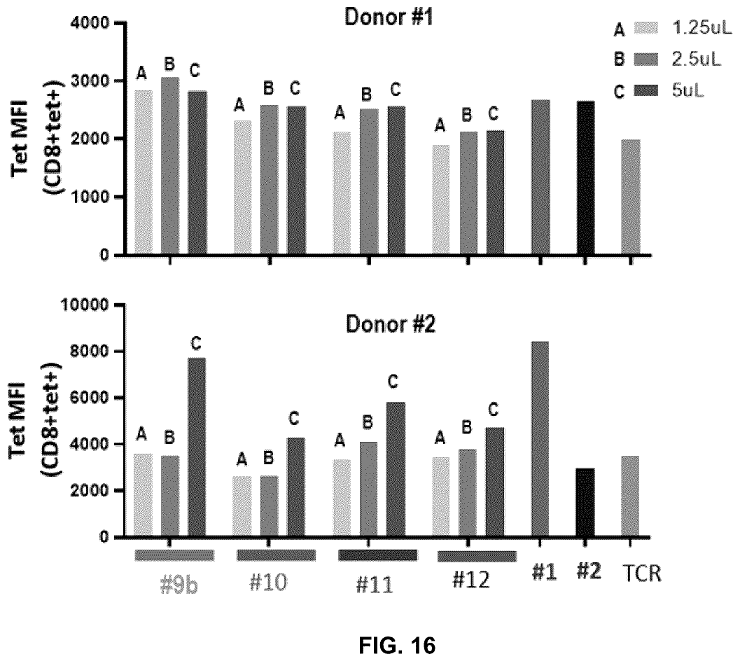

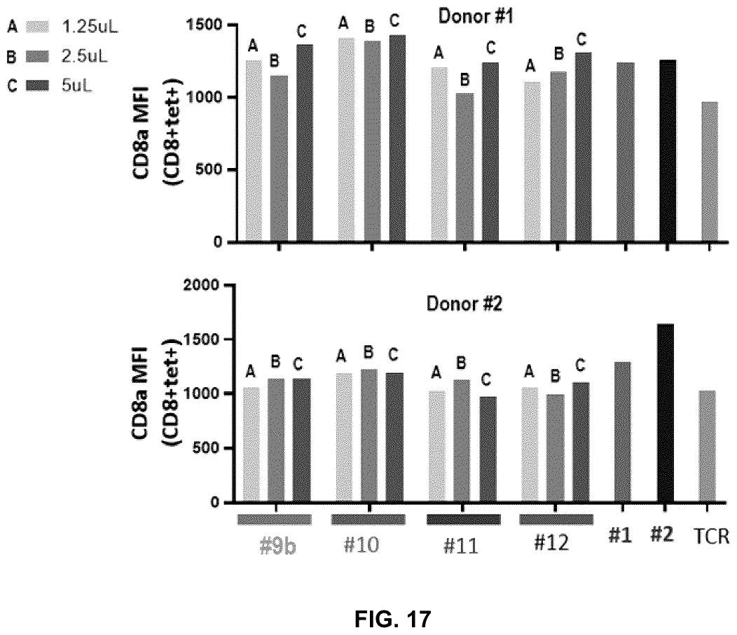

FIG. 16 shows Tet MFI of CD8+Tet+ cells from Donor #1 (upper panel) and Donor #2 (lower panel) transduced with Construct #1, #2, #8 (TCR), #9, #10, #11, or #12 at 1.25 μl, 2.5 μl, or 5 μl per 1×10⁶ cells. These results show tetramer MFI of CD8+tet+ cells varies among donors. FIG. 17 shows CD8α MFI of CD8+Tet+ cells from Donor #1 (upper panel) and Donor #2 (lower panel) transduced with Construct #1, #2, #8 (TCR), #9, #10, #11, or #12 at 1.25 μl, 2.5 μl, or 5 μl per 1×10⁶ cells. These results show that CD8α MFI of CD8+Tet+ are comparable among cells transduced with different constructs. FACS analysis was gated on live singlets, followed by CD3+, followed by CD4+CD8+, followed by CD4+CD8+Tet+, and followed by Tet MFI/CD8α MFI.

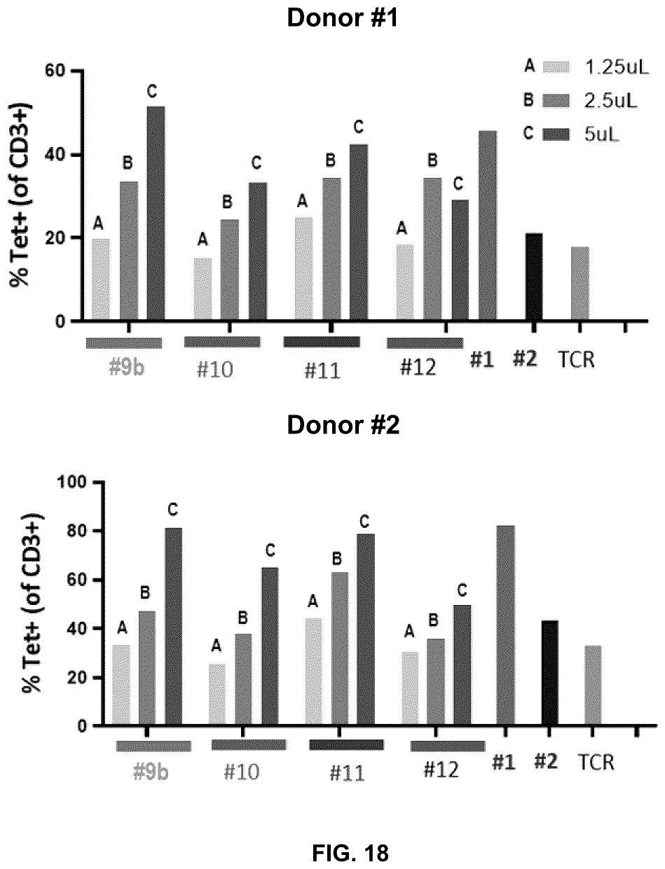

FIG. 18 shows % Tet+ of CD3+ cells from Donor #1 (upper panel) and Donor #2 (lower panel) transduced with Construct #1, #2, #8 (TCR), #9, #10, #11, or #12 at 1.25 μl, 2.5 μl, or 5 μl per 1×10⁶ cells. These results show higher frequencies of CD3+Tet+ in cells transduced with Construct #9 or #11 than that transduced with Construct #10 or #12. It appears more % Tet+CD3+ cells in cells transduced with Construct #10 (WPREmut2) than that transduced with Construct #2 (wild type WPRE) at 5 μl per 1×10⁶ cells. FACS analysis was gated on live singlets, followed by CD3+, followed by CD3+, and followed by Tet+.

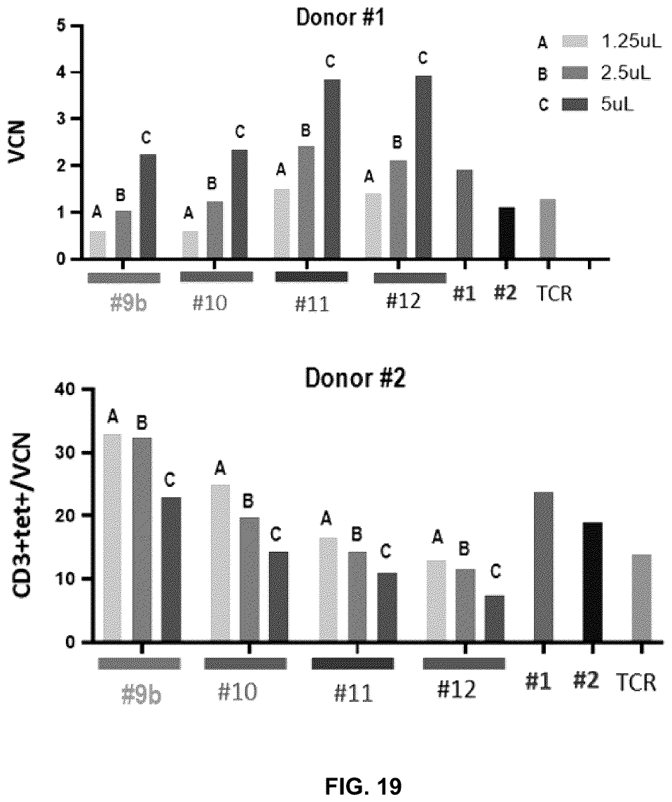

FIG. 19 (upper panel) shows vector copy number (VCN) of cells from Donor #1 transduced with Construct #1, #2, #8 (TCR), #9, #10, #11, or #12 at 1.25 μl, 2.5 μl, or 5 μl per 1×10⁶ cells. These results show higher VCN for cells transduced with Constructs #11 or #12 (may be due to higher titers) than that transduced with Construct #9 or #10. FIG. 19 (lower panel) shows CD3+Tet+/VCN of cells from Donor #1 transduced with Construct #1, #2, #8 (TCR), #9, #10, #11, or #12 at 1.25 μl, 2.5 μl, or 5 μl per 1×10⁶ cells. These results show higher CD3+Tet+/VCN in cells transduced with Construct #9 than that transduced with Construct #10, #11, or #12.

In sum, these results show (1) higher % CD8+CD4+ cells obtained by transducing cells with vectors expressing CD8α and TCR with wild type WPRE (Construct #1) and WPREmut2 (Construct #9) than that transduced with Construct #10, #11 or #12; (2) % CD8+CD4+Tet+ cells was comparable among cells transduced with different constructs; (3) dose dependent increase in % tetramer, e.g., 5 μl per 1×10⁶ cells showed better results than 1.25 μl and 2.5 μl per 1×10⁶ cells; (4) % CD8+ cells comparable among cells transduced with different constructs; (5) higher frequencies of CD8+Tet+ in cells transduced with Construct #9, #11, or #12 than that transduced with Construct #10; (6) higher frequencies of CD3+Tet+ in cells transduced with Construct #9 or #11 than that transduced with Construct #10 or #12; (7) higher VCN in cells transduced with Construct #11 or #12 than that transduced with Construct #9 or #10; and (8) higher CD3+tet+/VCN in cells transduced with Construct #9 than that transduced with Construct #10, #11, or #12.

T cell products transduced with viral vector expressing a transgenic TCR and modified CD8 co-receptor showed superior cytotoxicity and increased cytokine production against target positive cell lines.

### Example 5

### Tumor Death Assay

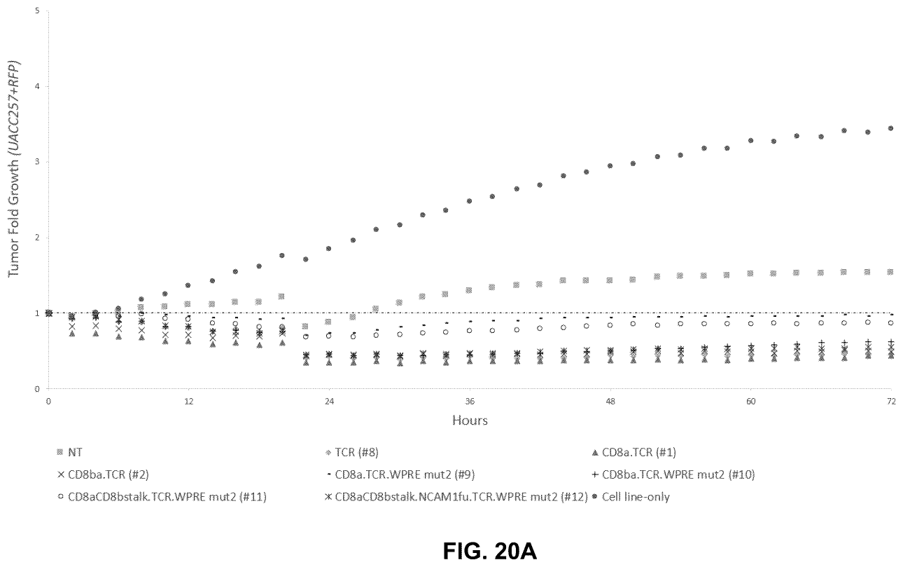

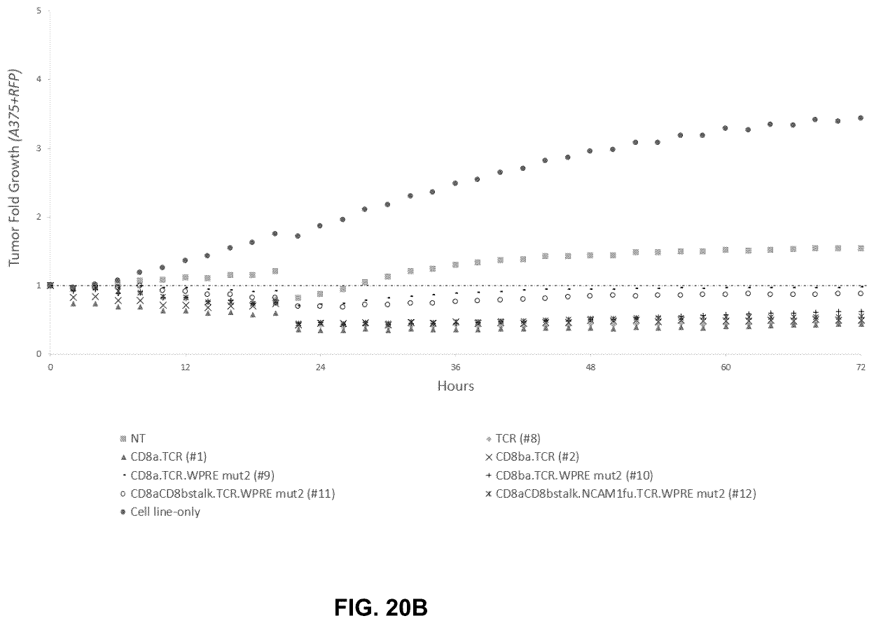

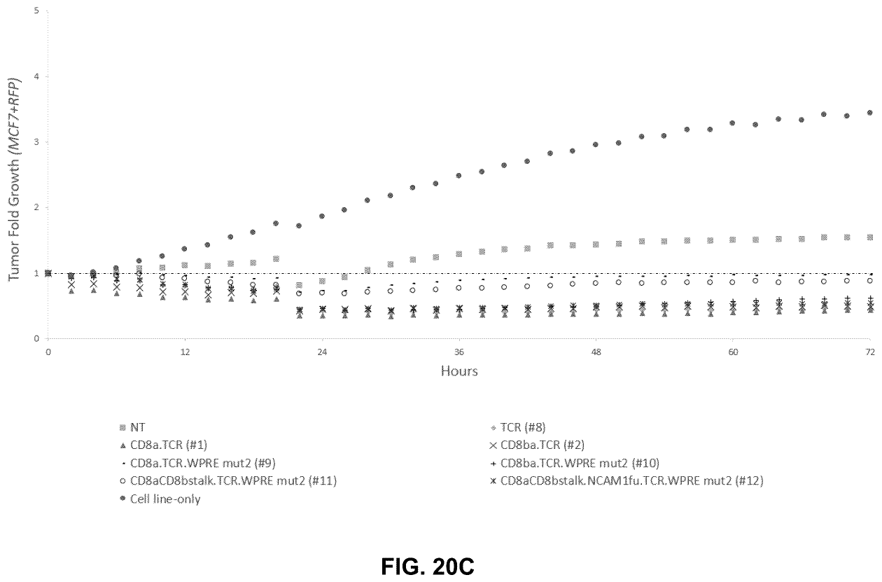

FIG. 20A-C depicts data showing that constructs (#10, #11, & #12) are comparable to TCR-only in mediating cytotoxicity against target positive cells lines expressing antigen at different levels (UACC257 at 1081 copies per cell and A375 at 50 copies per cell).

TABLE 7

| Tumor Cell Line | Antigen Positivity |
|---|---|
| UACC257 | High |
| A375 | Low |
| MCF7 | Negative |

Construct #9 loses tumor control over time against the low target antigen expressing A375 cell line.

### Example 6

### IFNγ Secretion Assay

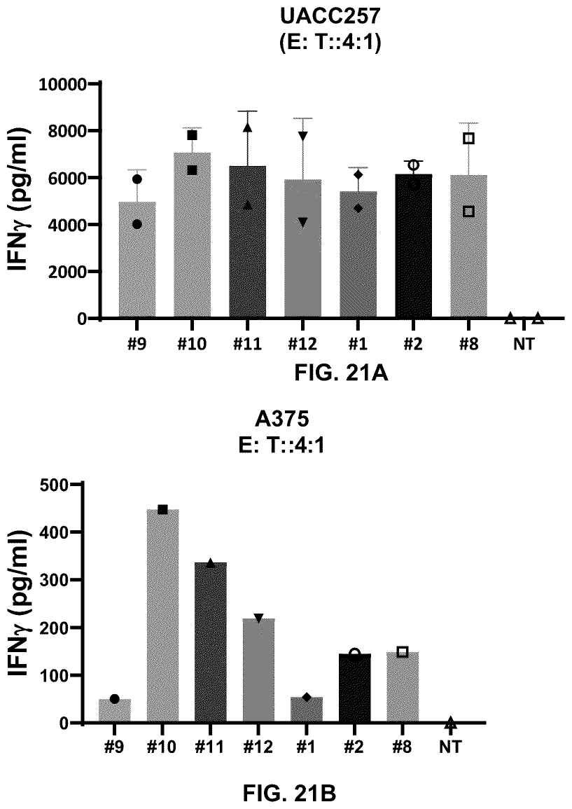

IFNγ secretion was measured in UACC257 and A375 cells lines. IFNγ secretion in response in UACC257 cell line was comparable among constructs. However, in the A375 cell line, Construct #10 showed higher IFNγ secretion than other constructs. IFNγ quantified in the supernatants from Incucyte plates. FIG. 21A-B.

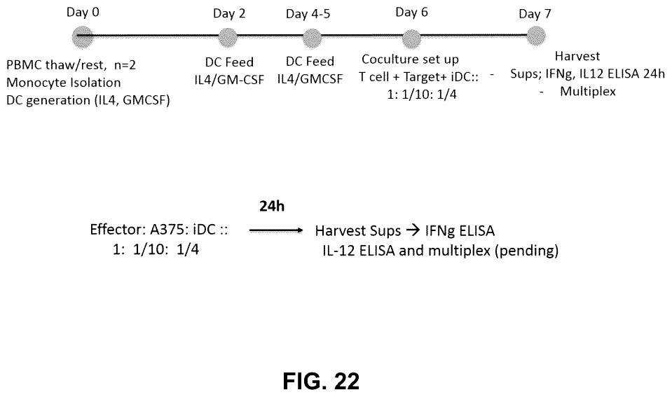

FIG. 22 depicts an exemplary experiment design to assess Dendritic Cell (DC) maturation and cytokine secretion by PBMC-derived T cell products in response to exposure to target positive tumor cell lines UACC257 and A375.

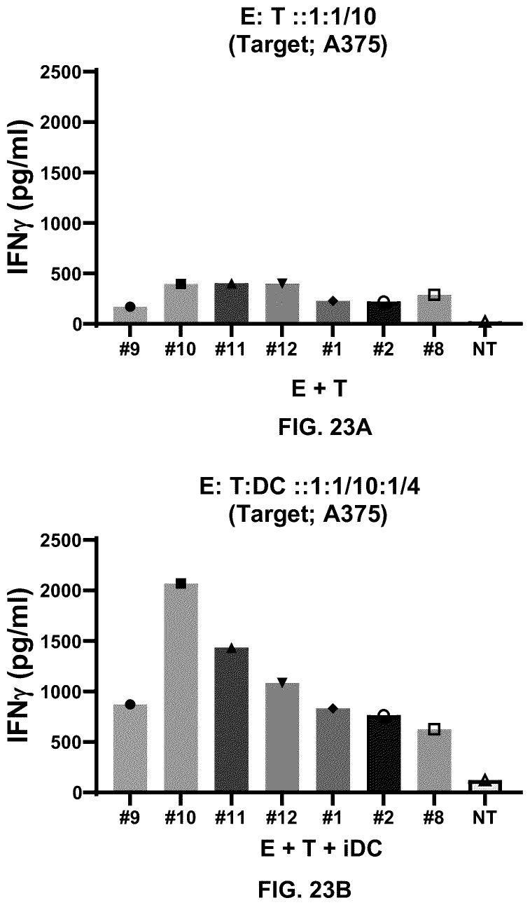

IFNγ secretion in response to A375 increases in the presence of immature DC (iDCs). In the tri-cocultures with iDCs, IFNγ secretion is higher in Construct #10 compared to the other constructs. However, comparing Construct #9 with Construct #11 expressing wild type and modified CD8 coreceptor sequences respectively, T cells transduced with #11 induced stronger cytokine response measured as IFNγ quantified in the culture supernatants of three-way cocultures using donor D600115, E:T:iDC:: 1:1/10:1/4. FIG. 23A-B.

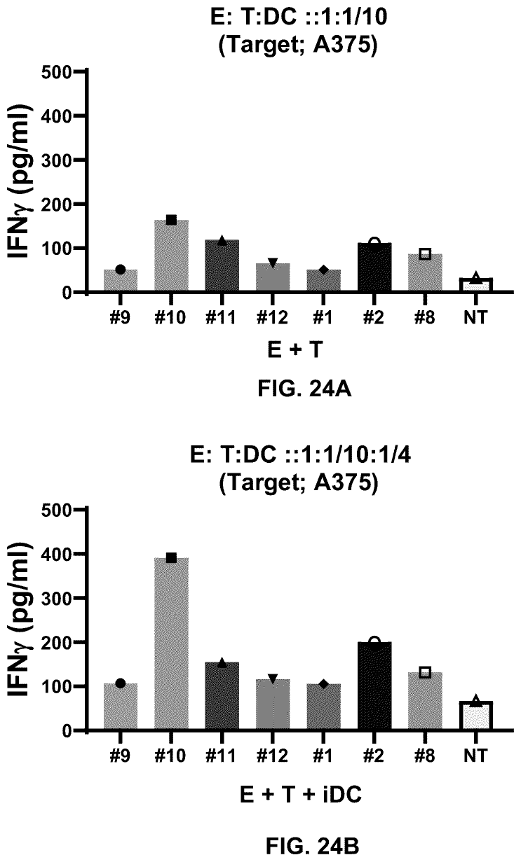

IFNγ secretion in response to A375 increases in the presence of iDCs. In the tri-cocultures with iDCs, IFNγ secretion was higher in Construct #10 compared to the other constructs. IFNγ quantified in the supernatants from DC cocultures D150081, E:T:iDC:: 1:1/10:1/4. FIG. 24A-B

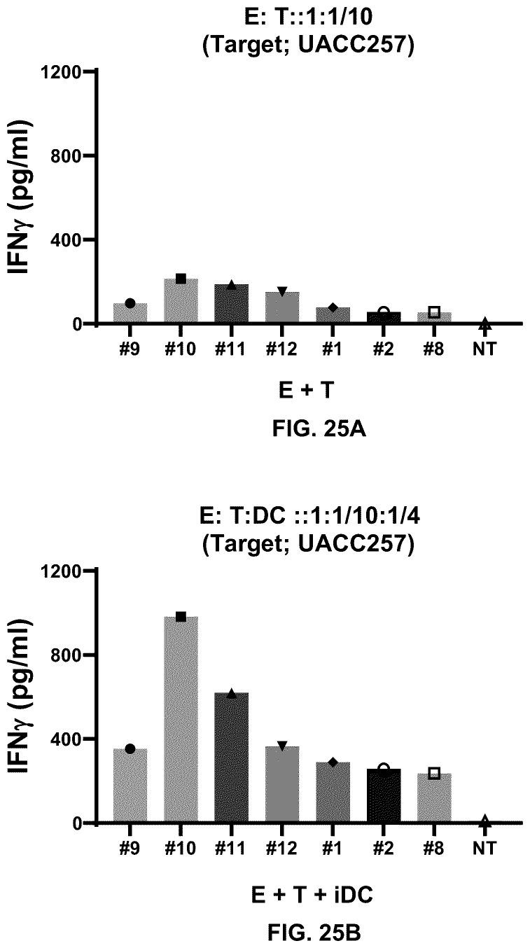

IFNγ secretion in response to UACC257 increases in the presence of iDCs. In the tri-cocultures with iDCs, IFNγ secretion is higher in Construct #10 compared to the other constructs. However, comparing Construct #9 with Construct #11 expressing wild type and modified CD8 coreceptor sequences respectively, T cells transduced with Construct #11 induced stronger cytokine response measured as IFNγ quantified in the culture supernatants of three-way cocultures using donor D600115, E:T:iDC::1:1/10:1/4. FIG. 25A-B. These results demonstrate that T cell products co-expressing a transgenic TCR and CD8 co-receptor (a heterodimer or modified CD8α homodimer) are able to license DCs in the microenvironment through antigen cross presentation and therefore hold the potential to mount a stronger anti-tumor response and modulate the tumor microenvironment.

### Example 7

### Vector Screening (Constructs #13-#21)

### Viral Titers

FIG. 5B shows viral titer of Constructs #10, #10n (new batch), #11, #11n (new batch), #13-#21, and TCR only as a control.

### T Cell Manufacturing

### Activation

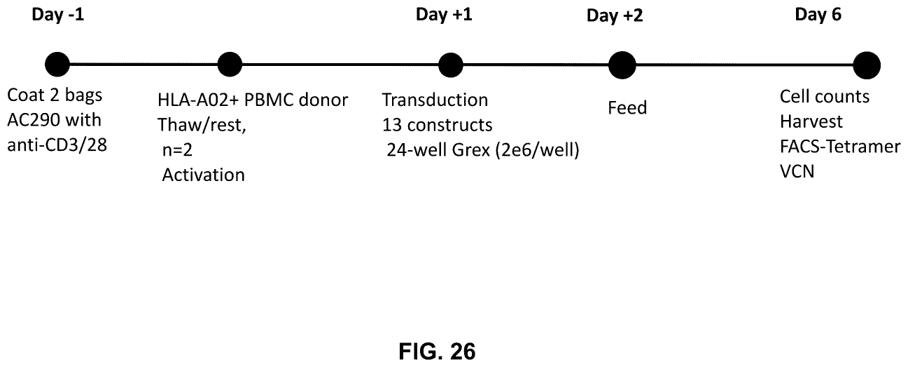

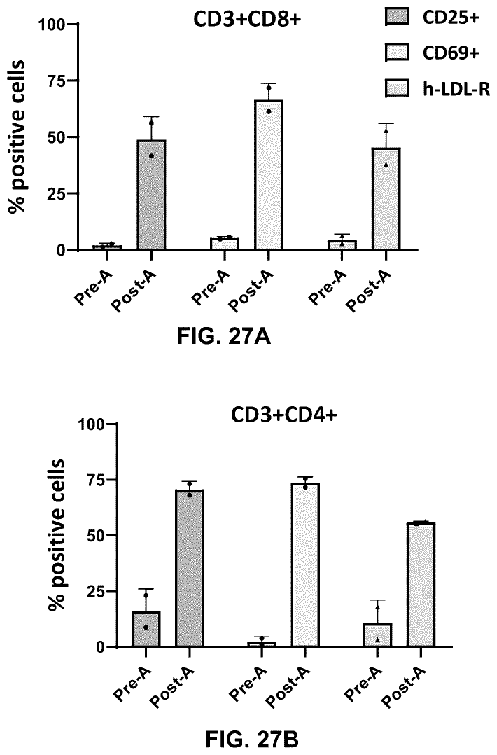

FIG. 26 shows that, on Day +0, PBMCs obtained from two HLA-A02+ donors (Donor #1 and Donor #2) were thawed and rested. Cells were activated in bags (AC290) coated with anti-CD3 and anti-CD28 antibodies in the absence of serum. Activation markers, e.g., CD25, CD69, and human low density lipoprotein receptor (H-LDL-R) are in CD8+ and CD4+ cells, were subsequently measured. FIG. 27A shows that % CD3+CD8+CD25+ cells, % CD3+CD8+CD69+ cells, and % CD3+CD8+H-LDL-R+ cells increase after activation (Post-A) as compared with that before activation (Pre-A). Similarly, FIG. 27B shows that % CD3+CD4+CD25+ cells, % CD3+CD4+CD69+ cells, and % CD3+CD4+H-LDL-R+ cells increase after activation (Post-A) as compared with that before activation (Pre-A). These results support the activation of PBMCs.

### Transduction

FIG. 26 shows that, on Day +1, activated PBMCs were transduced with viral vectors, e.g., Constructs #8, #10, #10n, #11, #11n, and #13-#21, in G-Rex® 24-well plates at about 2×10⁶ cells/well in the absence of serum. The amounts of virus used for transduction are shown in Table 8.

TABLE 8

| Constructs | Virus Volume/1 × 10⁶ cells |
|---|---|
| #10n, #11n, #13-#21 | 0.3 μl, 1.1 μl, 3.3 μl, 10 μl, 30 μl |
| #8 (TCR), #10 | 2.5 μl |
| #11 | 1.25 μl |
| NT | — |

### Expansion

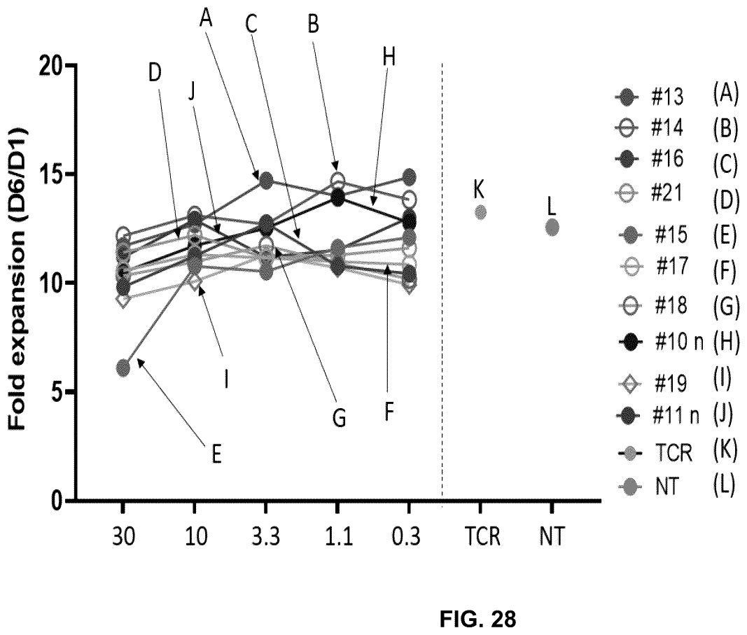

FIG. 26 shows that, on Day +2, transduced PBMCs were expanded in the absence of serum. On Day +6, cells were harvested for subsequent analysis, e.g., FACS-Tetramer and vector copy number (VCN) and were cryopreserved. FIG. 28 shows fold expansion on Day +6 of transduced T cell products. Viabilities of cells is greater than 90% on Day +6.

### Characterization of T Cell Products

Cell counts, FACS-dextramers, and vector copy numbers (VCN) were determined. Tetramer panels may comprise live/dead cells, CD3, CD8α, CD8β, CD4, and peptide/MHC tetramers, e.g., PRAME-004 (SLLQHLIGL) (SEQ ID NO: 147)/MHC tetramers. FACS analysis was gated on live singlets, followed by CD3+, followed by CD4+CD8+, followed by CD4+CD8+Tetramer(Tet)+ and CD8+Tet+.

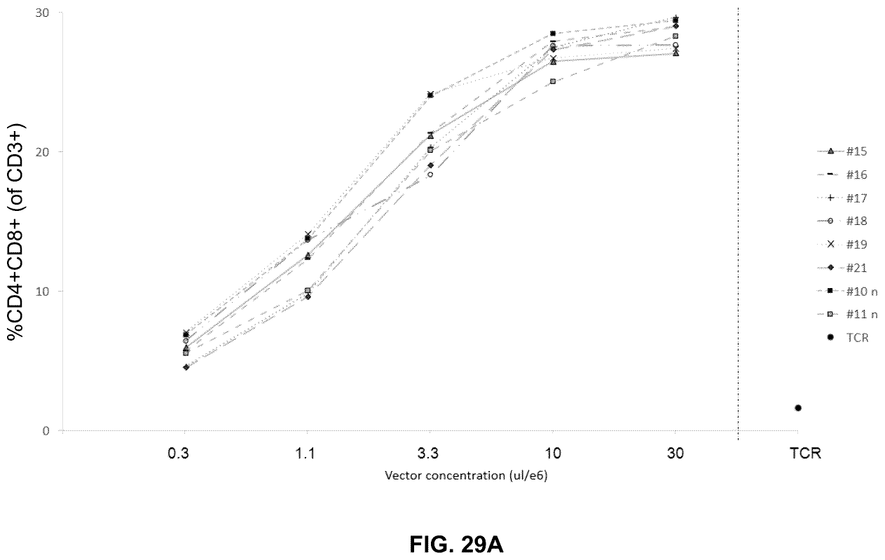

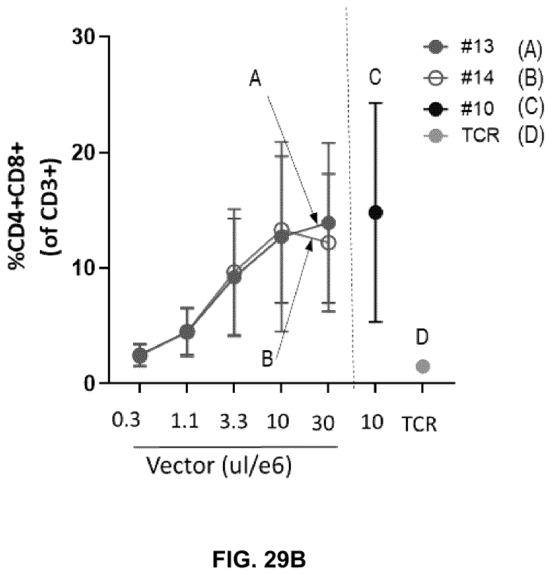

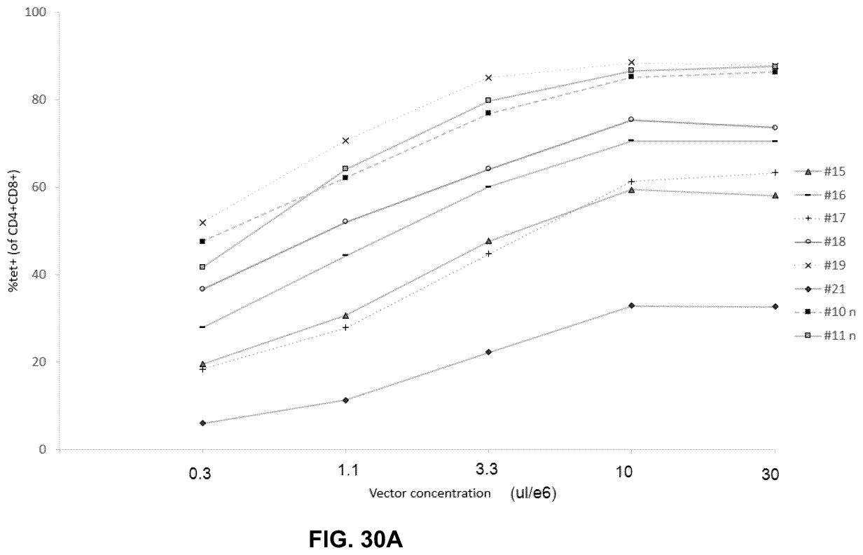

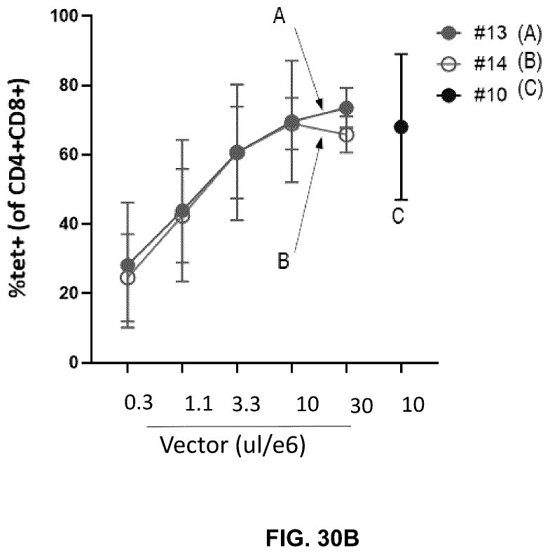

FIG. 29A and FIG. 29B shows % CD8+CD4+ cells transduced with Construct #10, #10n, #11, #13-#21 at 0.3 μl, 1.1 μl, 3.3 μl, 10 μl or 30 μl per 1×10⁶ cells. These results show comparable frequencies of CD8+CD4+ cells obtained by transduction with all vectors tested. Construct #8 (TCR only) serves as negative control. FIG. 30A and FIG. 30B shows % Tet of CD8+CD4+ cells from transduced with Construct #10, #10n, #11, #13-#21 at 0.3 μl, 1.1 μl, 3.3 μl, 10 μl or 30 μl per 1×10⁶ cells. These results show that there was a trend towards higher frequencies of CD4+CD8+tet+ in CD8(31 isoforms (Constructs #10 and #18) compared to CD8β3 isoforms (Construct #16) and CD8β5 isoforms (Constructs #15 and #17). FACS analysis was gated on live singlets, followed by CD3+, followed by CD4+CD8+, and followed by CD4+CD8+Tet+.

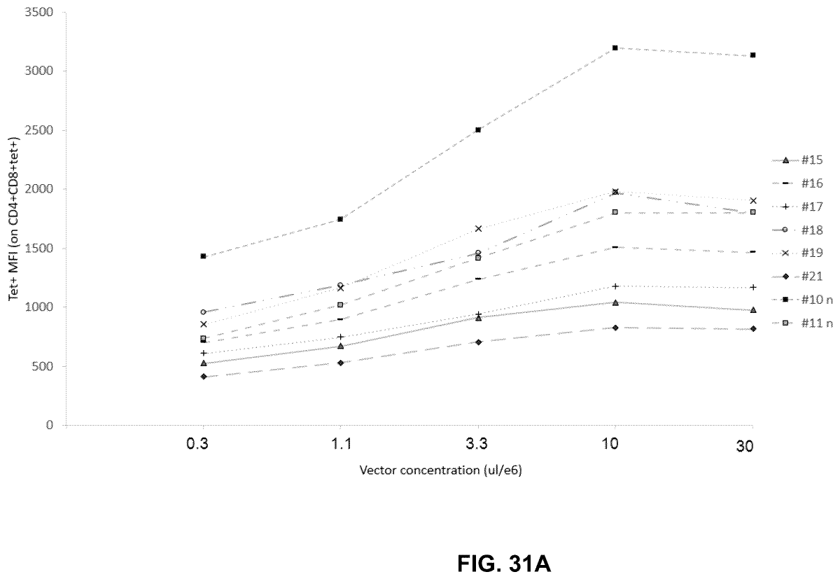

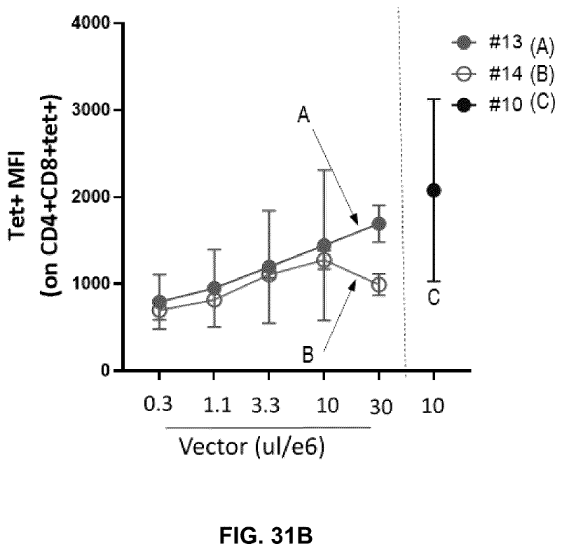

FIG. 31A and FIG. 31B shows Tet MFI of CD8+CD4+Tet+ cells from transduced with Construct #10, #10n, #11, #13-#21 at 0.3 μl, 1.1 μl, 3.3 μl, 10 μl or 30 μl per 1×10⁶ cells. These results show a trend towards higher tetramer MFI on CD4+CD8+Tet+ population in CD8β1 isoforms (Constructs #10 and #18) compared to CD8β3 isoforms (Construct #16) and CD8β5 isoforms (Constructs #15 and #17).

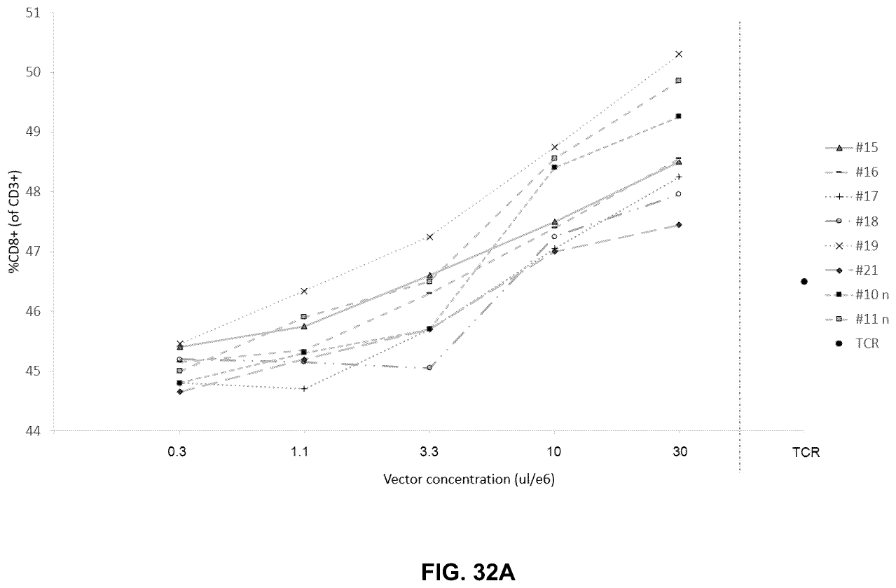

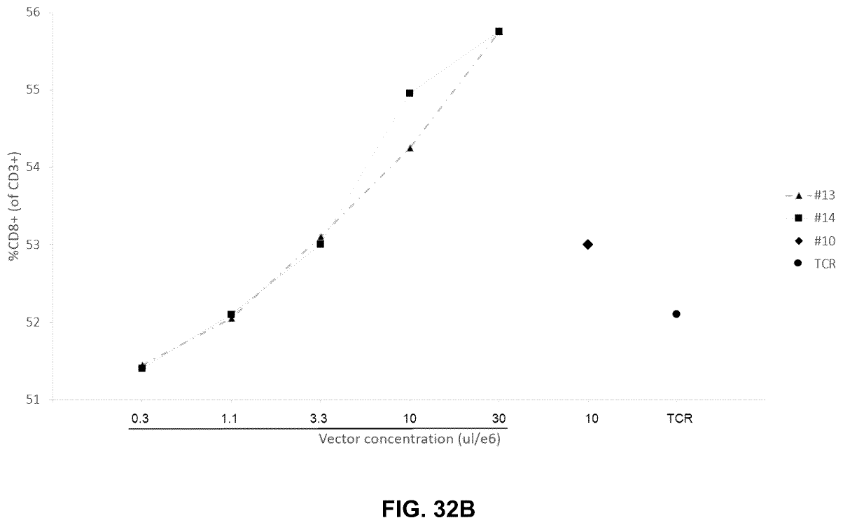

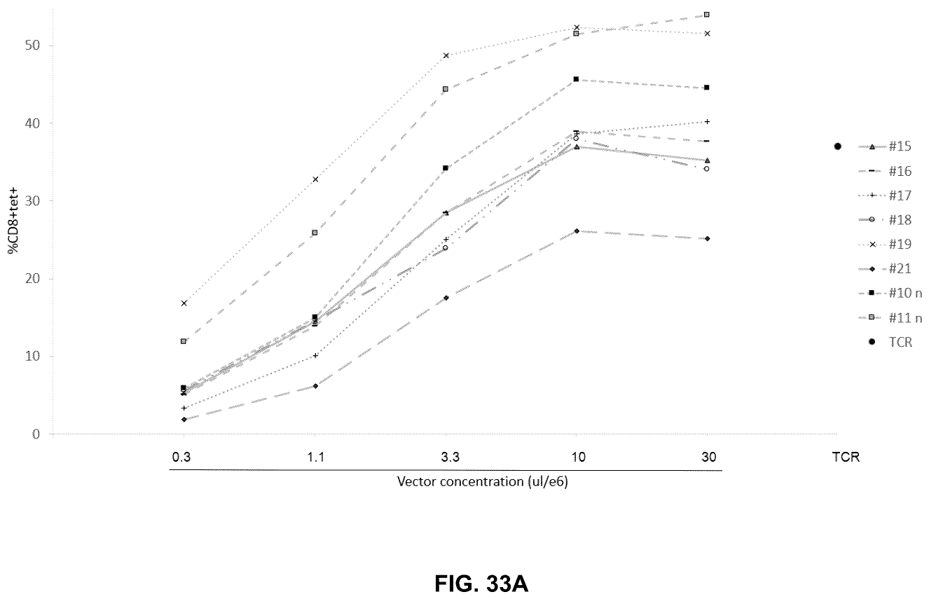

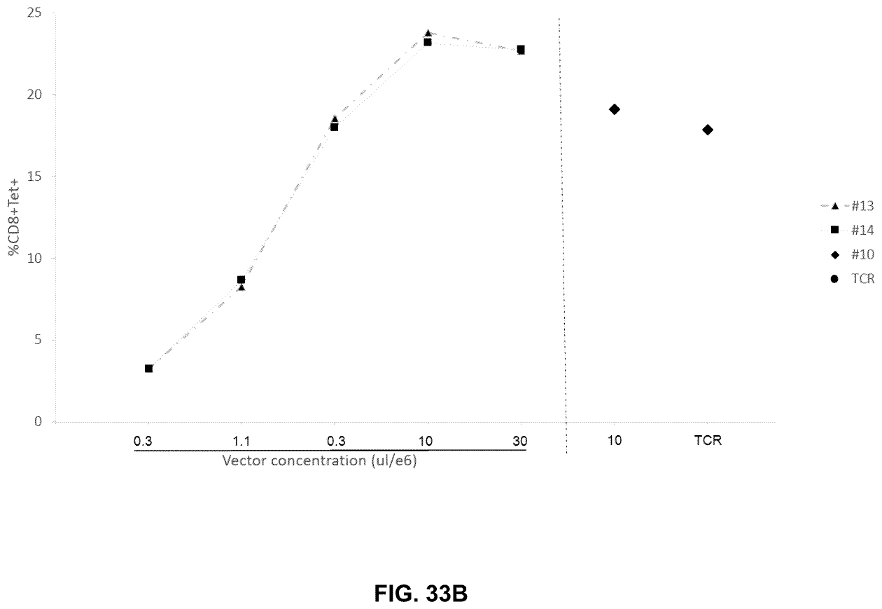

FIG. 32A and FIG. 32B show CD8 frequencies (% CD8+CD4− of CD3+) in cells transduced with Construct #10, #10n, #11, #13-#21 at 0.3 μl, 1.1 μl, 3.3 μl, 10 μl or 30 μl per 1×10⁶ cells. These results show no difference in the CD8 frequencies among the constructs. FIG. 33A and FIG. 33B shows % CD8+Tet+(of CD3+) cells transduced with Construct #10, #10n, #11, #13-#21 at 0.3 μl, 1.1 μl, 3.3 μl, 10 μl or 30 μl per 1×10⁶ cells. These results show slightly higher frequencies of CD8+Tet+(of CD3+) in cells transduced with Construct #10 than those transduced with the other constructs. FACS analysis was gated on live singlets, followed by CD3+, followed by CD8+CD4−, and followed by Tet+.

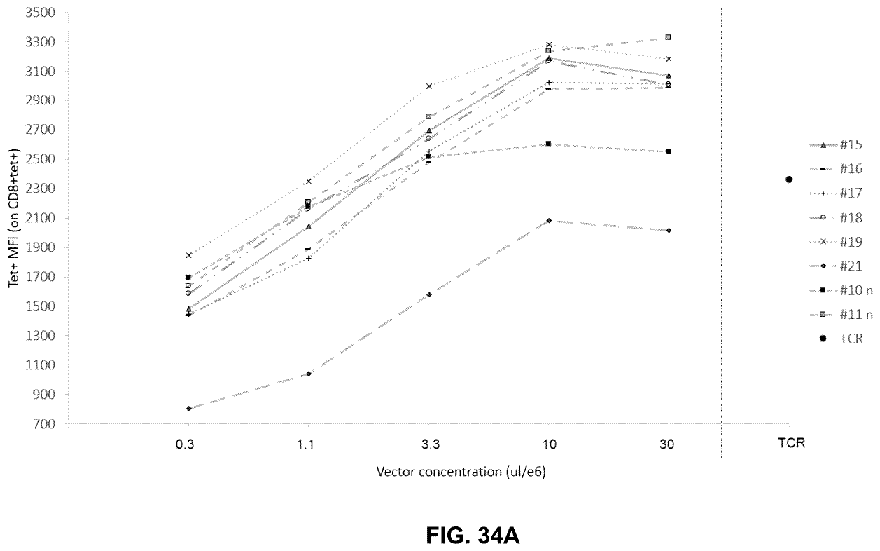

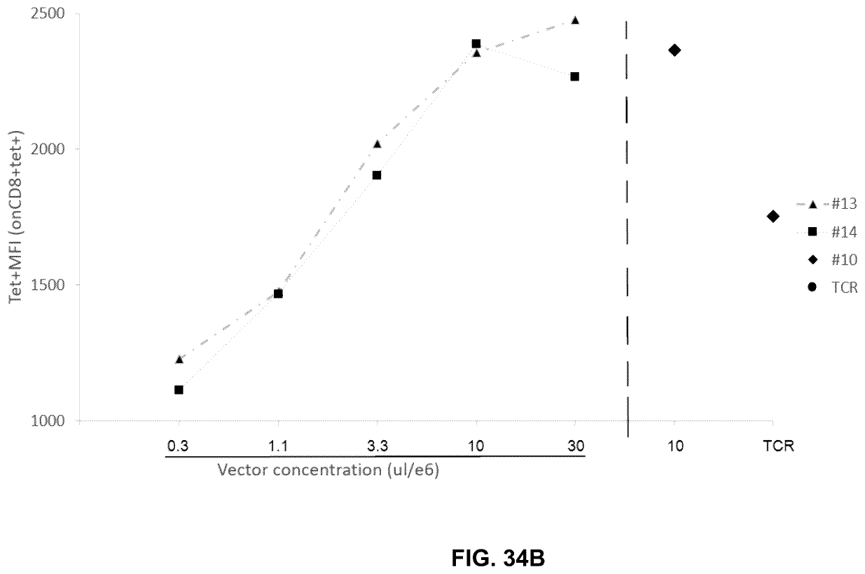

FIG. 34A and FIG. 34B shows Tet MFI of CD8+Tet+ cells transduced with Construct #10, #10n, #11, #13-#21 at 0.3 μl, 1.1 μl, 3.3 μl, 10 μl or 30 μl per 1×10⁶ cells. These results show tetramer MFI of CD8+tet+ cells was comparable among CD8β1 (Constructs #18 and #10), CD8β5 (Constructs #15 and #17), and CD8β3 (Construct #16) isoforms, while Construct #21 expressed lower tetramer MFI.

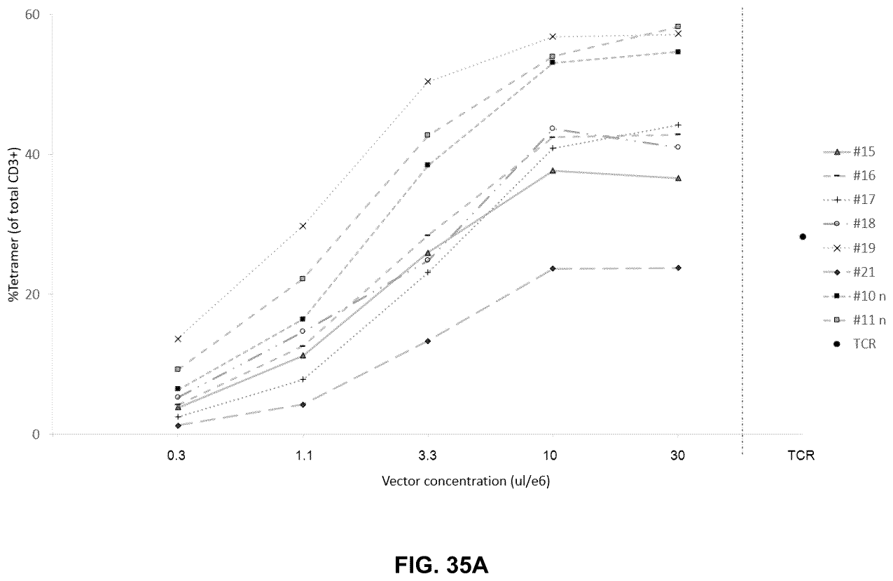

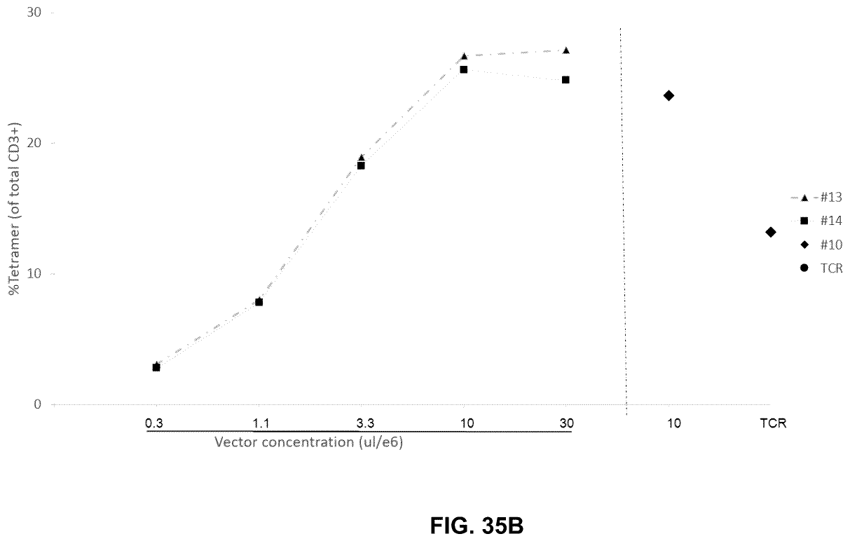

FIG. 35A and FIG. 35B shows % Tet+ of CD3+ cells transduced with Construct #10, #10n, #11, #13-#21 at 0.3 μl, 1.1 μl, 3.3 μl, 10 μl or 30 μl per 1×10⁶ cells. These results show higher frequencies of CD3+Tet+ in cells transduced with Construct #10 (CD8β1) compared to those transduced with CD8β3 (Construct #16) and CD8β5 (Constructs #15 and #17). FACS analysis was gated on live singlets, followed by CD3+, and followed by Tet+.

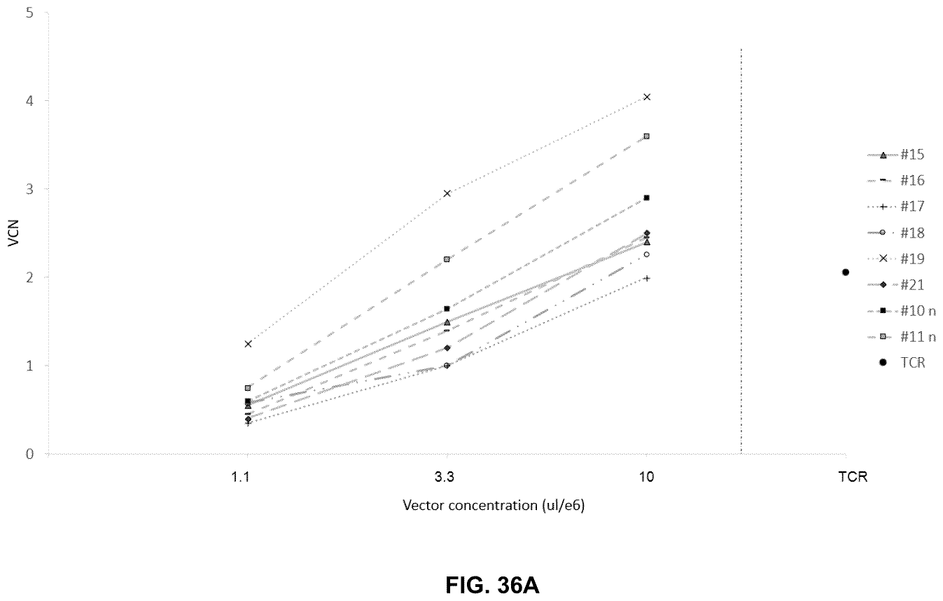

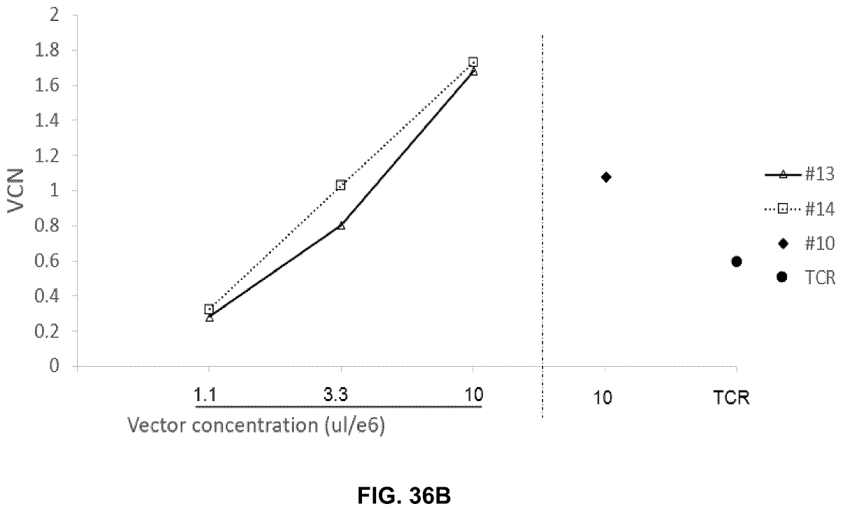

FIG. 36A and FIG. 36B shows vector copy number (VCN) of cells transduced with Construct #10, #10n, #11, #13-#21 at 0.3 μl, 1.1 μl, 3.3 μl, 10 μl or 30 μl per 1×10⁶ cells. These results show comparable ability of all constructs to integrate and express CD8/TCR genes.

In sum, these results show (1) viral vectors with CD8β1, CD8β3 and CD8β5 isoforms had good transducing titers; (2) all constructs were capable of successful manufacturing (e.g., high viability, fold expansions in the range of 6−12); (3) frequencies of CD3+tet+ among CD8β isoforms: CD8β1 (Construct #10) was greater than CD8β3 (Construct #16) and CD8β5 (Constructs #15 and #17), with Construct #21 showing the lowest values; (4) frequency of CD3+tet+ in Constructs #11 and #19 (m1CD8α (SEQ ID NO: 7)) showed the highest values; and (5) saturation in % CD3+tet+, % CD8+tet+ and % CD4+CD8+tet+ observed at 10 μl/e6. Optimal vector dose ranges between 3.3−10 μl/e6 for all constructs.

### Example 7

### Mid-Scale Vector Screening (Constructs #13-#19)

T Cell Manufacturing

Activation/Transduction

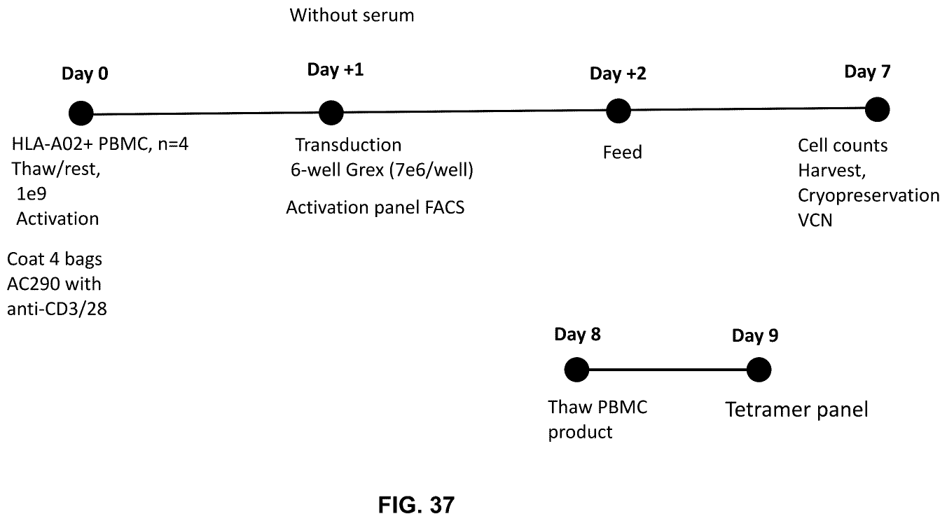

FIG. 37 shows that, on Day +0, PBMCs obtained from four HLA-A02+ donors were thawed and rested. Cells were activated in bags (AC290) coated with anti-CD3 and anti-CD28 antibodies in the absence of serum. On Day +1, activated PBMCs were transduced with viral vectors, e.g., Constructs #8, #10n, #11n, and #13-#19, in G-Rex® 6-well plates at about 7×10⁶ cells/well in the absence of serum. The amounts of virus used for transduction are shown in Table 9.

TABLE 9

| Constructs | Virus Volume/1 × 10⁶ cells |
|---|---|
| #13-19 | 2.5 μl and 5 μl |
| #10n and #11n | 2.5 μl and 5 μl |
| #8 (TCR) | 2.5 μl |
| NT | — |

### Expansion

FIG. 37 shows that, on Day +2, transduced PBMCs were expanded in the absence of serum. On Day +7, cells were harvested for subsequent analysis, e.g., FACS-Tetramer and vector copy number (VCN) and were cryopreserved. Fold expansion on Day +7 was comparable for all constructs (approximately 30-fold expansion). Viabilities of cells is greater than 90% on Day +7.

### Characterization of T Cell Products

Cell counts, FACS-dextramers, and vector copy numbers (VCN) were determined. Tetramer panels may comprise live/dead cells, CD3, CD8α, CD8β, CD4, and peptide/MHC tetramers, e.g., PRAME-004 (SLLQHLIGL) (SEQ ID NO: 147)/MHC tetramers. FACS analysis was gated on live singlets, followed by CD3+, followed by CD4+CD8+, followed by CD4+CD8+Tetramer(Tet)+ and CD8+Tet+.

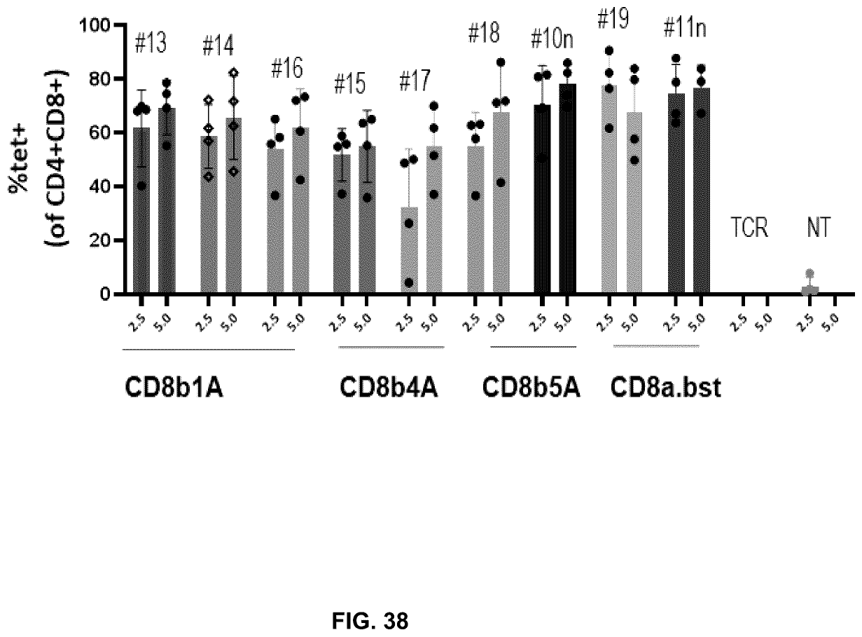

Similar to results described in Example 6, comparable frequencies of CD8+CD4+ cells were obtained by transduction with Construct #10n, #11n, #13-#19 at 2.5 μl or 5.0 μl per 1×10⁶ cells. Construct #8 (TCR only) serves as negative control. FIG. 38 shows % Tet of CD8+CD4+ cells transduced with Construct #10n, #11n, #13-#19 at 2.5 μl or 5.0 μl per 1×10⁶ cells. Similar to results described in Example 6, these results show that there was a trend towards higher frequencies of CD4+CD8+tet+ in CD8β1 isoforms (Construct #10n) compared to CD8β3 isoforms (Constructs #13, #14, #16) and CD8β5 isoforms (Constructs #15 and #17). FACS analysis was gated on live singlets, followed by CD3+, followed by CD4+CD8+, and followed by Tet+.

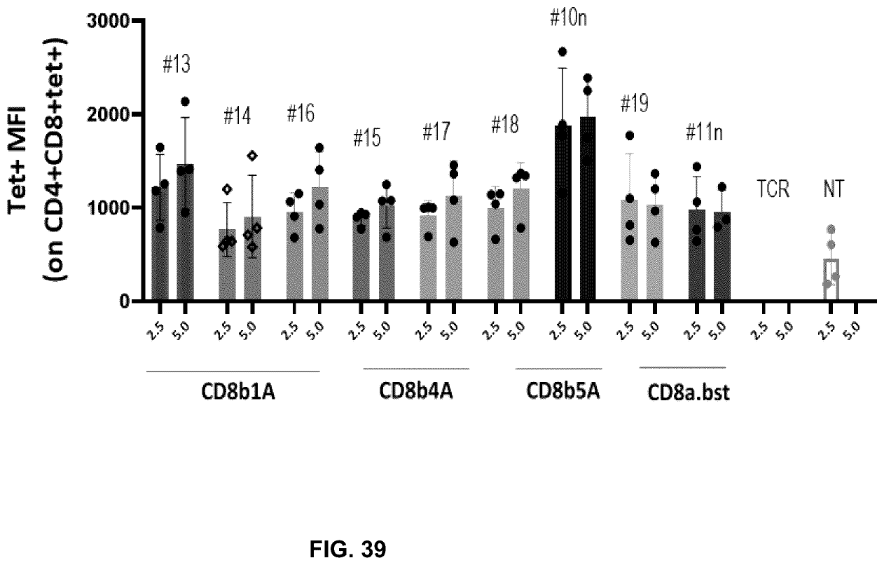

FIG. 39 shows Tet MFI of CD8+CD4+Tet+ cells from transduced with Construct #10n, #11n, #13-#19 at 2.5 μl or 5.0 μl per 1×10⁶ cells. These results show higher tetramer MFIs on CD4+CD8+Tet+ population in CD8β1 isoforms (Construct #10n) compared to CD8β3 isoforms (Construct #13) and CD8β5 isoforms (Constructs #15 and #17).

Similar to results described in Example 6, results show no difference in the CD8 frequencies (% CD8+CD4− of CD3+) in cells transduced with Construct #10n, #11n, #13-#19 at 2.5 μl or 5.0 μl per 1×10⁶ cells among the constructs (data not shown). Comparable frequencies of CD8+Tet+(of CD3+) in cells transduced with Construct #10n, #11n, #13-#19 at 2.5 μl or 5.0 μl per 1×10⁶ cells (data not shown). FACS analysis was gated on live singlets, followed by CD3+, followed by CD8+CD4−, and followed by Tet+.

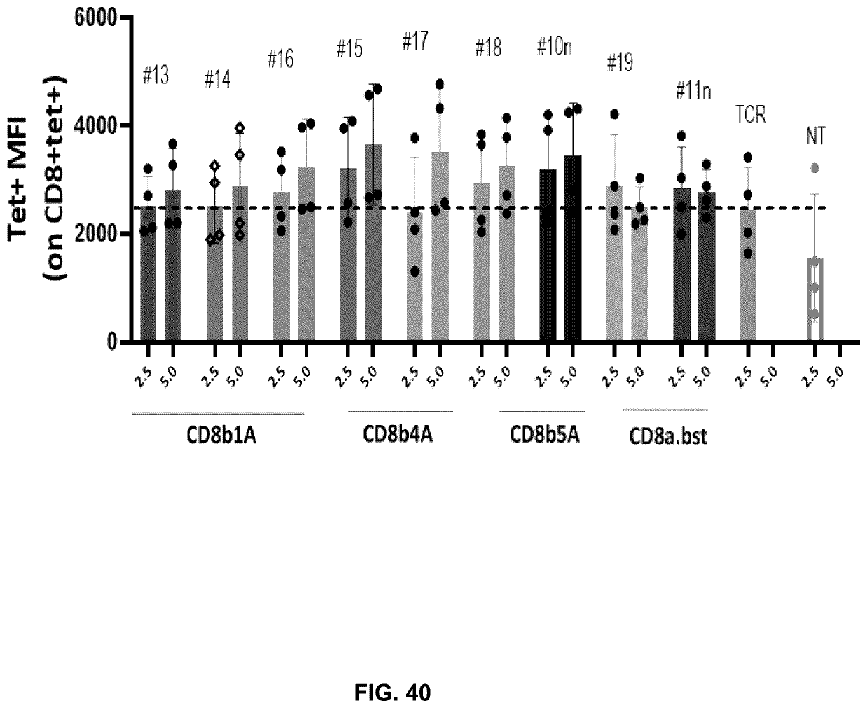

FIG. 40 shows Tet MFI of CD8+Tet+ cells transduced with Construct #10n, #11n, #13-#19 at 2.5 μl or 5.0 μl per 1×10⁶ cells. These results show tetramer MFI of CD8+tet+ cells was comparable among CD8β1 (Constructs #18 and #10) and CD8β5 (Construct #15) isoforms, while CD8β3 (Constructs #13, #14, and #16) isoforms expressed lower tetramer MFI.

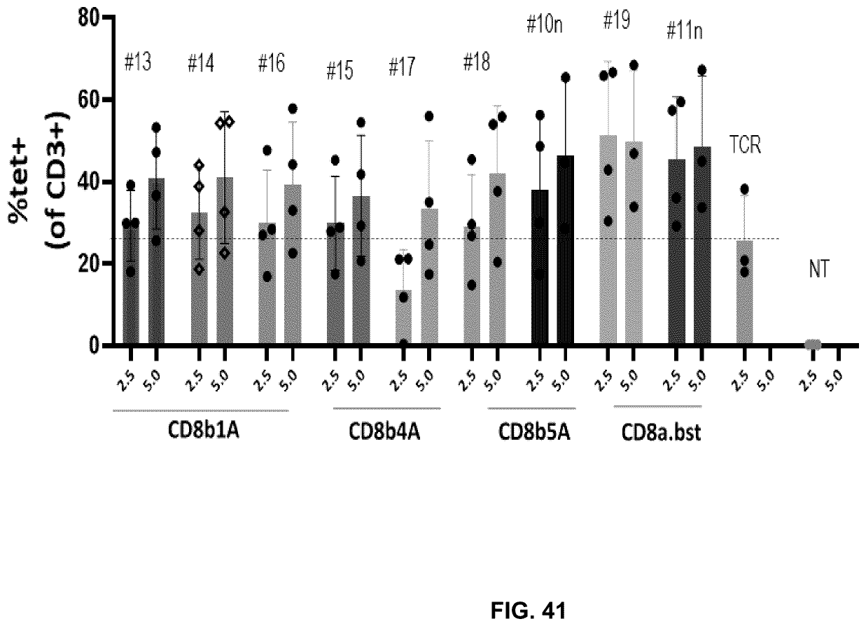

FIG. 41 shows % Tet+ of CD3+ cells transduced with Construct #10n, #11n, #13-#19 at 2.5 μl or 5.0 μl per 1×10⁶ cells. These results show slightly higher frequencies of CD3+Tet+ in cells transduced with Construct #10 (CD8β1) compared to those transduced with CD8β3 (Constructs #13, #14, and #16) and CD8β5 (Construct #15). FACS analysis was gated on live singlets, followed by CD3+, and followed by Tet+. Slightly higher total CD3+tet+ cell counts were observed in PBMC transduced with Construct #10 CD8β1) compared to those transduced with CD8β3 (Constructs #13, #14, and #16) and CD8β5 (Construct #15) (data not shown).

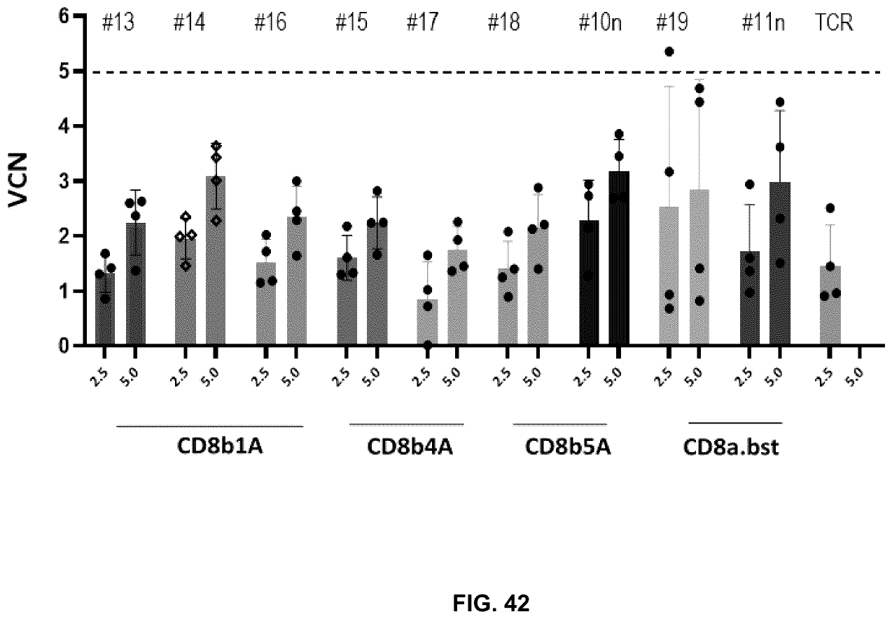

FIG. 42 shows vector copy number (VCN) of cells transduced with Construct #10n, #11n, #13-#19 at 2.5 μl or 5.0 μl per 1×10⁶ cells. These results show vector copies per cell remained below 5 in PBMC product derived using each individual construct at vector dose of 2.5 μl or 5.0 μl per 1×10⁶ cells.

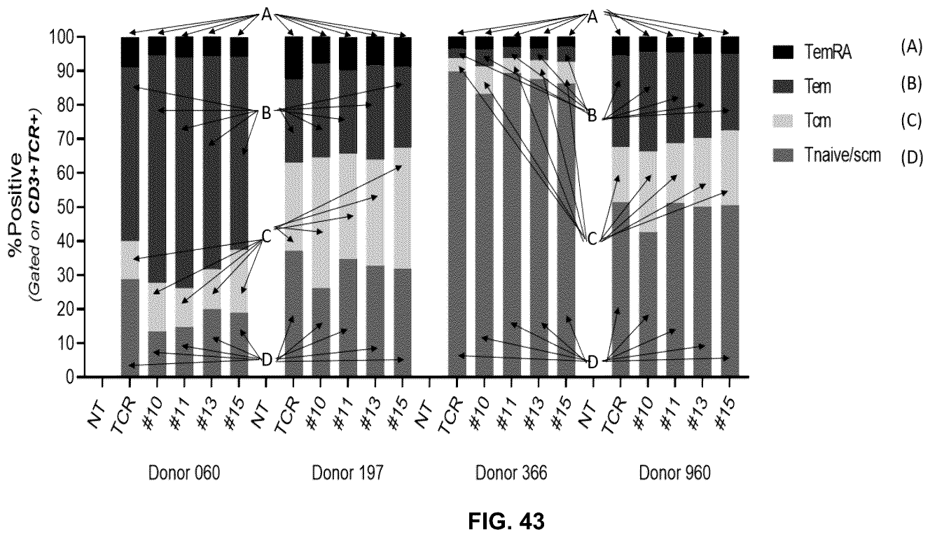

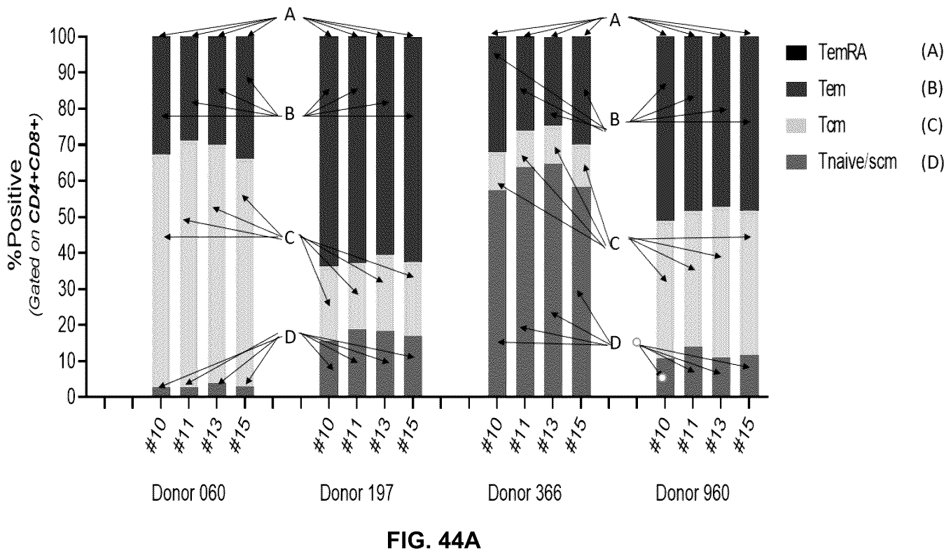

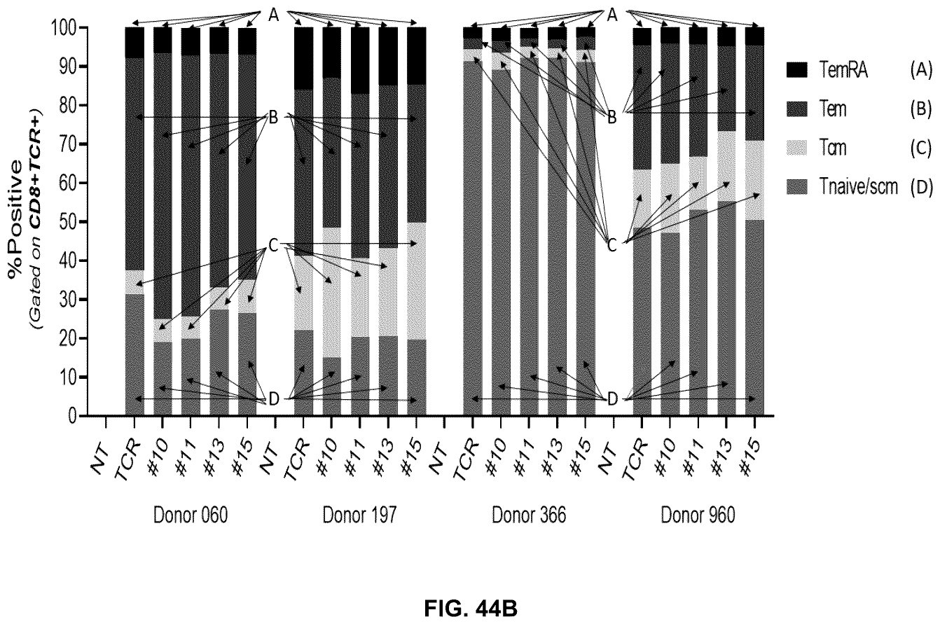

FIG. 43 shows the % T cell subsets in cells transduced with Construct #10, #11, #13, and #15 for each donor. Construct #8 (TCR only) and non-transduced cells were used as controls. These results show that TCR-only condition has slightly more naïve cells compared to the other constructs, consistent with lower fold-expansion. FIG. 44A and FIG. 44B shows % T cell subsets in cells transduced with Construct #10, #11, #13, and #15 for each donor. Construct #8 (TCR only) and non-transduced cells were used as controls. FACS analysis was gated on CD4+CD8+ for FIG. 44A and on CD4−CD8+TCR+ for FIG. 44B. These results show donor-to-donor variability between frequencies of T cell memory subsets but little difference in the frequencies of Tₙₐᵢᵥₑ and T_{cm} between constructs.

In sum, these results show (1) viability and fold expansions were comparable among all constructs at day 7; (2) slightly higher frequency of CD3+tet+ observed in CD8β1 (Construct #10) compared to CD8β3 (Constructs #13, #14, and #16) and CD8β5 (Constructs #15 and #17); (3) vector copies per cell <5 for majority of the constructs at 2.5-5 μl/10⁶ dose; and (4) donor-to-donor variability between frequencies of T cell memory subsets but generally, Construct #10 has less naïve but more Tcm cells than the other β isoform constructs.

### Example 8

### Tumor Death Assay—Constructs #10, #11, #13 & #15

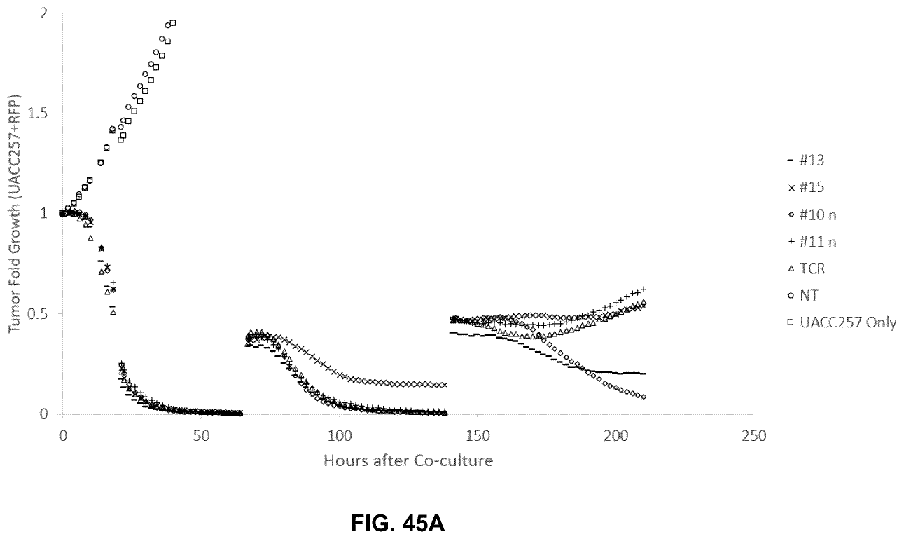

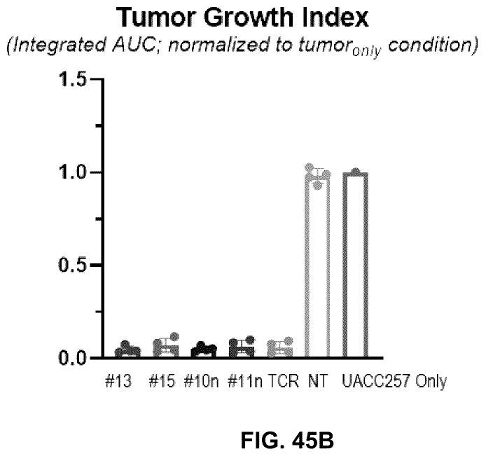

FIGS. 45A and 45B depicts data showing that Constructs #13 and #10 are comparable to TCR-only in mediating cytotoxicity against UACC257 target positive cells lines expressing high levels of antigen (1081 copies per cell). Construct #15 was also effective but slower in killing compared to Constructs #13 and #10. The effector:target ratio used to generate these results was 4:1. Similar results were obtained with a 2:1 effector:target ratio (data not shown).

### Example 9

### IFNγ Secretion Assay—Constructs #10, #11, #13 & #15

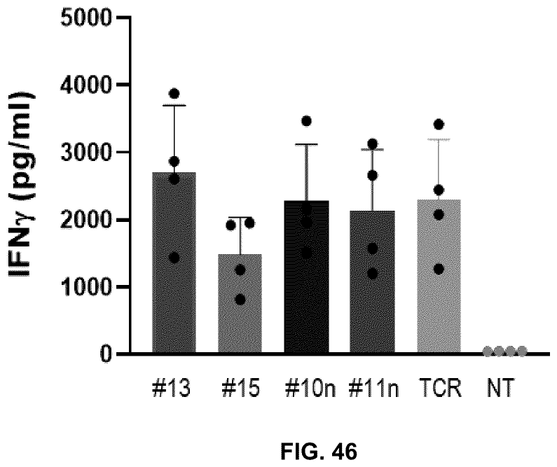

IFNγ secretion was measured in the UACC257 cells line. FIG. 46 shows IFNγ secretion in response in UACC257 cell line was higher with Construct #13 compared to Construct #10. IFNγ quantified in the supernatants from Incucyte plates. The effector:target ratio used to generate these results was 4:1. Similar results were obtained with a 2:1 effector:target ratio (data not shown).

### Example 10

### ICI Marker Expression—Constructs #10, #11, #13 & #15

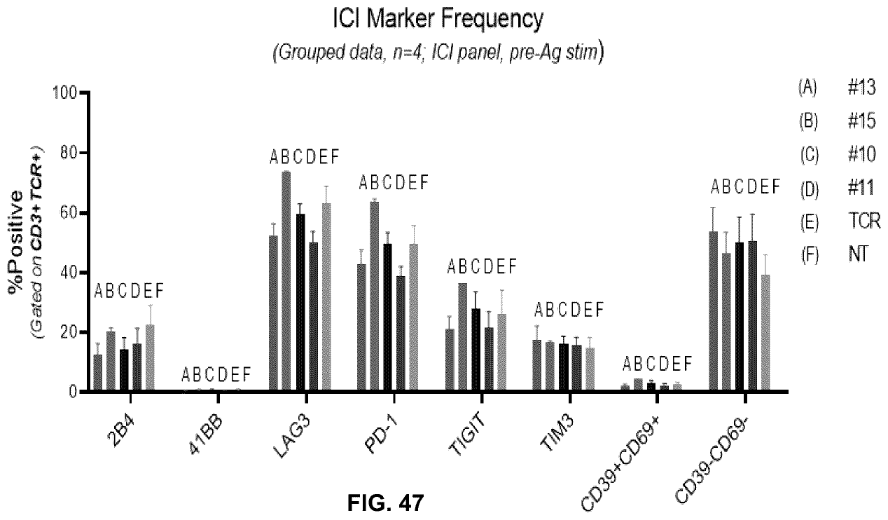

ICI marker frequency (2B4, 41BB, LAG3, PD-1, TIGIT, TIM3, CD39+CD69+, and CD39-CD69−) was measured. FIG. 47 shows Construct #15 has higher expression of LAG3, PD-1, and TIGIT compared to other constructs, followed by Construct #10.

### Example 11

### Cytokine Expression—Constructs #10, #11, #13 & #15

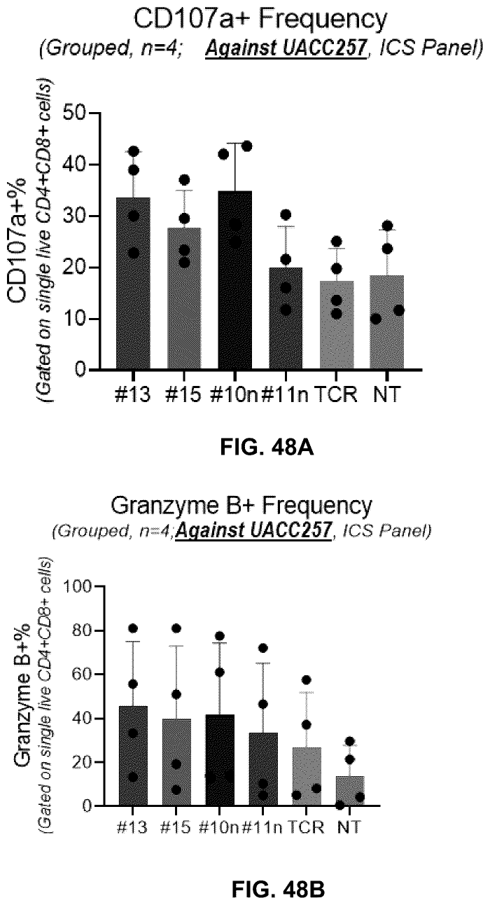

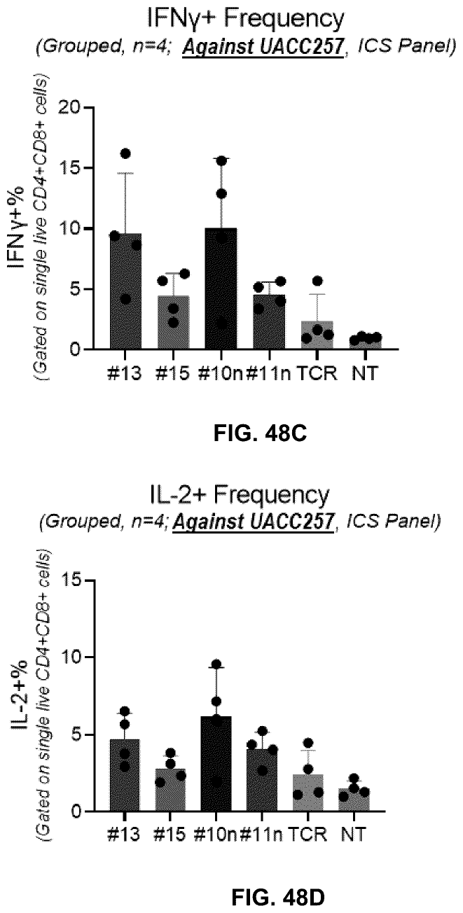

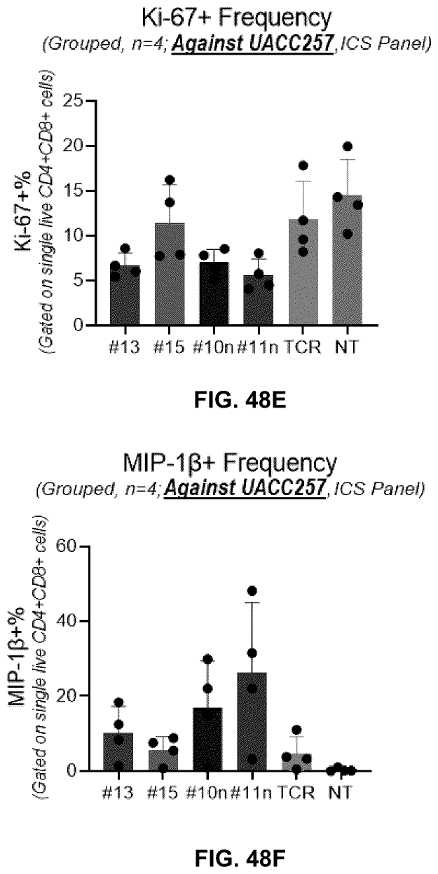

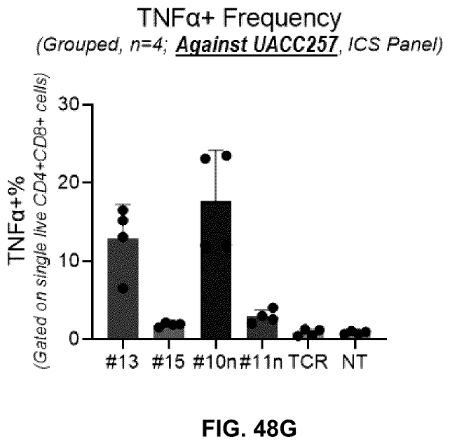

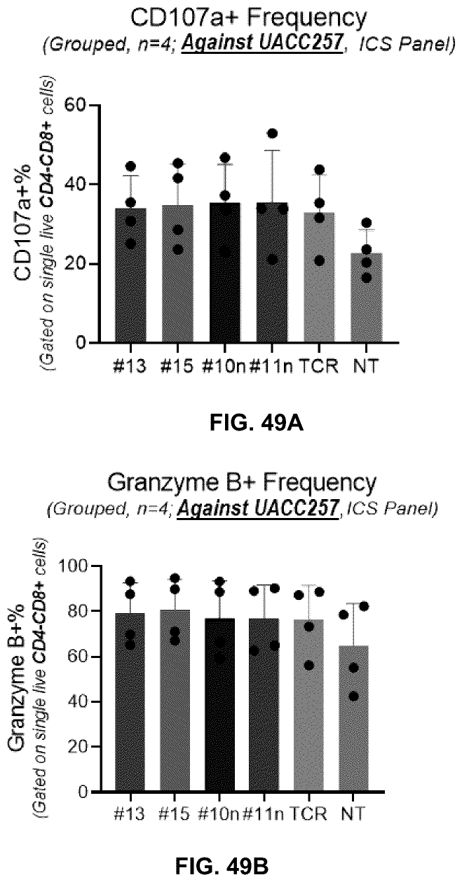

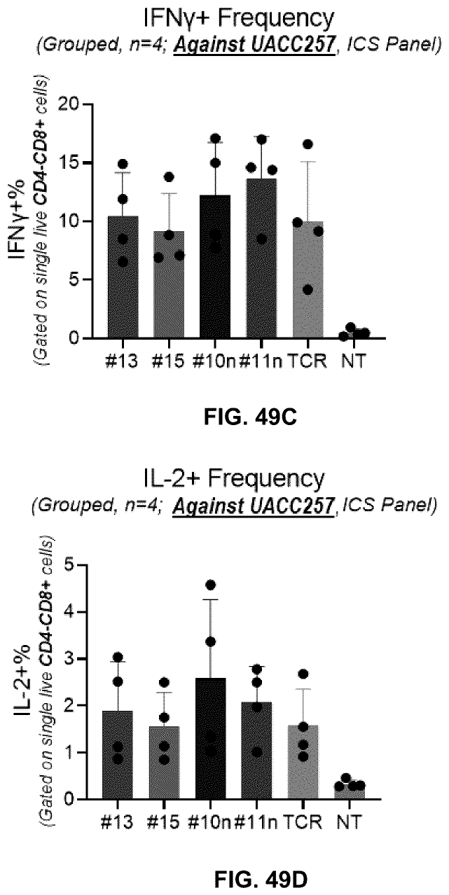

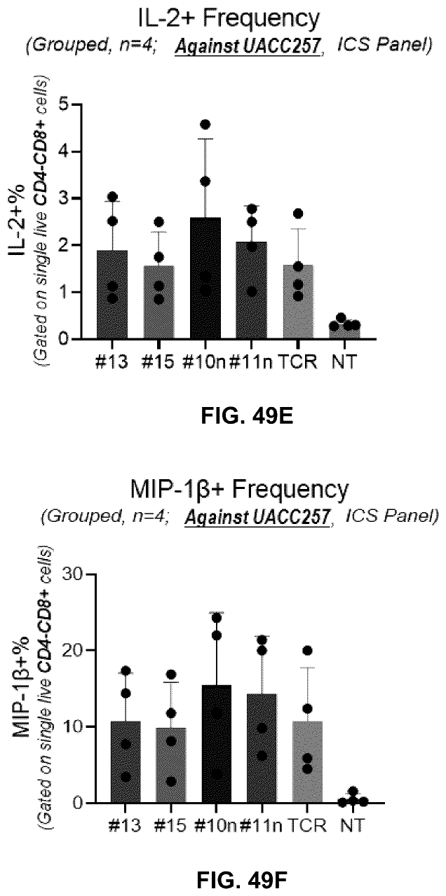

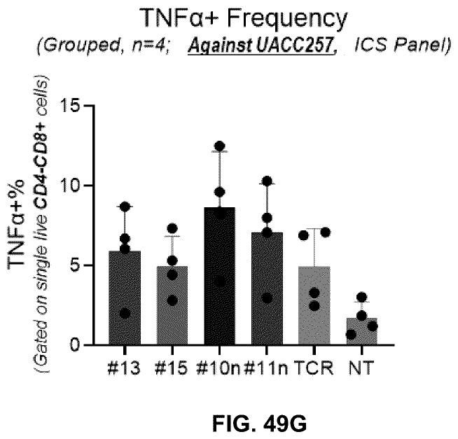

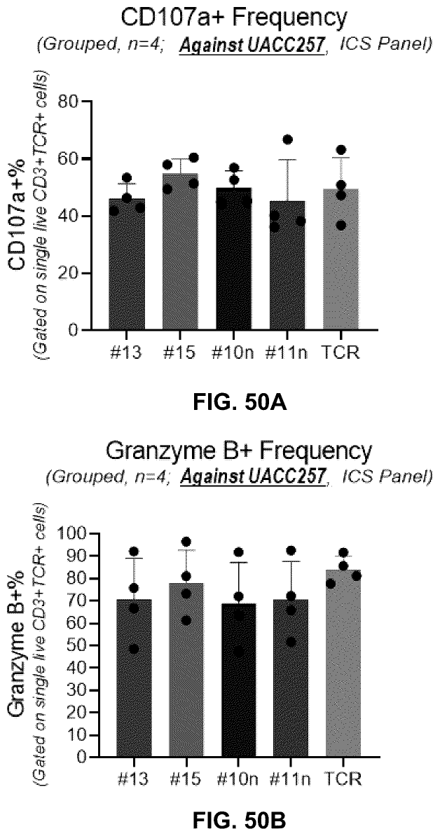

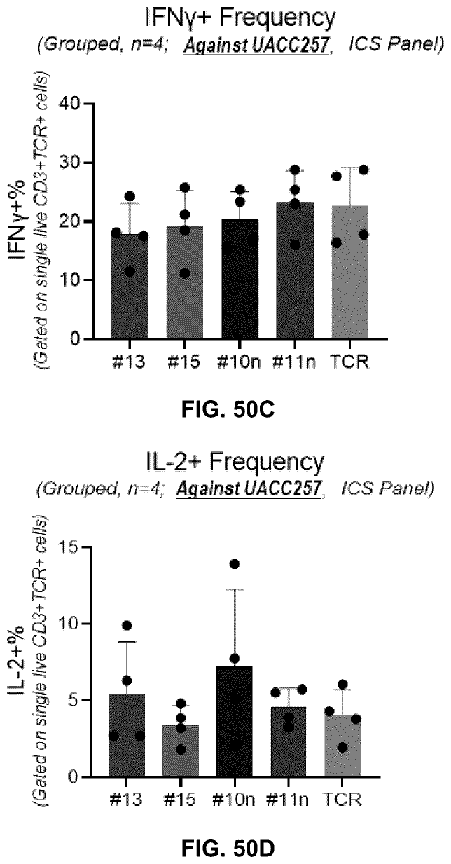

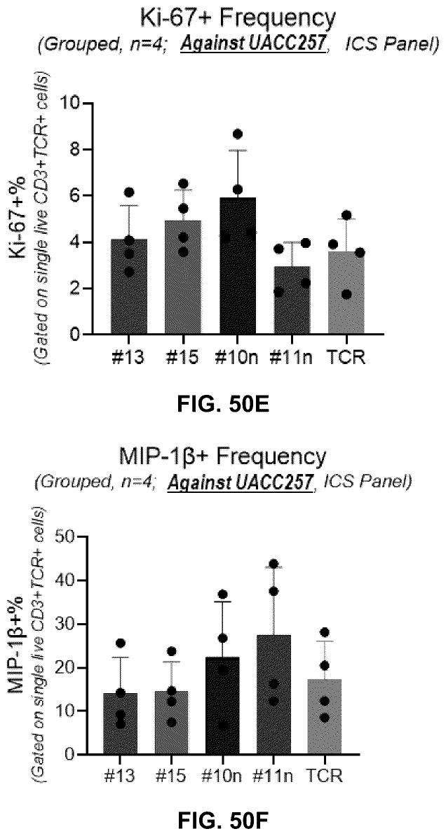

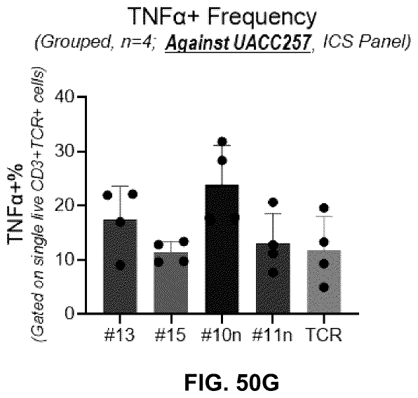

Expression of various cytokines was measured in UACC257 cells co-cultured at a 4:1 E:T ratio with PBMC transduced with Constructs #10, #11, #13, and #15. FIG. 48A-48G show increased expression of IFNγ, IL-2, and TNFα with CD4+CD8+ cells transduced with construct #10 (WT signal peptide, CD8β1) compared to other constructs. FACS analysis was gated on CD3+CD4+CD8+ cells against UACC257, 4:1 E:T. FIG. 49A-49G show increased expression of IFNγ, IL-2, MIP-1β, and TNFα with CD4−CD8+ cells transduced with construct #10 (WT signal peptide, CD8β1) compared to other constructs. FACS analysis was gated on CD3+CD4−CD8+ cells against UACC257, 4:1 E:T. FIG. 50A-50G show increased expression of IL-2 and TNFα with CD3+TCR+ cells transduced with construct #10 (WT signal peptide, CD8β1) compared to other constructs. MIP-1β expression is highest in Construct #11 (similar results when gated on CD4+CD8+ cells). FACS analysis was gated on CD3+TCR+ cells against UACC257, 4:1 E:T.

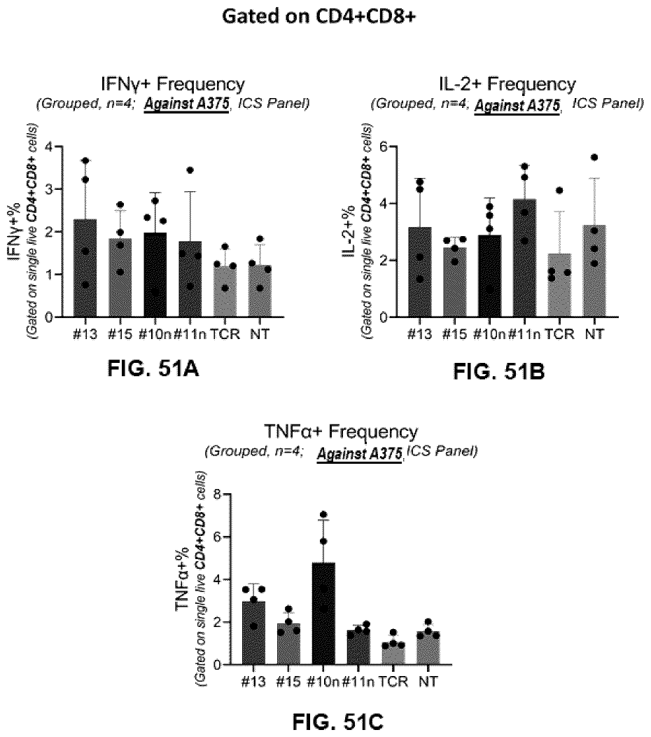

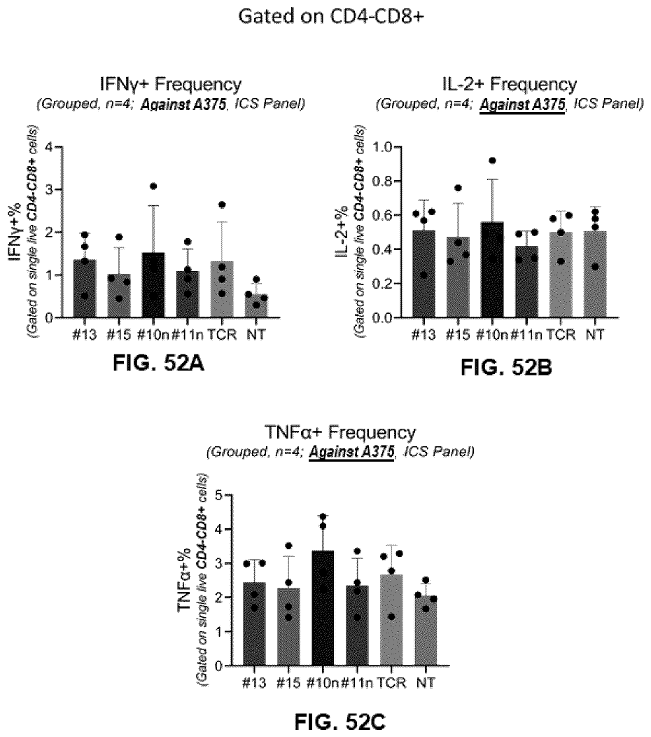

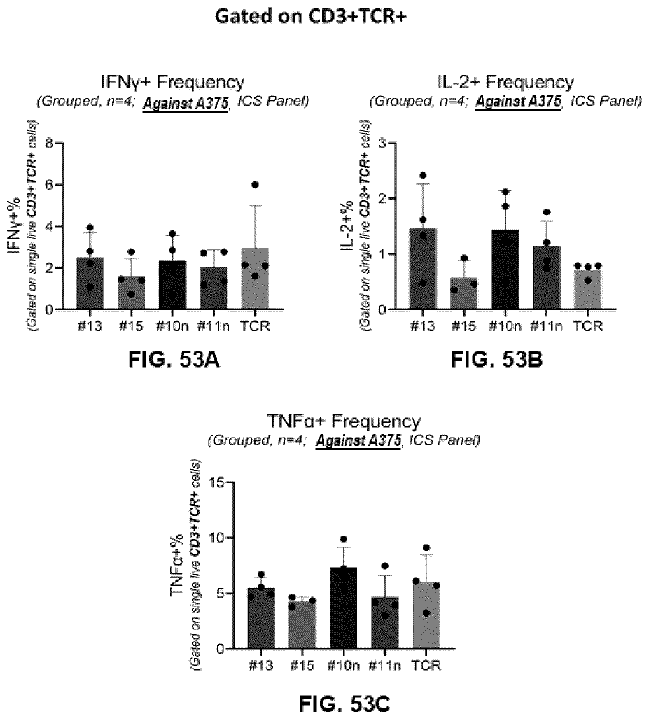

Expression of various cytokines was measured in A375 cells co-cultured at a 4:1 E:T ratio with PBMC transduced with Constructs #10, #11, #13, and #15. FIG. 51A-51C show results from FACS analysis gated on CD4+CD8+ cells against A375, 4:1 E:T. FIG. 52A-52C show results from FACS analysis gated on CD4−CD8+ cells against A375, 4:1 E:T. FIG. 53A-53C show results from FACS analysis gated on CD3+TCR+ cells against A375, 4:1 E:T. Overall, results were more variable when cells are co-cultured with A375+RFP, but similar trends are observed compared to activation by UACC257+RFP.

### Example 12

### Large-Scale Vector Screening (Constructs #10, #11, #13, #16, #18, #19)

### T Cell Manufacturing

### Activation/Transduction

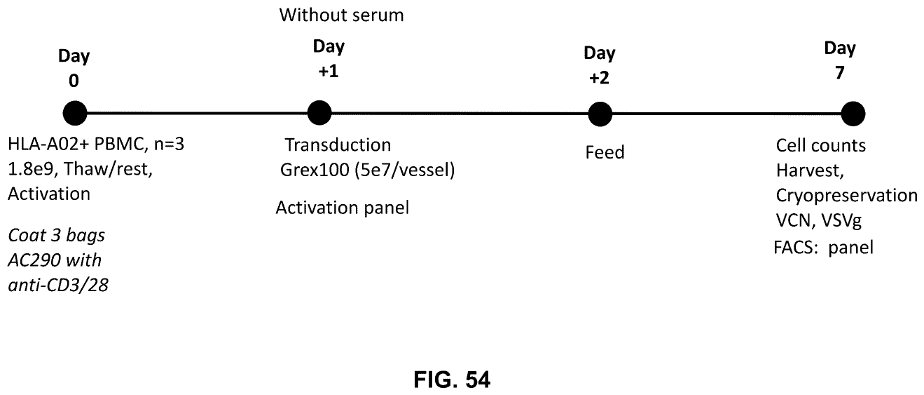

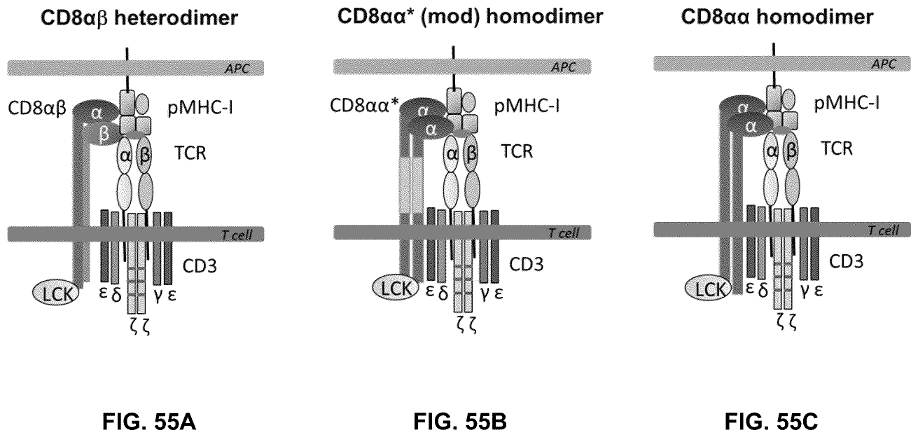

FIG. 54 shows that, on Day +0, PBMCs obtained from three HLA-A02+ donors were thawed and rested. Cells were activated in bags (AC290) coated with anti-CD3 and anti-CD28 antibodies in the absence of serum. On Day +1, activated PBMCs were transduced with viral vectors, e.g., Constructs #8, #10n, #11n, #13, #16, #18, and #19 in G-Rex® 100 cell culture vessels at about 5×10⁷ cells/vessel in the absence of serum. The amounts of virus used for transduction are shown in Table 10.

TABLE 10

| Constructs | Virus Volume/1 × 10⁶ cells |
|---|---|
| #13, #16, #18, #10n | 5 μl |
| #19 and #11n | 2.5 μl |
| #8 (TCR) | 2.5 μl |
| NT | — |

### Expansion

FIG. 54 shows that, on Day +2, transduced PBMCs were expanded in the absence of serum. On Day +7, cells were harvested for subsequent analysis, e.g., FACS-Tetramer and vector copy number (VCN) and were cryopreserved. Fold expansion on Day +7 was comparable for all constructs (approximately 30-fold expansion). Viabilities of cells is greater than 90% on Day +7.

### Characterization of T Cell Products

Cell counts, FACS-dextramers, and vector copy numbers (VCN) were determined. Tetramer panels may comprise live/dead cells, CD3, CD8α, CD8β, CD4, and peptide/MHC tetramers, e.g., PRAME-004 (SLLQHLIGL) (SEQ ID NO: 147)/MHC tetramers. FACS analysis was gated on live singlets, followed by CD3+, followed by CD4+CD8+, followed by CD4+CD8+Tetramer(Tet)+ and CD8+Tet+.

Tumor death assays and cytokine expression in the presence and absence of autologous immature dendritic cells was also measured.

The results were consistent with the prior examples and are summarized in Table 11.

TABLE 11

| | | | | | | TCR only |
|---|---|---|---|---|---|---|
| | | Construct | Construct | Construct | Construct | Construct |
| | Parameters | #10 | #13 | #11 | #19 | #8 |
| Manufacturing | Viabilities | >90% | >90% | >90% | >90% | >90% |
| | Fold Expansion d7 | 28.7 ± 11% | 28.6 ± 11% | 31.6 ± 13% | 29.6 ± 13% | 30.1 ± 11% |
| | Transgene expression | 46.9 ± 12% | 42 ± 9.8% | 41 ± 12% | 48.2 ± 14% | 22.8 ± 8% |
| | (% CD3+Tet+), | | | | | |
| | mean ± SD | | | | | |
| | Vector Copy Number | 3.3 ± 0.6% | 2.6 ± 0.7% | 2.0 ± 0.8% | 3.1 ± 1.8% | 1.7 ± 0.7% |
| Functionality | Multiple rounds of | +++ | +++ | +++ | +++ | +++ |
| | killing with UACC | | | | | |
| | Cytokine secretion | +++ | +++ | ++ | ++ | ++ |
| | (24 h, with UACC); | | | | | |
| | IFN-g, TNF-a, IL-2 | | | | | |
| | Cytokine secretion; | +++ | +++ | + | + | +/− |
| | CD4+CD8+TCR+ | | | | | |
| | (16 h, UACC); ICS | | | | | |
| | DC licensing assay | +++ | +++ | + | + | + |
| | (PBMC product) | | | | | |
| | IL-12, TNF-a & IL-6 | | | | | |
| | 3D Spheroid Assay | +++ | N/A | +++ | N/A | ++ |

### Example 13

### DC Licensing by CD4 Cells Expressing Constructs of the Present Disclosure

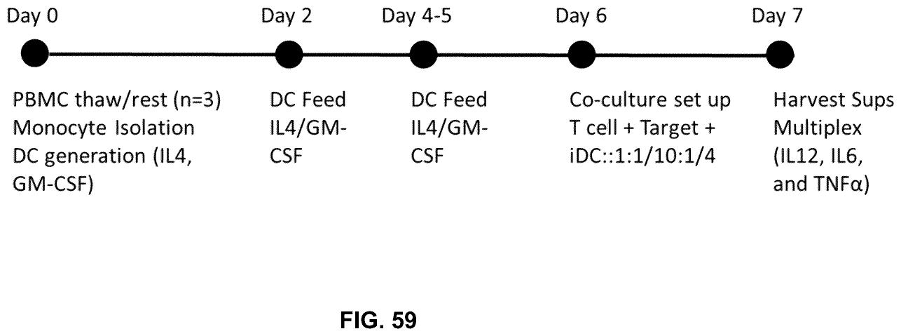

FIG. 59 shows a scheme of determining the levels of cytokine secretion by dendritic cells (DC) in the presence of PBMCs transduced with constructs of the present disclosure and in the presence of target cells, e.g., UACC257 cells. Briefly, Day 0, PBMCs (n=3) were thawed and rested, followed by monocyte isolation and autologous immature DCs (iDC) generation in the presence of IL-4 and GM-CSF; Day 2 and Day 4−5, DC were fed in the presence of IL-4 and GM-CSF; Day 6, iDC (+DC) were co-cultured with PBMC transduced with Construct #13, #16, #10n, #18, #11n, or #19 (Effector) and UACC257 cells (Target) at a ratio of Effector: Target: iDC=1: 1/10: 1/4 or without iDC (−DC), PBMCs transduced with TCR only, PBMCs without transduction (NT), PBMCs treated with iDC and LPS, and iDC only serve as controls; and Day 7 (after co-culturing for 24 hours), supernatants from the co-cultures were harvested, followed by cytokine profiling including, e.g., IL-12, IL-6, and TNF-α, using Multiplex.

Increased secretion of pro-inflammatory cytokines in tri-cocultures of autologous immature dendritic cells, UACC257 tumor cell line, and CD4+ T cell product expressing CD8αβ heterodimer and TCR (Construct #10) compared with that expressing CD8α* homodimer, in which the stalk region is replaced with CD8β stalk region, and TCR (Construct #11).

To determine the ability of CD4+ T cells expressing Constructs #10 or #11 to license DC, bulk PBMCs were transduced with Constructs #10 or #11, followed by selection of CD8+ and CD4+ cells from the product. Tri-cocultures of PBMCs, CD8+CD4− selected-product, or CD4+CD8+ selected-product with UACC257 tumor cell line in the presence or absence of autologous immature dendritic cells (iDCs) for 24 h followed by cytokine quantification of IL-12, TNF-α and IL-6 using Multiplex; iDCs alone or with LPS as controls, N=4−7, mean±SD, P values based on 2way ANOVA.

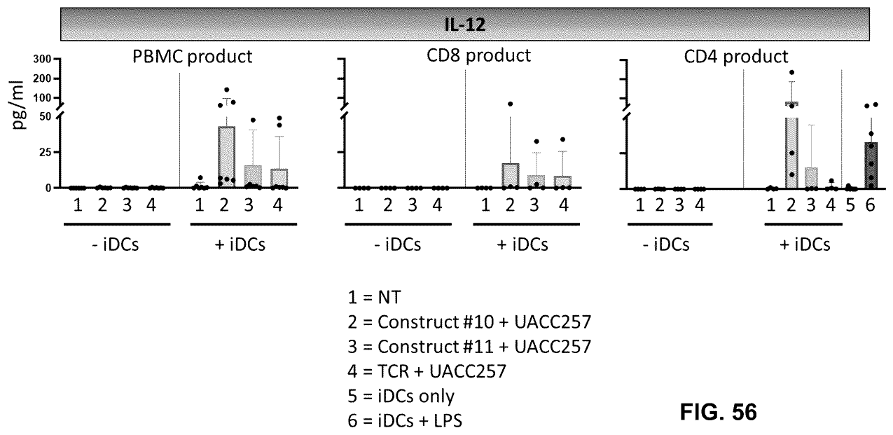

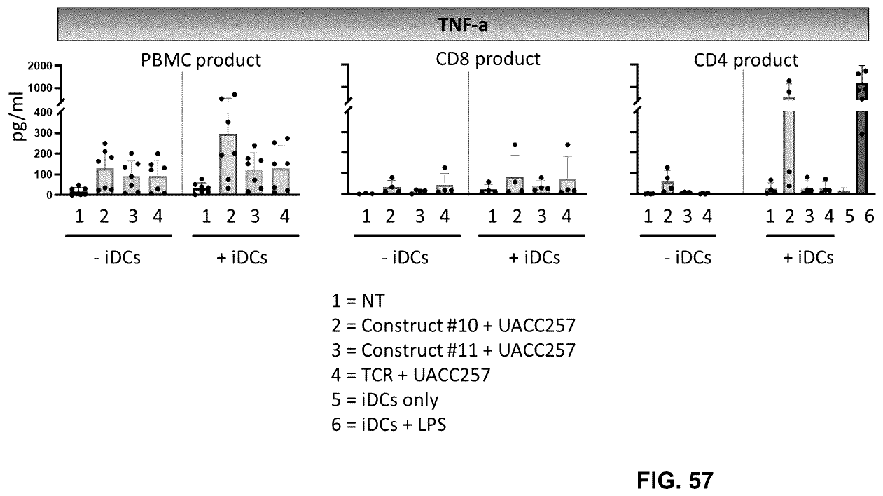

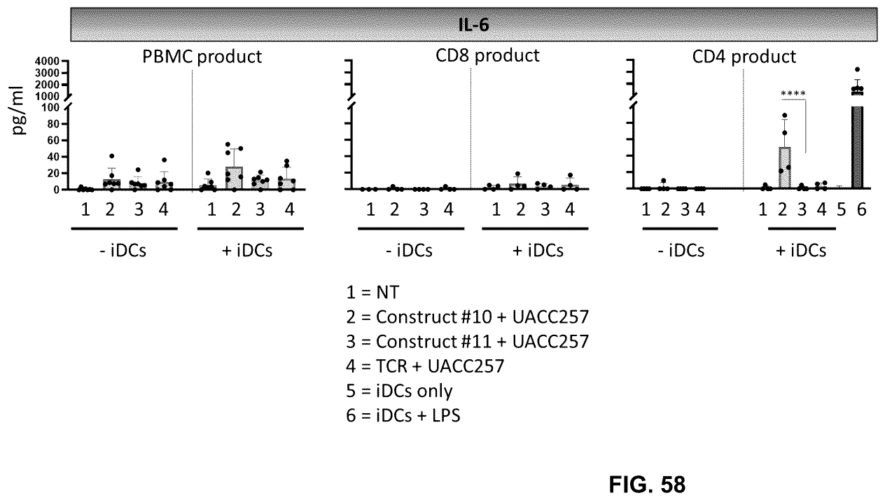

In the presence of immature dendritic cells (iDCs) and UACC257 cells, CD4+ T cells expressing Construct #10 (CD4+CD8+ T cells) performed better by inducing higher levels of IL-12 (FIG. 56), TNF-α (FIG. 57), and IL-6 (FIG. 58) secreted by dendritic cells (DC) than CD4+ T cells expressing Construct #11. On the other hand, the levels of IL-12, TNF-α, and IL-6 were comparable between CD8+ T cells expressing Constructs #10 and #11 (CD8+CD4− T cells). These results suggest that CD4+ T cells expressing CD8αβ heterodimer and TCR (Construct #10) may be a better product than CD4+ T cells expressing CD8α* homodimer and TCR (Construct #11) in DC licensing. The negative controls include the cytokine levels obtained (1) in the absence of iDCs (-iDCs), (2) in the presence of non-transduced T cells (NT)+UACC257 cells, and (3) in the presence of T cells transduced with TCR only (TCR)+UACC257 cells. The positive control includes the cytokine levels obtained from iDCs treated with lipopolysaccharide (LPS), which can activate DC.

### Example 14

### Assessment of DC Maturation and Cytokine Secretion by PBMC Products in Response to UACC257 Targets

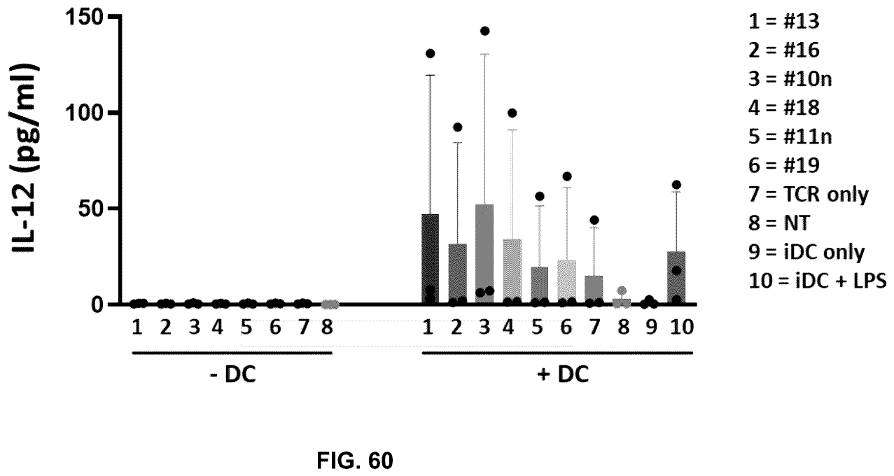

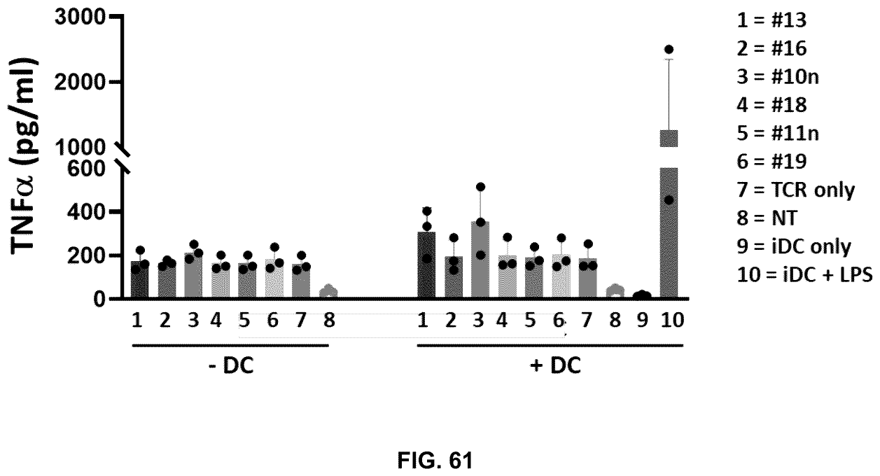

FIG. 60 shows IL-12 secretion levels induced by co-culturing PBMCs transduced with constructs of the present disclosure in the presence or absence of iDC and target cells, e.g., UACC257 cells. For example, IL-12 secretion was increased by co-culturing PBMCs transduced with Constructs #10 and 13 in the presence of iDC (+DC) and UACC257, as compared with that by co-culturing PBMCs transduced with TCR only. Increase of IL-12 secretion suggests (1) polarization towards Th1 cell-mediated immunity including TNF-α production (see, FIG. 61), (2) T cell proliferation, (3) IFN-γ production, and (4) cytolytic activity of cytotoxic T lymphocytes (CTLs).

FIG. 61 shows TNF-α secretion levels induced by co-culturing PBMCs transduced with constructs of the present disclosure in the presence or absence of iDC and target cells, e.g., UACC257 cells. For example, TNF-α secretion was increased by co-culturing PBMCs transduced with Constructs #10 and 13 in the presence of iDC (+DC) and UACC257, as compared with that by co-culturing PBMCs transduced with TCR only.

The increased IL-6 secretion (in addition to IL-12, TNF-α) may signify dendritic cell maturation, which may be augmented by CD40−CD40L interactions between CD4+ T cells and DCs. DC maturation and subsequent cytokine secretion may aid in modulation of the proinflammatory environment.

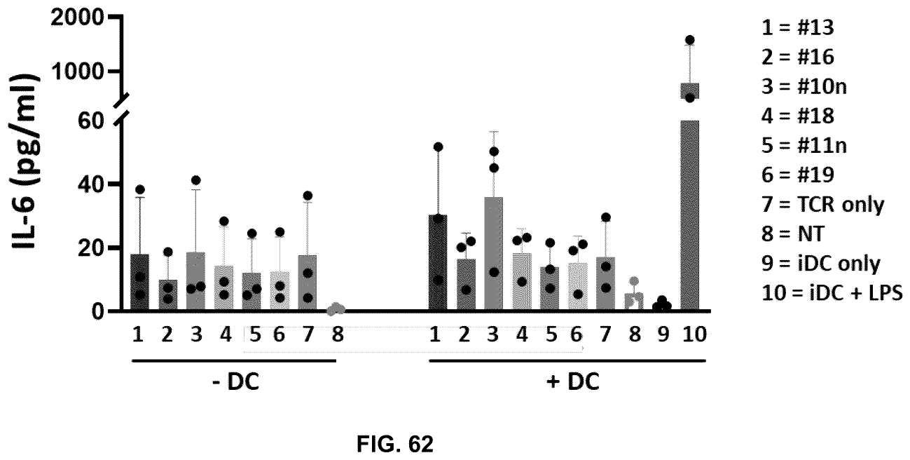

FIG. 62 shows IL-6 secretion levels induced by co-culturing PBMCs transduced with constructs of the present disclosure in the presence or absence of iDC and target cells, e.g., UACC257 cells. For example, IL-6 secretion was increased by co-culturing PBMCs transduced with Constructs #10 and 13 in the presence of iDC (+DC) and UACC257, as compared with that by co-culturing PBMCs transduced with TCR only.

These results show that PBMC products containing CD4+ T cells co-expressing transgenic TCR and CD8 co-receptor (CD8αβ heterodimer or CD8α homodimer) may license DCs in the microenvironment through antigen cross presentation to modulate the tumor microenvironment by, e.g., increasing IL-12, IL-6, and TNF-α secretion.

Table 12 shows comparison between constructs based on manufacturability and functionality.

TABLE 12

| | | Construct | Construct | Construct | Construct | TCR |
|---|---|---|---|---|---|---|
| | Parameters | #10 | #13 | #11 | #19 | only |
| Manufacturability | Viabilities | >90% | >90% | >90% | >90% | >90% |
| | Fold expansion | 28.7 ± 11% | 28.6 ± 11% | 31.6 ± 13% | 29.6 ± 13% | 30.1 ± 11% |
| | on Day 7 | | | | | |
| | Transgene | 46.9 ± 12% | 42 ± 9.8% | 41 ± 12% | 48.2 ± 14% | 22.8 ± 8% |
| | expression | | | | | |
| | (% CD3+Tet+) | | | | | |
| | mean ± SD | | | | | |
| | Vector copy | 3.3 ± 0.6% | 2.6 ± 0.7% | 2.0 ± 0.8% | 3.1 ± 1.8% | 1.7 ± 0.7% |
| | number | | | | | |
| Functionality | Multiple rounds | +++ | +++ | +++ | +++ | +++ |
| | of killing with | | | | | |
| | UACC257 cells | | | | | |
| | Cytokine | +++ | +++ | ++ | ++ | ++ |
| | secretion (24 h, | | | | | |
| | with UACC257 | | | | | |
| | cells); IFN-γ, | | | | | |
| | TNF-α, IL-2 | | | | | |
| | Cytokine | +++ | +++ | + | + | +/− |
| | secretion; | | | | | |
| | CD4+CD8+TCR+ | | | | | |
| | (16 h with | | | | | |
| | UACC257 cells); | | | | | |
| | ICS | | | | | |
| | DC licensing | +++ | +++ | + | + | + |
| | assay (PBMC | | | | | |
| | product) | | | | | |
| | IL-12, TNF-α, | | | | | |
| | and IL-6 | | | | | |
| | 3D spheroid assay | +++ | N/A | +++ | N/A | ++ |

Notes:
“+++” = best response; “++” = good response; “+” = average response; “+/−” = poor response.

Table 13 shows construct comparison and ranking (the smaller the number the better).

TABLE 13

| | Construct | Construct | Construct | Construct |
|---|---|---|---|---|
| Parameters | #10 | #13 | #11 | #19 |
| Manufacturability | 1 | 1 | 1 | 1 |
| Functionality | 1 | 1 | 2 | 2 |
| PBMC | | | | |
| Functionality | 1 | 1 | 1 | 1 |
| CD8 | | | | |
| Functionality | 1 | 1 | 3 | 3 |
| CD4 | | | | |
| Time delay* | 1 | 1 | 1 | 1 |
| Total | 5 | 5 | 8 | 8 |

*Time delay here refers to any delay from, for example, GMP Vector manufacturing or any delay due to incomplete data set, which may add delay in implementation of constructs in clinical trials.

In sum, while manufacturability in terms of, e.g., viability, fold expansion, transgene expression, and vector copy number, may be equally good, as ranked 1, among cells transduced with Construct #10, #11, #13, or #19, functionality in terms of, e.g., cell killing, cytokine secretion, DC licensing, and 3D spheroid forming ability, of cells transduced with Construct #10 and #13 may be better, as ranked 1, than those transduced with Construct #11 and #19, as ranked 1-3.

### Example 15

### EC50 Assays

To determine the efficacy of T cells transduced with constructs of the present disclosure, e.g., Constructs #10 and #11, against target cells, EC50s were determined based on the levels of IFNγ produced by the transduced cells in the presence of PRAME peptide-pulsed T2 cells.

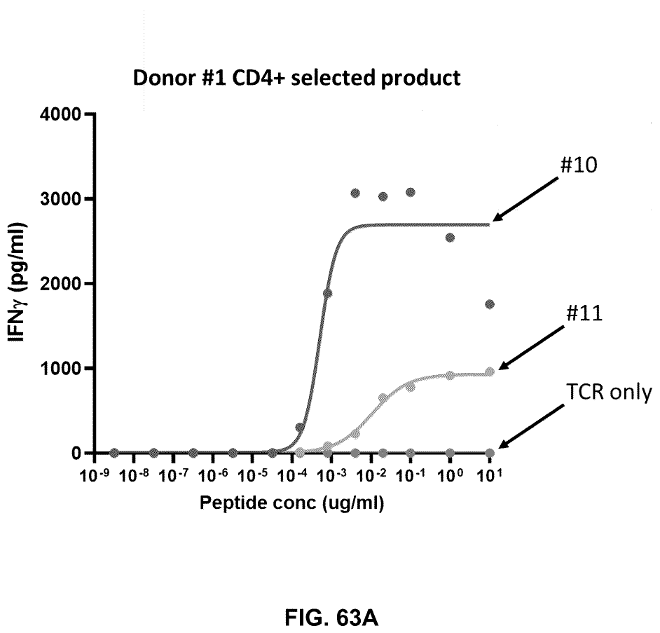

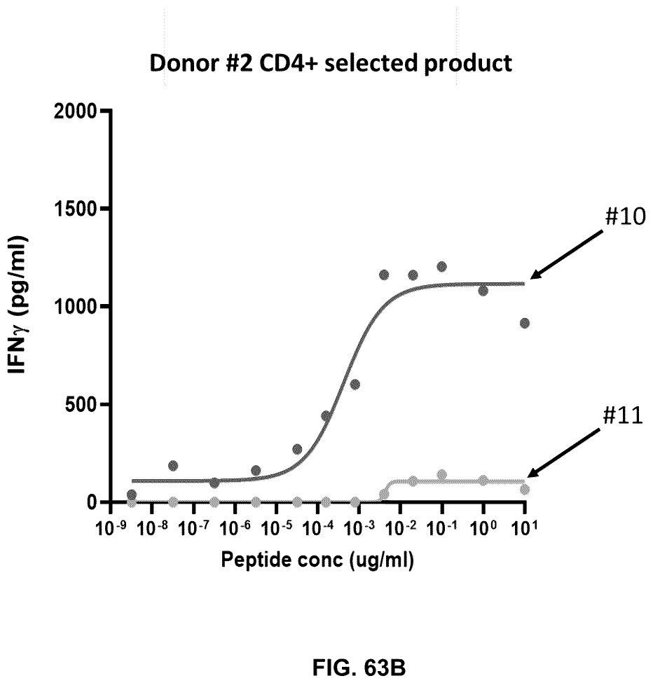

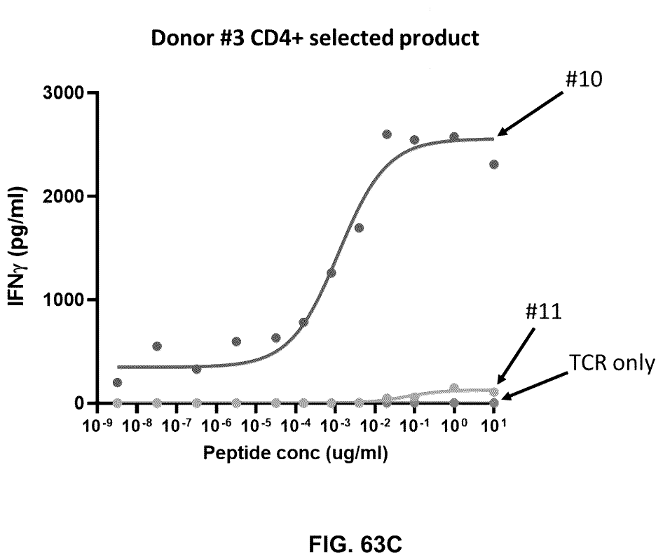

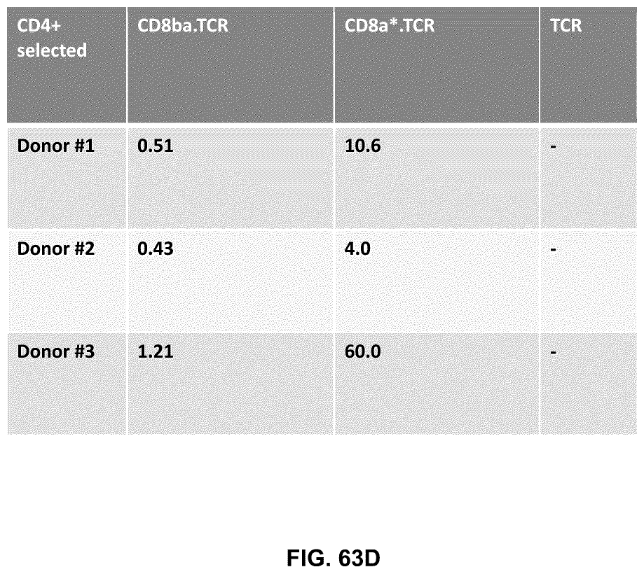

For example, to compare EC50s of CD4+ selected T cells transduced with Construct #10 (CD8αβ-TCR), Construct #11 (m1CD8α-TCR), or Construct #8 (TCR only), CD4+ selected products (TCR+ normalized) were co-cultured with PRAME peptide-pulsed T2 cells at defined concentrations at E:T ratio of 1:1 for 24 h. IFNγ levels were quantified in the supernatants after 24 h. FIGS. 63A-63C show IFNγ levels produced by the transduced CD4+ selected T cells obtained from Donor #1, #2, and #3, respectively. In general, CD4+ selected T cells transduced with Construct #10 were more sensitive to PRAME antigen as compared with that transduced with Construct #11 (m1CD8α TCR+CD4 T cells), as indicated by lower EC50 values (ng/ml) of CD4+ selected T cells transduced with Construct #10 than that transduced with Construct #11 (FIG. 63D). No response was observed among TCR+CD4+ cells (FIGS. 63A-63D). These results suggest that CD8α(3 heterodimer may impart increased avidity to CD8αβTCR+CD4+ T cells as compared to m1CD8α homodimer, leading to better efficacy against target cells.

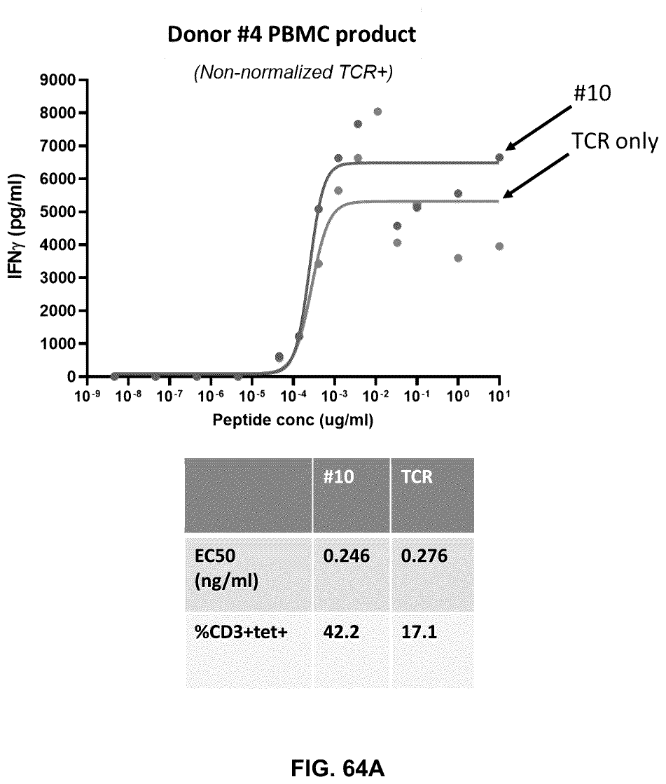

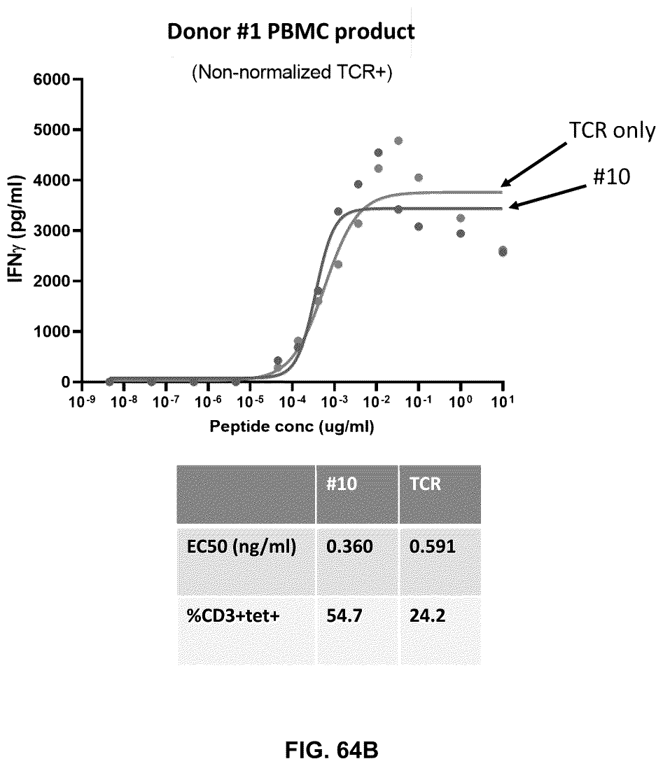

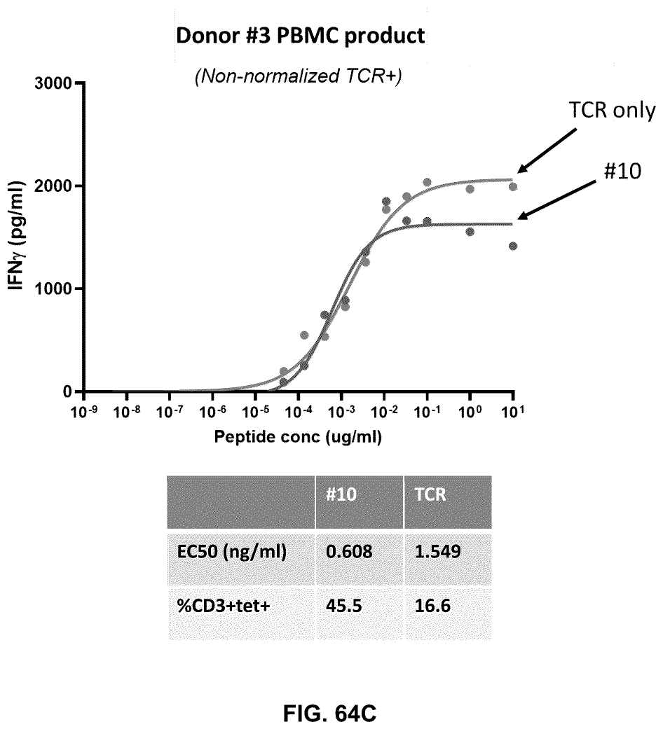

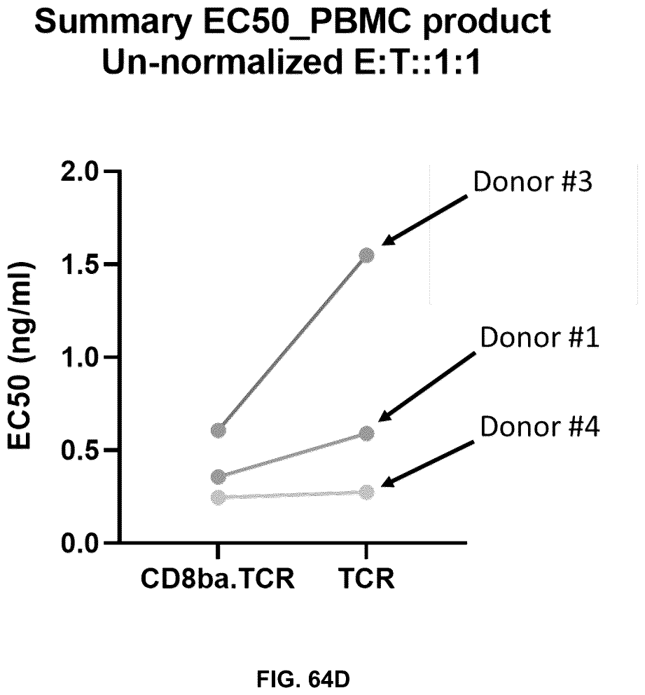

Similar experiments were performed using PBMC obtained from Donor #1, #3, and #4. Briefly, PBMC products (TCR+ non-normalized) were co-cultured with PRAME peptide-pulsed T2 cells at defined concentrations at E:T ratio of 1:1 for 24 h. IFNγ levels were quantified in the supernatants after 24 h. FIGS. 64A-64C show IFNγ levels produced by the transduced PBMC obtained from Donor #4, #1, and #3, respectively. Donor-to-donor variability was observed in the EC50 values. For example, while Donor #3 (FIGS. 64C and 64D) shows lower EC50 of PBMC transduced with Construct #10 as compared with that transduced with TCR only, Donors #1 (FIG. 64B) and #4 (FIG. 64A) show comparable EC50s between Construct #10 and TCR only (FIG. 64D). Thus, the increased avidity and efficacy observed in CD4+ selected T cell products expressing TCR and CD8αβ heterodimer as compared with that expressing TCR only may be obtained but to lesser extent when using PBMC products.

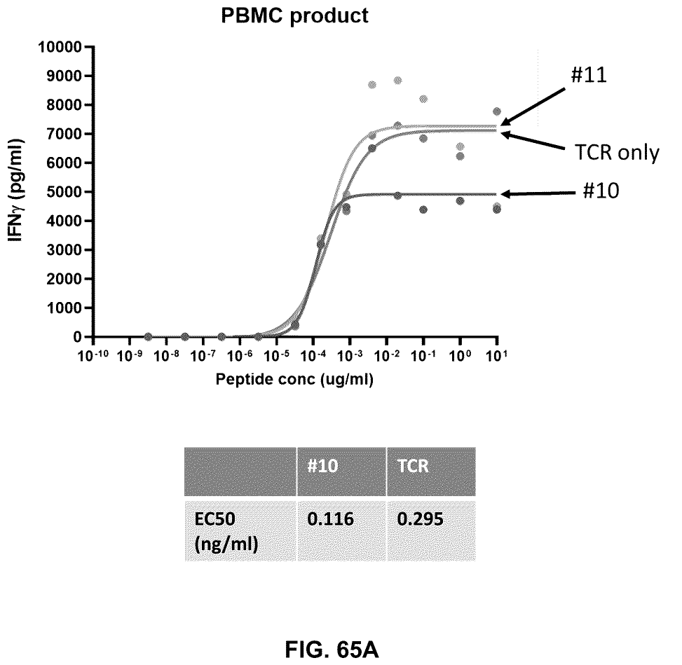

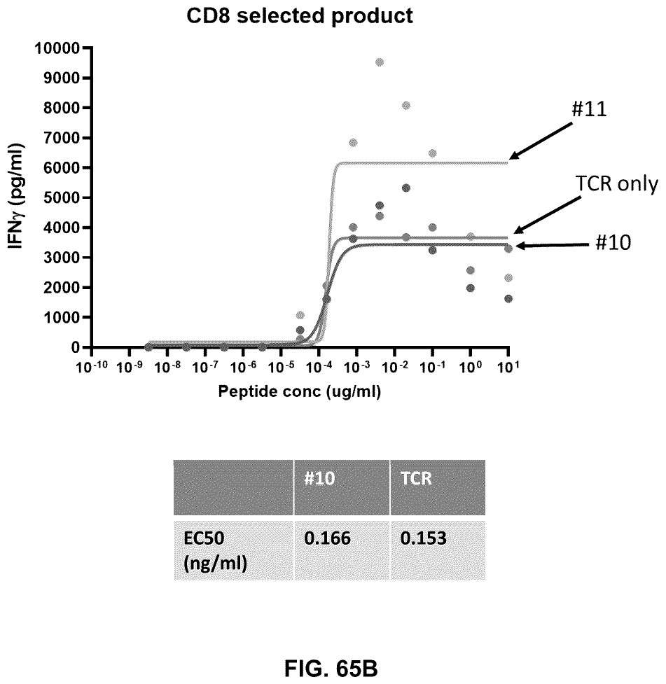

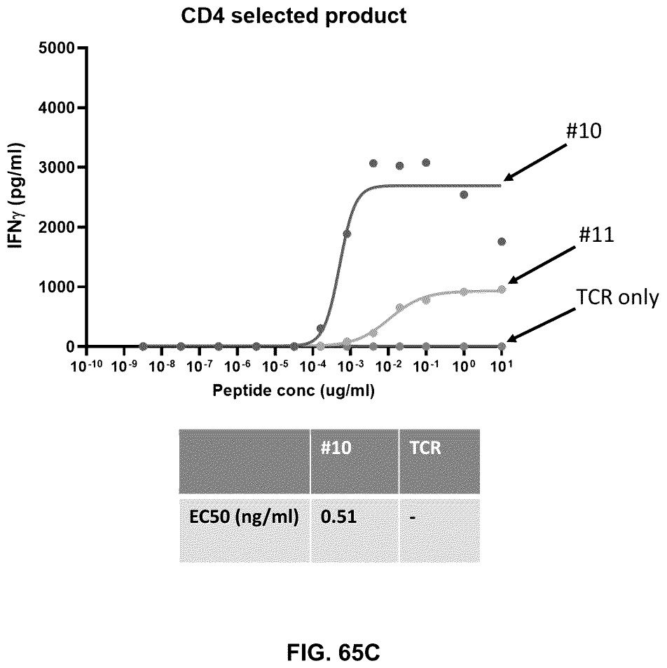

To compare EC50s of different T cell products obtained from the same donor, PBMC products, CD8+ selected products, and CD4+ selected products obtained from a single donor were co-cultured with PRAME peptide-pulsed T2 cells (TCR+ normalized) at defined concentrations at E:T ratio of 1:1 for 24 h. IFNγ levels were quantified in the supernatants after 24 h. FIGS. 65A-65C show that IFNγ levels produced by PBMC products (FIG. 65A), CD8+ selected products (FIG. 65B), and CD4+ selected products (FIG. 65C), respectively. Consistently, EC50 of CD4+ selected T cells transduced with Construct #10 was lower than that transduced with Construct #11 or TCR only (FIG. 65C), while EC50s of the transduced PBMC and CD8+ selected T cells were comparable between Construct #10 and TCR only transduction. Thus, the increased avidity and efficacy observed in CD4+ selected T cell products expressing TCR and CD8αβ heterodimer as compared with that expressing TCR and m1CD8α homodimer or with that expressing TCR only may be obtained but to lesser extent when using PBMC products or CD8+ selected T cell products.

All references cited in this specification are herein incorporated by reference as though each reference was specifically and individually indicated to be incorporated by reference. The citation of any reference is for its disclosure prior to the filing date and should not be construed as an admission that the present disclosure is not entitled to antedate such reference by virtue of prior invention.

It will be understood that each of the elements described above, or two or more together may also find a useful application in other types of methods differing from the type described above. Without further analysis, the foregoing will so fully reveal the gist of the present disclosure that others can, by applying current knowledge, readily adapt it for various applications without omitting features that, from the standpoint of prior art, fairly constitute essential characteristics of the generic or specific embodiments of this disclosure set forth in the appended claims. The foregoing embodiments are presented by way of example only; the scope of the present disclosure is to be limited only by the following claims.

What is claimed is:

1. A nucleic acid comprising a nucleic acid sequence encoding:
(i) a T-cell receptor (TCR) comprising a TCR α chain and a TCR β chain; and
(ii) a CD8 polypeptide comprising an amino acid sequence which is at least 95% identical to SEQ ID NO: 5, 258, 259, 262, or a variant thereof.

2. The nucleic acid of claim 1, wherein the TCR α chain and the TCR β chain are selected from SEQ ID NO: and 16, 17 and 18, 19 and 20, 21 and 22, 23 and 24, 25 and 26, 27 and 28, 29 and 30, 31 and 32, 33 and 34, 35 and 36, 37 and 38, 39 and 40, 41 and 42, 43 and 44, 45 and 46, 47 and 48, 49 and 50, 51 and 52, 53 and 54, 55 and 56, 57 and 58, 59 and 60, 61 and 62, 63 and 64, 65 and 66, 67 and 68, 69 and 70, 71 and 303, 304 and 74, 75 and 76, 77 and 78, 79 and 80, 81 and 82, 83 and 84, 85 and 86, 87 and 88, 89 and 90, and 91 and 92.

3. The nucleic acid of claim 1, wherein the nucleic acid sequence is at least 80%, 90%, 91%, 92%, 93%, 94%, 95%, 96%, 97%, 98%, 99%, or 100% identical to the nucleic acid sequence of SEQ ID NO: 267, 269, 271, 273, 275, 277, 279, 281, 283, 285, 287, 289, 291, 295, 297, 299, or 301.

4. A polypeptide encoded by the nucleic acid of claim 1.

5. A polypeptide comprising an amino acid sequence which is at least 80%, 90%, 91%, 92%, 93%, 94%, 95%, 96%, 97%, 98%, 99%, or 100% identical to the amino acid sequence of SEQ ID NO: 5, 258, 259, 262, or a variant thereof.

6. A vector comprising the nucleic acid of claim 1.

7. The vector of claim 6, wherein the vector further comprises a nucleic acid encoding a 2A peptide or an internal ribosome entry site (IRES) positioned between:
(i) the nucleic acid encoding the TCR α chain and the nucleic acid encoding the TCR β chain;
(ii) the nucleic acid encoding the TCR α chain and the nucleic acid encoding the CD8 polypeptide; and/or
(iii) the nucleic acid encoding the TCR β chain and the nucleic acid encoding the CD8 polypeptide.

8. The vector of claim 7, wherein the 2A peptide is P2A (SEQ ID NO: 93), T2A (SEQ ID NO: 94), E2A (SEQ ID NO: 95), or F2A (SEQ ID NO: 96).

9. The vector of claim 6, wherein the vector further comprises a posttranscriptional regulatory element (PRE) sequence selected from a Woodchuck PRE (WPRE), Woodchuck PRE (WPRE) mutant 1, Woodchuck PRE (WPRE) mutant 2, and hepatitis B virus (HBV) PRE (HPRE).

10. The vector of claim 9, wherein the post-transcriptional regulatory element (PRE) sequence is (i) Woodchuck PRE (WPRE) mutant 1 comprising the amino acid sequence of SEQ ID NO: 256; or (ii) Woodchuck PRE (WPRE) mutant 2 comprising the amino acid sequence of SEQ ID NO: 257.

11. The vector of claim 6, wherein the vector further comprises a promoter selected from cytomegalovirus (CMV) promoter, phosphoglycerate kinase (PGK) promoter, myelin basic protein (MBP) promoter, glial fibrillary acidic protein (GFAP) promoter, modified MoMuLV LTR comprising myeloproliferative sarcoma virus enhancer (MNDU3), Ubiquitin C promoter, EF-1 alpha promoter, and Murine Stem Cell Virus (MSCV) promoter.

12. The vector of claim 6, wherein the vector is a viral vector or a non-viral vector.

13. The vector of claim 12, wherein the viral vector is selected from adenoviruses, poxviruses, alphaviruses, arenaviruses, flaviviruses, rhabdoviruses, retroviruses, lentiviruses, herpesviruses, paramyxoviruses, picornaviruses, and combinations thereof.

14. The vector of claim 12, wherein the viral vector is pseudotyped with an envelope protein of a virus selected from the native feline endogenous virus (RD114), a version of RD114 (RD114TR), gibbon ape leukemia virus (GaLV), a version of GaLV (GaLV-TR), amphotropic murine leukemia virus (MLV 4070A), baculovirus (GP64), vesicular stomatitis virus (VSV-G), fowl plague virus (FPV), Ebola virus (EboV), or baboon retroviral envelope glycoprotein (BaEV), and lymphocytic choriomeningitis virus (LCMV).

15. The vector of claim 6, wherein the vector is a lentiviral vector.

16. The vector of claim 6, wherein the vector further comprises a nucleic acid encoding a chimeric antigen receptor (CAR).

17. AT cell transduced with the nucleic acid of claim 1.

18. The T cell of claim 17, wherein the T cell is an αβ T cell, γδ T cell, and/or natural killer T cell.

19. The T cell of claim 18, wherein the αβ T cell is a CD4+ T cell or a CD8+ T cell.

20. The T cell of claim 18, wherein the γδ T cell is a Vγ9Vδ2+ T cell.

21. A T cell expressing the polypeptide of claim 4.

22. The T cell of claim 21, wherein said T cell is an αβ T cell, a γδ T cell, and/or a natural killer T cell.

23. A composition comprising the T cell of claim 21.

24. The composition of claim 23, wherein the composition is a pharmaceutical composition.

25. The composition of claim 23, wherein the composition further comprises an adjuvant, excipient, carrier, diluent, buffer, stabilizer, or a combination thereof.

26. A method of preparing T cells for immunotherapy comprising isolating T cells from a blood sample of a human subject, activating the isolated T cells, transducing the activated T cells with the nucleic acid of claim 1, and expanding the transduced T cells.

27. A method of treating a patient who has cancer, comprising administering to the patient the composition of claim 23, wherein the cancer is selected from the group consisting of non-small cell lung cancer, small cell lung cancer, melanoma, liver cancer, breast cancer, uterine cancer, Merkel cell carcinoma, pancreatic cancer, gallbladder cancer, bile duct cancer, colorectal cancer, urinary bladder cancer, kidney cancer, leukemia, ovarian cancer, esophageal cancer, brain cancer, gastric cancer, and prostate cancer.

28. A method of eliciting an immune response in a patient who has cancer, comprising administering to the patient the composition of claim 23, wherein the cancer is selected from the group consisting of non-small cell lung cancer, small cell lung cancer, melanoma, liver cancer, breast cancer, uterine cancer, Merkel cell carcinoma, pancreatic cancer, gallbladder cancer, bile duct cancer, colorectal cancer, urinary bladder cancer, kidney cancer, leukemia, ovarian cancer, esophageal cancer, brain cancer, gastric cancer, and prostate cancer.
